(19) United States
(12) Patent Application Publication
(10) Pub. No.: US 2026/0060002 A1
(43) Pub. Date: Feb. 26, 2026

(54) ORGANIC ELECTROLUMINESCENT COMPOUND, A PLURALITY OF HOST MATERIALS AND ORGANIC ELECTROLUMINESCENT DEVICE COMPRISING THE SAME

(71) Applicant: DuPont Specialty Materials Korea Ltd., Chungcheongnam-do (KR)

(72) Inventors: Hee-Ryong KANG, Gyeonggi-do (KR); Sang-Hee CHO, Gyeonggi-do (KR); So-Young JUNG, Gyeonggi-do (KR); Jin-Ri HONG, Gyeonggi-do (KR); So-Mi PARK, Gyeonggi-do (KR)

(21) Appl. No.: 19/374,472

(22) Filed: Oct. 30, 2025

Related U.S. Application Data
(62) Division of application No. 17/488,130, filed on Sep. 28, 2021, now Pat. No. 12,520,718.

(30) Foreign Application Priority Data
Oct. 19, 2020 (KR) 10-2020-0135064
Aug. 13, 2021 (KR) 10-2021-0107451

(51) Int. Cl. H10K 85/60; H10K 50/11; H10K 101/00

(52) U.S. Cl. CPC H10K 85/636; H10K 85/622; H10K 85/623; H10K 85/624; H10K 85/626; H10K 85/654; H10K 85/6572; H10K 85/6574; H10K 85/6576; H10K 50/11; H10K 2101/90

(57) ABSTRACT

The present disclosure relates to an organic electroluminescent compound represented by formula 2′, a plurality of host materials comprising at least one first host compound and at least one second host compound, and an organic electroluminescent device comprising the same. By comprising the organic electroluminescent compound or a specific combination of compounds of the present disclosure, it is possible to provide an organic electroluminescent device having improved driving voltage, luminous efficiency, power efficiency, and/or lifespan characteristics.

### CLAIM OF BENEFIT OF PRIOR APPLICATION

This application is a division of U.S. patent application Ser. No. 17/488,130 filed Sep. 28, 2021, which is the Convention Filing of KR10-2020-0135064 filed Oct. 19, 2020 and KR10-2021-0107451 filed Aug. 13, 2021, all of which are incorporated by reference herein in their entirety.

### TECHNICAL FIELD

The present disclosure relates to an organic electroluminescent compound, a plurality of host materials and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. An OLED having high luminous efficiency and/or long lifespan is required for long time use and high resolution of a display.

In order to enhance luminous efficiency, driving voltage and/or lifespan, various materials or concepts for an organic layer of an organic electroluminescent device have been proposed. However, they were not satisfactory in practical use. Therefore, it is required to develop OLEDs having more improved performance, such as improved driving voltage, luminous efficiency, power efficiency, and/or lifespan characteristics, compared to the OLEDs previously disclosed.

Korean Patent Application Laying-Open No. 2018-0038834 and Korean Patent Publication No. 2079239 disclose compounds comprising phenanthrene and azine, but fail to specifically disclose organic electroluminescent compounds or a specific combination of host materials claimed herein.

### DISCLOSURE OF INVENTION

### Technical Problem

The objective of the present disclosure is to provide a compound having a new structure suitable for using it as an organic electroluminescent material. Another objective of the present disclosure is to provide an organic electroluminescent device having a low driving voltage, high luminous efficiency, high power efficiency, and/or excellent lifespan characteristics by comprising a plurality of host materials including a specific combination of compounds.

### Solution to Problem

As a result of intensive research to solve the above technical problems, the present inventors found that the above objective can be achieved by an electroluminescent compound represented by the following formula 2′. In addition, the present inventors found that the above objective can be achieved by a plurality of host materials comprising at least one first host compound and at least one second host compound, wherein the first host compound is represented by the following formula 1, and the second host compound is represented by the following formula 2.

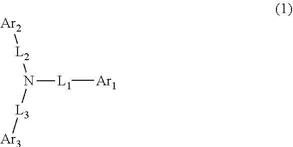

In formula 1,
L₁ to L₃ each independently represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₁ to Ar₃ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30)aliphatic ring(s) and a (C6-C30) aromatic ring(s), or -Lₐ-N(Arₐ)(Ar_{b});
Lₐ each independently represents a single bond, a substituted or unsubstituted (C6-C30)arylene, or a substituted or unsubstituted (3- to 30-membered) heteroarylene; and
Arₐ and Ar_{b} each independently represent hydrogen, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; and
with the proviso that the case where all of L₁ to L₃ represent single bonds, and all of Ar₁ to Ar₃ represent hydrogen is excluded;

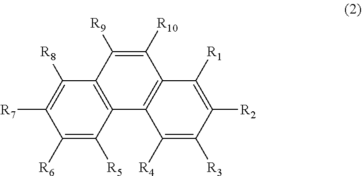

In formula 2,
R₁ to R₁₀ are each independently represented by

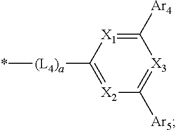

or represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L_{b}-N(Ar_{c})(Ar_{d}); or may be linked to an adjacent substituent(s) to form a ring(s), with a proviso that at least one of R₁ to R₁₀ is represented by

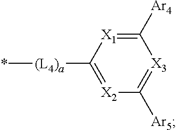

L_{b} each independently represents a single bond, a substituted or unsubstituted (C6-C30)arylene, or a substituted or unsubstituted (3- to 30-membered)heteroarylene;
Ar_{c} and Ar_{d} each independently represent hydrogen, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl;
L₄ represents a single bond, a substituted or unsubstituted (C6-C30)cycloalkylene, a substituted or unsubstituted (C6-C30)arylene, or a substituted or unsubstituted (3- to 30-membered)heteroarylene;
X₁ to X₃ each independently represent N or CH, with a proviso that at least one of X₁ to X₃ is N;
Ar₄ and Ar₅ each independently represent a substituted or unsubstituted (C6-C30)cycloalkyl, a substituted or unsubstituted (C6-C30) aryl, a substituted or unsubstituted (3- to 30-membered) heteroaryl, or a substituted or unsubstituted fused ring group of a (C3-C30) aliphatic ring(s) and a (C6-C30) aromatic ring(s); and
a represents an integer of 1 to 3, where if a is an integer of 2 or more, each of L₄ may be the same or different.

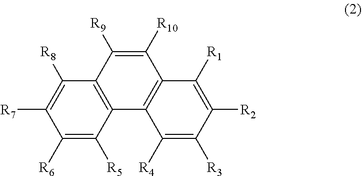

In formula 2′,
R₁ to R₁₀ are each independently represented by

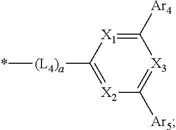

or represent hydrogen, deuterium, a halogen, a cyano, a substituted or unsubstituted (C1-C30) alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s), with a proviso that at least one of R₁ to R₁₀ is represented by

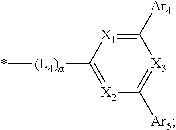

L₄ represents a single bond, a substituted or unsubstituted (C6-C30)cycloalkylene, or a substituted or unsubstituted (C10)arylene;
all of X₁ to X₃ represent N;
Ar₄ and Ar₅ each independently represent a substituted or unsubstituted (C6-C30)cycloalkyl, a substituted or unsubstituted (C6-C30)aryl, a substituted or unsubstituted (3- to 30-membered)heteroaryl, or a substituted or unsubstituted fused ring group of a (C3-C30) aliphatic ring(s) and a (C6-C30) aromatic ring(s), and Ar₄ and Ar₅ are the same as or different from each other;
with a proviso that when each of Ar₄ and Ar₅ represents an unsubstituted phenyl, an unsubstituted naphthyl, an unsubstituted biphenyl, an unsubstituted terphenyl, an unsubstituted phenanthrenyl, an unsubstituted triphenylenyl, an unsubstituted 1-dibenzofuranyl, an unsubstituted 1-dibenzothiophenyl, a phenyl substituted with a 1-dibenzofuranyl(s), a phenyl substituted with a 1-dibenzothiophenyl(s), a phenyl substituted with a 9-carbazolyl(s), an unsubstituted carbazolyl, a substituted or unsubstituted dibenzodioxynyl, a dibenzothiophenyl fused to benzofuran, or a dibenzothiophenyl fused to benzothiophene, and L₄ represents a single bond, L₄ is not linked at the position of R₉ or R₁₀;
with a proviso that when L₄ represents a single bond and is linked at the position of R₂ or R₇, Ar₄ and Ar₅ each independently represent a phenyl unsubstituted or substituted with a (C6-C30)cycloalkyl(s); a naphthyl unsubstituted or substituted with a dibenzofuranyl(s), a dibenzothiophenyl(s) or a phenanthrenyl(s); an unsubstituted biphenyl; an unsubstituted terphenyl (except for the structure

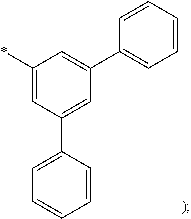 [SMILES: *c1cc(-c2ccccc2)cc(-c2ccccc2)c1]

an unsubstituted phenanthrenyl; an unsubstituted triphenylenyl; an unsubstituted chrysenyl; an unsubstituted (C22)aryl; an unsubstituted 9,9-diphenylfluorenyl; an unsubstituted 7,7-dimethylbenzofluorenyl; an unsubstituted 9,9-diphenylsilafluorenyl; an fluorenyl substituted with a phenyl(s) and a (C6-C30)cycloalkyl(s); a fused ring group of a (C3-C10) aliphatic ring(s) and a (C6-C12) aromatic ring(s) substituted with a methyl(s); an unsubstituted phenylnaphthyl; an unsubstituted naphthylphenyl; an unsubstituted dibenzofuranyl; or an unsubstituted dibenzothiophenyl; and
a represents an integer of 1 to 3, where if a is an integer of 2 or more, each of L₄ may be the same or different.

### Advantageous Effects of Invention

The organic electroluminescent compound according to the present disclosure exhibits performance suitable for using it in an organic electroluminescent device. In addition, by comprising a plurality of host materials according to the present, an organic electroluminescent device having a low driving voltage, high luminous efficiency, high power efficiency, and/or excellent lifespan characteristics compared to conventional organic electroluminescent devices is provided, and it is possible to produce a display or a lighting device using the same.

### Mode for the Invention

Hereinafter, the present disclosure will be described in detail. However, the following description is intended to explain the present disclosure, and is not meant to restrict the scope of the present disclosure.

The term “organic electroluminescent compound” in the present disclosure means a compound that may be used in an organic electroluminescent device. The organic electroluminescent compound may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. Such at least two compounds may be comprised in the same layer or different layers, and may be mixture-evaporated or co-evaporated, or may be individually evaporated.

The term “a plurality of host materials” in the present disclosure means an organic electroluminescent material comprising a combination of at least two host materials. It may mean both a material before being comprised in an organic electroluminescent device (for example, before vapor deposition) and a material after being comprised in an organic electroluminescent device (for example, after vapor deposition). A plurality of host materials of the present disclosure may be comprised in any light-emitting layer constituting an organic electroluminescent device, and at least two compounds comprised in the plurality of host materials of the present disclosure may be comprised together in one light-emitting layer or may respectively be comprised in different light-emitting layers. When at least two host materials are comprised in one layer, for example, they may be mixture-evaporated to form a layer, or may be separately co-evaporated at the same time to form a layer.

Herein, the term “(C1-C30)alkyl(ene)” in the present disclosure is meant to be a linear or branched alkyl(ene) having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C2-C30)alkenyl” in the present disclosure is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” in the present disclosure is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl(ene)” is meant to be a mono- or polycyclic hydrocarbon having 3 to 30 ring backbone carbon atoms, in which the number of carbon atoms is preferably 3 to 20, and more preferably 3 to 10. The above cycloalkyl may include cyclopropyl, cyclobutyl, cyclopentyl, cyclohexyl, cyclopentylmethyl, cyclohexylmethyl, adamantyl, etc. The term “(3- to 7-membered)heterocycloalkyl” in the present disclosure is meant to be a cycloalkyl having 3 to 7, preferably 5 to 7 ring backbone atoms, and including at least one heteroatom selected from the group consisting of B, N, O, S, Si, and P, preferably at least one heteroatom selected from the group consisting of O, S, and N. The above heterocycloalkyl may include tetrahydrofuran, pyrrolidine, thiolane, tetrahydropyran, etc. The term “(C6-C30)aryl(ene)” in the present disclosure is meant to be a monocyclic or fused ring radical derived from an aromatic hydrocarbon having 6 to 30 ring backbone carbon atoms, and may be partially saturated. The number of ring backbone carbon atoms is preferably 6 to 25, and more preferably 6 to 18. The above aryl may comprise a spiro structure. The above aryl may include phenyl, biphenyl, terphenyl, naphthyl, binaphthyl, phenylnaphthyl, naphthylphenyl, phenylterphenyl, fluorenyl, phenylfluorenyl, diphenylfluorenyl, dimethylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, tetramethyldihydrophenanthrenyl, etc. Specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenylyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a] fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10, 10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” in the present disclosure is meant to be an aryl(ene) having 3 to 30 ring backbone atoms and including at least one heteroatom(s) selected from the group consisting of B, N, O, S, Si, and P. The number of heteroatoms is preferably 1 to 4. The above heteroaryl(ene) may be a monocyclic ring or a fused ring condensed with at least one benzene ring; may be partially saturated; may be one formed by linking at least one heteroaryl or aryl group to a heteroaryl(ene) group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzonaphthofuranyl, benzophenanthrofuranyl, benzonaphthothiophenyl, dibenzoselenophenyl, naphthobenzofuranyl, naphthobenzothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzophenanthrothiophenyl, benzoisoxazolyl, benzoxazolyl, phenanthrooxazolyl, phenanthrothiazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, benzotriazolephenazinyl, imidazopyridinyl, chromenoquinazolinyl, thiochromenoquinazolinyl, dimethylbenzoperimidinyl, indolocarbazolyl, indenocarbazolyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzothio[3,2-d]pyrazinyl, 9-benzofuro[3,2-d]pyrazinyl, 2-benzothio[3,2-d]pyrazinyl, 6-benzofuro[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. In the present disclosure, the term “halogen” includes F, Cl, Br, and I.

In addition, “ortho (o-)”, “meta (m-)”, and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

In addition, “substituted” in the expression “substituted or unsubstituted” in the present disclosure means that a hydrogen atom in a certain functional group is replaced with another atom or another functional group (i.e., a substituent), and also includes that hydrogen atom is replaced with a group formed by a linkage of two or more of the above substituents. For example, the “a group formed by a linkage of two or more substituents” may be pyridine-triazine. That is, pyridine-triazine may be interpreted as one heteroaryl substituent, or as substituents in which two heteroaryl substituents are linked. In formulas of the present disclosure, the substituent(s) of the substituted alkyl(ene), the substituted alkenyl, the substituted aryl(ene), the substituted heteroaryl(ene), the substituted cycloalkyl(ene), the substituted heterocycloalkyl, the substituted alkoxy, the substituted trialkylsilyl, the substituted dialkylarylsilyl, the substituted alkyldiarylsilyl, the substituted triarylsilyl, and the substituted fused ring group of an aliphatic ring(s) and an aromatic ring(s), each independently, represents at least one selected from the group consisting of deuterium; a halogen; a cyano; a carboxyl; a nitro; a hydroxyl; a phosphine oxide; a (C1-C30) alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with at least one of deuterium and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a mono- or di-(C6-C30)arylamino; a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituents, each independently, are at least one selected from the group consisting of deuterium, a (C1-C20)alkyl, a (C6-C20)cycloalkyl, a (5- to 25-membered)heteroaryl unsubstituted or substituted with a (C6-C25)aryl(s), and a (C6-C25)aryl. According to another embodiment of the present disclosure, the substituents, each independently, are at least one selected from the group consisting of deuterium, a (C1-C10)alkyl, a (C6-C15)cycloalkyl, a (5- to 20-membered)heteroaryl unsubstituted or substituted with a (C6-C18)aryl(s), and a (C6-C25)aryl. Specifically, the substituents, each independently, may be deuterium, a methyl, a tert-butyl, an adamantyl, a phenyl, a naphthyl, a biphenyl, a phenanthrenyl, a chrysenyl, a phenylfluorenyl, a dibenzofuranyl, a dibenzothiophenyl, a phenylsilafluorenyl, etc.

In the formulas of the present disclosure, a ring formed by a linkage of adjacent substituents means that at least two adjacent substituents are linked to each other to form a substituted or unsubstituted, mono or polycyclic, (3- to 30-membered) alicyclic ring or aromatic ring, or the combination thereof. In addition, the formed ring may comprise at least one heteroatom selected from B, N, O, S, Si, and P, preferably at least one heteroatom selected from N, O, and S. According to one embodiment of the present disclosure, the number of the ring backbone atoms is 5 to 20. According to another embodiment of the present disclosure, the number of the ring backbone atoms is 5 to 15. For example, the ring may be a substituted or unsubstituted, benzene ring, a naphthalene ring, a phenanthrene ring, a fluorene ring, an indene ring, an indole ring, a benzoindole ring, a benzofuran ring, a benzothiophene ring, a dibenzothiophene ring, a dibenzofuran ring, a carbazole ring, etc.

In the formulas of the present disclosure, heteroaryl, heteroarylene, and heterocycloalkyl may each independently comprise at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (5-to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

The plurality of host materials according to the present disclosure comprises the first host material including the compound represented by formula 1 and the second host material including the compound represented by formula 2, and may be included in a light-emitting layer of the organic electroluminescent device according to the present disclosure.

Hereinafter, the compound represented by formula 1 will be described in more detail.

In formula 1, L₁ to L₃ each independently represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene. According to one embodiment of the present disclosure, L₁ to L₃ each independently represent a single bond, or a substituted or unsubstituted (C6-C12)arylene. According to another embodiment of the present disclosure, L₁ to L₃ each independently represent a single bond, or an unsubstituted (C6-C12)arylene. For example, L₁ to L₃ may each independently represent a single bond, a phenylene, a naphthylene, etc.

In formula 1, Ar₁ to Ar₃ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered) heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -Lₐ-N(Arₐ)(Ar_{b}). According to one embodiment of the present disclosure, Ar₁ to Ar₃ each independently represent a substituted or unsubstituted (C6-C30)aryl, a substituted or unsubstituted (3- to 30-membered)heteroaryl, a substituted or unsubstituted (C3-C10)cycloalkyl, or -Lₐ-N(Arₐ)(Ar_{b}). According to another embodiment of the present disclosure, Ar₁ to Ar₃ each independently represent a (C6-C30)aryl unsubstituted or substituted with at least one of deuterium, a (C1-C6)alkyl(s), and a (C6-C20)aryl(s); a (3- to 30-membered)heteroaryl unsubstituted or substituted with a (C6-C15)aryl(s); an unsubstituted (C3-C10)cycloalkyl; or -Lₐ-N(Arₐ)(Ar_{b}). For example, Ar1 to Ar3 may each independently represent a phenyl, a phenyl substituted with deuterium, a phenyl substituted with a methyl(s), a phenyl substituted with a tert-butyl(s), a phenyl substituted with a phenylfluorenyl(s), a naphthyl, a phenylnaphthyl, a biphenyl, a terphenyl, an anthracenyl, a phenanthrenyl, a fluoranthenyl, a tetramethyltetrahydrophenanthrenyl, a dimethylfluorenyl, a methylphenylfluorenyl, a diphenylfluorenyl, a dimethylbenzofluorenyl, a spirobifluorenyl, a (C22)aryl, a phenylpyridyl, a benzofuranyl, a benzimidazolyl substituted with a phenyl(s), a dibenzofuranyl, a dibenzothiophenyl, a dibenzofuranyl substituted with a phenyl(s), a carbazolyl substituted with a phenyl(s), a dibenzocarbazolyl, a benzonaphthofuranyl, a benzonaphthothiophenyl, a phenoxazinyl, a phenanthrooxazolyl, a phenanthrooxazolyl substituted with a phenyl(s), a phenanthrooxazolyl substituted with a phenanthrenyl(s), a phenanthrothiazolyl substituted with a phenyl(s), a phenantrothiazolyl substituted with a biphenyl(s), a benzene fused phenanthrooxazolyl substituted with a phenyl(s), a (14-membered) nitrogen-containing heteroaryl substituted with a methyl(s), a (23-membered) nitrogen-containing heteroaryl unsubstituted or substituted with a phenyl(s), a benzene fused (23-membered) nitrogen-containing heteroaryl, a (26-membered) nitrogen-containing heteroaryl unsubstituted or substituted with a phenyl(s), a benzene fused (26-membered) nitrogen-containing heteroaryl, a diphenylamino, etc.

Herein, Lₐ each independently represents a single bond, a substituted or unsubstituted (C6-C30)arylene, or a substituted or unsubstituted (3- to 30-membered)heteroarylene. According to one embodiment of the present disclosure, Lₐ each independently represents a single bond.

Arₐ and Ar_{b} each independently represent hydrogen, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl. According to one embodiment of the present disclosure, Arₐ and Ar_{b} each independently represent a substituted or unsubstituted (C6-C12)aryl. According to another embodiment of the present disclosure, Arₐ and Ar_{b} each independently represent an unsubstituted (C6-C12)aryl. For example, Arₐ and Ar_{b} may each independently represent a phenyl, etc.

In formula 1, the case where all of L₁ to L₃ represent single bonds, and all of Ar₁ to Ar₃ represent hydrogen is excluded.

According to one embodiment of the present disclosure, the formula 1 may be represented by at least one of the following formulas 1-1 to 1-12.

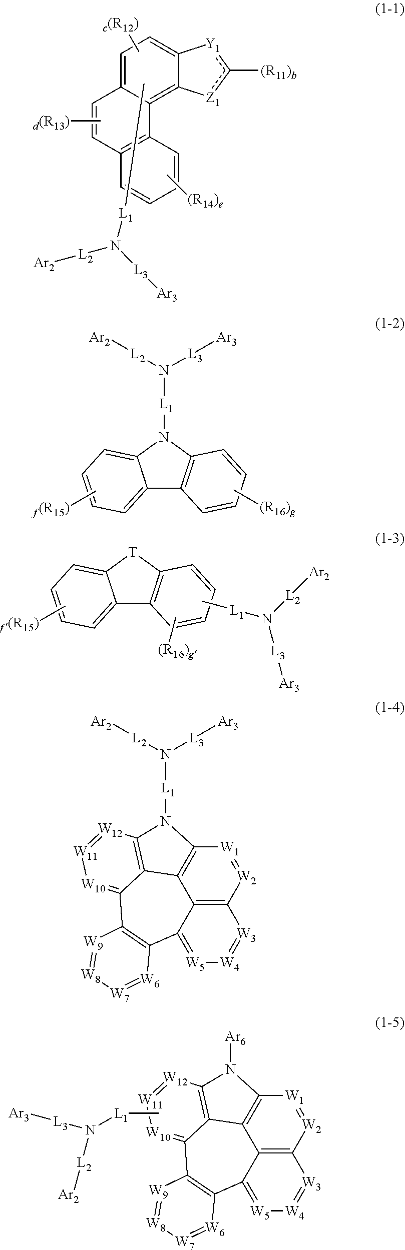

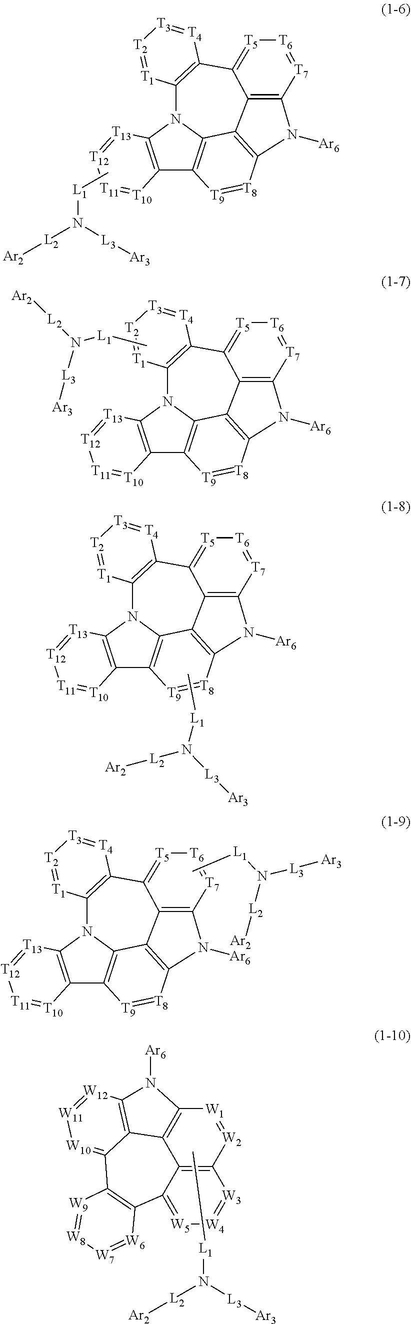

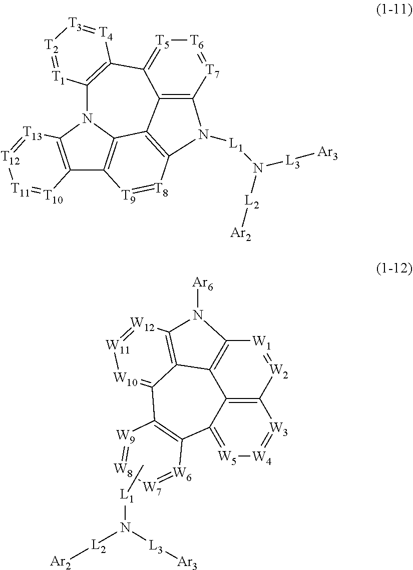

In formulas 1-1 to 1-12,
Y₁ and Z₁ each independently represent —N═, —NR₂₁—, —O— or —S—, with a proviso that one of Y₁ and Z₁ represents —N═, and the other of Y₁ and Z₁ represents —NR₂₁—, —O— or —S—;
T represents CR₂₂R₂₃, NR₂₄, O or S;
T₁ to T₁₃ and W₁ to W₁₂ each independently represent N or CV₁;

R₁₁ represents a substituted or unsubstituted (C6-C30)aryl, or a substituted or unsubstituted (3- to 30-membered)heteroaryl;

R₁₂ to R₁₆, R₂₁ to R₂₄ and V₁ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substitute or unsubstituted fused ring group of a (C3-C30) aliphatic ring(s) and a (C6-C30) aromatic ring(s), or -L_{c}-N(Arₑ)(Ar_{f}), or may be linked to an adjacent substituent(s) to form a ring(s);
L_{c} each independently represents a single bond, a substituted or unsubstituted (C6-C30)arylene, or a substituted or unsubstituted (3- to 30-membered)heteroarylene;
Arₑ and Ar_{f} each independently represent hydrogen, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl;
Ar₆ represents a substituted or unsubstituted (C6-C30)aryl, or a substituted or unsubstituted (3- to 30-membered)heteroaryl;
b represents an integer of 1, c and d each independently represent an integer of 1 or 2, e, f, g and f′ each independently represent an integer of 1 to 4, and g′ represents an integer of 1 to 3, where if c to g, f′ and g′ are an integer of 2 or more, each of R₁₂ to each of R₁₆ may be the same or different; and
Ar₂, Ar₃, and L₁ to L₃ are as defined in formula 1.

Hereinafter, the compound represented by formula 2 will be described in more detail.

In formula 2, R₁ to R₁₀ are each independently represented by

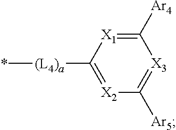

or represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L_{b}-N(Ar_{c})(Ar_{d}); or may be linked to an adjacent substituent(s) to form a ring(s), with a proviso that at least one of R₁ to R₁₀ is represented by

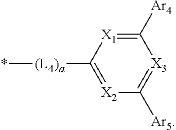

According to one embodiment of the present disclosure, R₁ to R₁₀ are each independently represented by

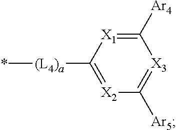

or represent hydrogen, deuterium, a substituted or unsubstituted (C6-C15)aryl, or a substituted or unsubstituted (5- to 15-membered)heteroaryl. According to another embodiment of the present disclosure, R₁ to R₁₀ are each independently represented by

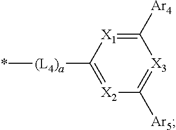

or represent hydrogen, an unsubstituted (C6-C15)aryl, or an unsubstituted (5-to 15-membered)heteroaryl.

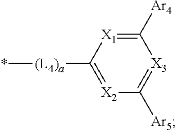

For example, R₁ to R₁₀ may be each independently represented by or represent hydrogen, a phenyl, a biphenyl, a naphthyl, a dibenzofuranyl, etc.

Herein, L_{b} each independently represents a single bond, a substituted or unsubstituted (C6-C30)arylene, or a substituted or unsubstituted (3- to 30-membered)heteroarylene; and Ar_{c} and Ar_{d} each independently represent hydrogen, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl.

In addition, herein, L₄ represents a single bond, a substituted or unsubstituted (C6-C30)cycloalkylene, a substituted or unsubstituted (C6-C30)arylene, or a substituted or unsubstituted (3- to 30-membered)heteroarylene. According to one embodiment of the present disclosure, L₄ represents a single bond, a substituted or unsubstituted (C6-C15)cycloalkylene, or a substituted or unsubstituted (C6-C20)arylene. According to another embodiment of the present disclosure, L₄ represents a single bond, an unsubstituted (C6-C15)cycloalkylene, or a (C6-C20)arylene unsubstituted or substituted with a (C6-C12)aryl(s). For example, L₄ may represent a single bond, an adamantylene, a phenylene, a naphthylene, a phenylene substituted with a phenyl(s), -phenyl-naphthyl-, etc.

X₁ to X₃ each independently represent N or CH, with a proviso that at least one of X₁ to X₃ is N. According to one embodiment of the present disclosure, all of X₁ to X₃ represent N.

In formula 2, Ar₄ and Ar₅ each independently represent a substituted or unsubstituted (C6-C30)cycloalkyl, a substituted or unsubstituted (C6-C30)aryl, a substituted or unsubstituted (3- to 30-membered)heteroaryl, or a substituted or unsubstituted fused ring group of a (C3-C30) aliphatic ring(s) and a (C6-C30) aromatic ring(s). According to one embodiment of the present disclosure, Ar₄ and Ar₅ each independently represent a substituted or unsubstituted (C6-C15)cycloalkyl, a substituted or unsubstituted (C6-C30)aryl, a substituted or unsubstituted (5-to 15-membered)heteroaryl, or a substituted or unsubstituted fused ring group of a (C3-C10) aliphatic ring(s) and a (C6-C12) aromatic ring(s). According to another embodiment of the present disclosure, Ar₄ and Ar₅ each independently represent an unsubstituted (C6-C30)cycloalkyl; a (C6-C30)aryl unsubstituted or substituted with at least one of a (C6-C15)cycloalkyl(s), a (C6-C20)aryl(s) and a (5- to 15-membered)heteroaryl(s); a (5-to 15-membered)heteroaryl unsubstituted or substituted with a (C6-C12)aryl(s); or a fused ring group of a (C3-C10) aliphatic ring(s) and a (C6-C12) aromatic ring(s) substituted with a (C1-C6)alkyl(s). For example, Ar₄ and Ar₅ may each independently represent an adamantyl; a phenyl unsubstituted or substituted with at least one of an adamantly(s), a phenyl(s), a naphthyl(s), a phenylfluorenyl(s), a chrysenyl(s), a dibenzofuranyl(s) and a dibenzothiophenyl(s); a naphthyl unsubstituted or substituted with a phenyl(s) and/or a dibenzofuranyl(s); a biphenyl; a terphenyl; a (C22)aryl; a phenanthrenyl; a triphenylenyl; a fluorenyl unsubstituted or substituted with at least one of a methyl(s), a phenyl and an adamantyl; a dibenzofuranyl; a dibenzothiophenyl; a diphenylsilafluorenyl; a fused ring group of (C3-C10) aliphatic ring(s) and (C6-C12) aromatic ring(s) substituted with a methyl(s), etc.

In formula 2,
a represents an integer of 1 to 3, where if a is an integer of 2 or more, each of L₄ may be the same or different.

According to one embodiment of the present disclosure, the formula 2 may be represented by at least one of the following formulas 2-1 to 2-5.

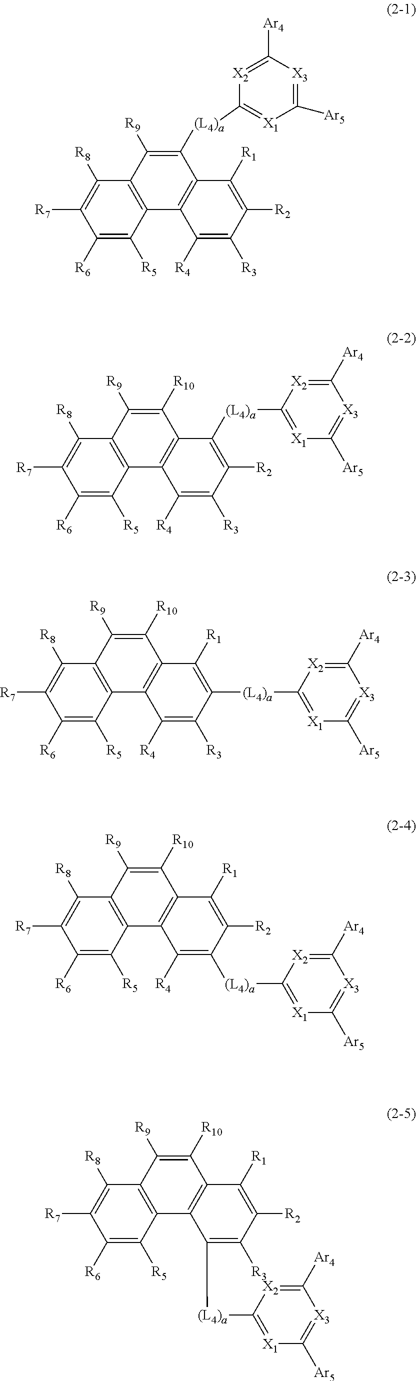

In formulas 2-1 to 2-5, R₁ to R₁₀, L₄, Ar₄, Ar₅, X₁ to X₃ and a are as defined in formula 2.

The compound represented by formula 1 may be at least one selected from the following compounds, but is not limited thereto.

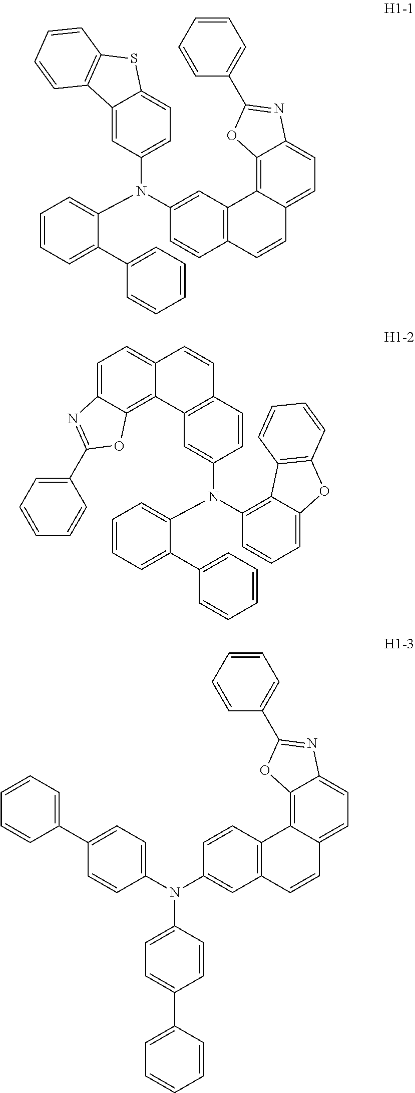

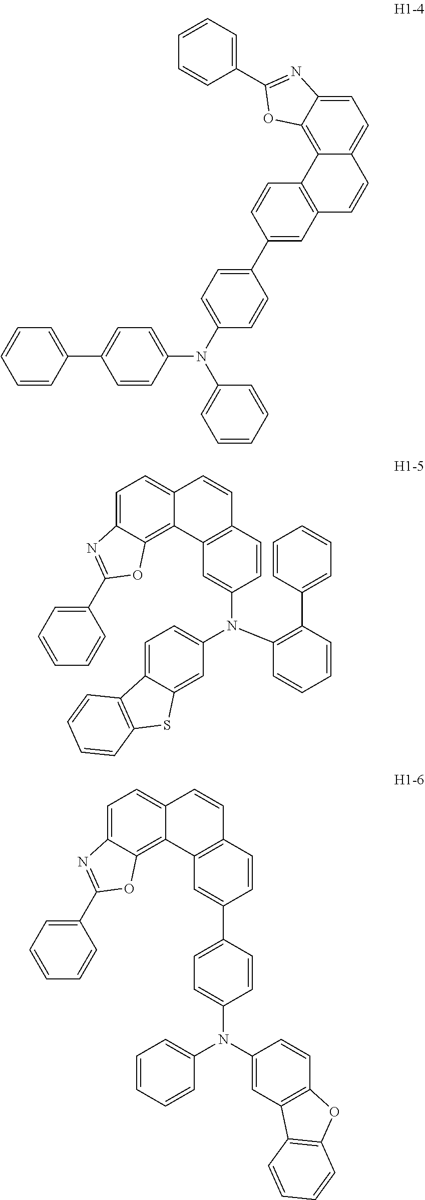

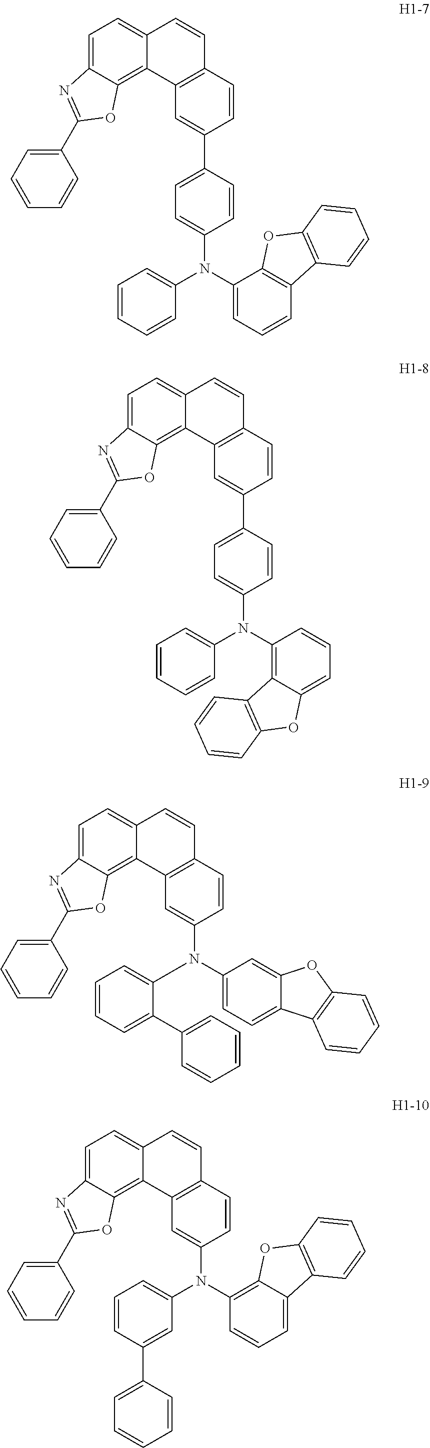

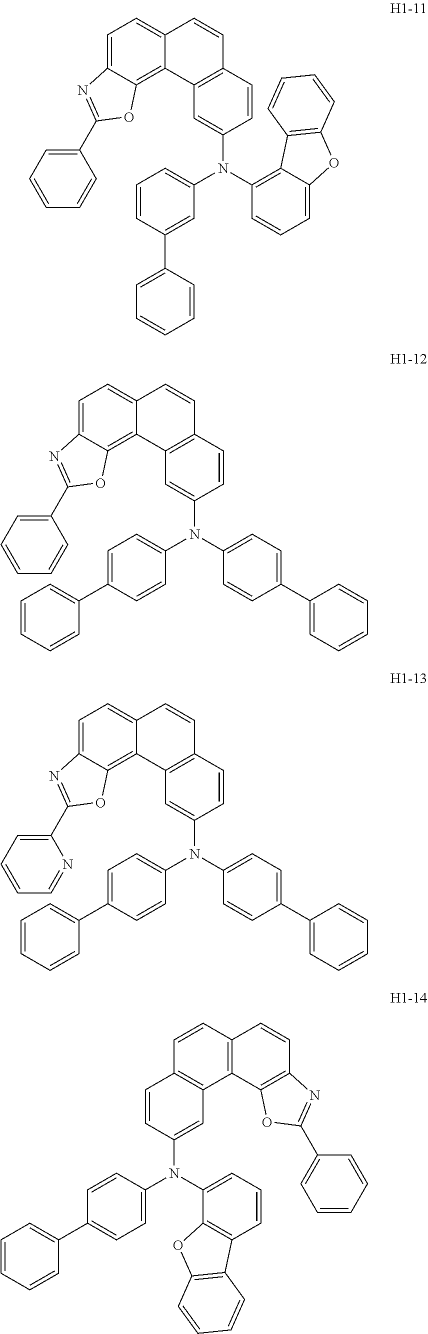 [SMILES: c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2ccc(N(c3ccc(-c4ccccc4)cc3)c3ccc4ccc5ccc6nc(-c7ccccn7)oc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1]

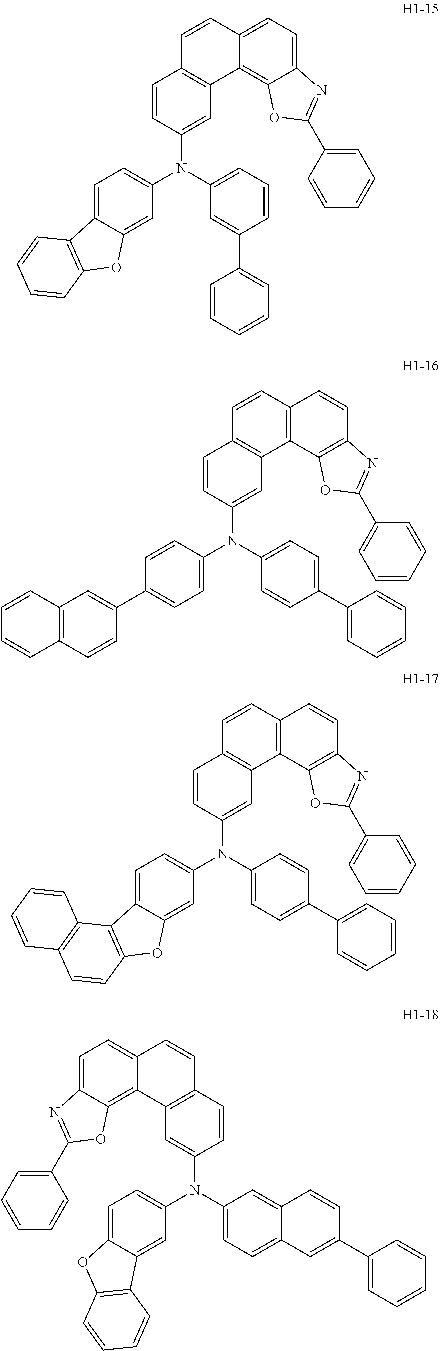 [SMILES: c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1]

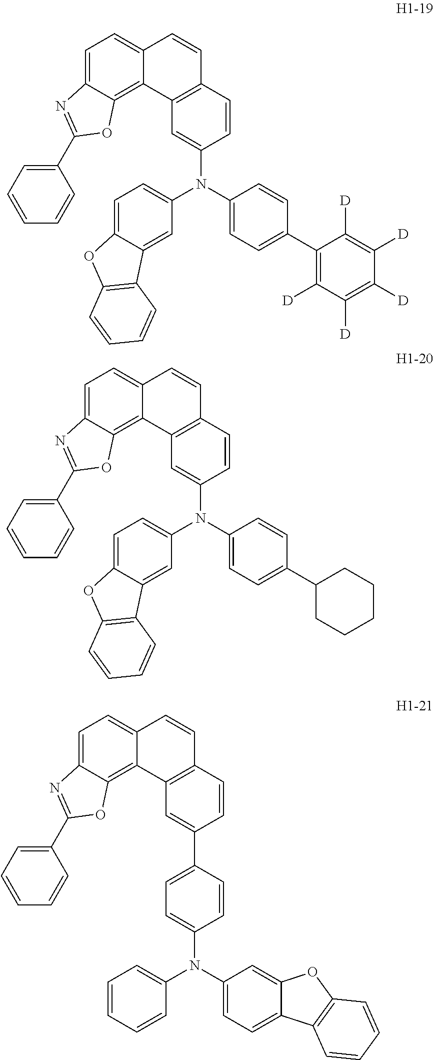

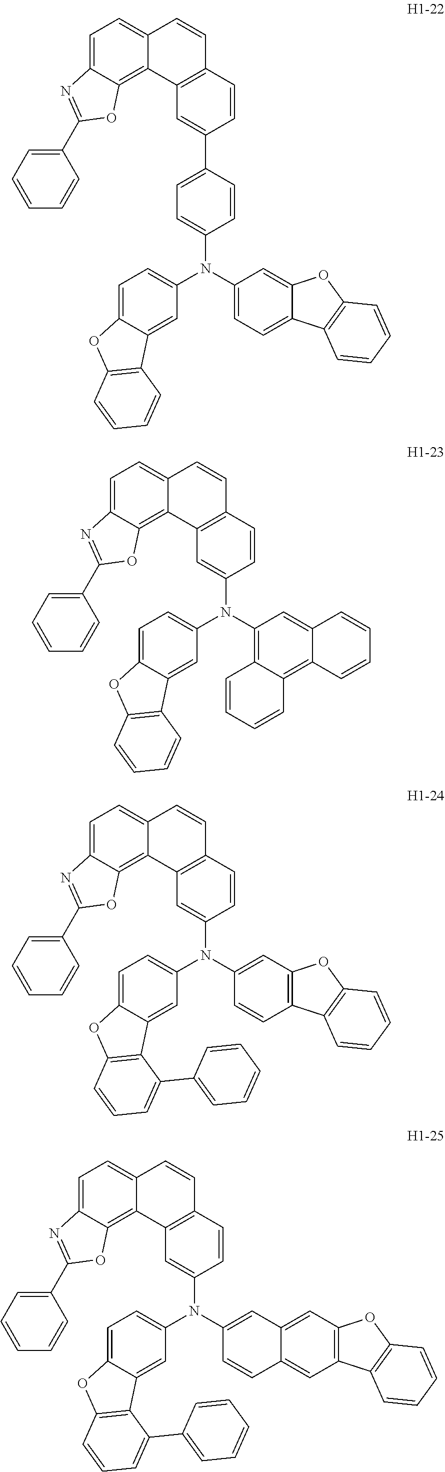

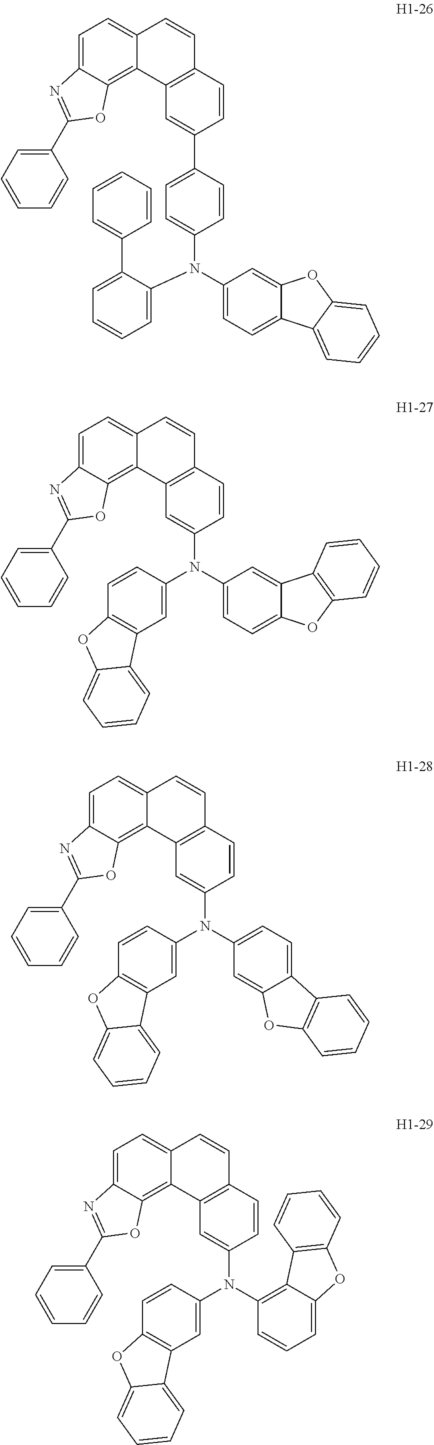 [SMILES: c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1]

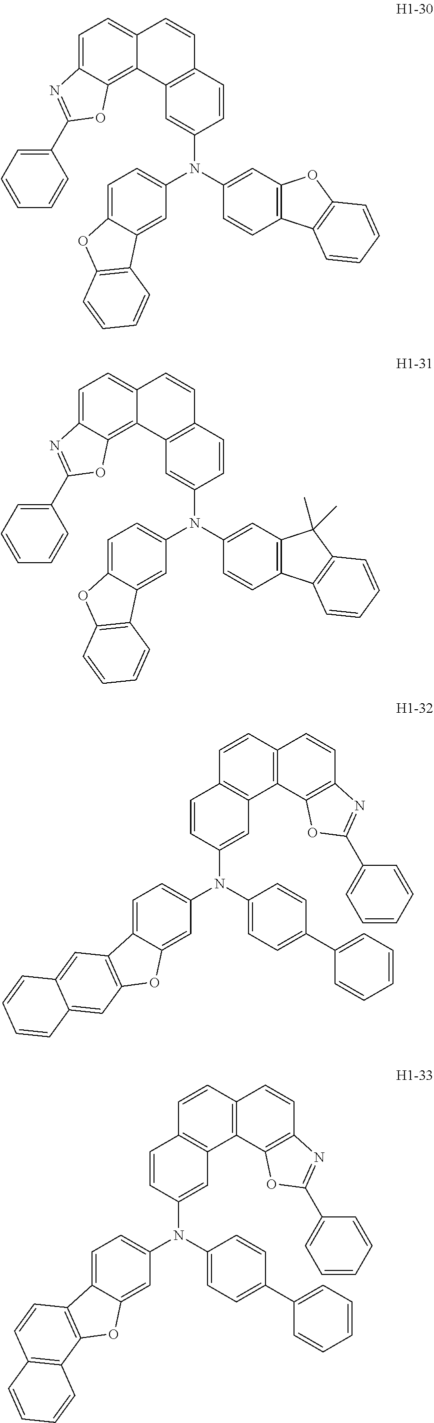

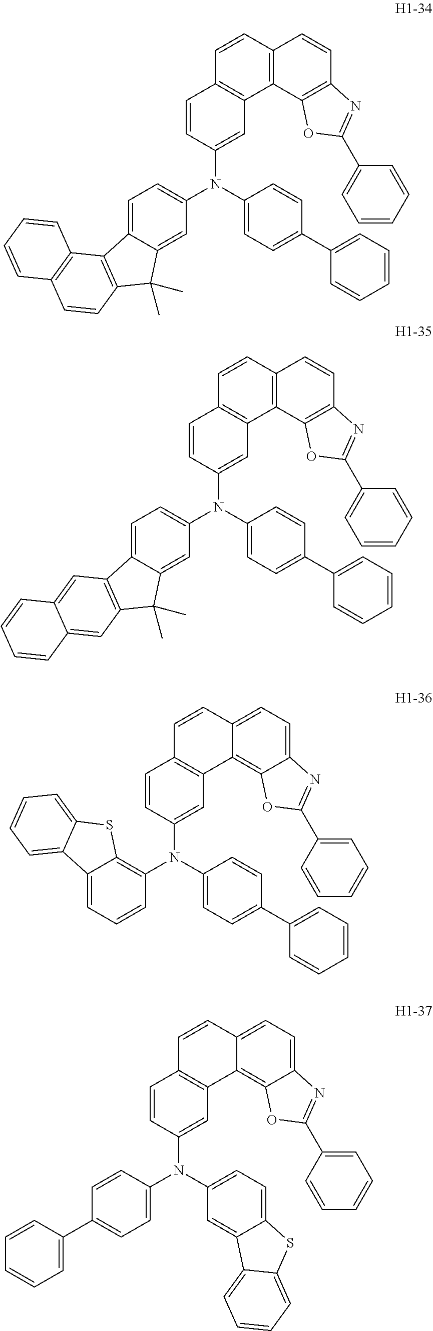 [SMILES: CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21 ; c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1]

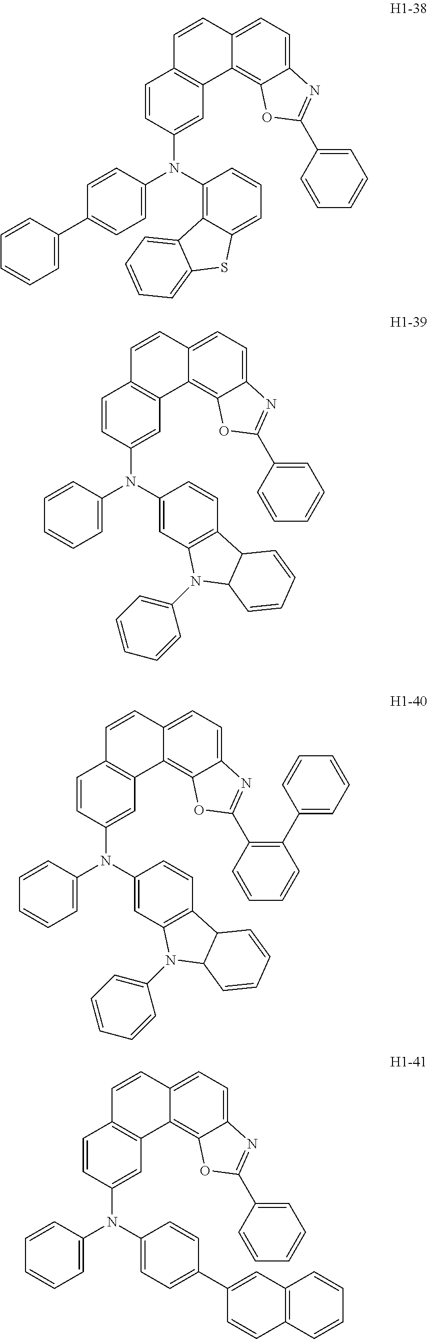

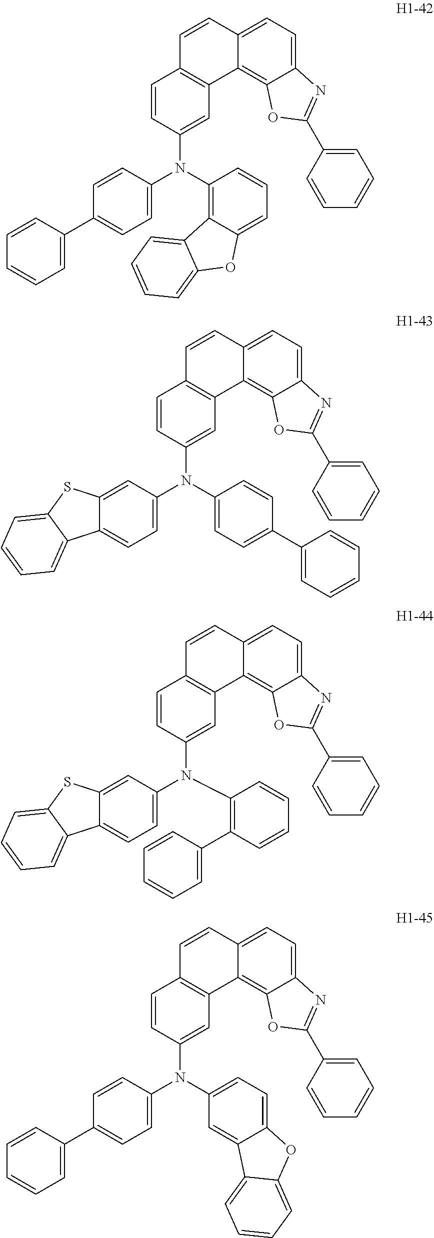

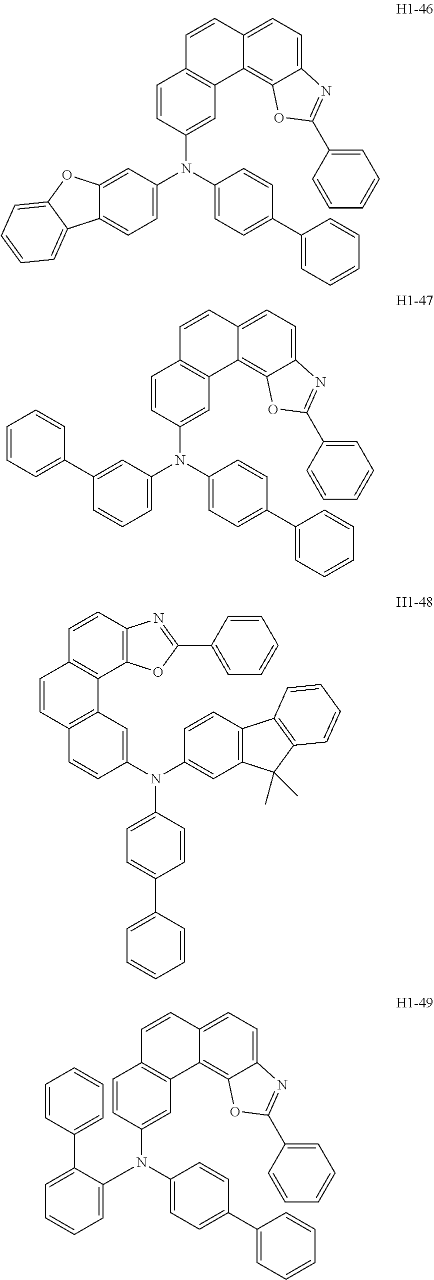

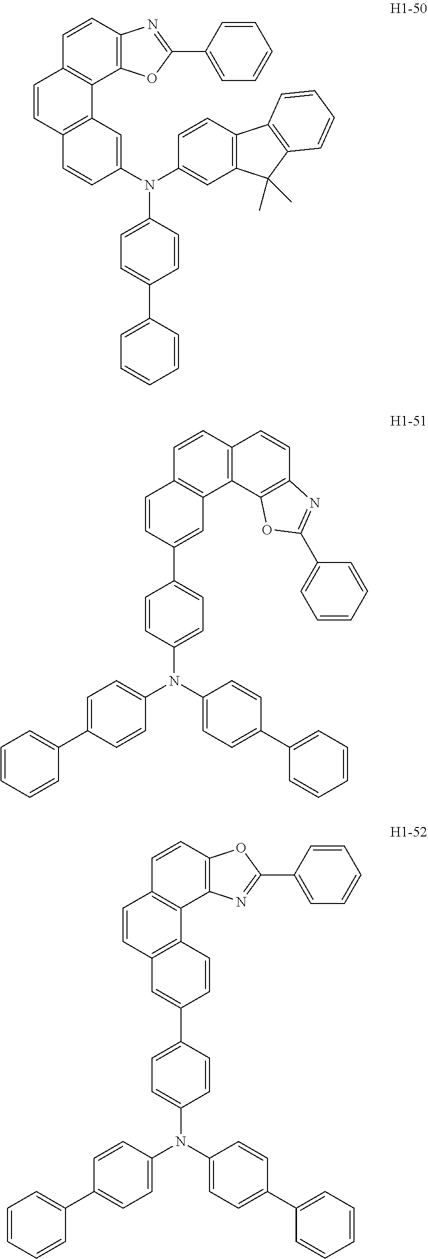 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1]

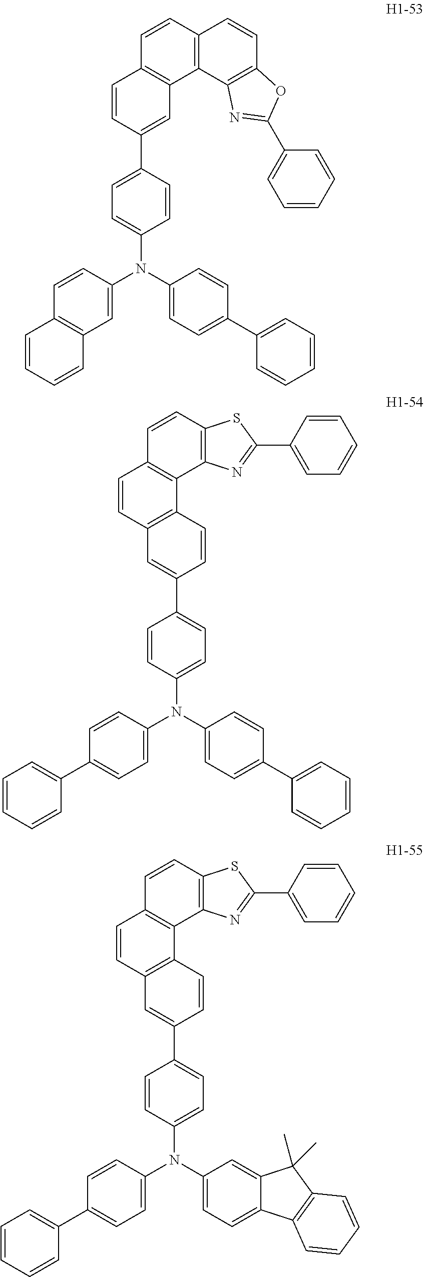

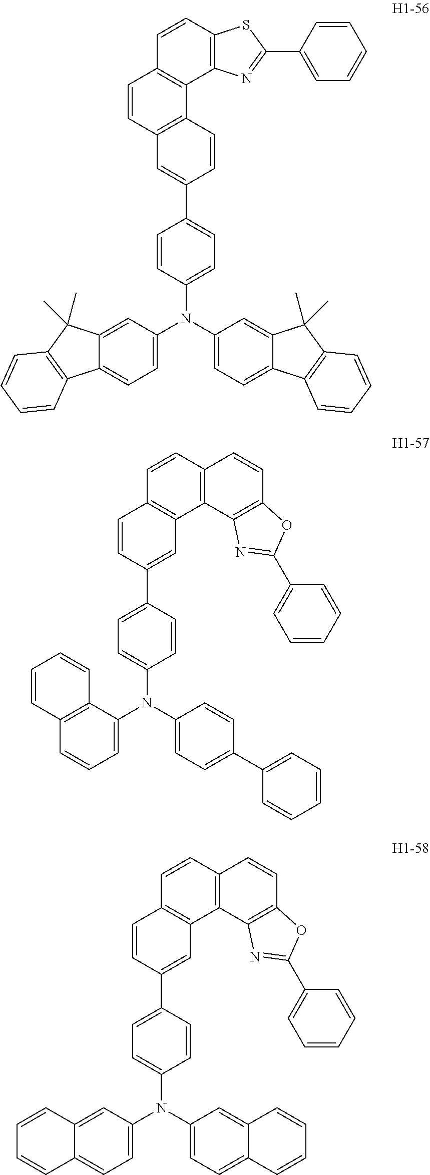 [SMILES: CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1]

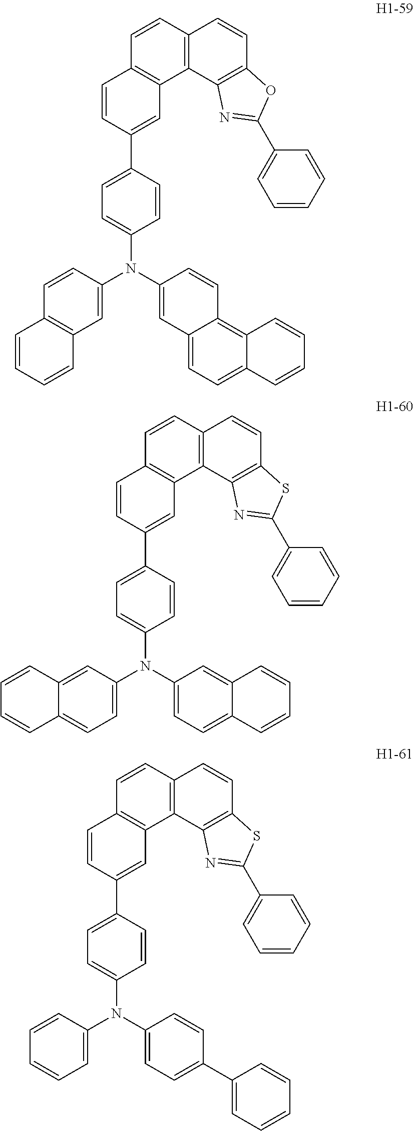

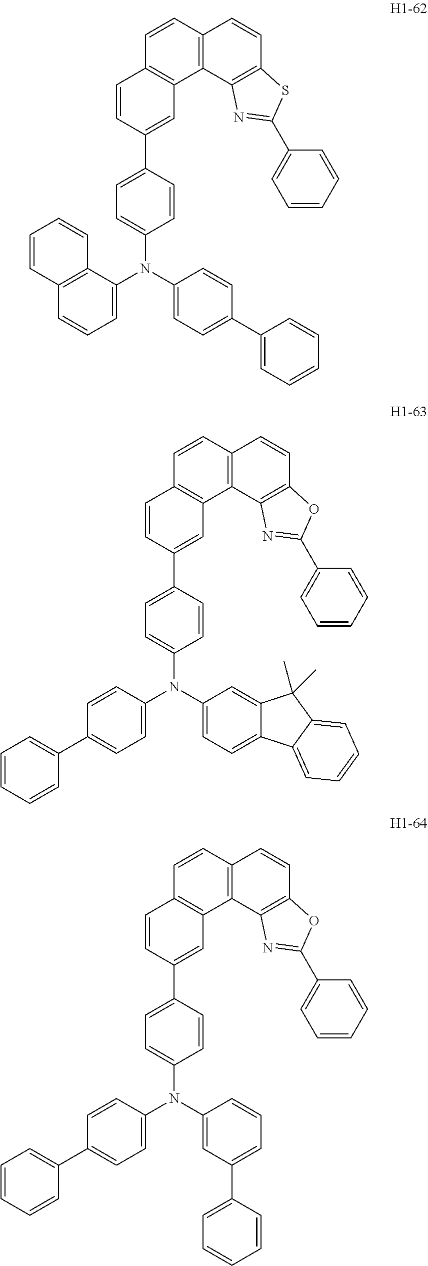 [SMILES: c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1]

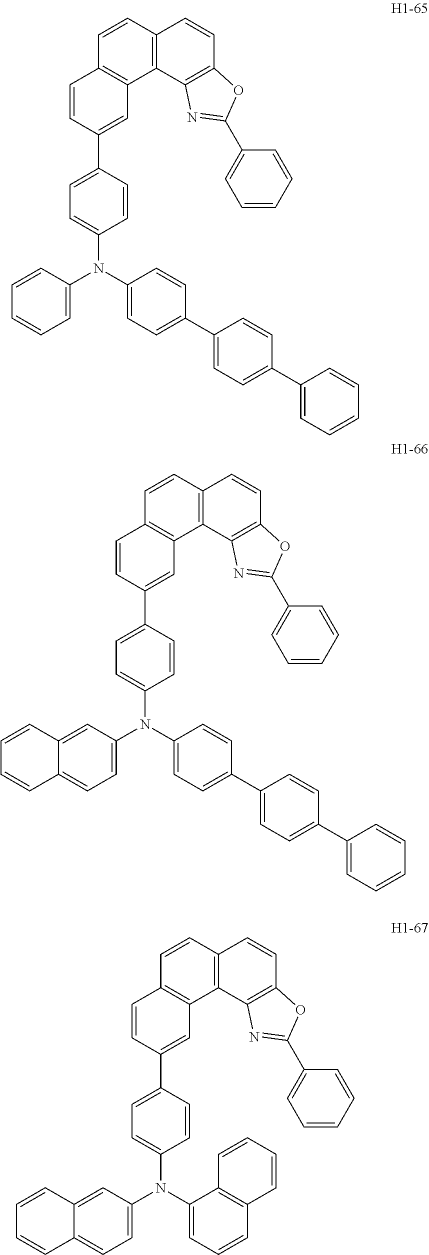 [SMILES: c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1]

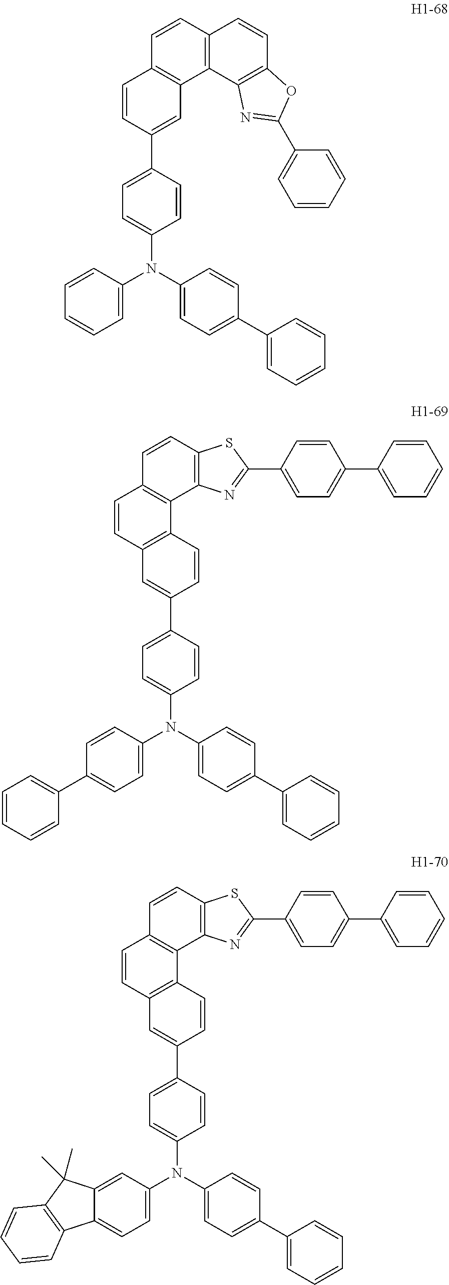 [SMILES: c1ccc(-c2ccc(N(c3ccccc3)c3ccc(-c4ccc5ccc6ccc7oc(-c8ccccc8)nc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21]

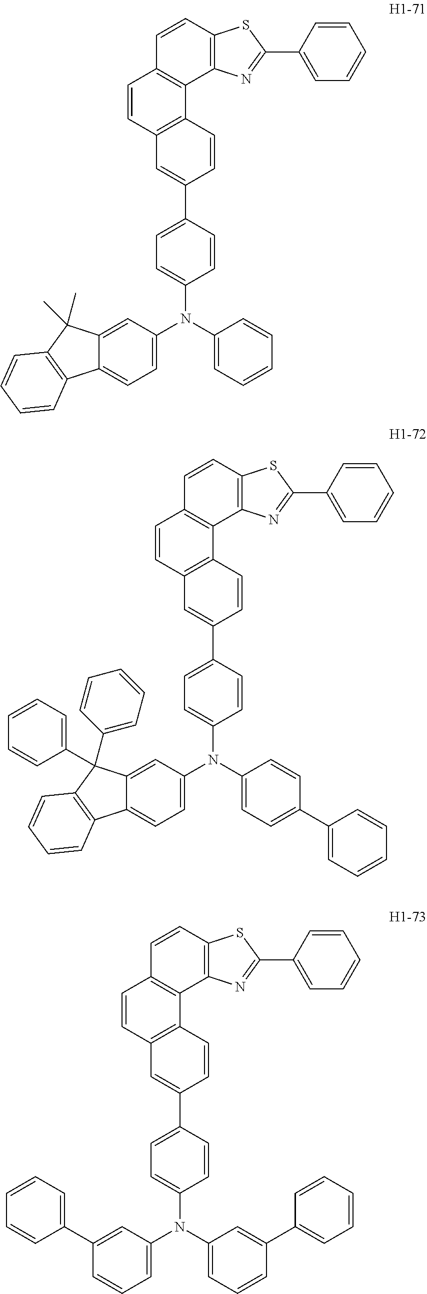 [SMILES: CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1]

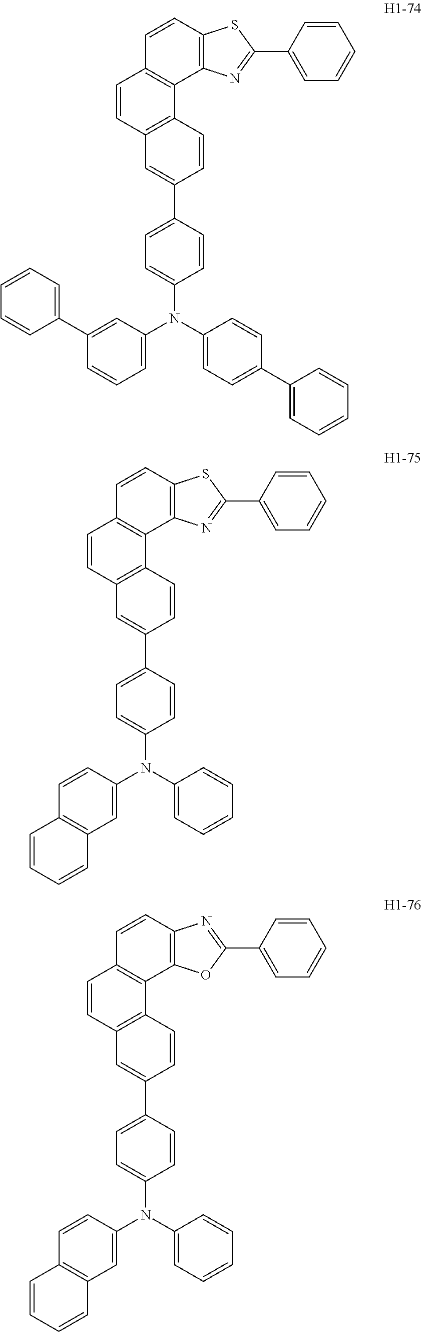

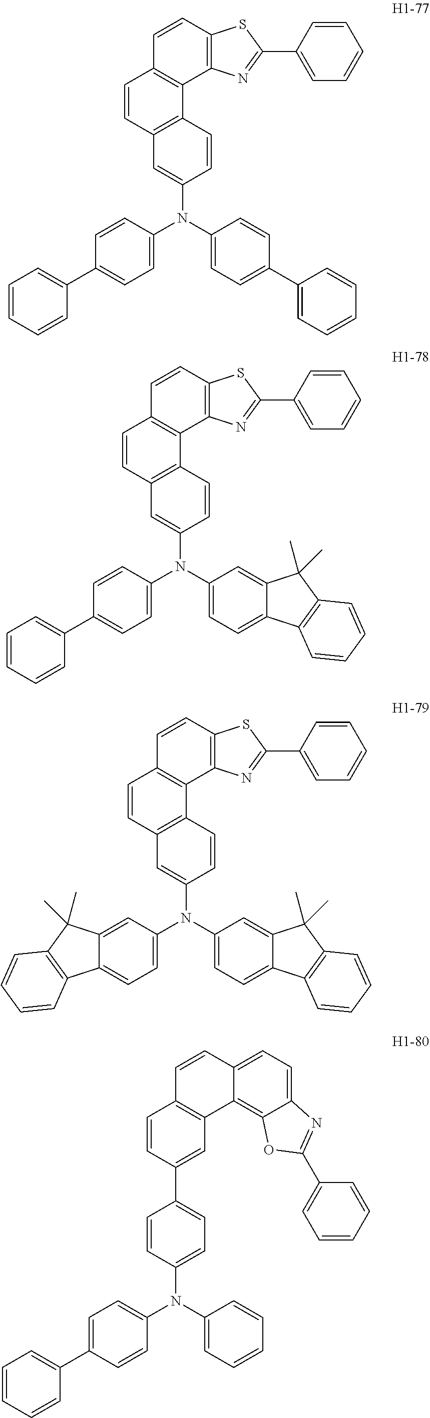

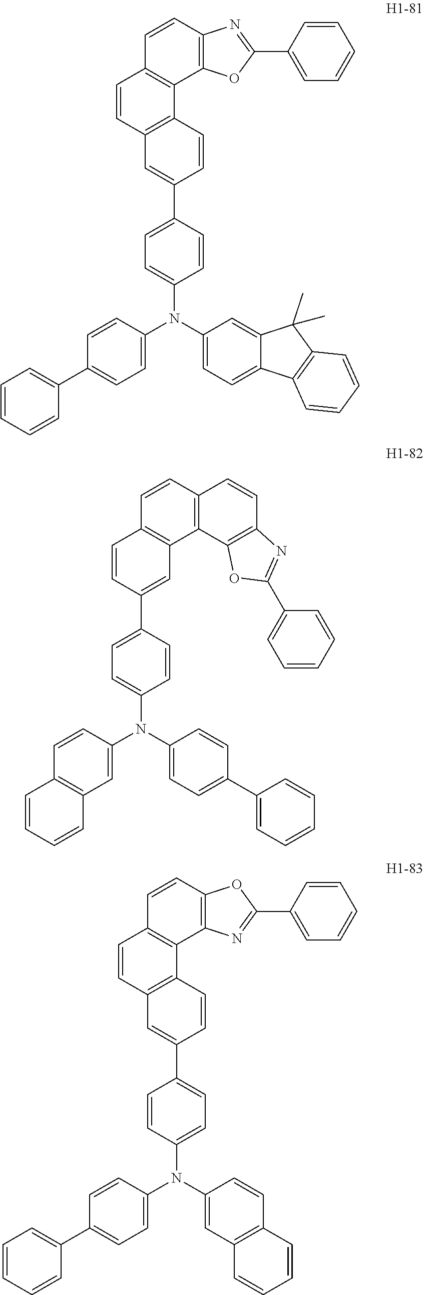

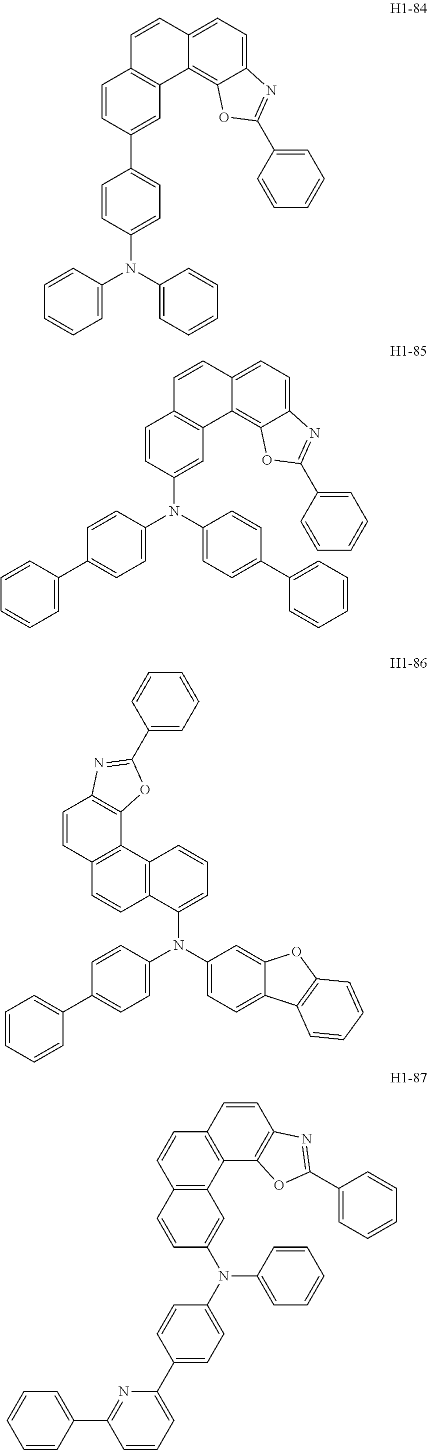

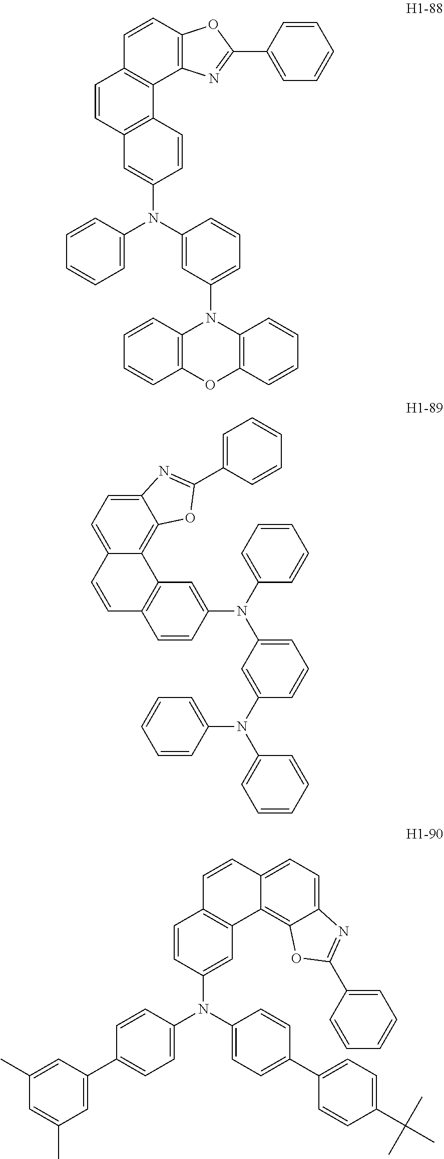

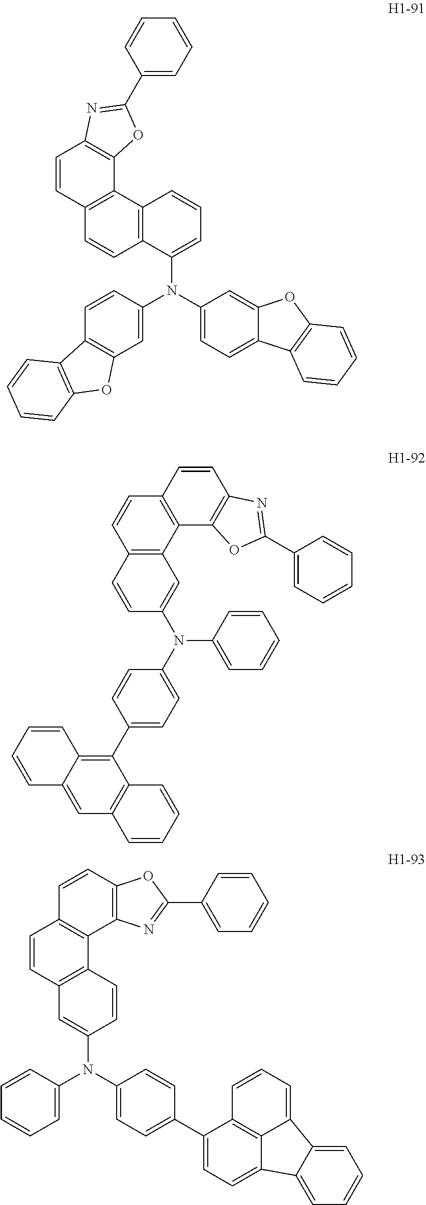

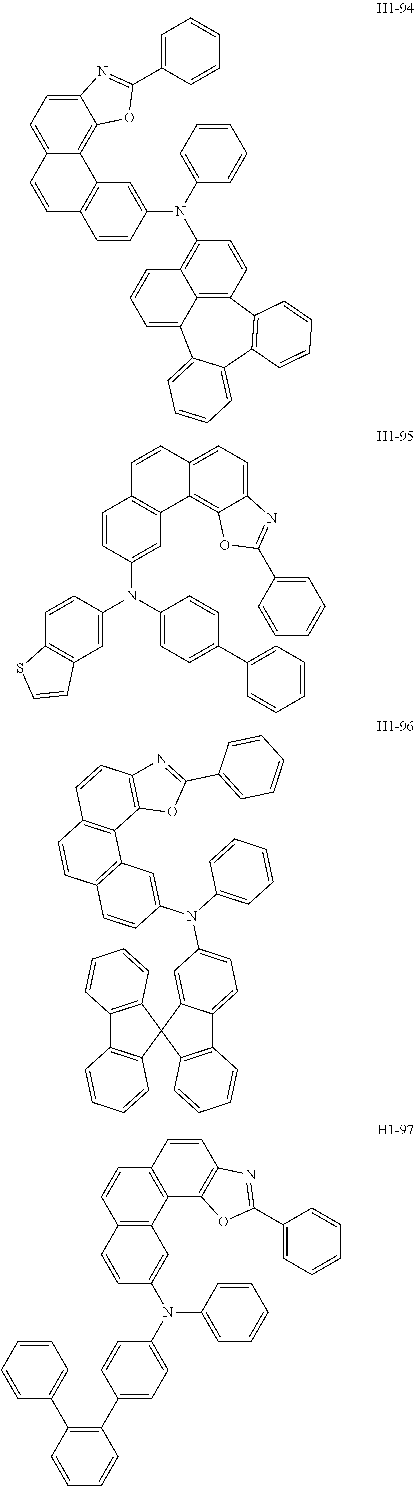

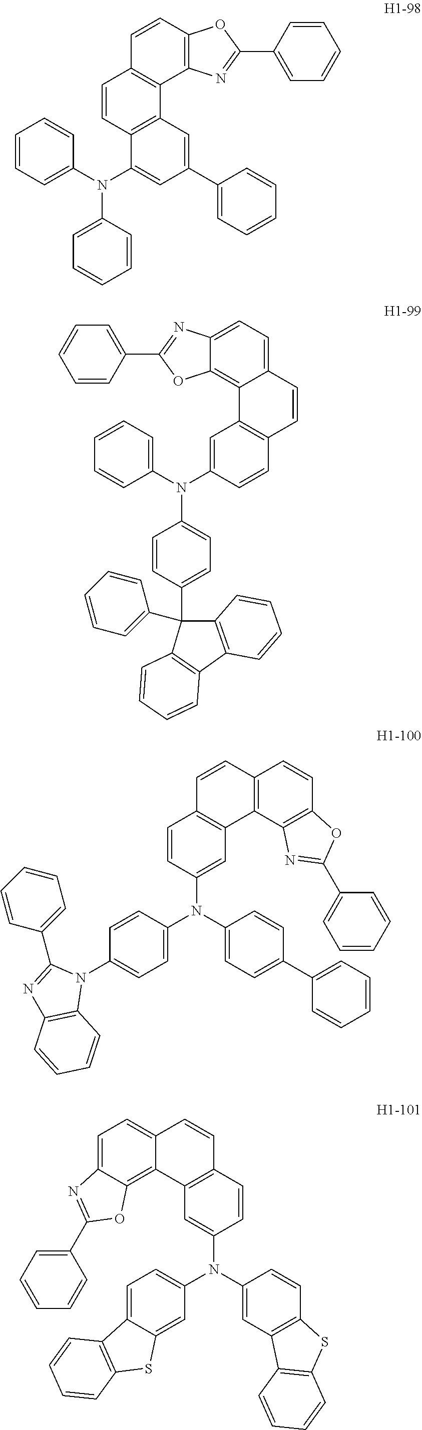

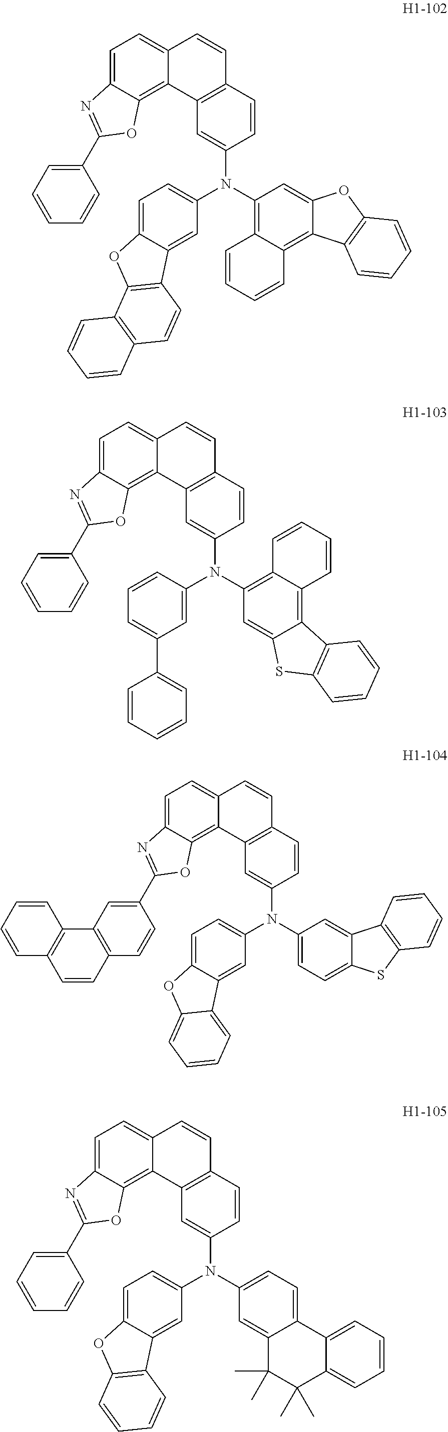 [SMILES: c1ccc(-c2nc3ccc4ccc5ccc(N(c6ccc7oc8c9ccccc9ccc8c7c6)c6cc7oc8ccccc8c7c7ccccc67)cc5c4c3o2)cc1 ; c1ccc(-c2cccc(N(c3ccc4ccc5ccc6nc(-c7ccccc7)oc6c5c4c3)c3cc4sc5ccccc5c4c4ccccc34)c2)cc1 ; c1ccc2c(c1)ccc1ccc(-c3nc4ccc5ccc6ccc(N(c7ccc8oc9ccccc9c8c7)c7ccc8sc9ccccc9c8c7)cc6c5c4o3)cc12 ; CC1(C)c2ccccc2-c2ccc(N(c3ccc4oc5ccccc5c4c3)c3ccc4ccc5ccc6nc(-c7ccccc7)oc6c5c4c3)cc2C1(C)C]

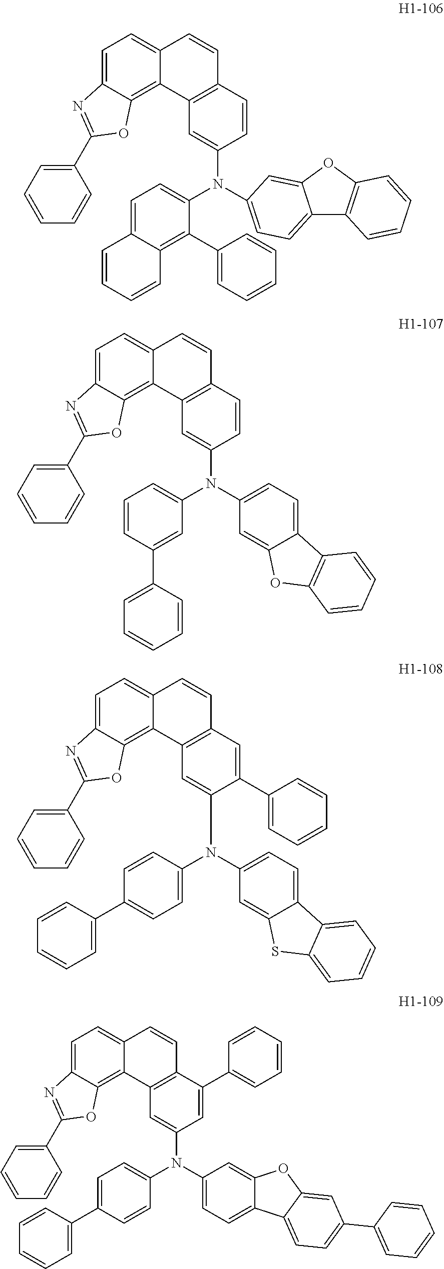

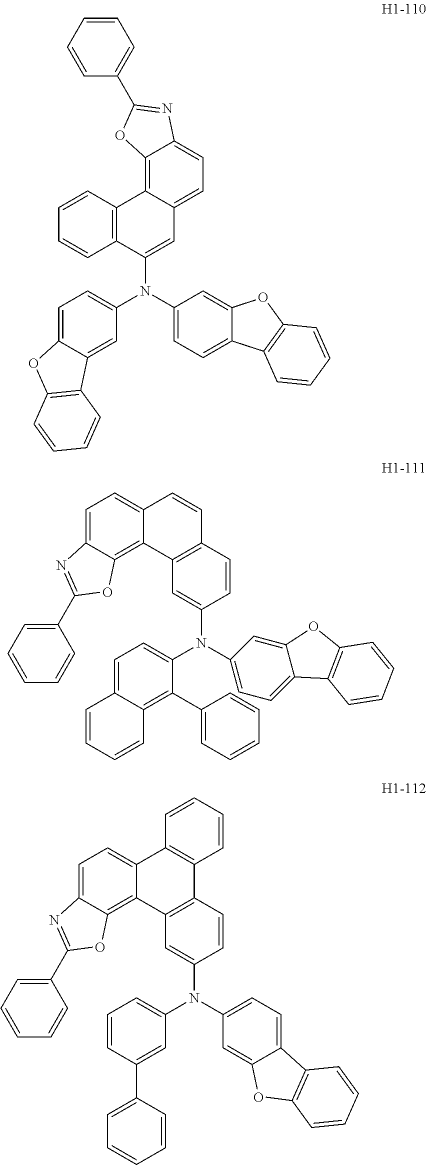 [SMILES: c1ccc(-c2nc3ccc4cc(N(c5ccc6c(c5)oc5ccccc56)c5ccc6oc7ccccc7c6c5)c5ccccc5c4c3o2)cc1 ; c1ccc(-c2nc3ccc4ccc5ccc(N(c6ccc7c(c6)oc6ccccc67)c6ccc7ccccc7c6-c6ccccc6)cc5c4c3o2)cc1 ; c1ccc(-c2cccc(N(c3ccc4c(c3)oc3ccccc34)c3ccc4c5ccccc5c5ccc6nc(-c7ccccc7)oc6c5c4c3)c2)cc1]

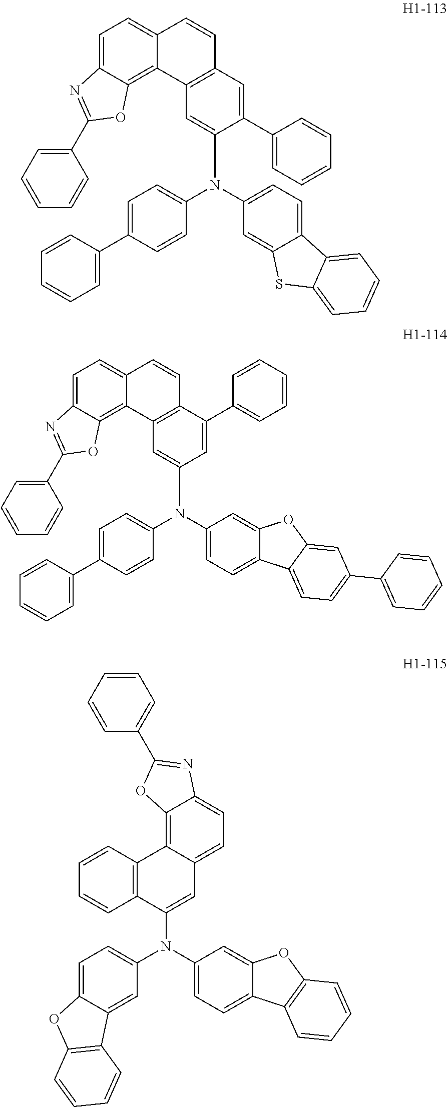 [SMILES: c1ccc(-c2ccc(N(c3ccc4c(c3)sc3ccccc34)c3cc4c(ccc5ccc6nc(-c7ccccc7)oc6c54)cc3-c3ccccc3)cc2)cc1 ; c1ccc(-c2ccc(N(c3ccc4c(c3)oc3cc(-c5ccccc5)ccc34)c3cc(-c4ccccc4)c4ccc5ccc6nc(-c7ccccc7)oc6c5c4c3)cc2)cc1 ; c1ccc(-c2nc3ccc4cc(N(c5ccc6c(c5)oc5ccccc56)c5ccc6oc7ccccc7c6c5)c5ccccc5c4c3o2)cc1]

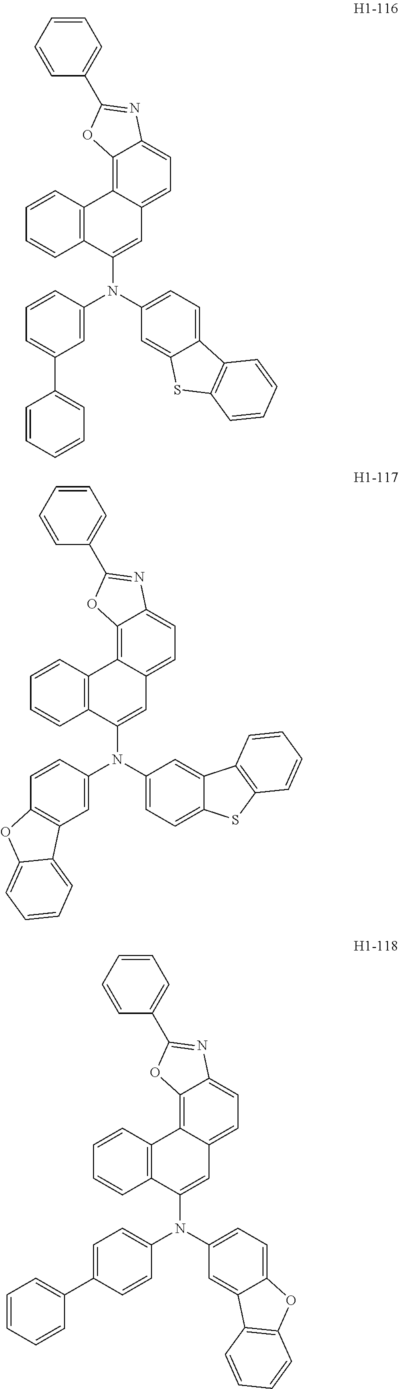

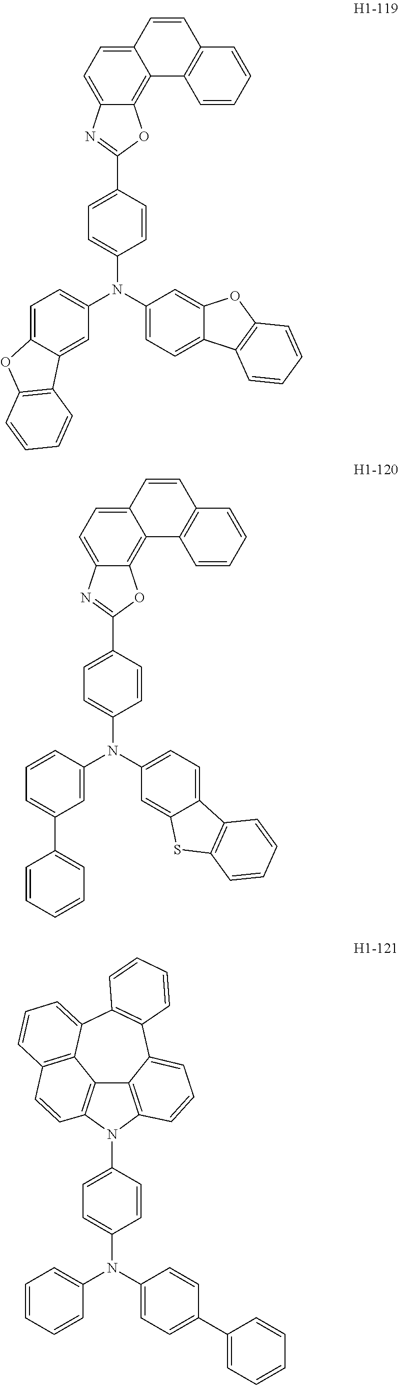 [SMILES: c1ccc2c(c1)ccc1ccc3nc(-c4ccc(N(c5ccc6c(c5)oc5ccccc56)c5ccc6oc7ccccc7c6c5)cc4)oc3c12 ; c1ccc(-c2cccc(N(c3ccc(-c4nc5ccc6ccc7ccccc7c6c5o4)cc3)c3ccc4c(c3)sc3ccccc34)c2)cc1 ; c1ccc(-c2ccc(N(c3ccccc3)c3ccc(-n4c5cccc6c5c5c7c(cccc7ccc54)-c4ccccc4-6)cc3)cc2)cc1]

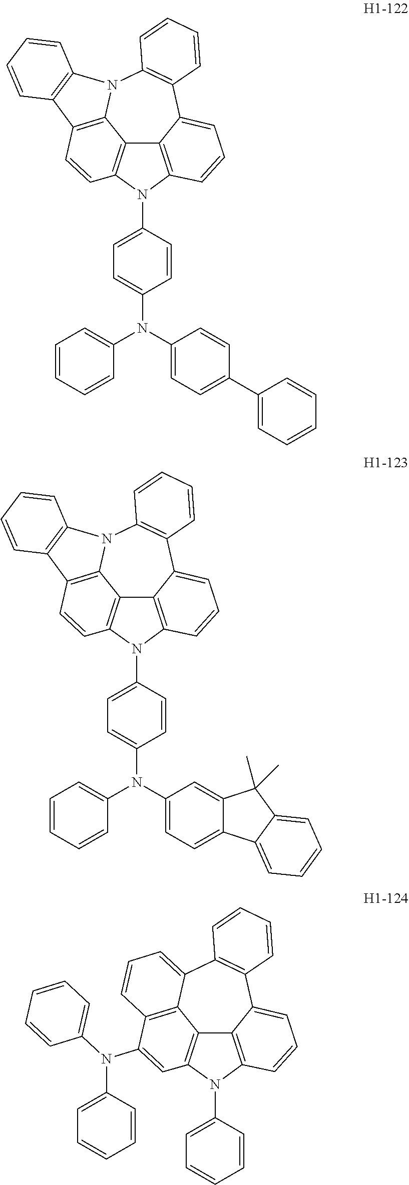

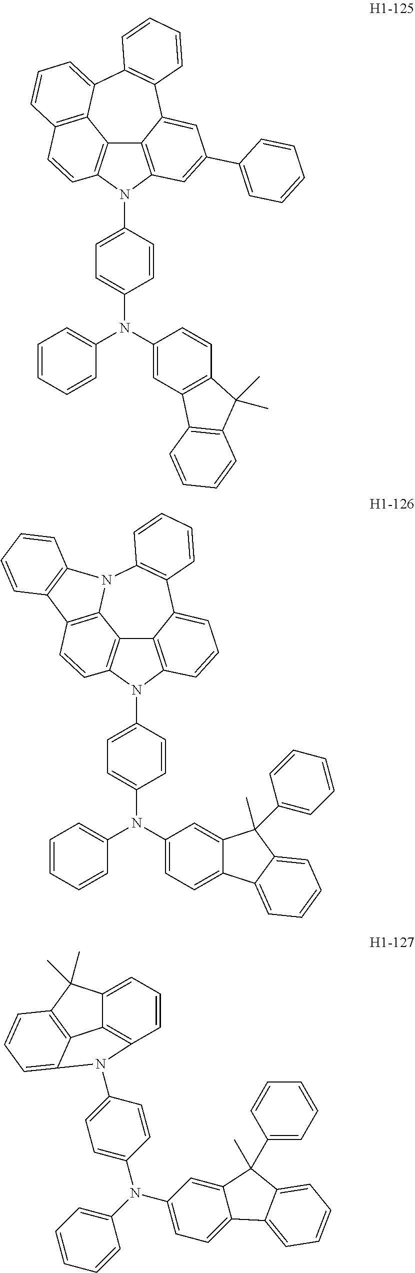

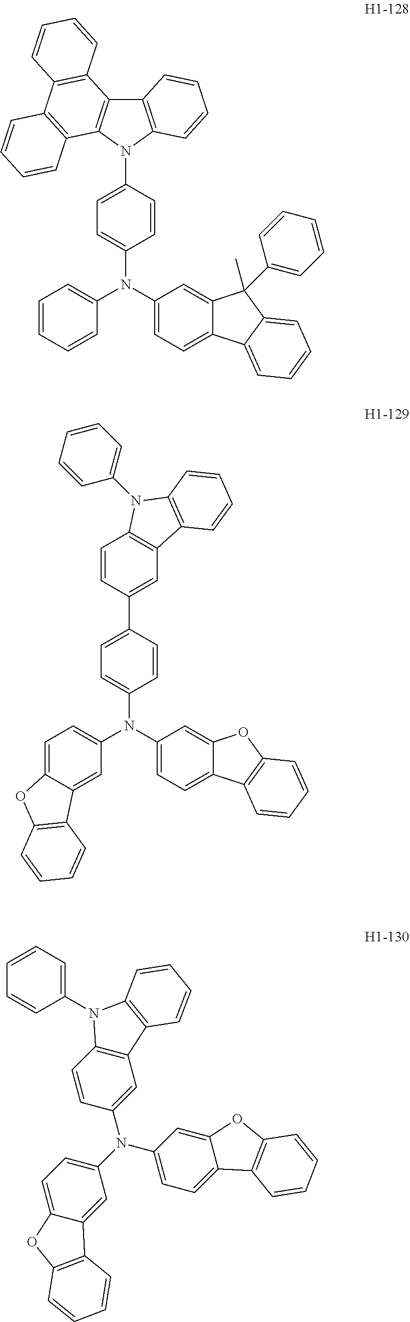 [SMILES: CC1(c2ccccc2)c2ccccc2-c2ccc(N(c3ccccc3)c3ccc(-n4c5ccccc5c5c6ccccc6c6ccccc6c54)cc3)cc21 ; c1ccc(-n2c3ccccc3c3cc(-c4ccc(N(c5ccc6c(c5)oc5ccccc56)c5ccc6oc7ccccc7c6c5)cc4)ccc32)cc1 ; c1ccc(-n2c3ccccc3c3cc(N(c4ccc5c(c4)oc4ccccc45)c4ccc5oc6ccccc6c5c4)ccc32)cc1]

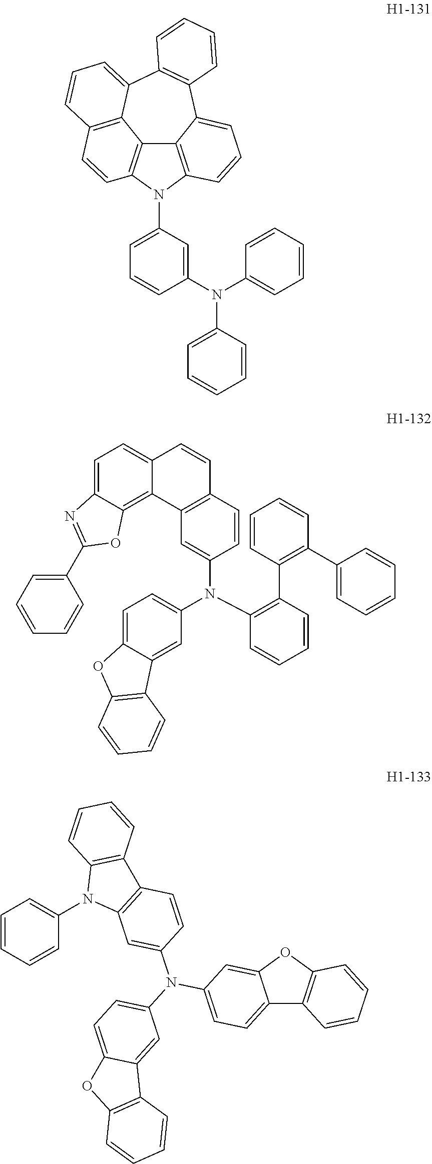 [SMILES: c1ccc(N(c2ccccc2)c2cccc(-n3c4cccc5c4c4c6c(cccc6ccc43)-c3ccccc3-5)c2)cc1 ; c1ccc(-c2nc3ccc4ccc5ccc(N(c6ccc7oc8ccccc8c7c6)c6ccccc6-c6ccccc6-c6ccccc6)cc5c4c3o2)cc1 ; c1ccc(-n2c3ccccc3c3ccc(N(c4ccc5c(c4)oc4ccccc45)c4ccc5oc6ccccc6c5c4)cc32)cc1]

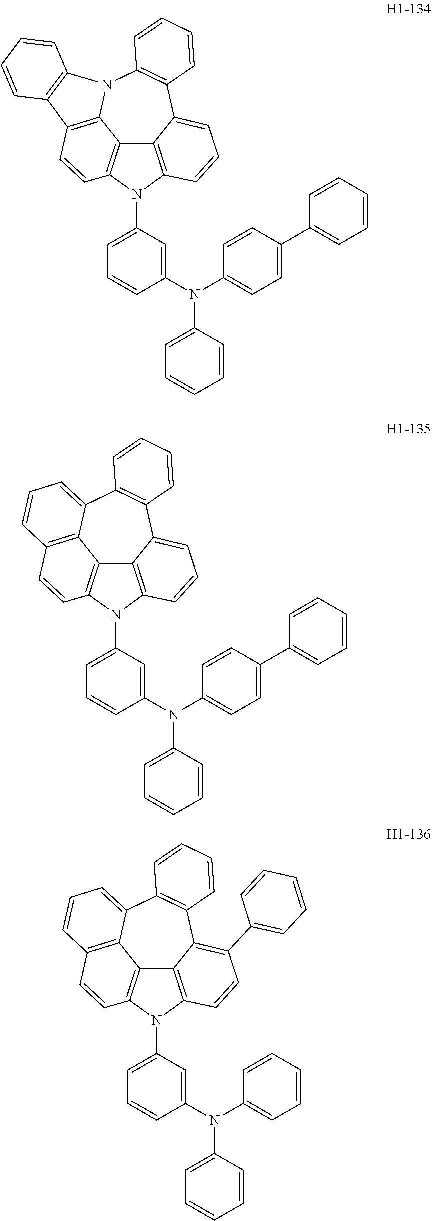

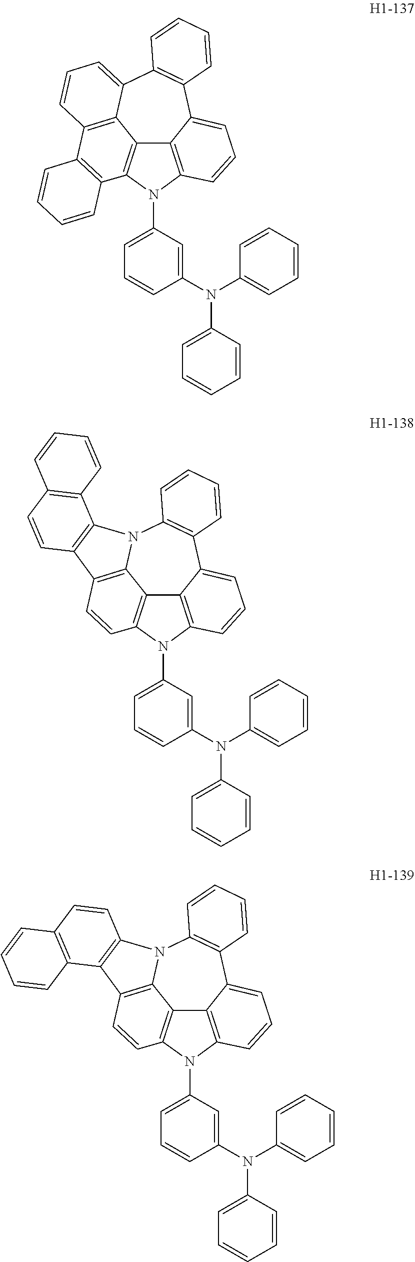

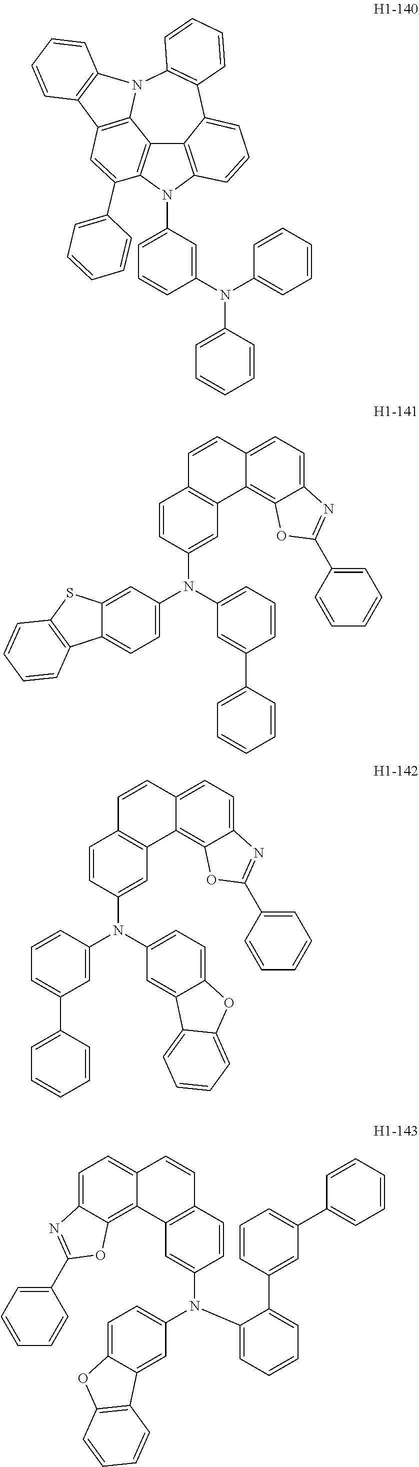

The compound represented by formula 2 may be at least one selected from the following compounds, but is not limited thereto.

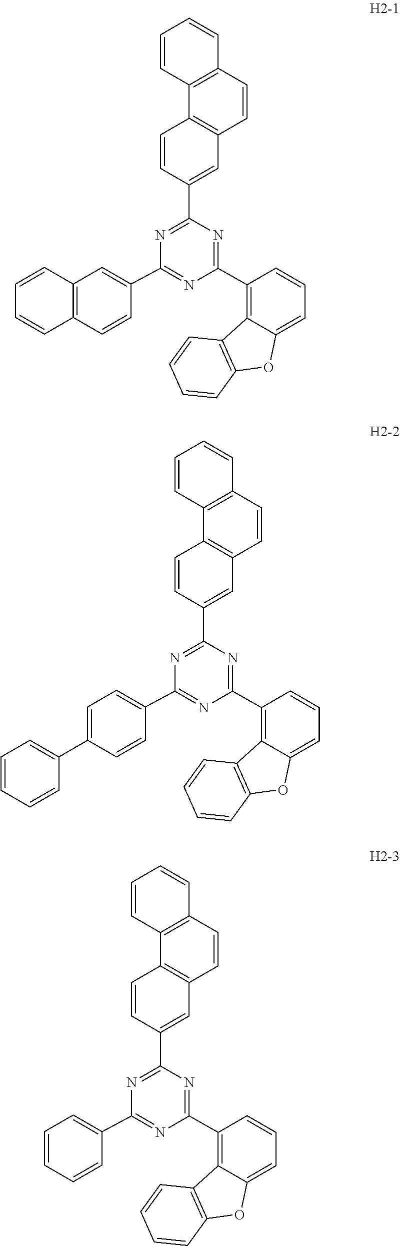

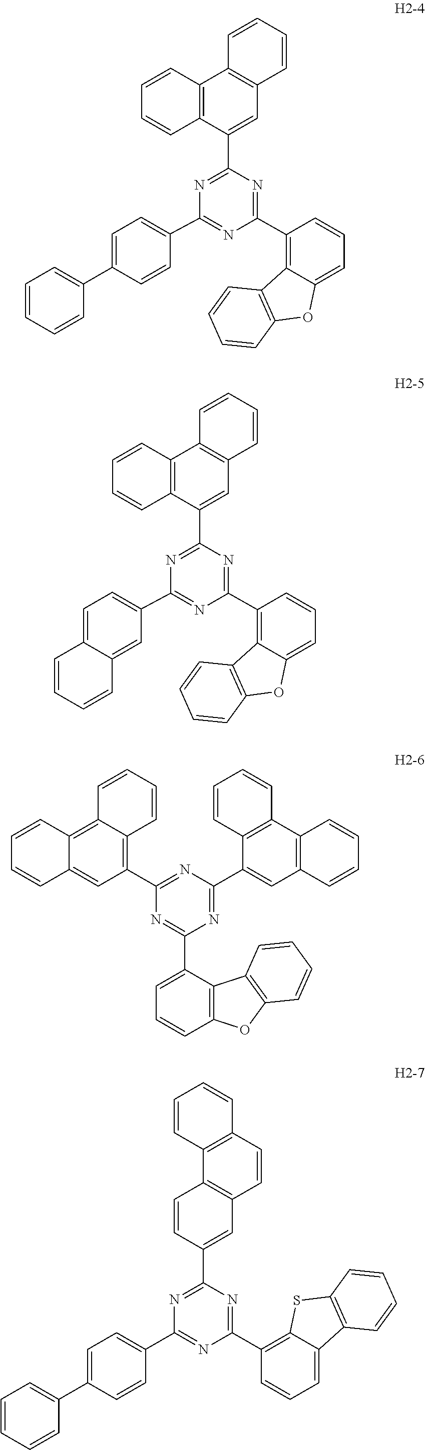 [SMILES: c1ccc(-c2ccc(-c3nc(-c4cc5ccccc5c5ccccc45)nc(-c4cccc5oc6ccccc6c45)n3)cc2)cc1 ; c1ccc2cc(-c3nc(-c4cc5ccccc5c5ccccc45)nc(-c4cccc5oc6ccccc6c45)n3)ccc2c1 ; c1ccc2c(c1)cc(-c1nc(-c3cc4ccccc4c4ccccc34)nc(-c3cccc4oc5ccccc5c34)n1)c1ccccc12 ; c1ccc(-c2ccc(-c3nc(-c4ccc5c(ccc6ccccc65)c4)nc(-c4cccc5c4sc4ccccc45)n3)cc2)cc1]

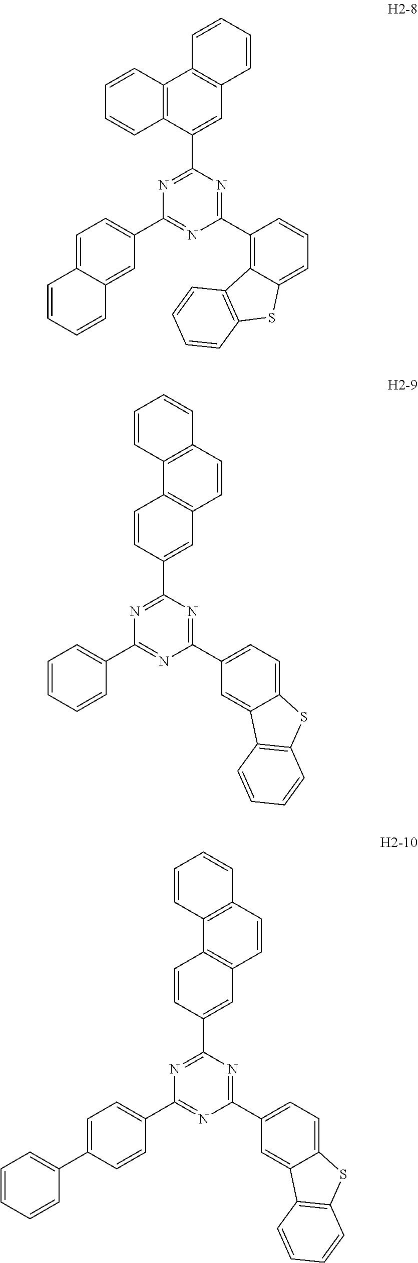 [SMILES: c1ccc2cc(-c3nc(-c4cc5ccccc5c5ccccc45)nc(-c4cccc5sc6ccccc6c45)n3)ccc2c1 ; c1ccc(-c2nc(-c3ccc4c(ccc5ccccc54)c3)nc(-c3ccc4sc5ccccc5c4c3)n2)cc1 ; c1ccc(-c2ccc(-c3nc(-c4ccc5c(ccc6ccccc65)c4)nc(-c4ccc5sc6ccccc6c5c4)n3)cc2)cc1]

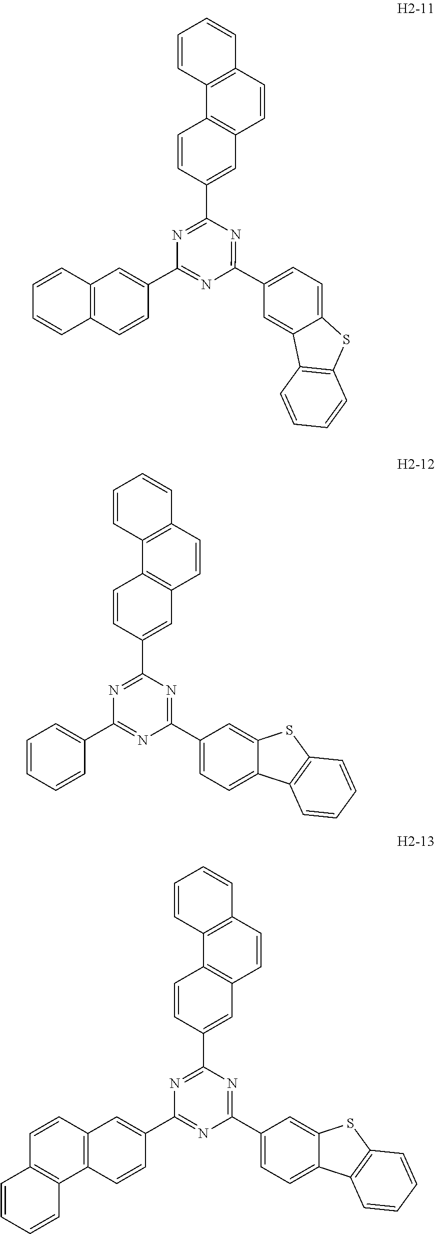

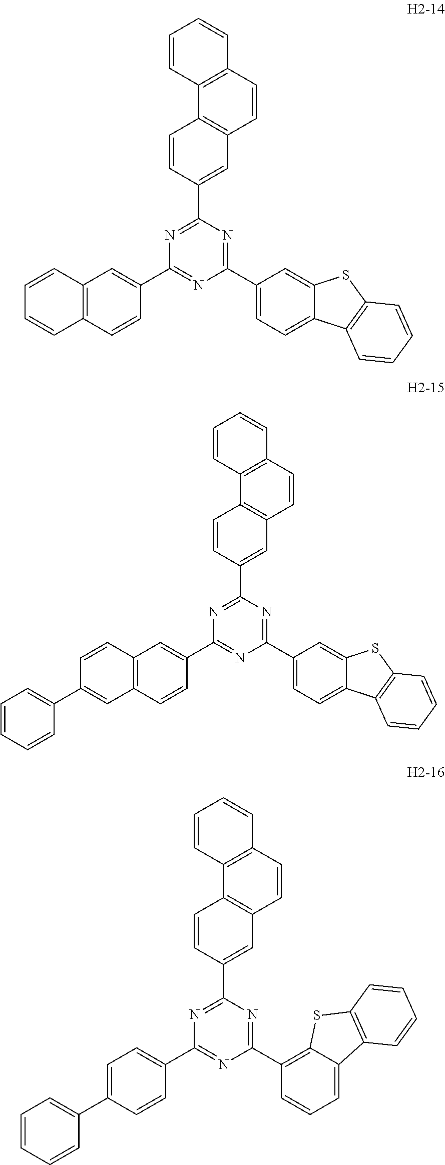 [SMILES: c1ccc2cc(-c3nc(-c4ccc5c(ccc6ccccc65)c4)nc(-c4ccc5c(c4)sc4ccccc45)n3)ccc2c1 ; c1ccc(-c2ccc3cc(-c4nc(-c5ccc6c(ccc7ccccc76)c5)nc(-c5ccc6c(c5)sc5ccccc56)n4)ccc3c2)cc1 ; c1ccc(-c2ccc(-c3nc(-c4ccc5c(ccc6ccccc65)c4)nc(-c4cccc5c4sc4ccccc45)n3)cc2)cc1]

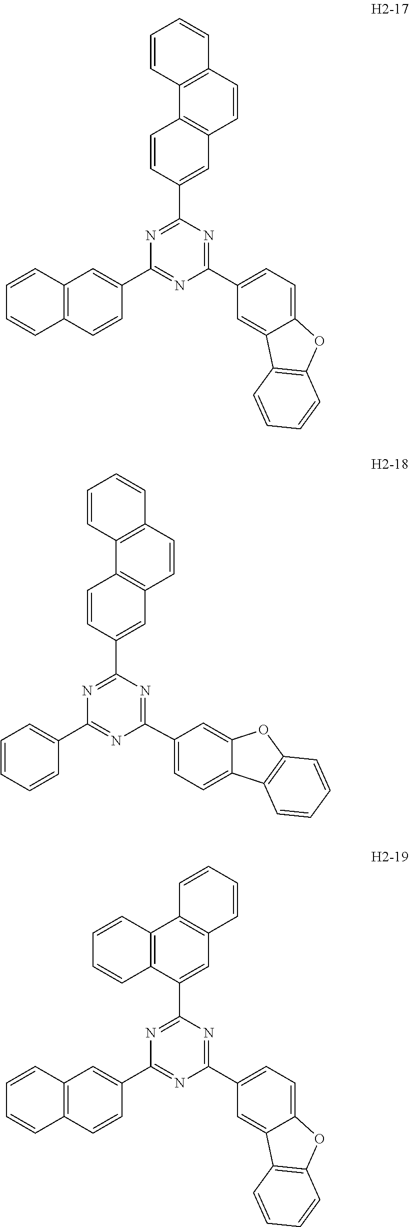 [SMILES: c1ccc2cc(-c3nc(-c4ccc5c(ccc6ccccc65)c4)nc(-c4ccc5oc6ccccc6c5c4)n3)ccc2c1 ; c1ccc(-c2nc(-c3ccc4c(ccc5ccccc54)c3)nc(-c3ccc4c(c3)oc3ccccc34)n2)cc1 ; c1ccc2cc(-c3nc(-c4ccc5oc6ccccc6c5c4)nc(-c4cc5ccccc5c5ccccc45)n3)ccc2c1]

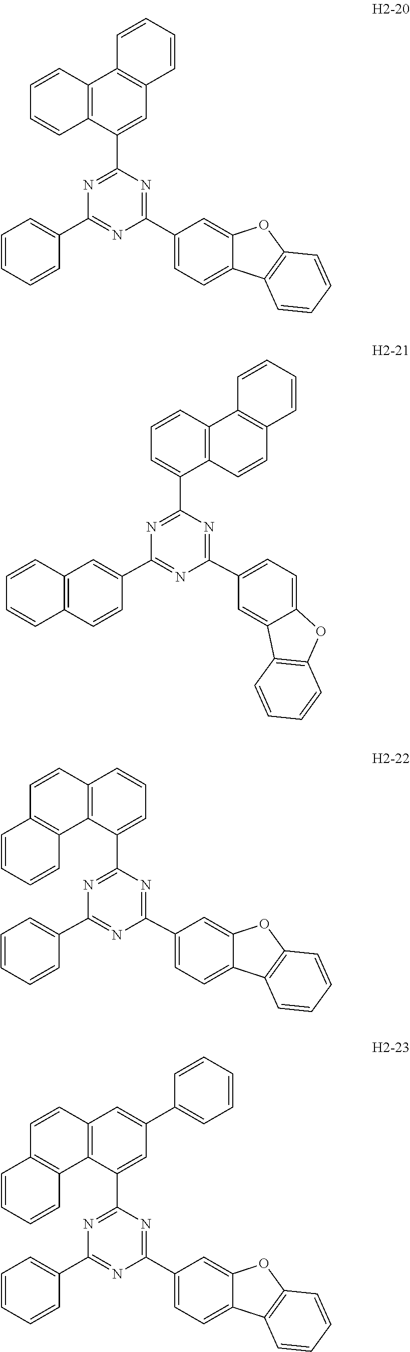 [SMILES: c1ccc(-c2nc(-c3ccc4c(c3)oc3ccccc34)nc(-c3cc4ccccc4c4ccccc34)n2)cc1 ; c1ccc2cc(-c3nc(-c4ccc5oc6ccccc6c5c4)nc(-c4cccc5c4ccc4ccccc45)n3)ccc2c1 ; c1ccc(-c2nc(-c3ccc4c(c3)oc3ccccc34)nc(-c3cccc4ccc5ccccc5c34)n2)cc1 ; c1ccc(-c2cc(-c3nc(-c4ccccc4)nc(-c4ccc5c(c4)oc4ccccc45)n3)c3c(ccc4ccccc43)c2)cc1]

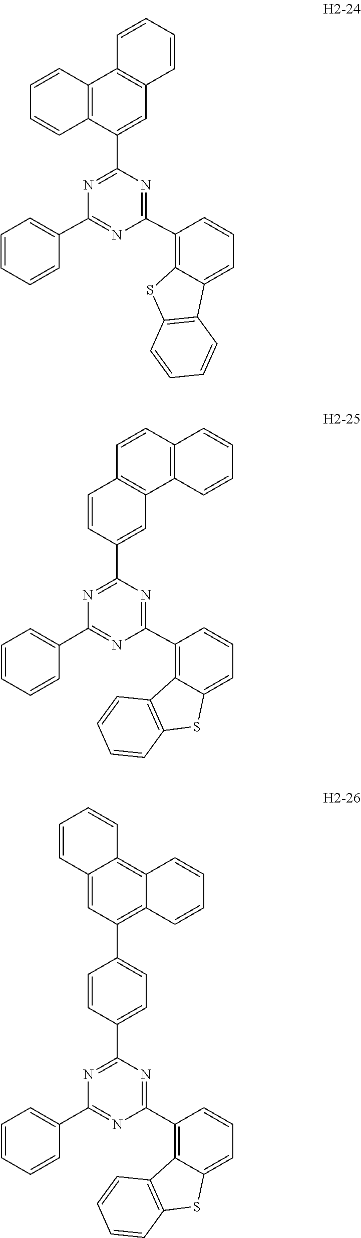 [SMILES: c1ccc(-c2nc(-c3cc4ccccc4c4ccccc34)nc(-c3cccc4c3sc3ccccc34)n2)cc1 ; c1ccc(-c2nc(-c3ccc4ccc5ccccc5c4c3)nc(-c3cccc4sc5ccccc5c34)n2)cc1 ; c1ccc(-c2nc(-c3ccc(-c4cc5ccccc5c5ccccc45)cc3)nc(-c3cccc4sc5ccccc5c34)n2)cc1]

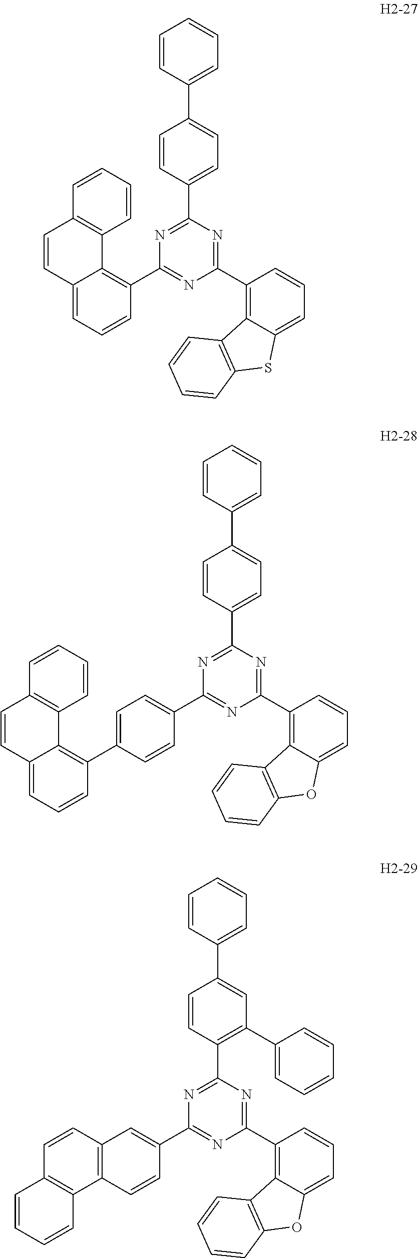 [SMILES: c1ccc(-c2ccc(-c3nc(-c4cccc5ccc6ccccc6c45)nc(-c4cccc5sc6ccccc6c45)n3)cc2)cc1 ; c1ccc(-c2ccc(-c3nc(-c4ccc(-c5cccc6ccc7ccccc7c56)cc4)nc(-c4cccc5oc6ccccc6c45)n3)cc2)cc1 ; c1ccc(-c2ccc(-c3nc(-c4ccc5c(ccc6ccccc65)c4)nc(-c4cccc5oc6ccccc6c45)n3)c(-c3ccccc3)c2)cc1]

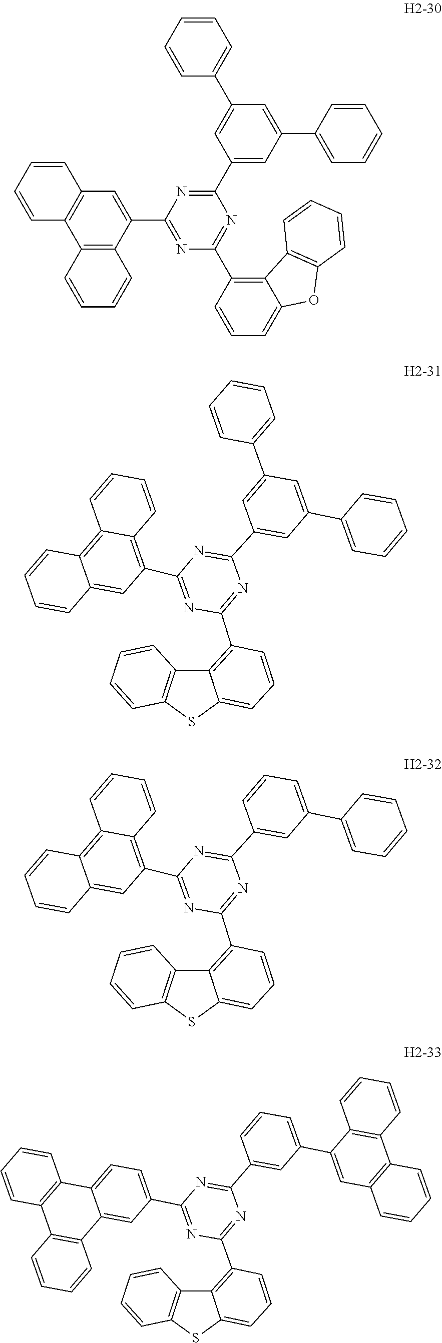

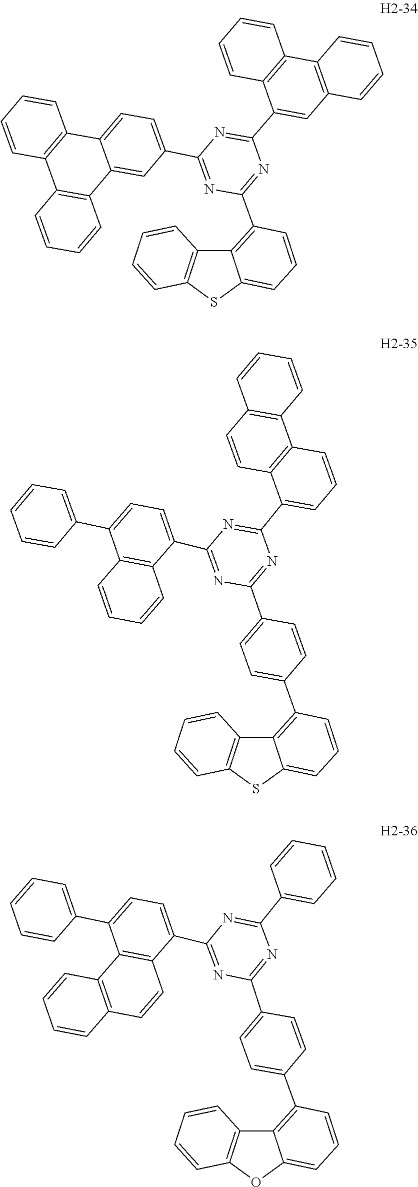 [SMILES: c1ccc2c(c1)cc(-c1nc(-c3ccc4c5ccccc5c5ccccc5c4c3)nc(-c3cccc4sc5ccccc5c34)n1)c1ccccc12 ; c1ccc(-c2ccc(-c3nc(-c4ccc(-c5cccc6sc7ccccc7c56)cc4)nc(-c4cccc5c4ccc4ccccc45)n3)c3ccccc23)cc1 ; c1ccc(-c2nc(-c3ccc(-c4cccc5oc6ccccc6c45)cc3)nc(-c3ccc(-c4ccccc4)c4c3ccc3ccccc34)n2)cc1]

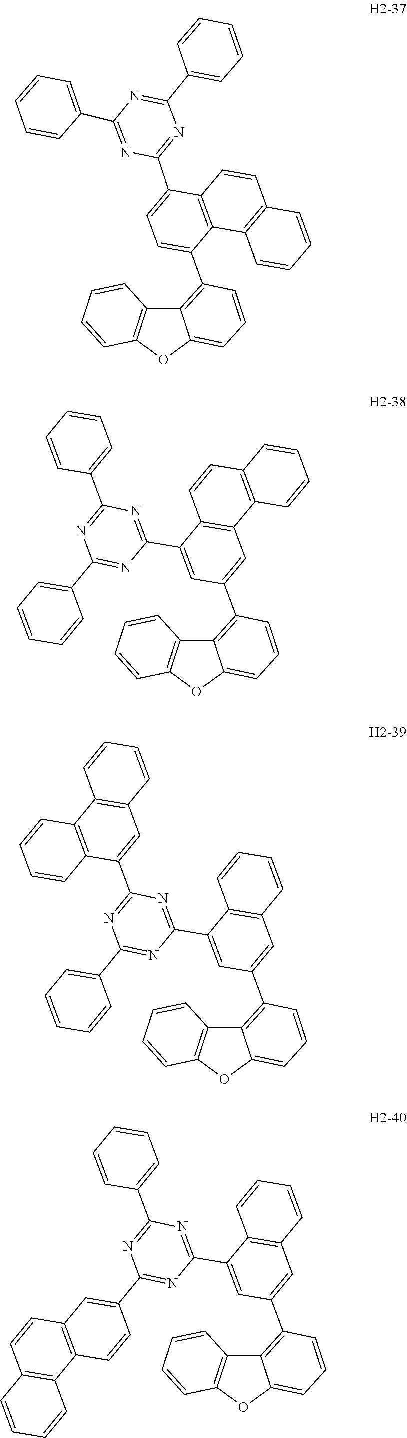 [SMILES: c1ccc(-c2nc(-c3ccccc3)nc(-c3ccc(-c4cccc5oc6ccccc6c45)c4c3ccc3ccccc34)n2)cc1 ; c1ccc(-c2nc(-c3ccccc3)nc(-c3cc(-c4cccc5oc6ccccc6c45)cc4c3ccc3ccccc34)n2)cc1 ; c1ccc(-c2nc(-c3cc(-c4cccc5oc6ccccc6c45)cc4ccccc34)nc(-c3cc4ccccc4c4ccccc34)n2)cc1 ; c1ccc(-c2nc(-c3ccc4c(ccc5ccccc54)c3)nc(-c3cc(-c4cccc5oc6ccccc6c45)cc4ccccc34)n2)cc1]

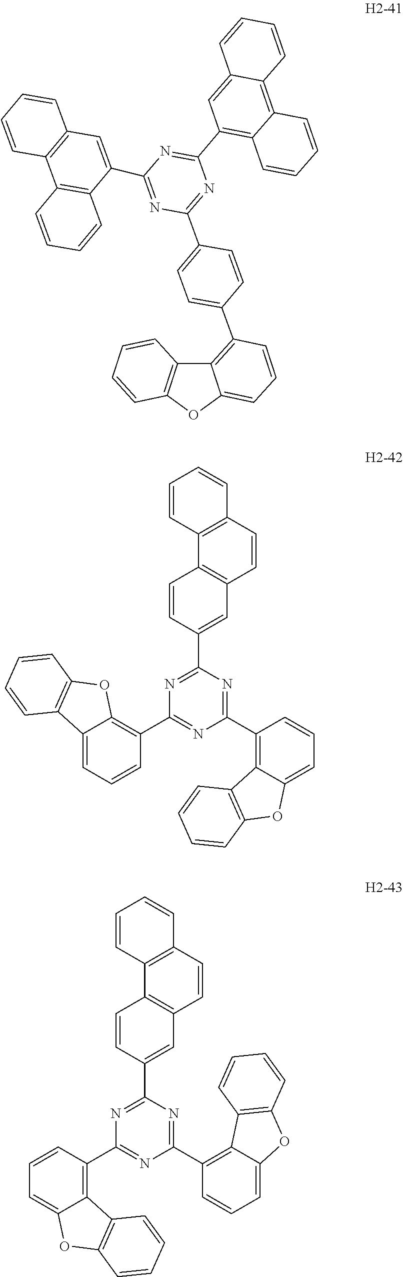 [SMILES: c1ccc2c(c1)cc(-c1nc(-c3ccc(-c4cccc5oc6ccccc6c45)cc3)nc(-c3cc4ccccc4c4ccccc34)n1)c1ccccc12 ; c1ccc2c(c1)ccc1cc(-c3nc(-c4cccc5c4oc4ccccc45)nc(-c4cccc5oc6ccccc6c45)n3)ccc12 ; c1ccc2c(c1)ccc1cc(-c3nc(-c4cccc5oc6ccccc6c45)nc(-c4cccc5oc6ccccc6c45)n3)ccc12]

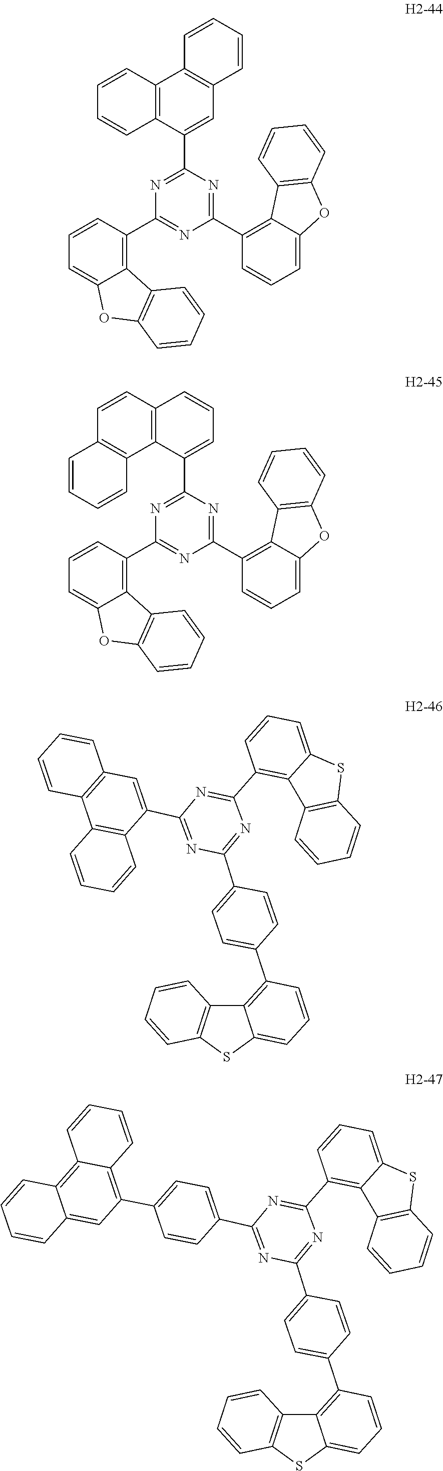 [SMILES: c1ccc2c(c1)cc(-c1nc(-c3cccc4oc5ccccc5c34)nc(-c3cccc4oc5ccccc5c34)n1)c1ccccc12 ; c1ccc2c(c1)ccc1cccc(-c3nc(-c4cccc5oc6ccccc6c45)nc(-c4cccc5oc6ccccc6c45)n3)c12 ; c1ccc2c(c1)cc(-c1nc(-c3ccc(-c4cccc5sc6ccccc6c45)cc3)nc(-c3cccc4sc5ccccc5c34)n1)c1ccccc12 ; c1ccc2c(c1)cc(-c1ccc(-c3nc(-c4ccc(-c5cccc6sc7ccccc7c56)cc4)nc(-c4cccc5sc6ccccc6c45)n3)cc1)c1ccccc12]

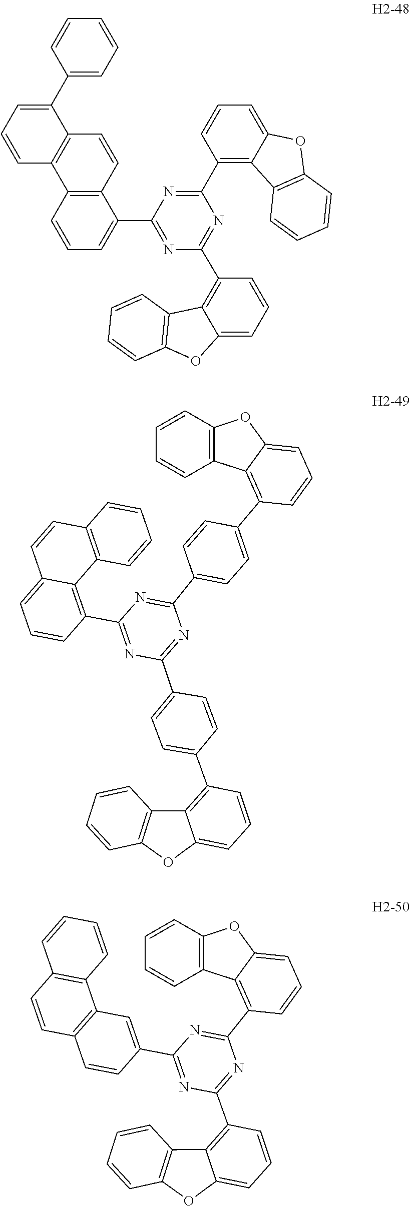 [SMILES: c1ccc(-c2cccc3c2ccc2c(-c4nc(-c5cccc6oc7ccccc7c56)nc(-c5cccc6oc7ccccc7c56)n4)cccc23)cc1 ; c1ccc2c(c1)ccc1cccc(-c3nc(-c4ccc(-c5cccc6oc7ccccc7c56)cc4)nc(-c4ccc(-c5cccc6oc7ccccc7c56)cc4)n3)c12 ; c1ccc2c(c1)ccc1ccc(-c3nc(-c4cccc5oc6ccccc6c45)nc(-c4cccc5oc6ccccc6c45)n3)cc12]

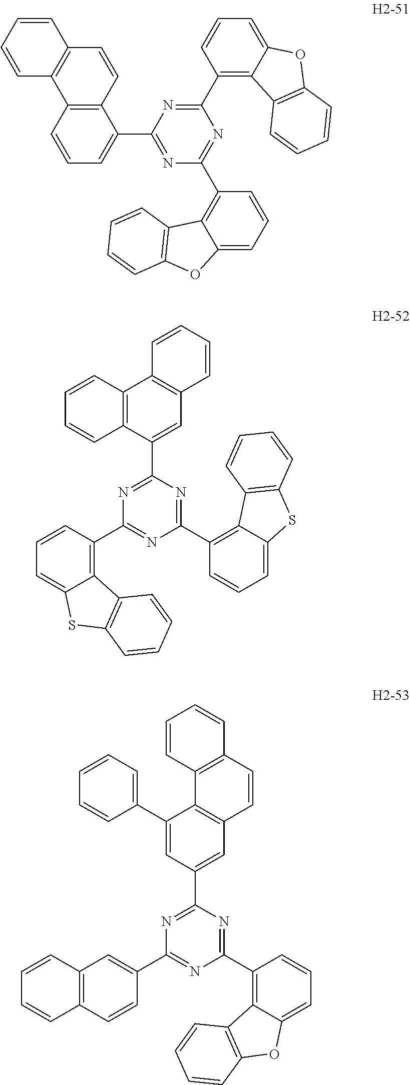 [SMILES: c1ccc2c(c1)ccc1c(-c3nc(-c4cccc5oc6ccccc6c45)nc(-c4cccc5oc6ccccc6c45)n3)cccc12 ; c1ccc2c(c1)cc(-c1nc(-c3cccc4sc5ccccc5c34)nc(-c3cccc4sc5ccccc5c34)n1)c1ccccc12 ; c1ccc(-c2cc(-c3nc(-c4ccc5ccccc5c4)nc(-c4cccc5oc6ccccc6c45)n3)cc3ccc4ccccc4c23)cc1]

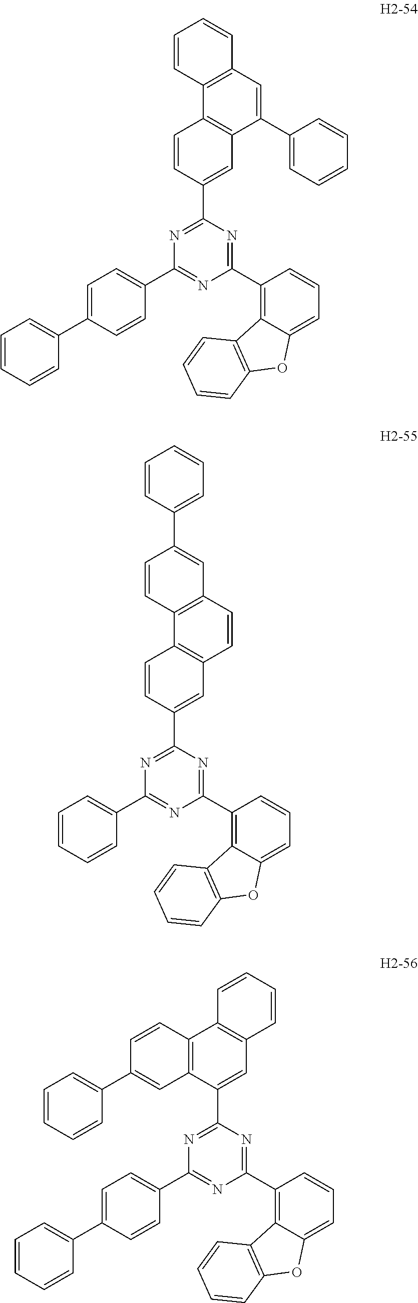 [SMILES: c1ccc(-c2ccc(-c3nc(-c4ccc5c(c4)c(-c4ccccc4)cc4ccccc45)nc(-c4cccc5oc6ccccc6c45)n3)cc2)cc1 ; c1ccc(-c2ccc3c(ccc4cc(-c5nc(-c6ccccc6)nc(-c6cccc7oc8ccccc8c67)n5)ccc43)c2)cc1 ; c1ccc(-c2ccc(-c3nc(-c4cc5ccccc5c5ccc(-c6ccccc6)cc45)nc(-c4cccc5oc6ccccc6c45)n3)cc2)cc1]

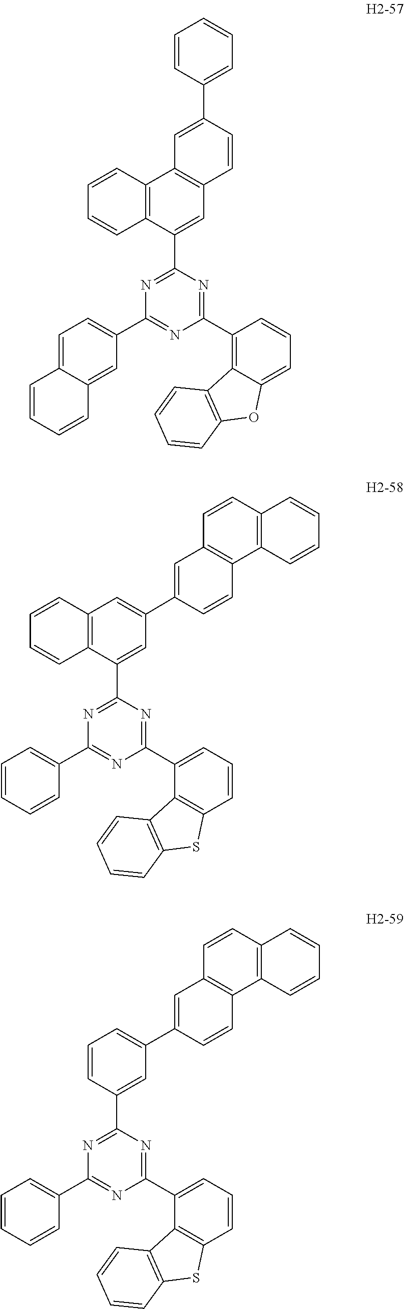 [SMILES: c1ccc(-c2ccc3cc(-c4nc(-c5ccc6ccccc6c5)nc(-c5cccc6oc7ccccc7c56)n4)c4ccccc4c3c2)cc1 ; c1ccc(-c2nc(-c3cc(-c4ccc5c(ccc6ccccc65)c4)cc4ccccc34)nc(-c3cccc4sc5ccccc5c34)n2)cc1 ; c1ccc(-c2nc(-c3cccc(-c4ccc5c(ccc6ccccc65)c4)c3)nc(-c3cccc4sc5ccccc5c34)n2)cc1]

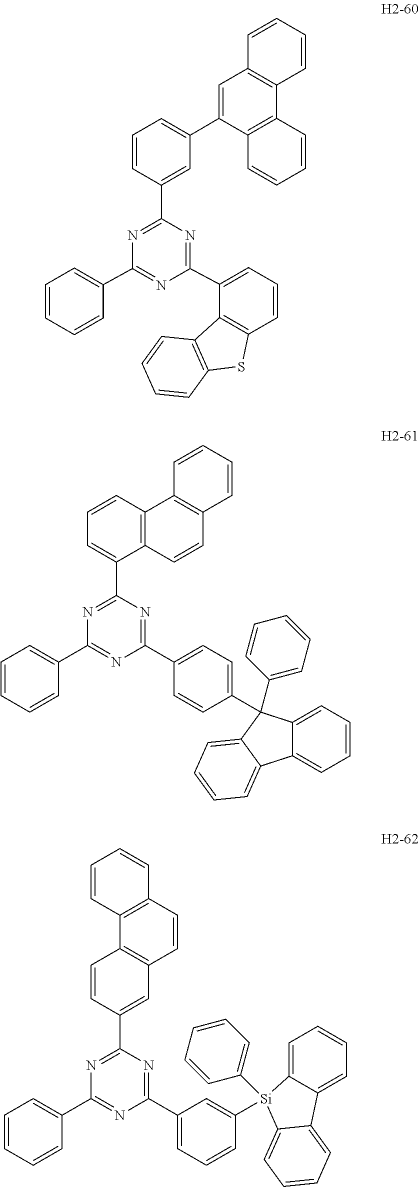 [SMILES: c1ccc(-c2nc(-c3cccc(-c4cc5ccccc5c5ccccc45)c3)nc(-c3cccc4sc5ccccc5c34)n2)cc1 ; c1ccc(-c2nc(-c3ccc(C4(c5ccccc5)c5ccccc5-c5ccccc54)cc3)nc(-c3cccc4c3ccc3ccccc34)n2)cc1 ; c1ccc(-c2nc(-c3cccc([Si]4(c5ccccc5)c5ccccc5-c5ccccc54)c3)nc(-c3ccc4c(ccc5ccccc54)c3)n2)cc1]

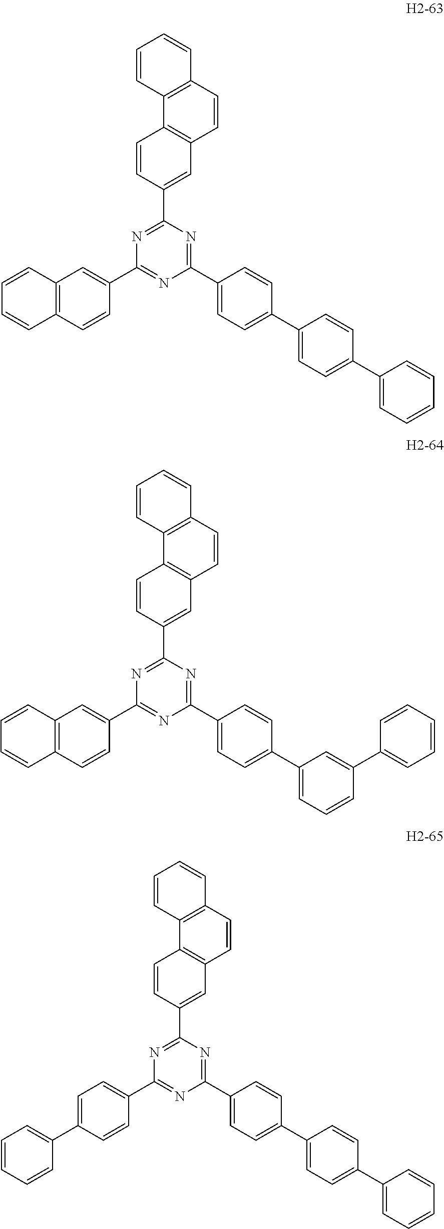 [SMILES: c1ccc(-c2ccc(-c3ccc(-c4nc(-c5ccc6ccccc6c5)nc(-c5ccc6c(ccc7ccccc76)c5)n4)cc3)cc2)cc1 ; c1ccc(-c2cccc(-c3ccc(-c4nc(-c5ccc6ccccc6c5)nc(-c5ccc6c(ccc7ccccc76)c5)n4)cc3)c2)cc1 ; c1ccc(-c2ccc(-c3ccc(-c4nc(-c5ccc(-c6ccccc6)cc5)nc(-c5ccc6c(ccc7ccccc76)c5)n4)cc3)cc2)cc1]

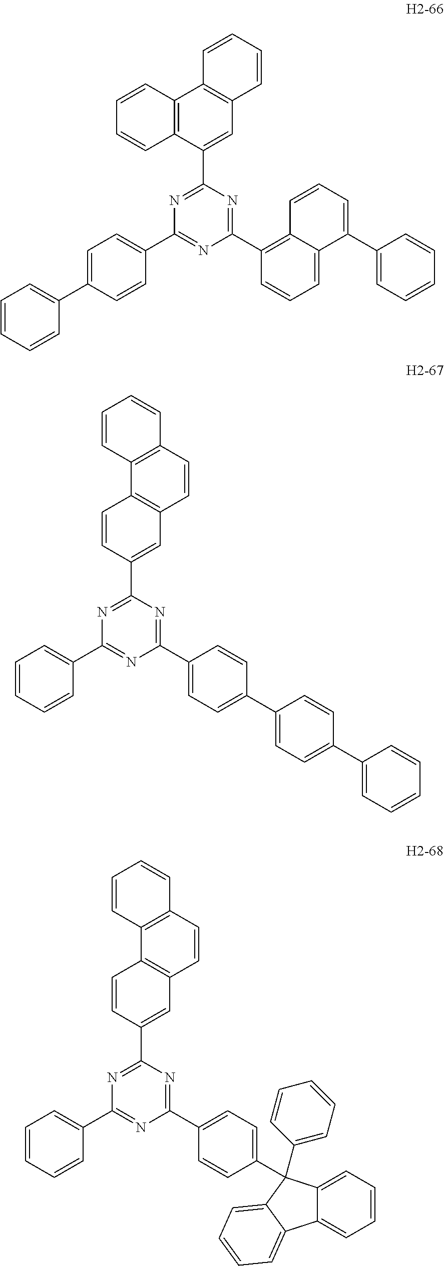 [SMILES: c1ccc(-c2ccc(-c3nc(-c4cccc5c(-c6ccccc6)cccc45)nc(-c4cc5ccccc5c5ccccc45)n3)cc2)cc1 ; c1ccc(-c2ccc(-c3ccc(-c4nc(-c5ccccc5)nc(-c5ccc6c(ccc7ccccc76)c5)n4)cc3)cc2)cc1 ; c1ccc(-c2nc(-c3ccc(C4(c5ccccc5)c5ccccc5-c5ccccc54)cc3)nc(-c3ccc4c(ccc5ccccc54)c3)n2)cc1]

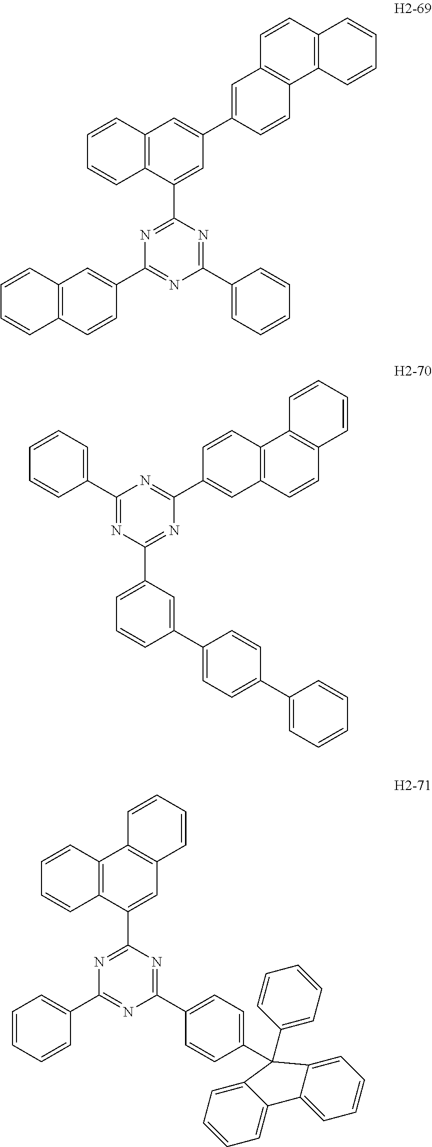 [SMILES: c1ccc(-c2nc(-c3ccc4ccccc4c3)nc(-c3cc(-c4ccc5c(ccc6ccccc65)c4)cc4ccccc34)n2)cc1 ; c1ccc(-c2ccc(-c3cccc(-c4nc(-c5ccccc5)nc(-c5ccc6c(ccc7ccccc76)c5)n4)c3)cc2)cc1 ; c1ccc(-c2nc(-c3ccc(C4(c5ccccc5)c5ccccc5-c5ccccc54)cc3)nc(-c3cc4ccccc4c4ccccc34)n2)cc1]

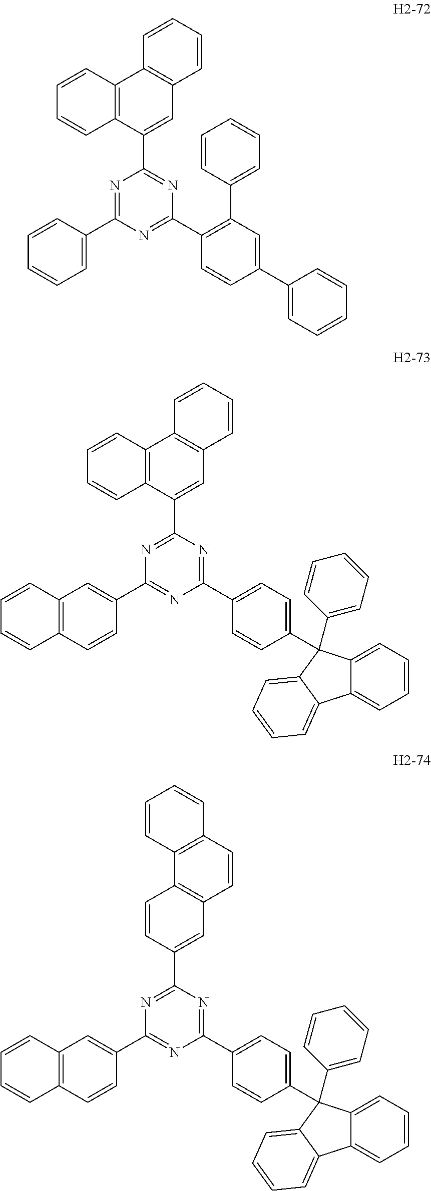 [SMILES: c1ccc(-c2ccc(-c3nc(-c4ccccc4)nc(-c4cc5ccccc5c5ccccc45)n3)c(-c3ccccc3)c2)cc1 ; c1ccc(C2(c3ccc(-c4nc(-c5ccc6ccccc6c5)nc(-c5cc6ccccc6c6ccccc56)n4)cc3)c3ccccc3-c3ccccc32)cc1 ; c1ccc(C2(c3ccc(-c4nc(-c5ccc6ccccc6c5)nc(-c5ccc6c(ccc7ccccc76)c5)n4)cc3)c3ccccc3-c3ccccc32)cc1]

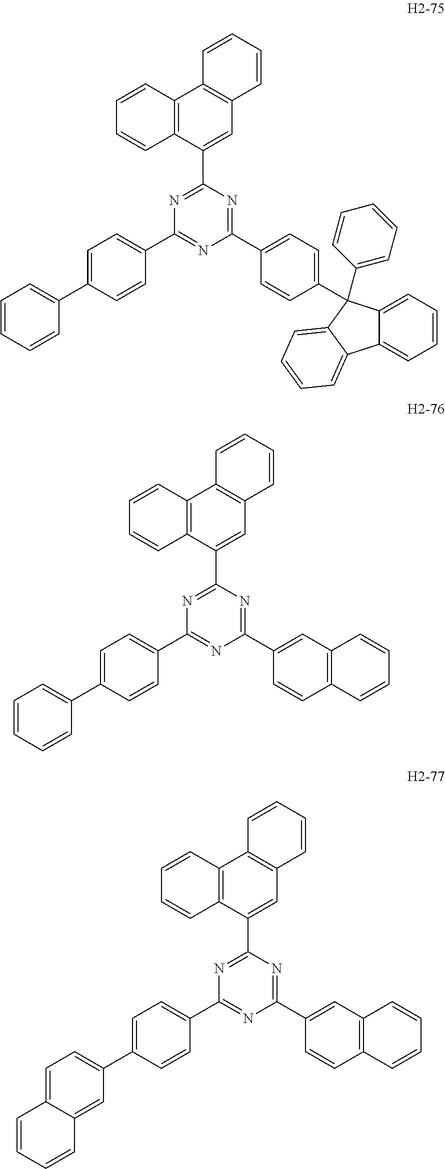 [SMILES: c1ccc(-c2ccc(-c3nc(-c4ccc(C5(c6ccccc6)c6ccccc6-c6ccccc65)cc4)nc(-c4cc5ccccc5c5ccccc45)n3)cc2)cc1 ; c1ccc(-c2ccc(-c3nc(-c4ccc5ccccc5c4)nc(-c4cc5ccccc5c5ccccc45)n3)cc2)cc1 ; c1ccc2cc(-c3ccc(-c4nc(-c5ccc6ccccc6c5)nc(-c5cc6ccccc6c6ccccc56)n4)cc3)ccc2c1]

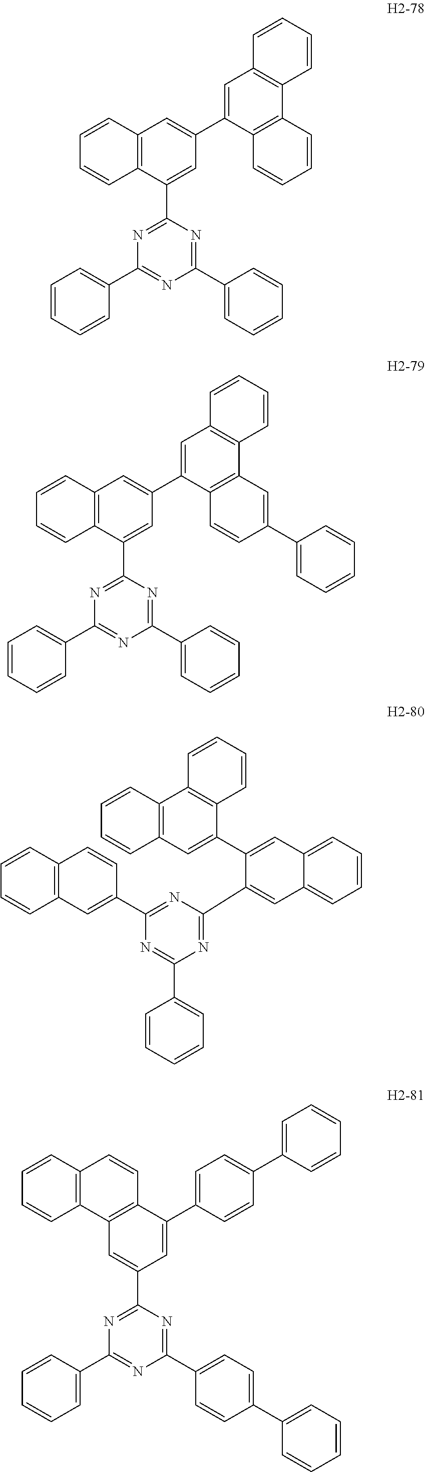

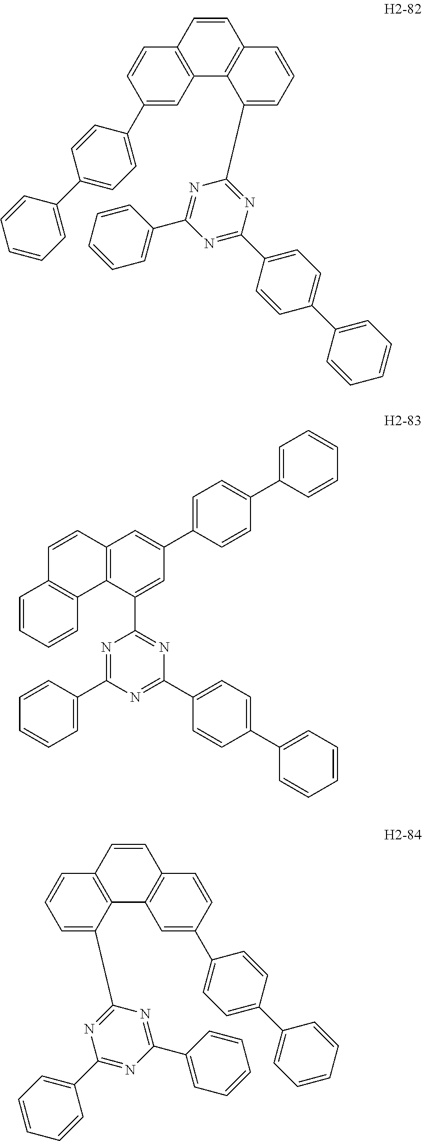 [SMILES: c1ccc(-c2ccc(-c3ccc4ccc5cccc(-c6nc(-c7ccccc7)nc(-c7ccc(-c8ccccc8)cc7)n6)c5c4c3)cc2)cc1 ; c1ccc(-c2ccc(-c3cc(-c4nc(-c5ccccc5)nc(-c5ccc(-c6ccccc6)cc5)n4)c4c(ccc5ccccc54)c3)cc2)cc1 ; c1ccc(-c2ccc(-c3ccc4ccc5cccc(-c6nc(-c7ccccc7)nc(-c7ccccc7)n6)c5c4c3)cc2)cc1]

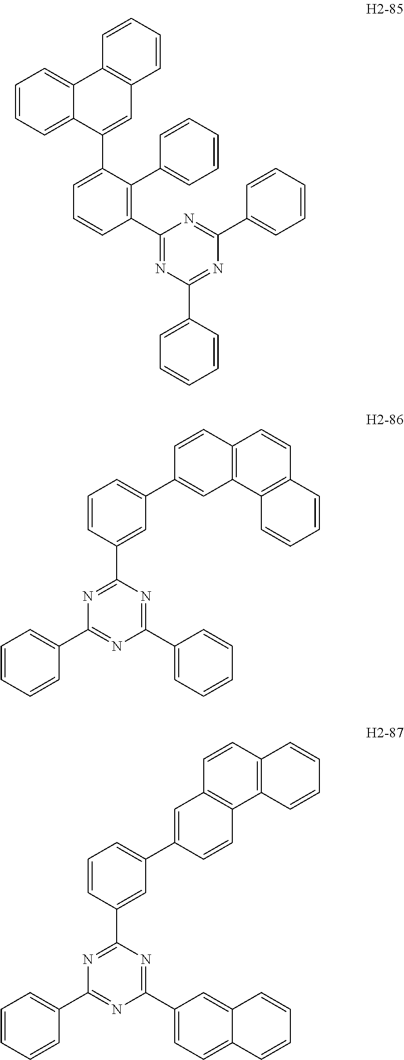

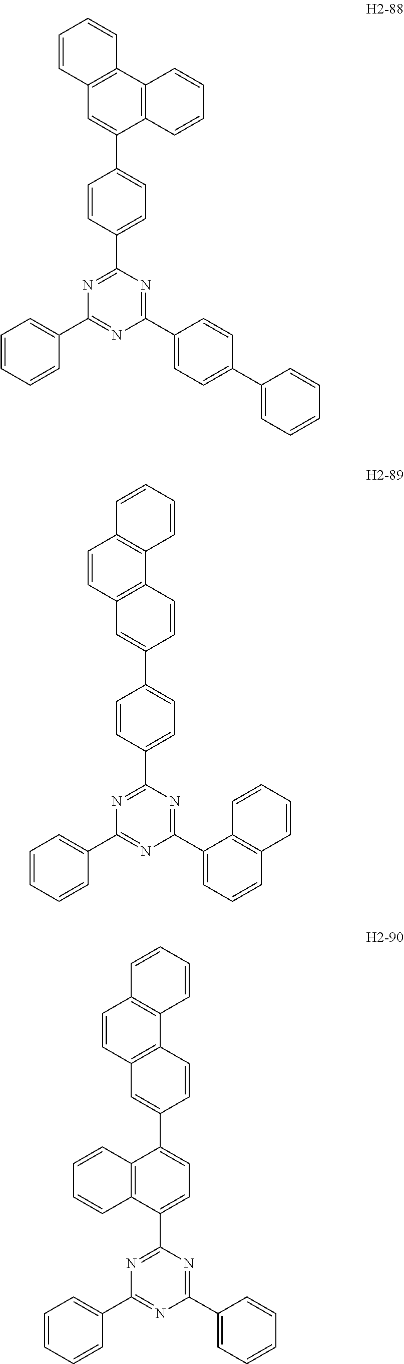 [SMILES: c1ccc(-c2ccc(-c3nc(-c4ccccc4)nc(-c4ccc(-c5cc6ccccc6c6ccccc56)cc4)n3)cc2)cc1 ; c1ccc(-c2nc(-c3ccc(-c4ccc5c(ccc6ccccc65)c4)cc3)nc(-c3cccc4ccccc34)n2)cc1 ; c1ccc(-c2nc(-c3ccccc3)nc(-c3ccc(-c4ccc5c(ccc6ccccc65)c4)c4ccccc34)n2)cc1]

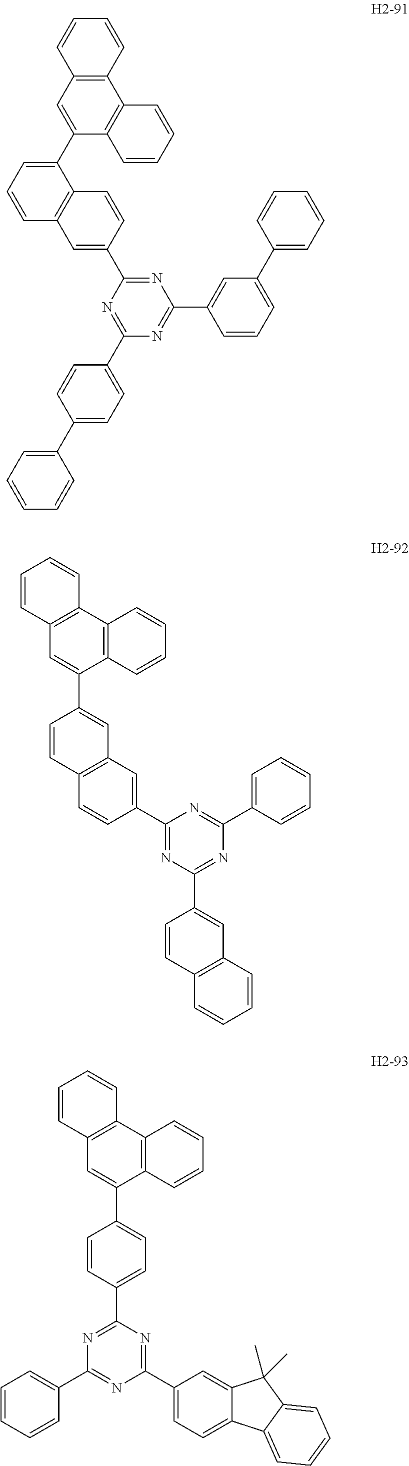 [SMILES: c1ccc(-c2ccc(-c3nc(-c4cccc(-c5ccccc5)c4)nc(-c4ccc5c(-c6cc7ccccc7c7ccccc67)cccc5c4)n3)cc2)cc1 ; c1ccc(-c2nc(-c3ccc4ccccc4c3)nc(-c3ccc4ccc(-c5cc6ccccc6c6ccccc56)cc4c3)n2)cc1 ; CC1(C)c2ccccc2-c2ccc(-c3nc(-c4ccccc4)nc(-c4ccc(-c5cc6ccccc6c6ccccc56)cc4)n3)cc21]

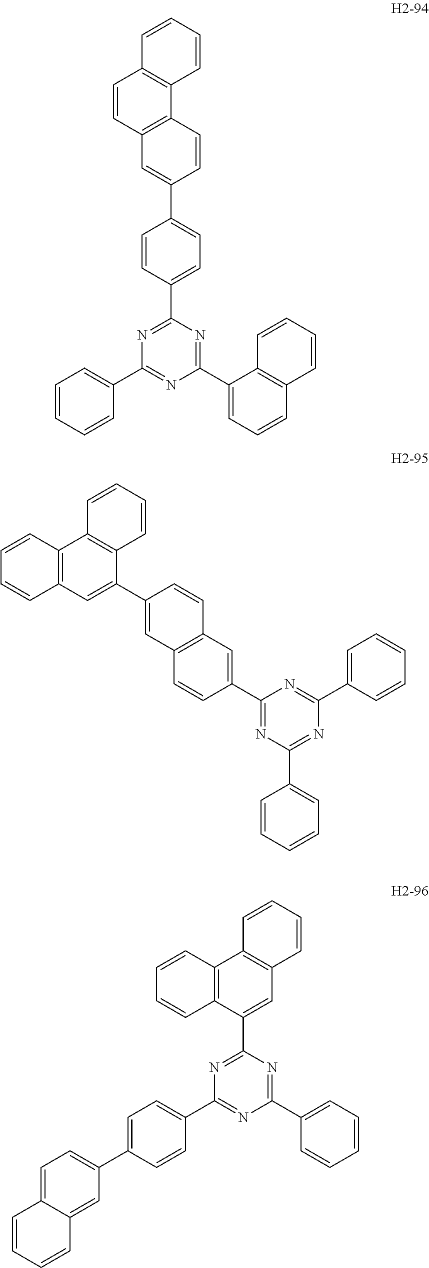 [SMILES: c1ccc(-c2nc(-c3ccc(-c4ccc5c(ccc6ccccc65)c4)cc3)nc(-c3cccc4ccccc34)n2)cc1 ; c1ccc(-c2nc(-c3ccccc3)nc(-c3ccc4cc(-c5cc6ccccc6c6ccccc56)ccc4c3)n2)cc1 ; c1ccc(-c2nc(-c3ccc(-c4ccc5ccccc5c4)cc3)nc(-c3cc4ccccc4c4ccccc34)n2)cc1]

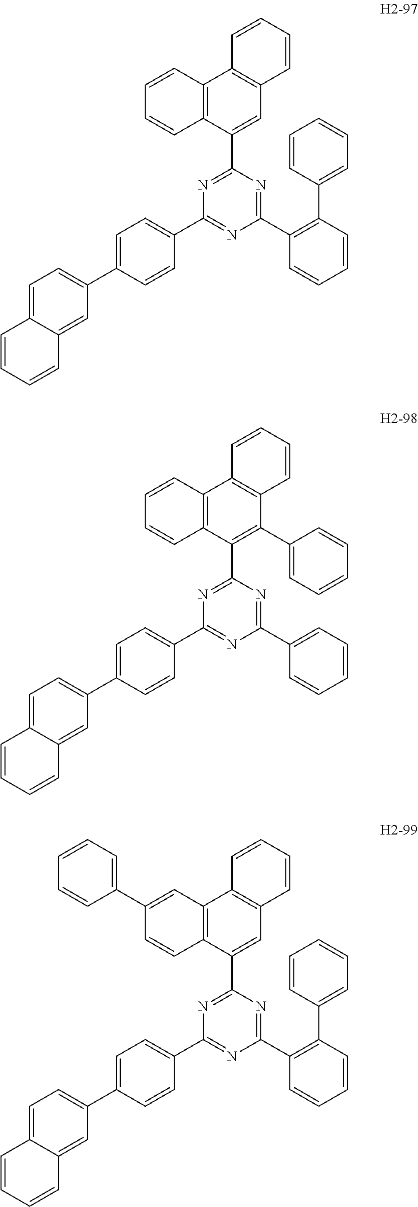 [SMILES: c1ccc(-c2ccccc2-c2nc(-c3ccc(-c4ccc5ccccc5c4)cc3)nc(-c3cc4ccccc4c4ccccc34)n2)cc1 ; c1ccc(-c2nc(-c3ccc(-c4ccc5ccccc5c4)cc3)nc(-c3c(-c4ccccc4)c4ccccc4c4ccccc34)n2)cc1 ; c1ccc(-c2ccc3c(-c4nc(-c5ccc(-c6ccc7ccccc7c6)cc5)nc(-c5ccccc5-c5ccccc5)n4)cc4ccccc4c3c2)cc1]

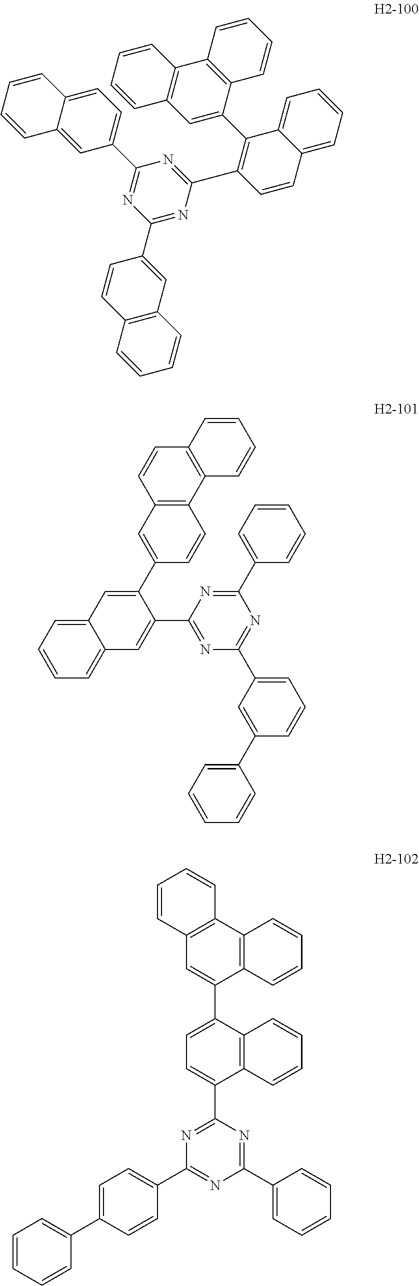 [SMILES: c1ccc2cc(-c3nc(-c4ccc5ccccc5c4)nc(-c4ccc5ccccc5c4-c4cc5ccccc5c5ccccc45)n3)ccc2c1 ; c1ccc(-c2cccc(-c3nc(-c4ccccc4)nc(-c4cc5ccccc5cc4-c4ccc5c(ccc6ccccc65)c4)n3)c2)cc1 ; c1ccc(-c2ccc(-c3nc(-c4ccccc4)nc(-c4ccc(-c5cc6ccccc6c6ccccc56)c5ccccc45)n3)cc2)cc1]

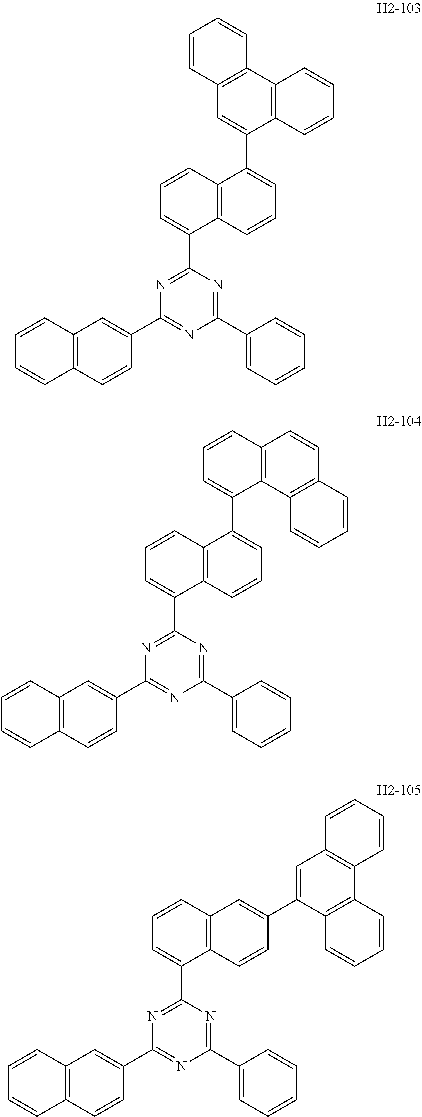 [SMILES: c1ccc(-c2nc(-c3ccc4ccccc4c3)nc(-c3cccc4c(-c5cc6ccccc6c6ccccc56)cccc34)n2)cc1 ; c1ccc(-c2nc(-c3ccc4ccccc4c3)nc(-c3cccc4c(-c5cccc6ccc7ccccc7c56)cccc34)n2)cc1 ; c1ccc(-c2nc(-c3ccc4ccccc4c3)nc(-c3cccc4cc(-c5cc6ccccc6c6ccccc56)ccc34)n2)cc1]

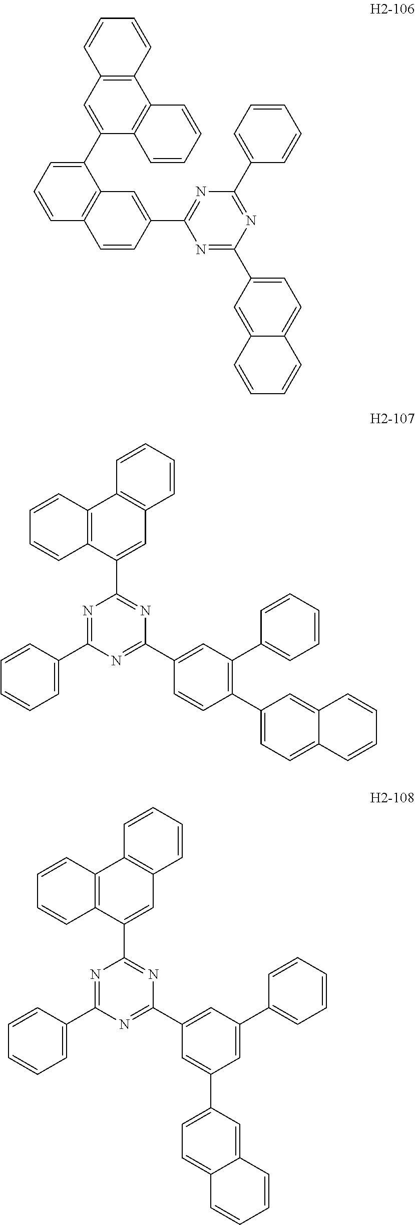 [SMILES: c1ccc(-c2nc(-c3ccc4ccccc4c3)nc(-c3ccc4cccc(-c5cc6ccccc6c6ccccc56)c4c3)n2)cc1 ; c1ccc(-c2nc(-c3ccc(-c4ccc5ccccc5c4)c(-c4ccccc4)c3)nc(-c3cc4ccccc4c4ccccc34)n2)cc1 ; c1ccc(-c2cc(-c3ccc4ccccc4c3)cc(-c3nc(-c4ccccc4)nc(-c4cc5ccccc5c5ccccc45)n3)c2)cc1]

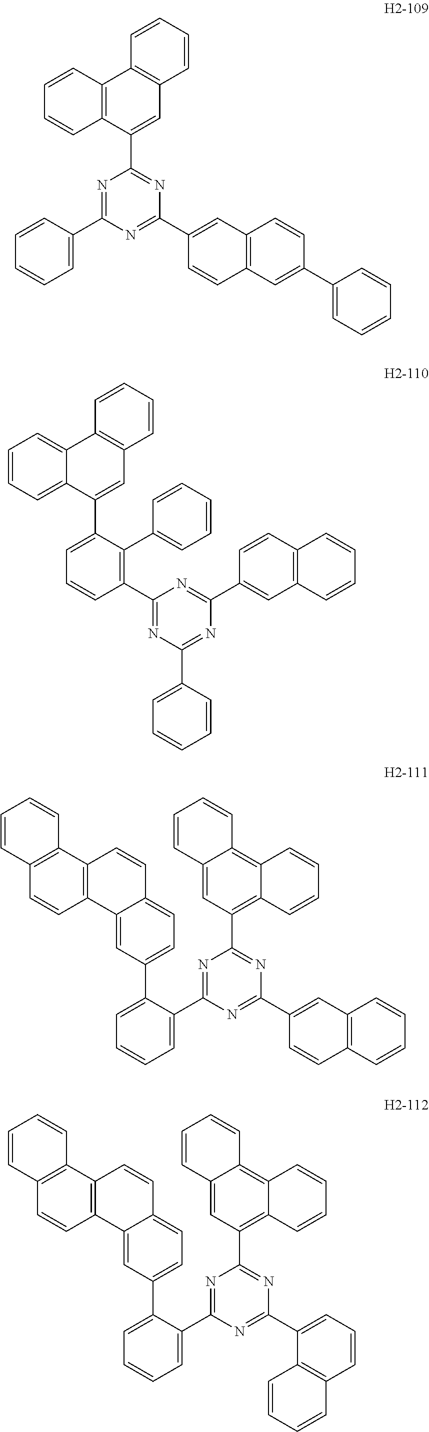 [SMILES: c1ccc(-c2ccc3cc(-c4nc(-c5ccccc5)nc(-c5cc6ccccc6c6ccccc56)n4)ccc3c2)cc1 ; c1ccc(-c2nc(-c3ccc4ccccc4c3)nc(-c3cccc(-c4cc5ccccc5c5ccccc45)c3-c3ccccc3)n2)cc1 ; c1ccc(-c2nc(-c3ccc4ccccc4c3)nc(-c3cc4ccccc4c4ccccc34)n2)c(-c2ccc3ccc4c5ccccc5ccc4c3c2)c1 ; c1ccc(-c2nc(-c3cccc4ccccc34)nc(-c3cc4ccccc4c4ccccc34)n2)c(-c2ccc3ccc4c5ccccc5ccc4c3c2)c1]

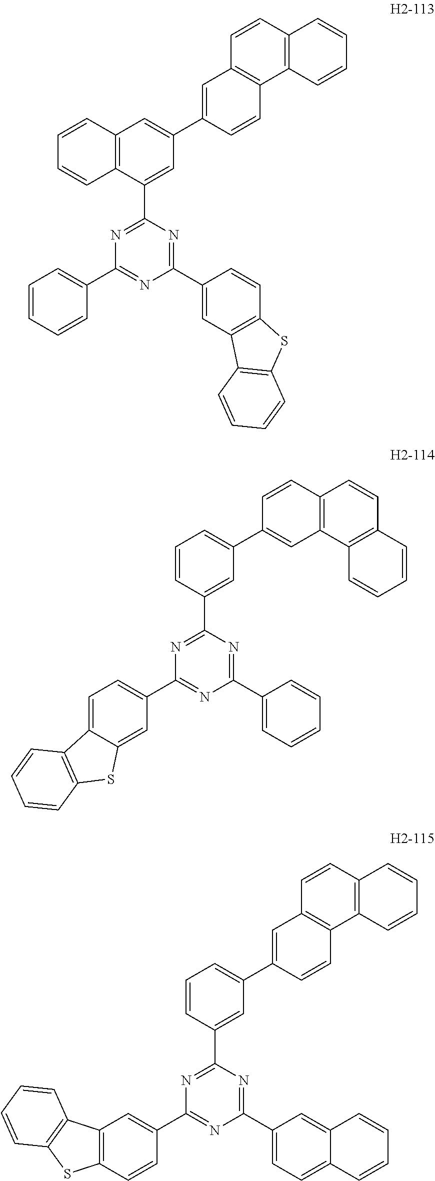 [SMILES: c1ccc(-c2nc(-c3ccc4sc5ccccc5c4c3)nc(-c3cc(-c4ccc5c(ccc6ccccc65)c4)cc4ccccc34)n2)cc1 ; c1ccc(-c2nc(-c3cccc(-c4ccc5ccc6ccccc6c5c4)c3)nc(-c3ccc4c(c3)sc3ccccc34)n2)cc1 ; c1cc(-c2ccc3c(ccc4ccccc43)c2)cc(-c2nc(-c3ccc4ccccc4c3)nc(-c3ccc4sc5ccccc5c4c3)n2)c1]

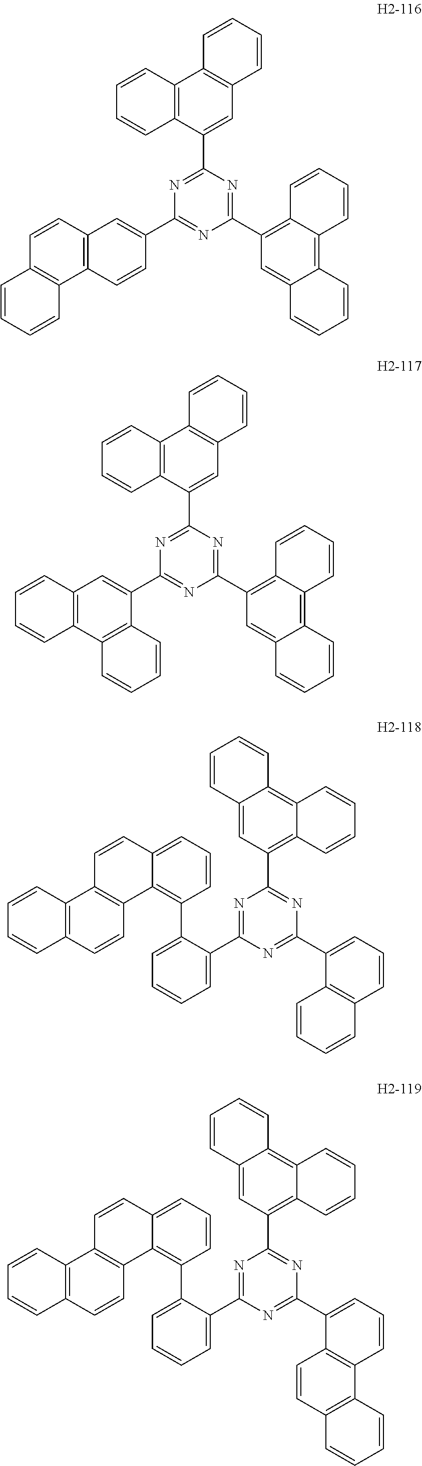 [SMILES: c1ccc2c(c1)ccc1cc(-c3nc(-c4cc5ccccc5c5ccccc45)nc(-c4cc5ccccc5c5ccccc45)n3)ccc12 ; c1ccc2c(c1)cc(-c1nc(-c3cc4ccccc4c4ccccc34)nc(-c3cc4ccccc4c4ccccc34)n1)c1ccccc12 ; c1ccc(-c2cccc3ccc4c5ccccc5ccc4c23)c(-c2nc(-c3cccc4ccccc34)nc(-c3cc4ccccc4c4ccccc34)n2)c1 ; c1ccc(-c2cccc3ccc4c5ccccc5ccc4c23)c(-c2nc(-c3cccc4c3ccc3ccccc34)nc(-c3cc4ccccc4c4ccccc34)n2)c1]

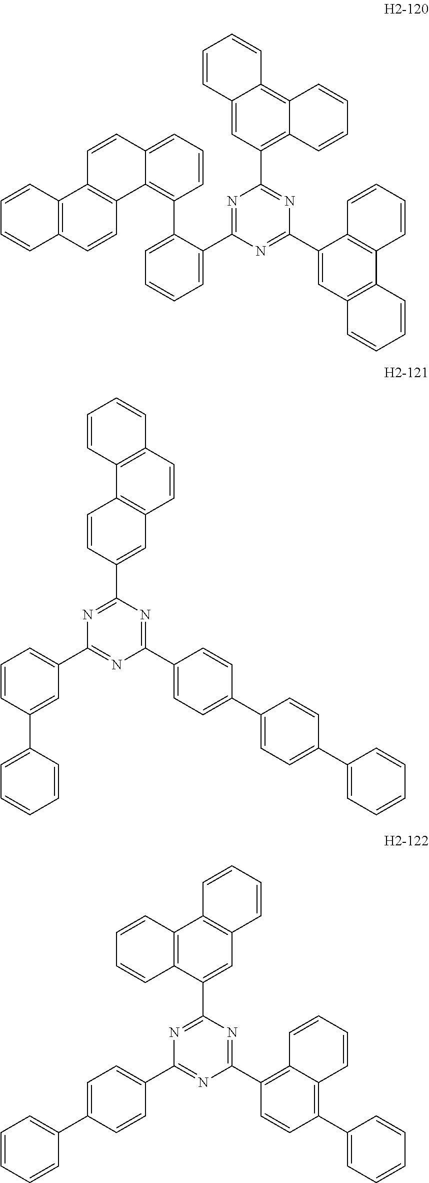 [SMILES: c1ccc(-c2cccc3ccc4c5ccccc5ccc4c23)c(-c2nc(-c3cc4ccccc4c4ccccc34)nc(-c3cc4ccccc4c4ccccc34)n2)c1 ; c1ccc(-c2ccc(-c3ccc(-c4nc(-c5cccc(-c6ccccc6)c5)nc(-c5ccc6c(ccc7ccccc76)c5)n4)cc3)cc2)cc1 ; c1ccc(-c2ccc(-c3nc(-c4ccc(-c5ccccc5)c5ccccc45)nc(-c4cc5ccccc5c5ccccc45)n3)cc2)cc1]

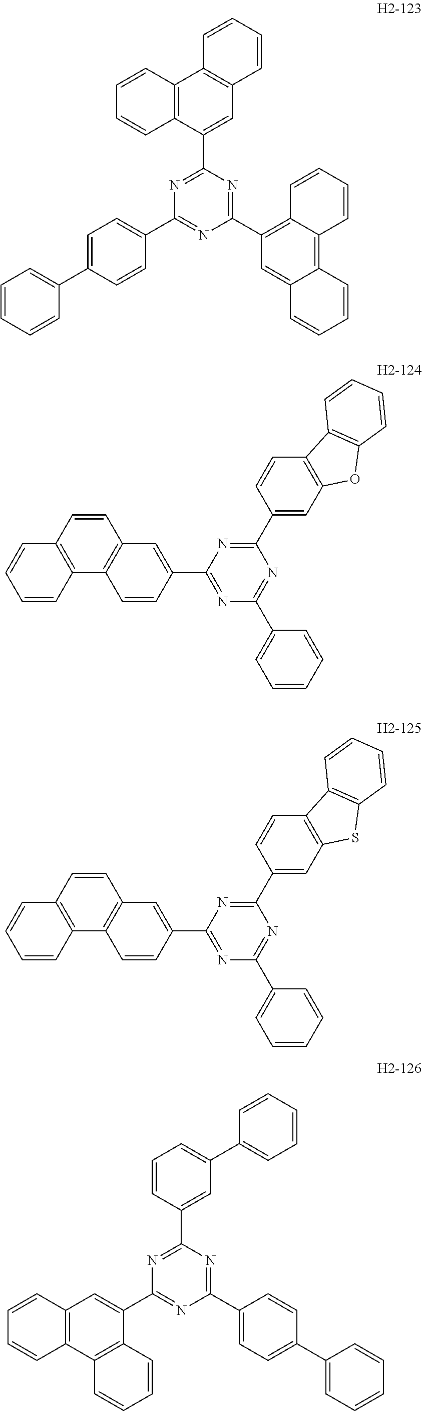 [SMILES: c1ccc(-c2ccc(-c3nc(-c4cc5ccccc5c5ccccc45)nc(-c4cc5ccccc5c5ccccc45)n3)cc2)cc1 ; c1ccc(-c2nc(-c3ccc4c(ccc5ccccc54)c3)nc(-c3ccc4c(c3)oc3ccccc34)n2)cc1 ; c1ccc(-c2nc(-c3ccc4c(ccc5ccccc54)c3)nc(-c3ccc4c(c3)sc3ccccc34)n2)cc1 ; c1ccc(-c2ccc(-c3nc(-c4cccc(-c5ccccc5)c4)nc(-c4cc5ccccc5c5ccccc45)n3)cc2)cc1]

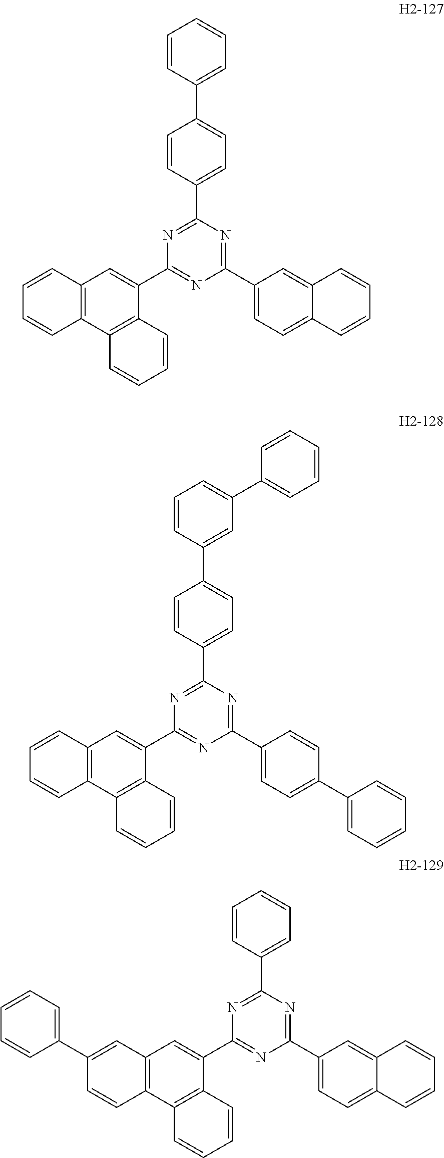 [SMILES: c1ccc(-c2ccc(-c3nc(-c4ccc5ccccc5c4)nc(-c4cc5ccccc5c5ccccc45)n3)cc2)cc1 ; c1ccc(-c2ccc(-c3nc(-c4ccc(-c5cccc(-c6ccccc6)c5)cc4)nc(-c4cc5ccccc5c5ccccc45)n3)cc2)cc1 ; c1ccc(-c2ccc3c(c2)cc(-c2nc(-c4ccccc4)nc(-c4ccc5ccccc5c4)n2)c2ccccc23)cc1]

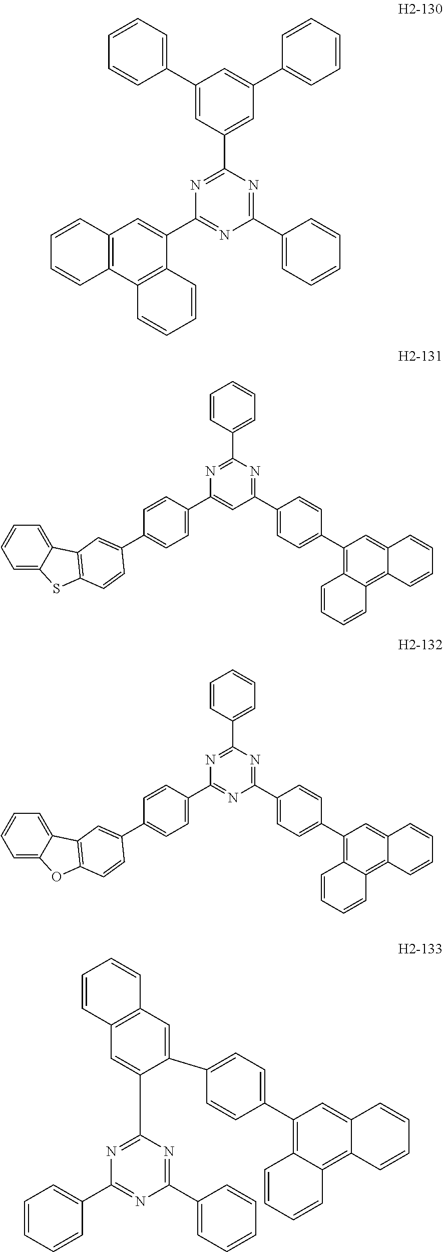 [SMILES: c1ccc(-c2cc(-c3ccccc3)cc(-c3nc(-c4ccccc4)nc(-c4cc5ccccc5c5ccccc45)n3)c2)cc1 ; c1ccc(-c2nc(-c3ccc(-c4ccc5sc6ccccc6c5c4)cc3)cc(-c3ccc(-c4cc5ccccc5c5ccccc45)cc3)n2)cc1 ; c1ccc(-c2nc(-c3ccc(-c4ccc5oc6ccccc6c5c4)cc3)nc(-c3ccc(-c4cc5ccccc5c5ccccc45)cc3)n2)cc1 ; c1ccc(-c2nc(-c3ccccc3)nc(-c3cc4ccccc4cc3-c3ccc(-c4cc5ccccc5c5ccccc45)cc3)n2)cc1]

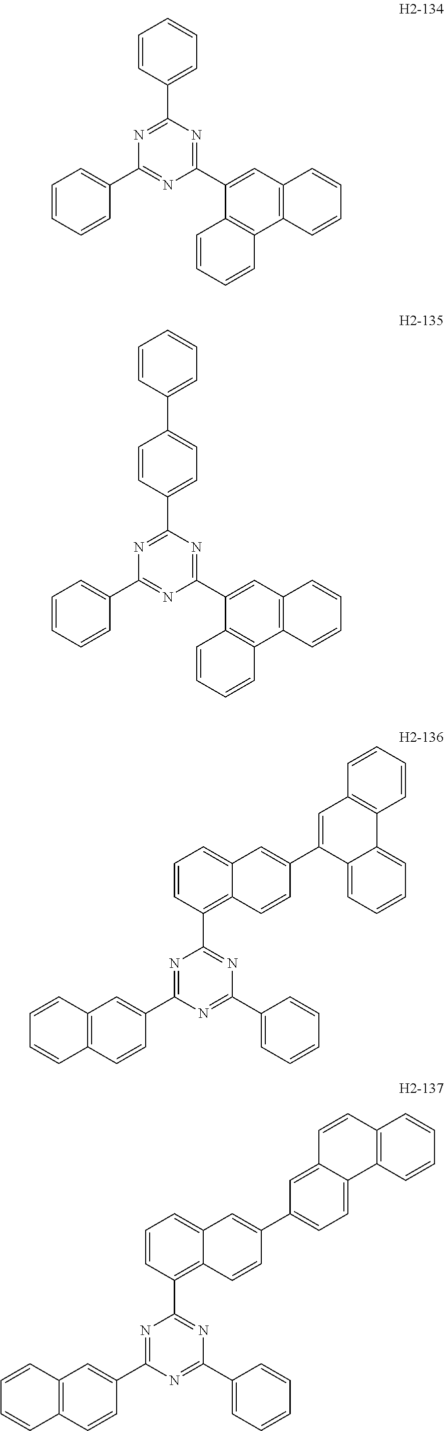 [SMILES: c1ccc(-c2nc(-c3ccccc3)nc(-c3cc4ccccc4c4ccccc34)n2)cc1 ; c1ccc(-c2ccc(-c3nc(-c4ccccc4)nc(-c4cc5ccccc5c5ccccc45)n3)cc2)cc1 ; c1ccc(-c2nc(-c3ccc4ccccc4c3)nc(-c3cccc4cc(-c5cc6ccccc6c6ccccc56)ccc34)n2)cc1 ; c1ccc(-c2nc(-c3ccc4ccccc4c3)nc(-c3cccc4cc(-c5ccc6c(ccc7ccccc76)c5)ccc34)n2)cc1]

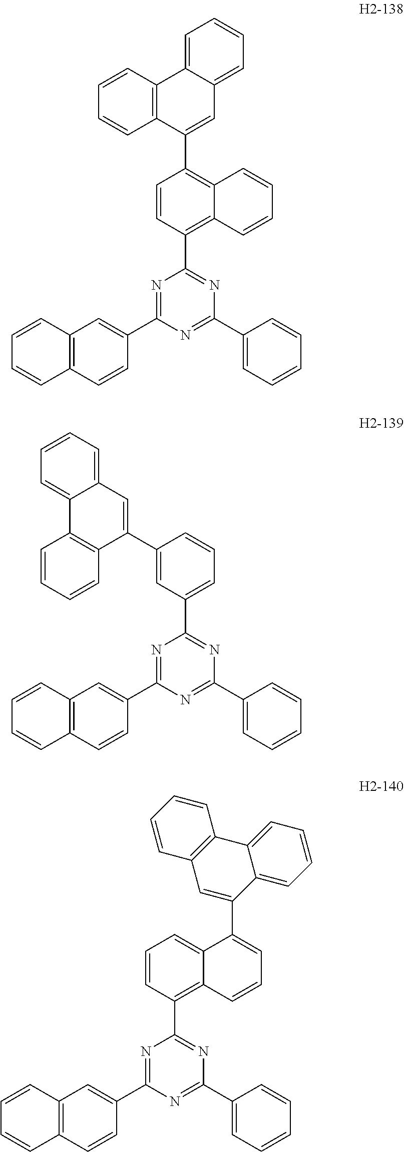 [SMILES: c1ccc(-c2nc(-c3ccc4ccccc4c3)nc(-c3ccc(-c4cc5ccccc5c5ccccc45)c4ccccc34)n2)cc1 ; c1ccc(-c2nc(-c3cccc(-c4cc5ccccc5c5ccccc45)c3)nc(-c3ccc4ccccc4c3)n2)cc1 ; c1ccc(-c2nc(-c3ccc4ccccc4c3)nc(-c3cccc4c(-c5cc6ccccc6c6ccccc56)cccc34)n2)cc1]

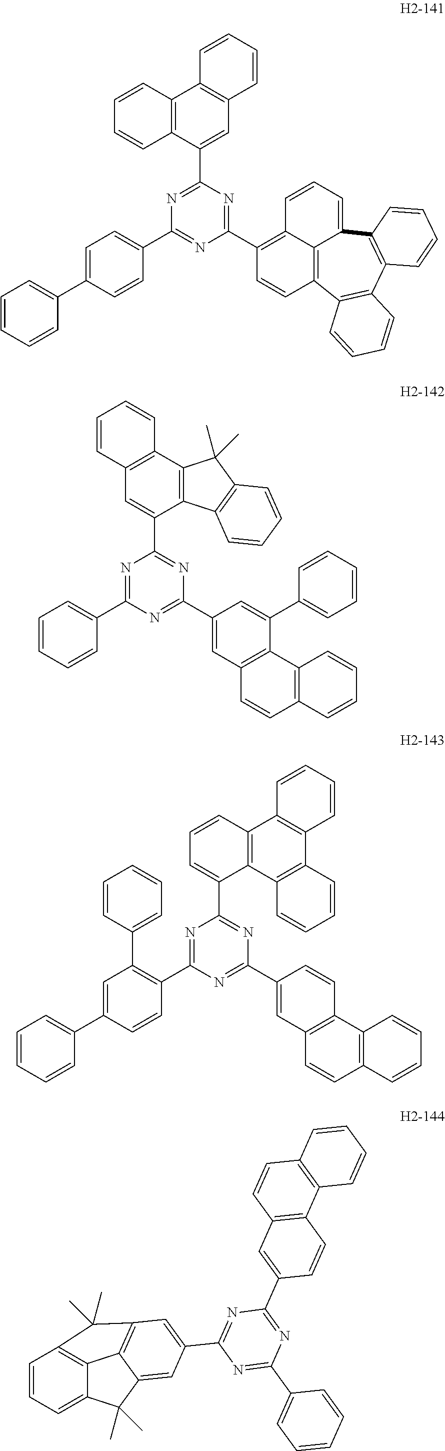

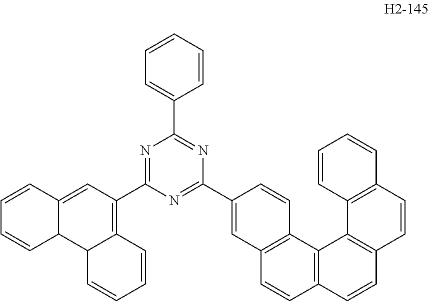 [SMILES: C1=CC2=CC(c3nc(-c4ccccc4)nc(-c4ccc5c(ccc6ccc7ccc8ccccc8c7c65)c4)n3)=C3C=CC=CC3C2C=C1]

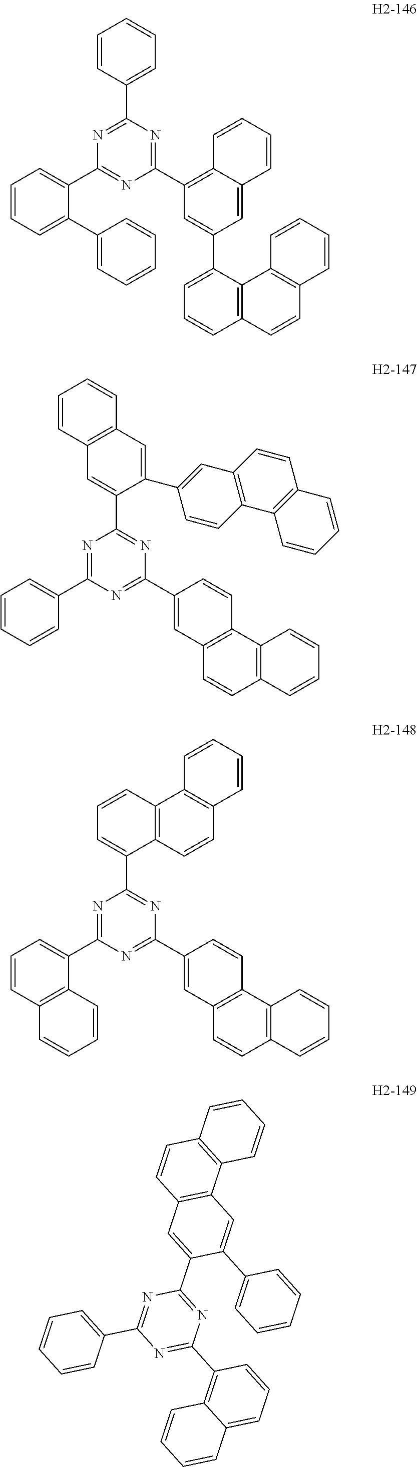

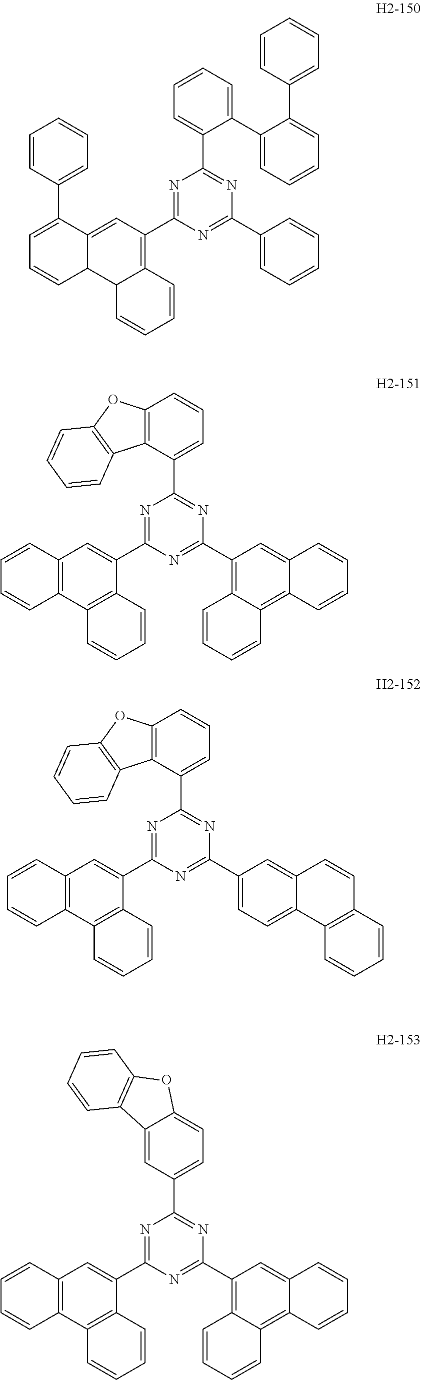 [SMILES: C1=CC2=C(c3nc(-c4ccccc4)nc(-c4ccccc4-c4ccccc4-c4ccccc4)n3)C=C3C(c4ccccc4)=CC=CC3C2C=C1 ; c1ccc2c(c1)cc(-c1nc(-c3cc4ccccc4c4ccccc34)nc(-c3cccc4oc5ccccc5c34)n1)c1ccccc12 ; c1ccc2c(c1)ccc1cc(-c3nc(-c4cc5ccccc5c5ccccc45)nc(-c4cccc5oc6ccccc6c45)n3)ccc12 ; c1ccc2c(c1)cc(-c1nc(-c3ccc4oc5ccccc5c4c3)nc(-c3cc4ccccc4c4ccccc34)n1)c1ccccc12]

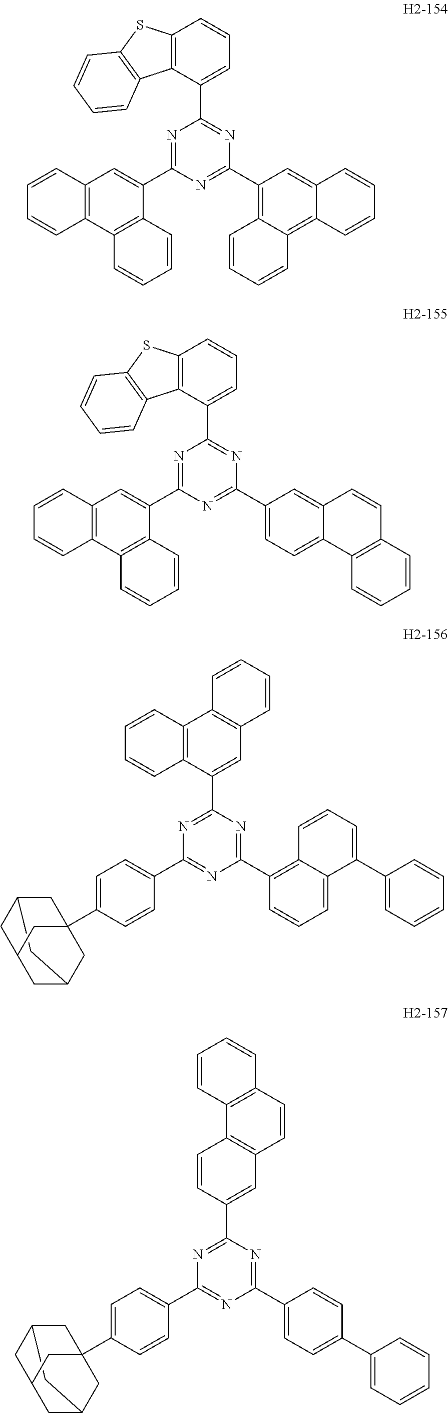 [SMILES: c1ccc2c(c1)cc(-c1nc(-c3cc4ccccc4c4ccccc34)nc(-c3cccc4sc5ccccc5c34)n1)c1ccccc12 ; c1ccc2c(c1)ccc1cc(-c3nc(-c4cc5ccccc5c5ccccc45)nc(-c4cccc5sc6ccccc6c45)n3)ccc12 ; c1ccc(-c2cccc3c(-c4nc(-c5ccc(C67CC8CC(CC(C8)C6)C7)cc5)nc(-c5cc6ccccc6c6ccccc56)n4)cccc23)cc1 ; c1ccc(-c2ccc(-c3nc(-c4ccc(C56CC7CC(CC(C7)C5)C6)cc4)nc(-c4ccc5c(ccc6ccccc65)c4)n3)cc2)cc1]

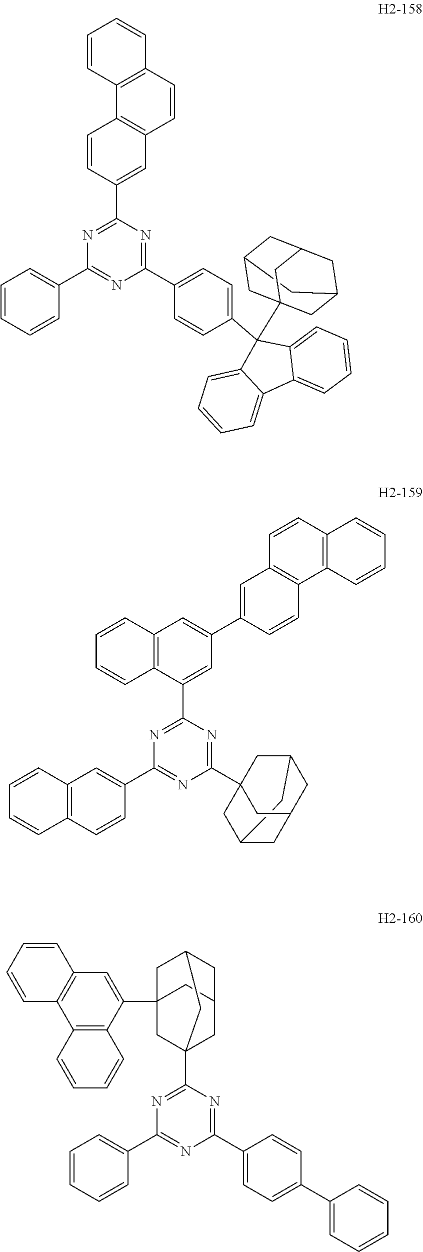 [SMILES: c1ccc(-c2nc(-c3ccc(C4(C56CC7CC(CC(C7)C5)C6)c5ccccc5-c5ccccc54)cc3)nc(-c3ccc4c(ccc5ccccc54)c3)n2)cc1 ; c1ccc2cc(-c3nc(-c4cc(-c5ccc6c(ccc7ccccc76)c5)cc5ccccc45)nc(C45CC6CC(CC(C6)C4)C5)n3)ccc2c1 ; c1ccc(-c2ccc(-c3nc(-c4ccccc4)nc(C45CC6CC(C4)CC(c4cc7ccccc7c7ccccc47)(C6)C5)n3)cc2)cc1]

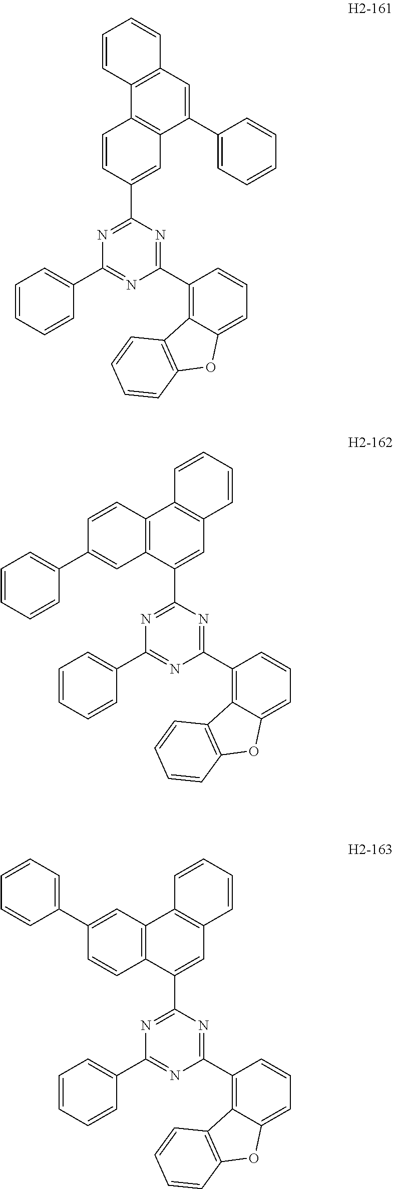 [SMILES: c1ccc(-c2nc(-c3ccc4c(c3)c(-c3ccccc3)cc3ccccc34)nc(-c3cccc4oc5ccccc5c34)n2)cc1 ; c1ccc(-c2ccc3c(c2)c(-c2nc(-c4ccccc4)nc(-c4cccc5oc6ccccc6c45)n2)cc2ccccc23)cc1 ; c1ccc(-c2ccc3c(-c4nc(-c5ccccc5)nc(-c5cccc6oc7ccccc7c56)n4)cc4ccccc4c3c2)cc1]

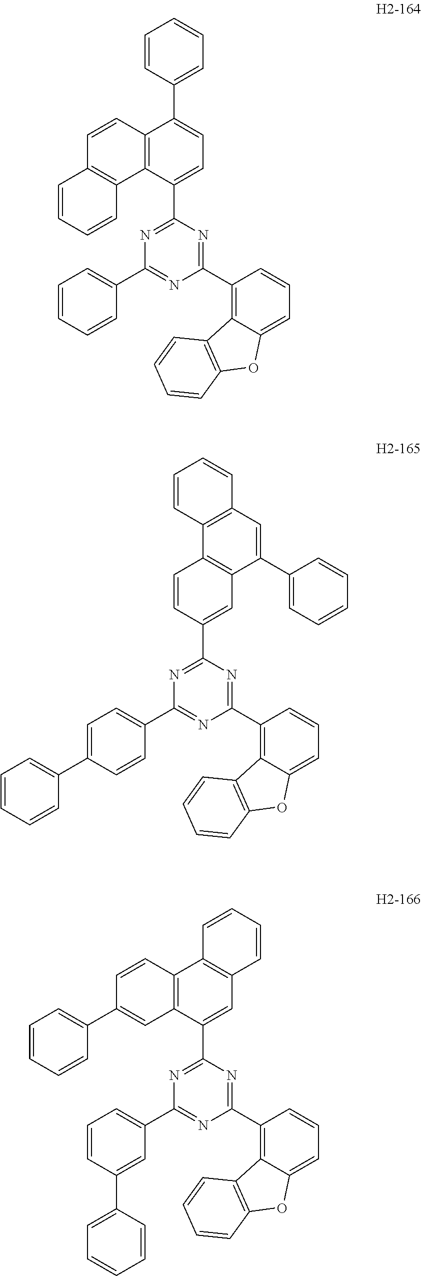 [SMILES: c1ccc(-c2nc(-c3cccc4oc5ccccc5c34)nc(-c3ccc(-c4ccccc4)c4ccc5ccccc5c34)n2)cc1 ; c1ccc(-c2ccc(-c3nc(-c4ccc5c(c4)c(-c4ccccc4)cc4ccccc45)nc(-c4cccc5oc6ccccc6c45)n3)cc2)cc1 ; c1ccc(-c2cccc(-c3nc(-c4cc5ccccc5c5ccc(-c6ccccc6)cc45)nc(-c4cccc5oc6ccccc6c45)n3)c2)cc1]

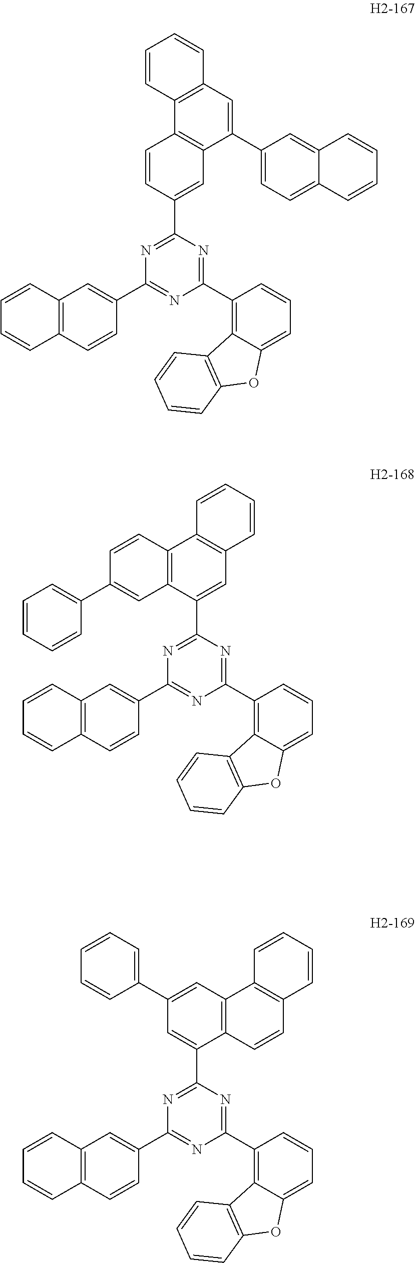 [SMILES: c1ccc2cc(-c3nc(-c4ccc5c(c4)c(-c4ccc6ccccc6c4)cc4ccccc45)nc(-c4cccc5oc6ccccc6c45)n3)ccc2c1 ; c1ccc(-c2ccc3c(c2)c(-c2nc(-c4ccc5ccccc5c4)nc(-c4cccc5oc6ccccc6c45)n2)cc2ccccc23)cc1 ; c1ccc(-c2cc(-c3nc(-c4ccc5ccccc5c4)nc(-c4cccc5oc6ccccc6c45)n3)c3ccc4ccccc4c3c2)cc1]

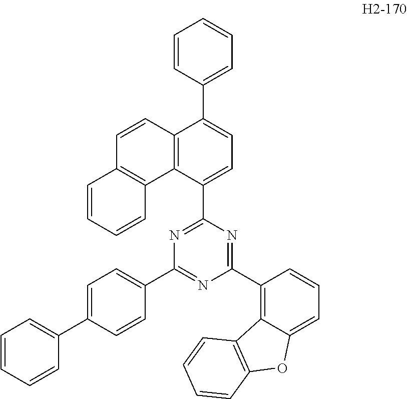 [SMILES: c1ccc(-c2ccc(-c3nc(-c4cccc5oc6ccccc6c45)nc(-c4ccc(-c5ccccc5)c5ccc6ccccc6c45)n3)cc2)cc1]

The combination of at least one of compounds H1-1 to H1-143 and at least one of compounds H2-1 to H2-170 may be used in an OLED.

In addition, the present disclosure provides the organic electroluminescent compound represented by the following formula 2′.

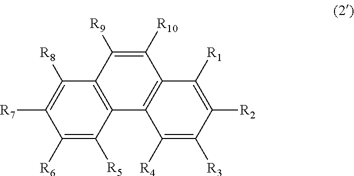

In formula 2′,
R₁ to R₁₀ are each independently represented by

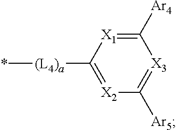

or represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s), with a proviso that at least one of R₁ to R₁₀ is represented by

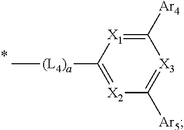

L₄ represents a single bond, a substituted or unsubstituted (C6-C30)cycloalkylene, or a substituted or unsubstituted (C10)arylene;
all of X₁ to X₃ represent N;
Ar₄ and Ar₅ each independently represent a substituted or unsubstituted (C6-C30)cycloalkyl, a substituted or unsubstituted (C6-C30)aryl, a substituted or unsubstituted (3- to 30-membered)heteroaryl, or a substituted or unsubstituted fused ring group of a (C3-C30) aliphatic ring(s) and a (C6-C30) aromatic ring(s), and Ar₄ and Ar₅ are the same as or different from each other;
with a proviso that when each of Ar₄ and Ar₅ represents an unsubstituted phenyl, an unsubstituted naphthyl, an unsubstituted biphenyl, an unsubstituted terphenyl, an unsubstituted phenanthrenyl, an unsubstituted triphenylenyl, an unsubstituted 1-dibenzofuranyl, an unsubstituted 1-dibenzothiophenyl, a phenyl substituted with a 1-dibenzofuranyl(s), a phenyl substituted with a 1-dibenzothiophenyl(s), a phenyl substituted with a 9-carbazolyl(s), an unsubstituted carbazolyl, a substituted or unsubstituted dibenzodioxynyl, a dibenzothiophenyl fused to benzofuran, or a dibenzothiophenyl fused to benzothiophene, and L₄ represents a single bond, L₄ is not linked at the position of R₉ or R₁₀;
with a proviso that when L₄ represents a single bond and is linked at the position of R₂ or R₇, Ar₄ and Ar₅ each independently represent a phenyl unsubstituted or substituted with a (C6-C30)cycloalkyl(s); a naphthyl unsubstituted or substituted with a dibenzofuranyl(s), a dibenzothiophenyl(s) or a phenanthrenyl(s); an unsubstituted biphenyl; an unsubstituted terphenyl (except for the structure

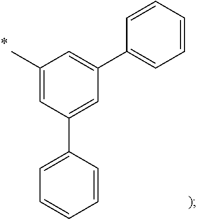 [SMILES: Cc1cc(-c2ccccc2)cc(-c2ccccc2)c1]

an unsubstituted phenanthrenyl; an unsubstituted triphenylenyl; an unsubstituted chrysenyl; an unsubstituted (C22)aryl; an unsubstituted 9,9-diphenylfluorenyl; an unsubstituted 7,7-dimethylbenzofluorenyl; an unsubstituted 9,9-diphenylsilafluorenyl; a fluorenyl substituted with a phenyl(s) and a (C6-C30)cycloalkyl(s); a fused ring group of a (C3-C10) aliphatic ring(s) and a (C6-C12) aromatic ring(s) substituted with a methyl(s); an unsubstituted phenylnaphthyl; an unsubstituted naphthylphenyl; an unsubstituted dibenzofuranyl; or an unsubstituted dibenzothiophenyl; and
a represents an integer of 1 to 3, where if a is an integer of 2 or more, each of L₄ may be the same or different.

The compound represented by formula 2′ may be at least one selected from the following compounds, but is not limited thereto.

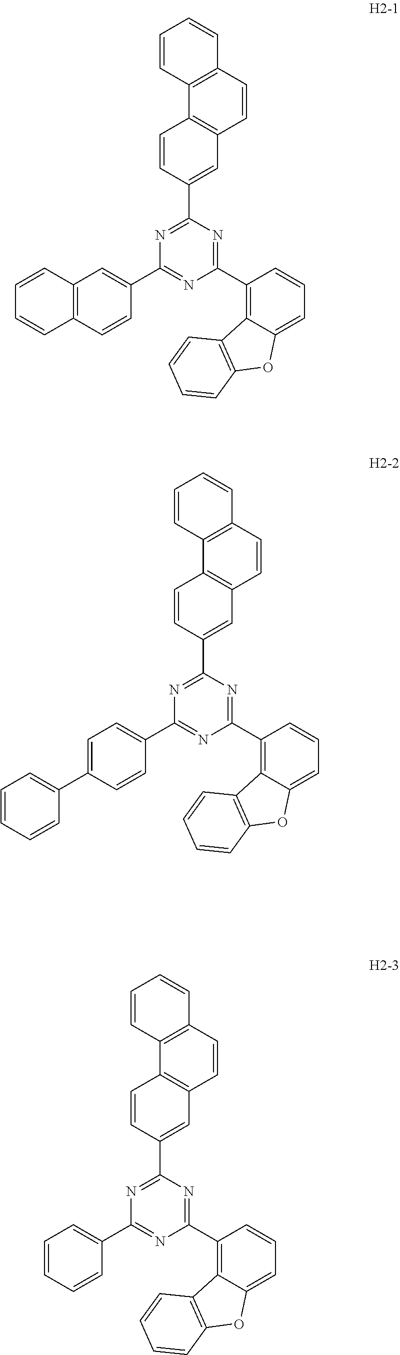 [SMILES: c1ccc2cc(-c3nc(-c4ccc5c(ccc6ccccc65)c4)nc(-c4cccc5oc6ccccc6c45)n3)ccc2c1 ; c1ccc(-c2ccc(-c3nc(-c4ccc5c(ccc6ccccc65)c4)nc(-c4cccc5oc6ccccc6c45)n3)cc2)cc1 ; c1ccc(-c2nc(-c3ccc4c(ccc5ccccc54)c3)nc(-c3cccc4oc5ccccc5c34)n2)cc1]

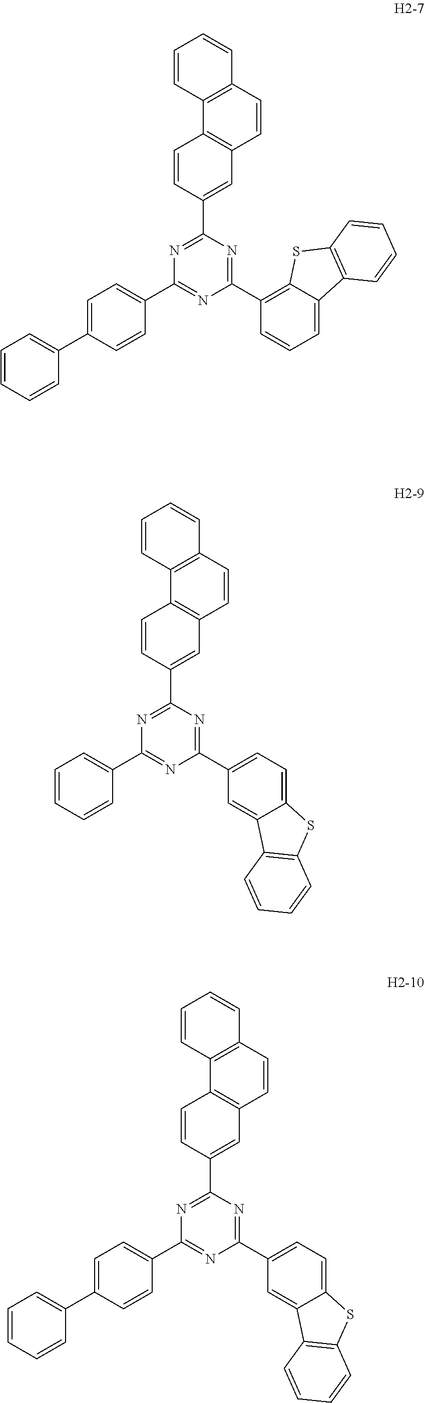 [SMILES: c1ccc(-c2ccc(-c3nc(-c4ccc5c(ccc6ccccc65)c4)nc(-c4cccc5c4sc4ccccc45)n3)cc2)cc1 ; c1ccc(-c2nc(-c3ccc4c(ccc5ccccc54)c3)nc(-c3ccc4sc5ccccc5c4c3)n2)cc1 ; c1ccc(-c2ccc(-c3nc(-c4ccc5c(ccc6ccccc65)c4)nc(-c4ccc5sc6ccccc6c5c4)n3)cc2)cc1]

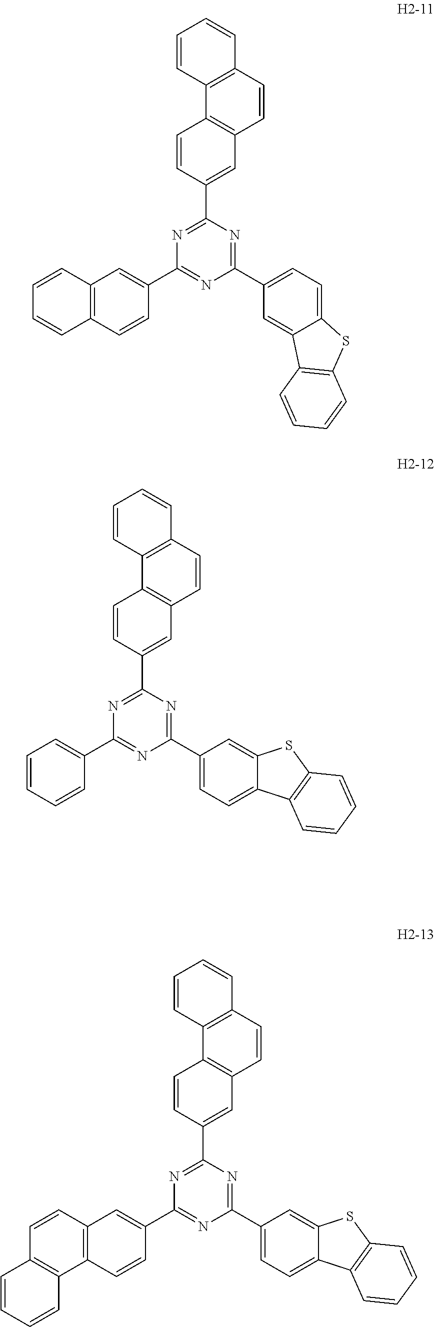 [SMILES: c1ccc2cc(-c3nc(-c4ccc5c(ccc6ccccc65)c4)nc(-c4ccc5sc6ccccc6c5c4)n3)ccc2c1 ; c1ccc(-c2nc(-c3ccc4c(ccc5ccccc54)c3)nc(-c3ccc4c(c3)sc3ccccc34)n2)cc1 ; c1ccc2c(c1)ccc1cc(-c3nc(-c4ccc5c(ccc6ccccc65)c4)nc(-c4ccc5c(c4)sc4ccccc45)n3)ccc12]

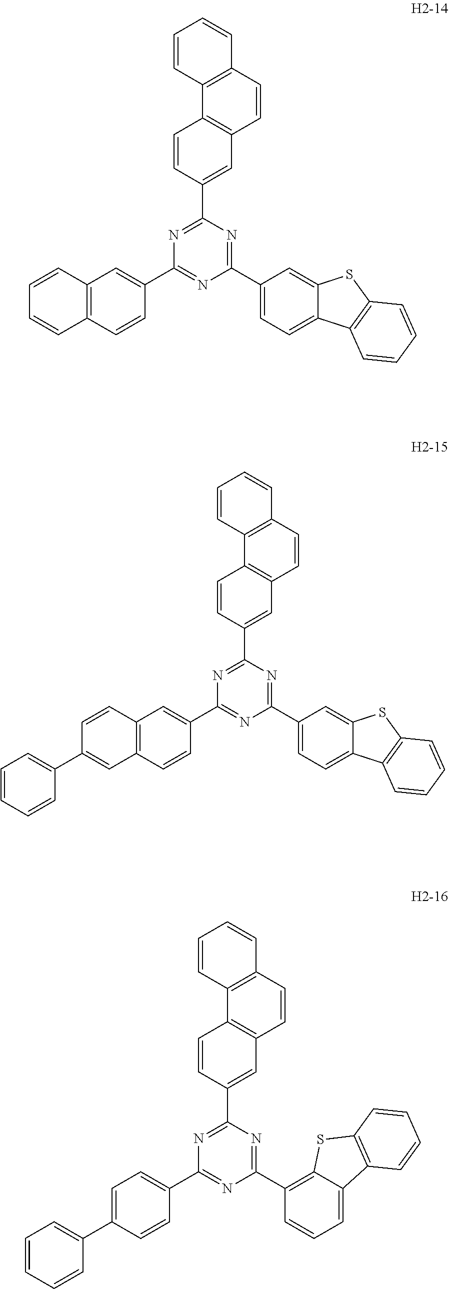 [SMILES: c1ccc2cc(-c3nc(-c4ccc5c(ccc6ccccc65)c4)nc(-c4ccc5c(c4)sc4ccccc45)n3)ccc2c1 ; c1ccc(-c2ccc3cc(-c4nc(-c5ccc6c(ccc7ccccc76)c5)nc(-c5ccc6c(c5)sc5ccccc56)n4)ccc3c2)cc1 ; c1ccc(-c2ccc(-c3nc(-c4ccc5c(ccc6ccccc65)c4)nc(-c4cccc5c4sc4ccccc45)n3)cc2)cc1]

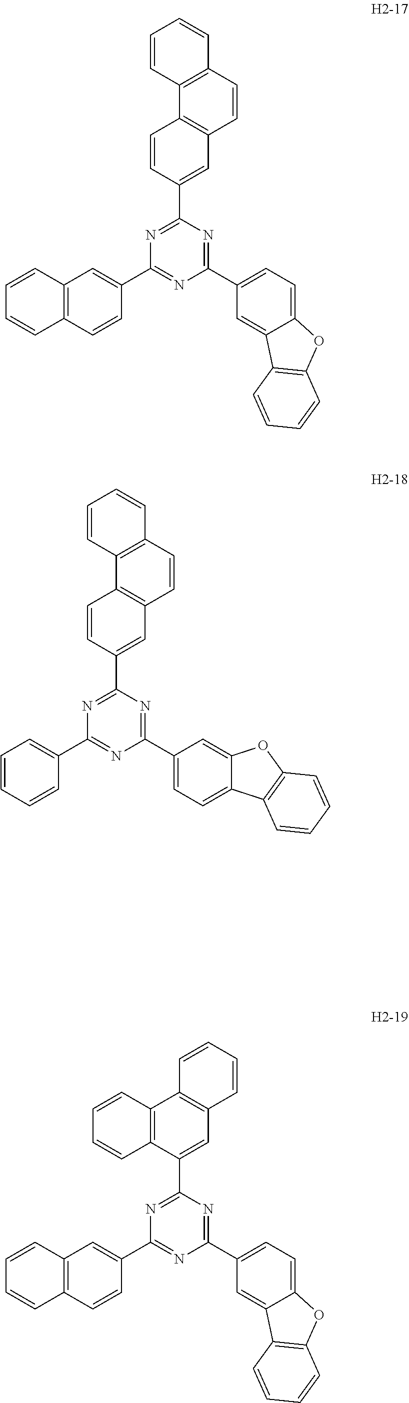 [SMILES: c1ccc2cc(-c3nc(-c4ccc5c(ccc6ccccc65)c4)nc(-c4ccc5oc6ccccc6c5c4)n3)ccc2c1 ; c1ccc(-c2nc(-c3ccc4c(ccc5ccccc54)c3)nc(-c3ccc4c(c3)oc3ccccc34)n2)cc1 ; c1ccc2cc(-c3nc(-c4ccc5oc6ccccc6c5c4)nc(-c4cc5ccccc5c5ccccc45)n3)ccc2c1]

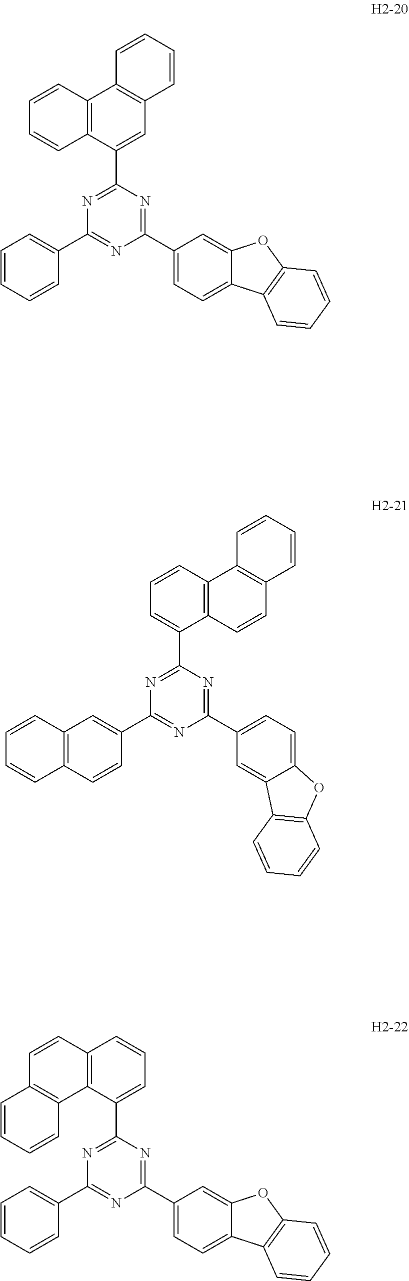 [SMILES: c1ccc(-c2nc(-c3ccc4c(c3)oc3ccccc34)nc(-c3cc4ccccc4c4ccccc34)n2)cc1 ; c1ccc2cc(-c3nc(-c4ccc5oc6ccccc6c5c4)nc(-c4cccc5c4ccc4ccccc45)n3)ccc2c1 ; c1ccc(-c2nc(-c3ccc4c(c3)oc3ccccc34)nc(-c3cccc4ccc5ccccc5c34)n2)cc1]

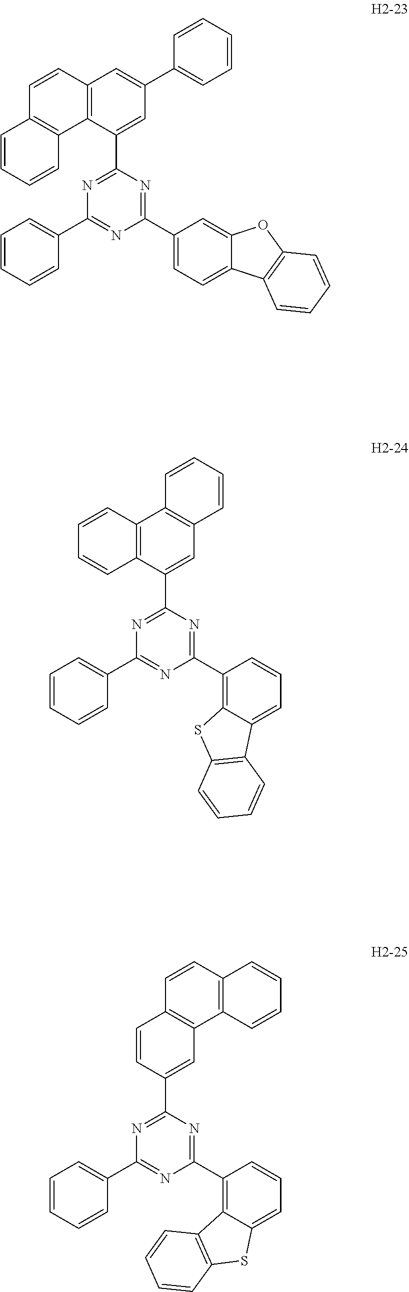 [SMILES: c1ccc(-c2cc(-c3nc(-c4ccccc4)nc(-c4ccc5c(c4)oc4ccccc45)n3)c3c(ccc4ccccc43)c2)cc1 ; c1ccc(-c2nc(-c3cc4ccccc4c4ccccc34)nc(-c3cccc4c3sc3ccccc34)n2)cc1 ; c1ccc(-c2nc(-c3ccc4ccc5ccccc5c4c3)nc(-c3cccc4sc5ccccc5c34)n2)cc1]

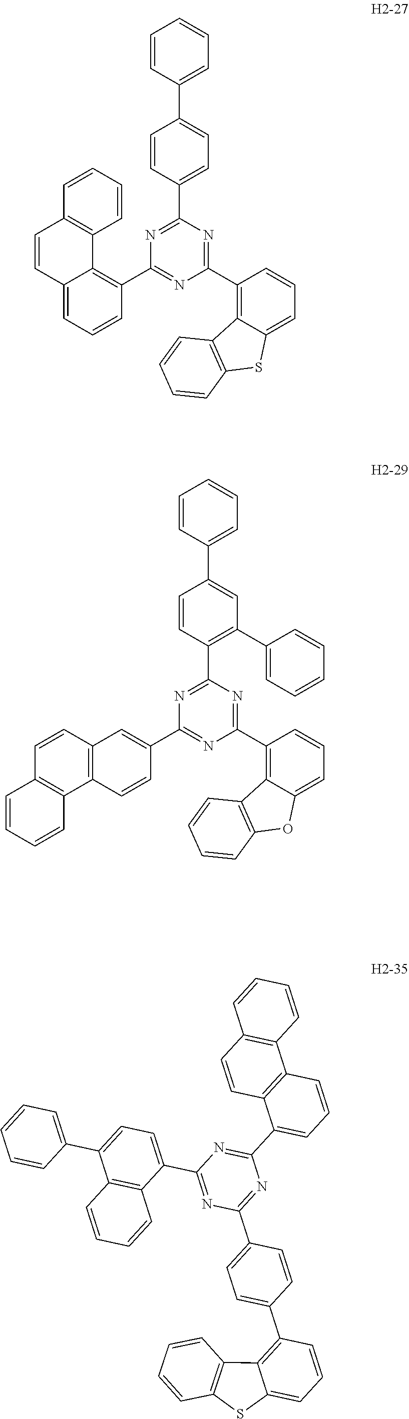 [SMILES: c1ccc(-c2ccc(-c3nc(-c4cccc5ccc6ccccc6c45)nc(-c4cccc5sc6ccccc6c45)n3)cc2)cc1 ; c1ccc(-c2ccc(-c3nc(-c4ccc5c(ccc6ccccc65)c4)nc(-c4cccc5oc6ccccc6c45)n3)c(-c3ccccc3)c2)cc1 ; c1ccc(-c2ccc(-c3nc(-c4ccc(-c5cccc6sc7ccccc7c56)cc4)nc(-c4cccc5c4ccc4ccccc45)n3)c3ccccc23)cc1]

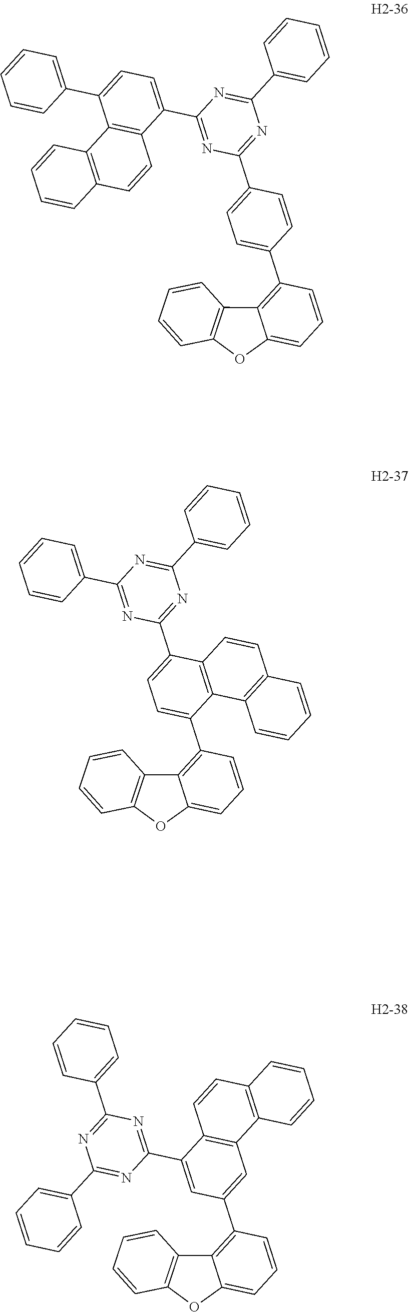 [SMILES: c1ccc(-c2nc(-c3ccc(-c4cccc5oc6ccccc6c45)cc3)nc(-c3ccc(-c4ccccc4)c4c3ccc3ccccc34)n2)cc1 ; c1ccc(-c2nc(-c3ccccc3)nc(-c3ccc(-c4cccc5oc6ccccc6c45)c4c3ccc3ccccc34)n2)cc1 ; c1ccc(-c2nc(-c3ccccc3)nc(-c3cc(-c4cccc5oc6ccccc6c45)cc4c3ccc3ccccc34)n2)cc1]

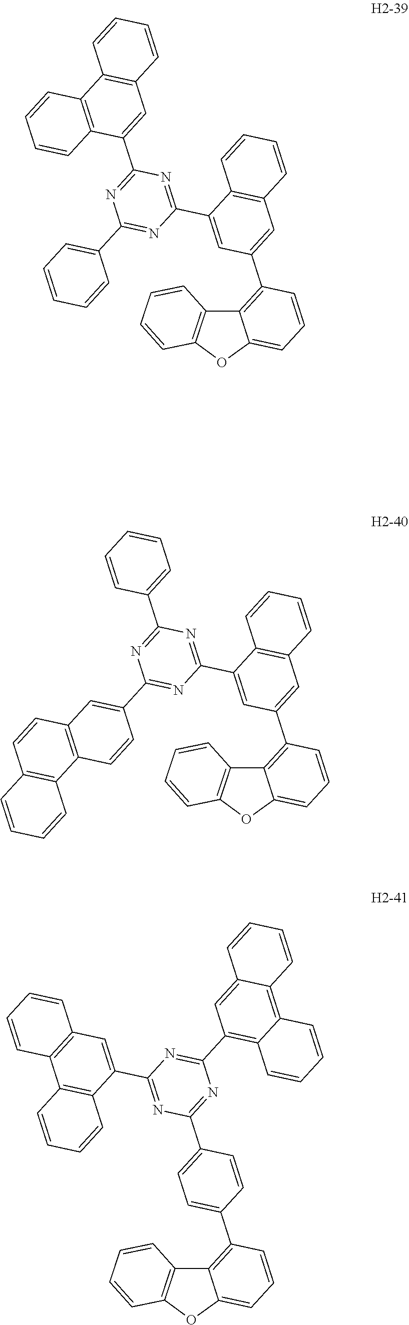 [SMILES: c1ccc(-c2nc(-c3cc(-c4cccc5oc6ccccc6c45)cc4ccccc34)nc(-c3cc4ccccc4c4ccccc34)n2)cc1 ; c1ccc(-c2nc(-c3ccc4c(ccc5ccccc54)c3)nc(-c3cc(-c4cccc5oc6ccccc6c45)cc4ccccc34)n2)cc1 ; c1ccc2c(c1)cc(-c1nc(-c3ccc(-c4cccc5oc6ccccc6c45)cc3)nc(-c3cc4ccccc4c4ccccc34)n1)c1ccccc12]

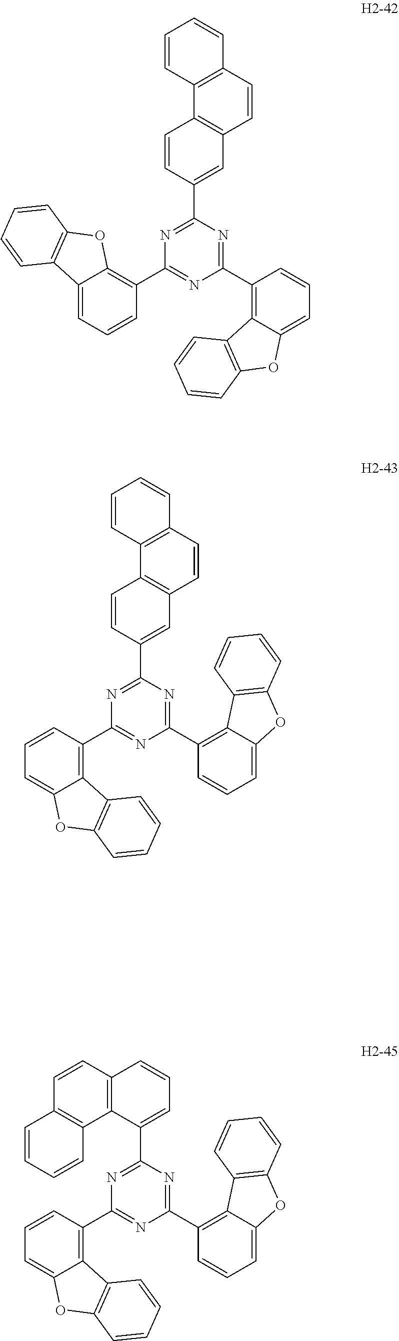 [SMILES: c1ccc2c(c1)ccc1cc(-c3nc(-c4cccc5c4oc4ccccc45)nc(-c4cccc5oc6ccccc6c45)n3)ccc12 ; c1ccc2c(c1)ccc1cc(-c3nc(-c4cccc5oc6ccccc6c45)nc(-c4cccc5oc6ccccc6c45)n3)ccc12 ; c1ccc2c(c1)ccc1cccc(-c3nc(-c4cccc5oc6ccccc6c45)nc(-c4cccc5oc6ccccc6c45)n3)c12]

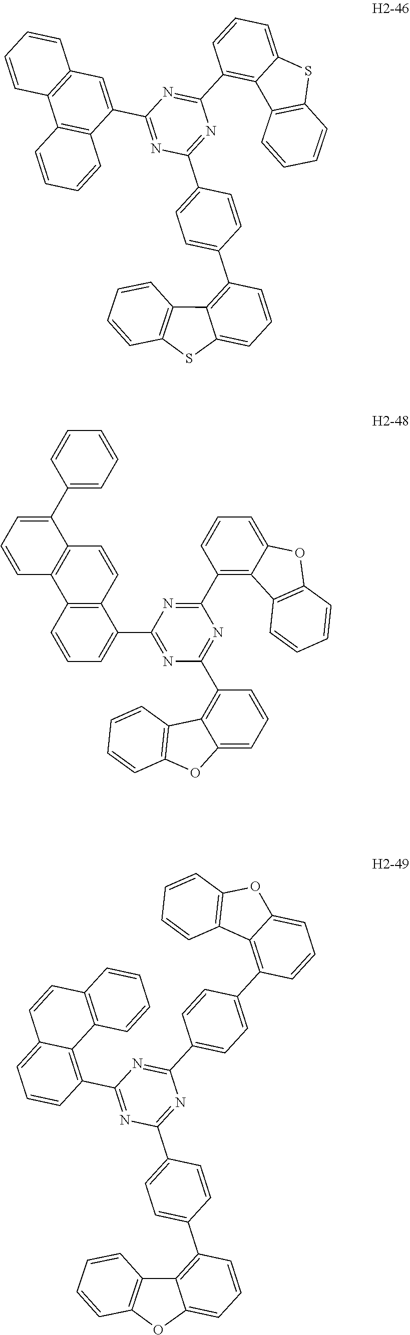 [SMILES: c1ccc2c(c1)cc(-c1nc(-c3ccc(-c4cccc5sc6ccccc6c45)cc3)nc(-c3cccc4sc5ccccc5c34)n1)c1ccccc12 ; c1ccc(-c2cccc3c2ccc2c(-c4nc(-c5cccc6oc7ccccc7c56)nc(-c5cccc6oc7ccccc7c56)n4)cccc23)cc1 ; c1ccc2c(c1)ccc1cccc(-c3nc(-c4ccc(-c5cccc6oc7ccccc7c56)cc4)nc(-c4ccc(-c5cccc6oc7ccccc7c56)cc4)n3)c12]

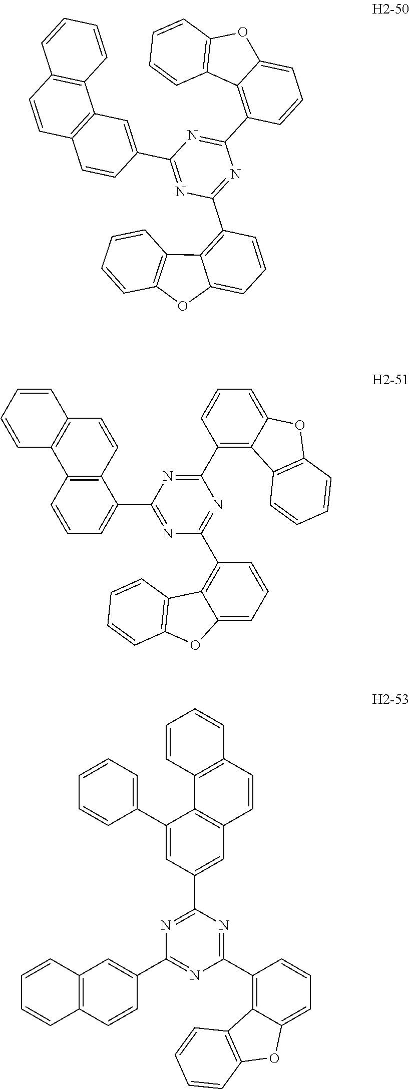 [SMILES: c1ccc2c(c1)ccc1ccc(-c3nc(-c4cccc5oc6ccccc6c45)nc(-c4cccc5oc6ccccc6c45)n3)cc12 ; c1ccc2c(c1)ccc1c(-c3nc(-c4cccc5oc6ccccc6c45)nc(-c4cccc5oc6ccccc6c45)n3)cccc12 ; c1ccc(-c2cc(-c3nc(-c4ccc5ccccc5c4)nc(-c4cccc5oc6ccccc6c45)n3)cc3ccc4ccccc4c23)cc1]

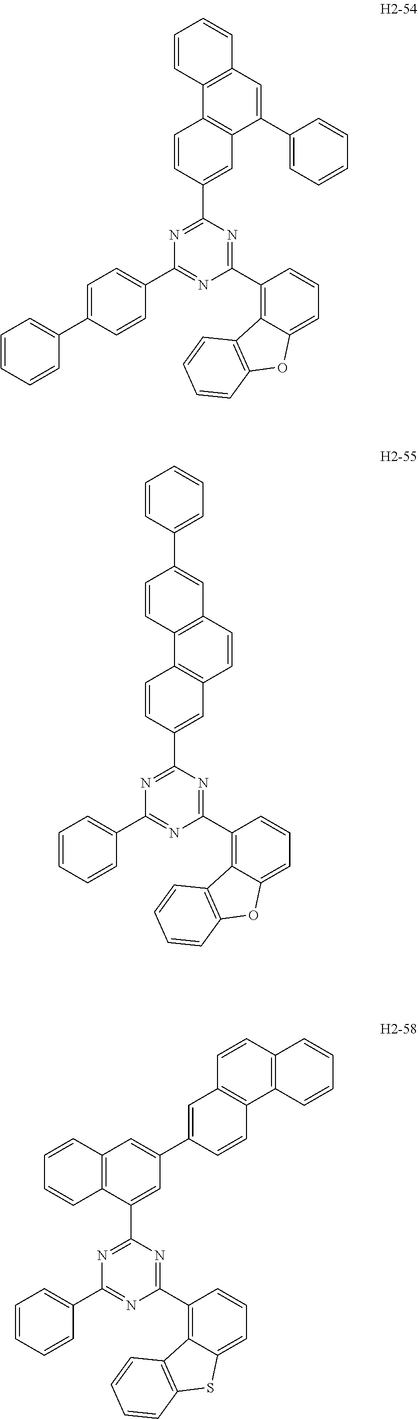 [SMILES: c1ccc(-c2ccc(-c3nc(-c4ccc5c(c4)c(-c4ccccc4)cc4ccccc45)nc(-c4cccc5oc6ccccc6c45)n3)cc2)cc1 ; c1ccc(-c2ccc3c(ccc4cc(-c5nc(-c6ccccc6)nc(-c6cccc7oc8ccccc8c67)n5)ccc43)c2)cc1 ; c1ccc(-c2nc(-c3cc(-c4ccc5c(ccc6ccccc65)c4)cc4ccccc34)nc(-c3cccc4sc5ccccc5c34)n2)cc1]

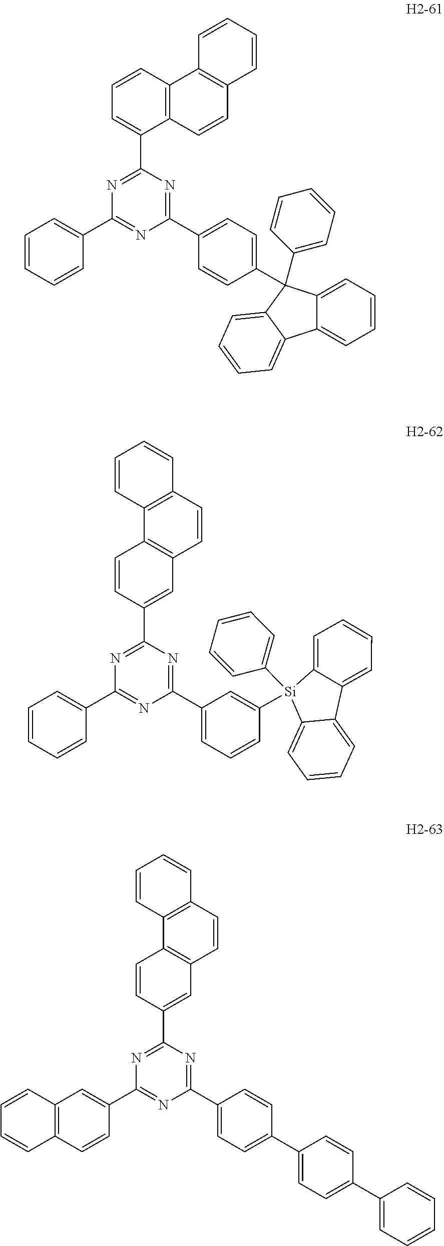 [SMILES: c1ccc(-c2nc(-c3ccc(C4(c5ccccc5)c5ccccc5-c5ccccc54)cc3)nc(-c3cccc4c3ccc3ccccc34)n2)cc1 ; c1ccc(-c2nc(-c3cccc([Si]4(c5ccccc5)c5ccccc5-c5ccccc54)c3)nc(-c3ccc4c(ccc5ccccc54)c3)n2)cc1 ; c1ccc(-c2ccc(-c3ccc(-c4nc(-c5ccc6ccccc6c5)nc(-c5ccc6c(ccc7ccccc76)c5)n4)cc3)cc2)cc1]

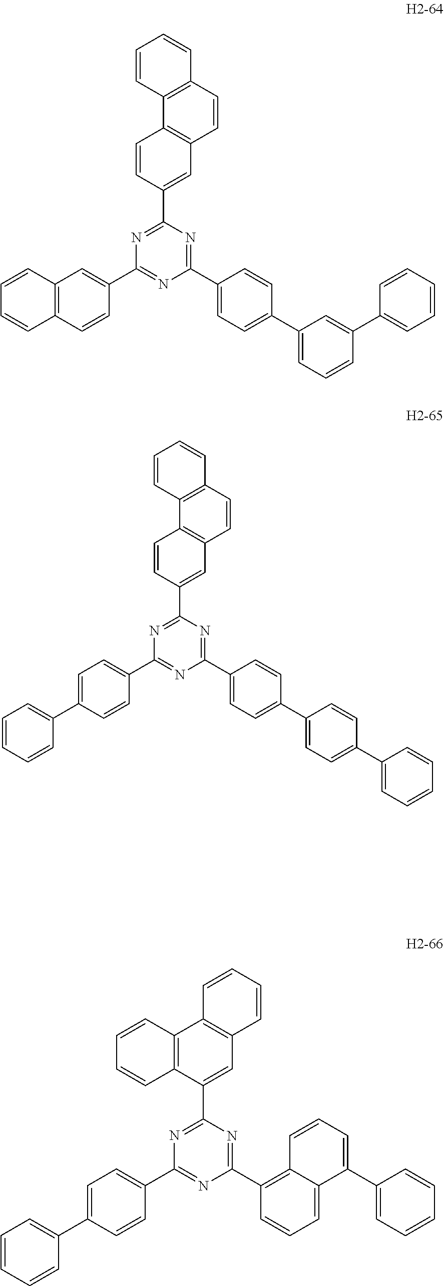 [SMILES: c1ccc(-c2cccc(-c3ccc(-c4nc(-c5ccc6ccccc6c5)nc(-c5ccc6c(ccc7ccccc76)c5)n4)cc3)c2)cc1 ; c1ccc(-c2ccc(-c3ccc(-c4nc(-c5ccc(-c6ccccc6)cc5)nc(-c5ccc6c(ccc7ccccc76)c5)n4)cc3)cc2)cc1 ; c1ccc(-c2ccc(-c3nc(-c4cccc5c(-c6ccccc6)cccc45)nc(-c4cc5ccccc5c5ccccc45)n3)cc2)cc1]

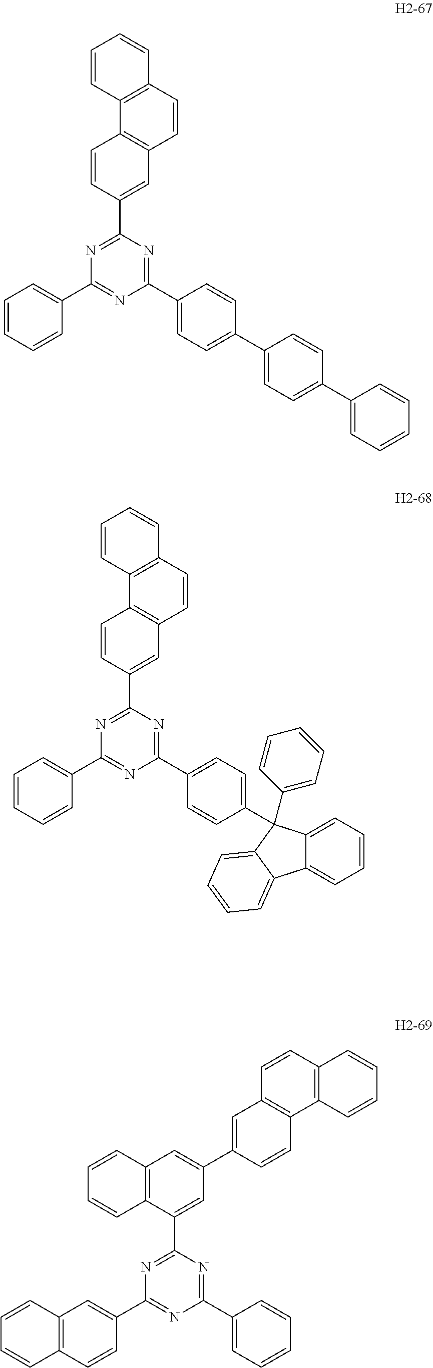 [SMILES: c1ccc(-c2ccc(-c3ccc(-c4nc(-c5ccccc5)nc(-c5ccc6c(ccc7ccccc76)c5)n4)cc3)cc2)cc1 ; c1ccc(-c2nc(-c3ccc(C4(c5ccccc5)c5ccccc5-c5ccccc54)cc3)nc(-c3ccc4c(ccc5ccccc54)c3)n2)cc1 ; c1ccc(-c2nc(-c3ccc4ccccc4c3)nc(-c3cc(-c4ccc5c(ccc6ccccc65)c4)cc4ccccc34)n2)cc1]

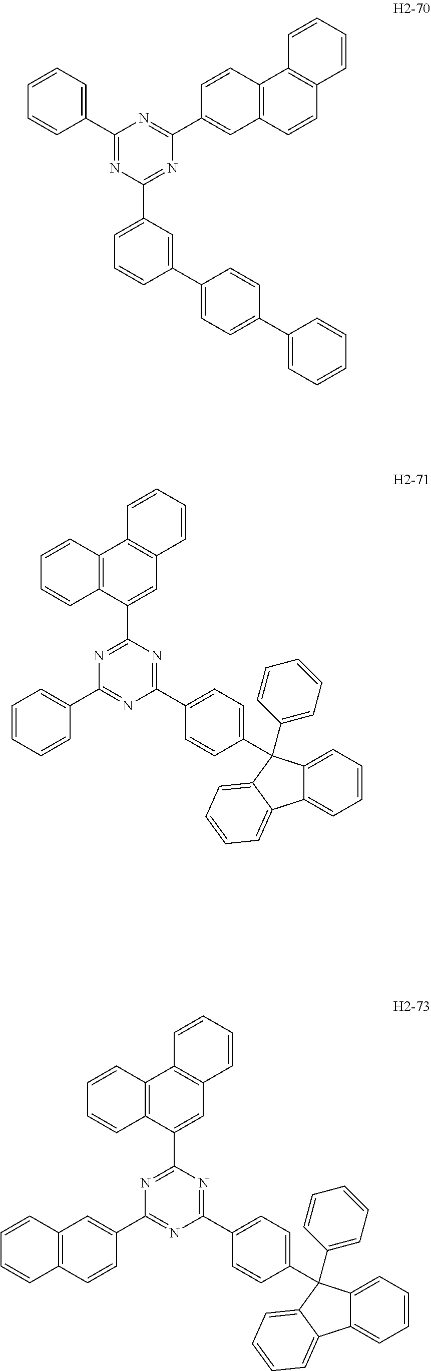 [SMILES: c1ccc(-c2ccc(-c3cccc(-c4nc(-c5ccccc5)nc(-c5ccc6c(ccc7ccccc76)c5)n4)c3)cc2)cc1 ; c1ccc(-c2nc(-c3ccc(C4(c5ccccc5)c5ccccc5-c5ccccc54)cc3)nc(-c3cc4ccccc4c4ccccc34)n2)cc1 ; c1ccc(C2(c3ccc(-c4nc(-c5ccc6ccccc6c5)nc(-c5cc6ccccc6c6ccccc56)n4)cc3)c3ccccc3-c3ccccc32)cc1]

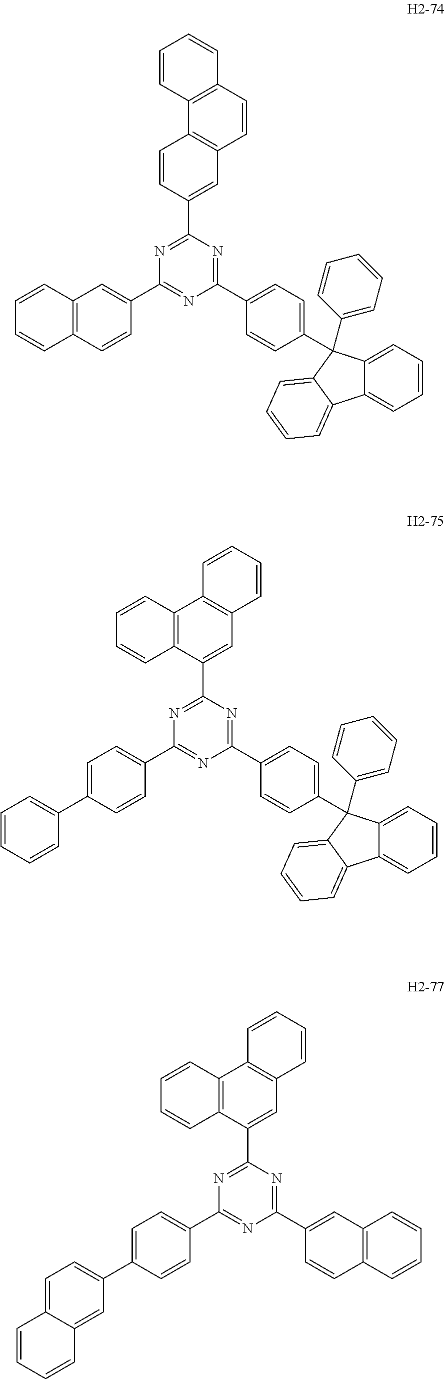 [SMILES: c1ccc(C2(c3ccc(-c4nc(-c5ccc6ccccc6c5)nc(-c5ccc6c(ccc7ccccc76)c5)n4)cc3)c3ccccc3-c3ccccc32)cc1 ; c1ccc(-c2ccc(-c3nc(-c4ccc(C5(c6ccccc6)c6ccccc6-c6ccccc65)cc4)nc(-c4cc5ccccc5c5ccccc45)n3)cc2)cc1 ; c1ccc2cc(-c3ccc(-c4nc(-c5ccc6ccccc6c5)nc(-c5cc6ccccc6c6ccccc56)n4)cc3)ccc2c1]

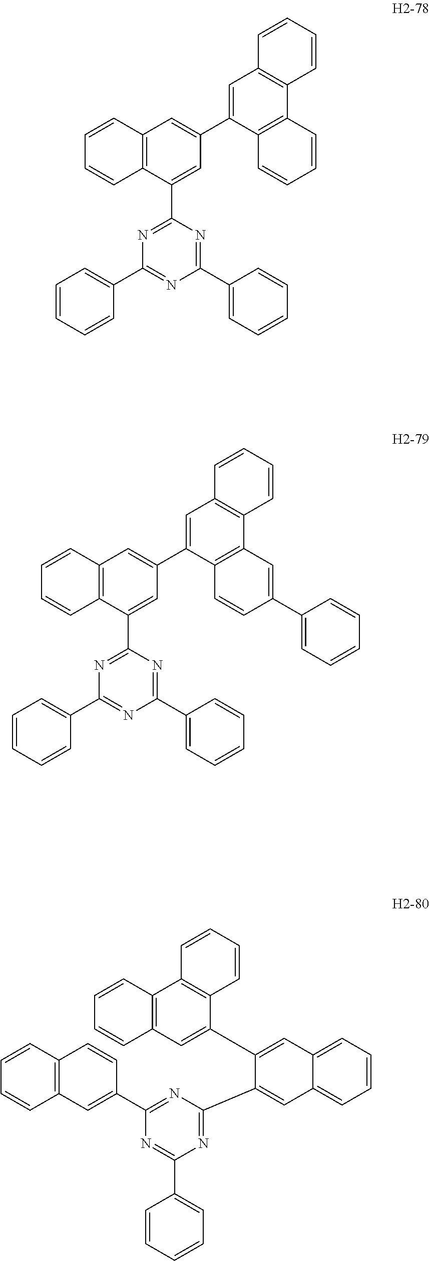 [SMILES: c1ccc(-c2nc(-c3ccccc3)nc(-c3cc(-c4cc5ccccc5c5ccccc45)cc4ccccc34)n2)cc1 ; c1ccc(-c2ccc3c(-c4cc(-c5nc(-c6ccccc6)nc(-c6ccccc6)n5)c5ccccc5c4)cc4ccccc4c3c2)cc1 ; c1ccc(-c2nc(-c3ccc4ccccc4c3)nc(-c3cc4ccccc4cc3-c3cc4ccccc4c4ccccc34)n2)cc1]

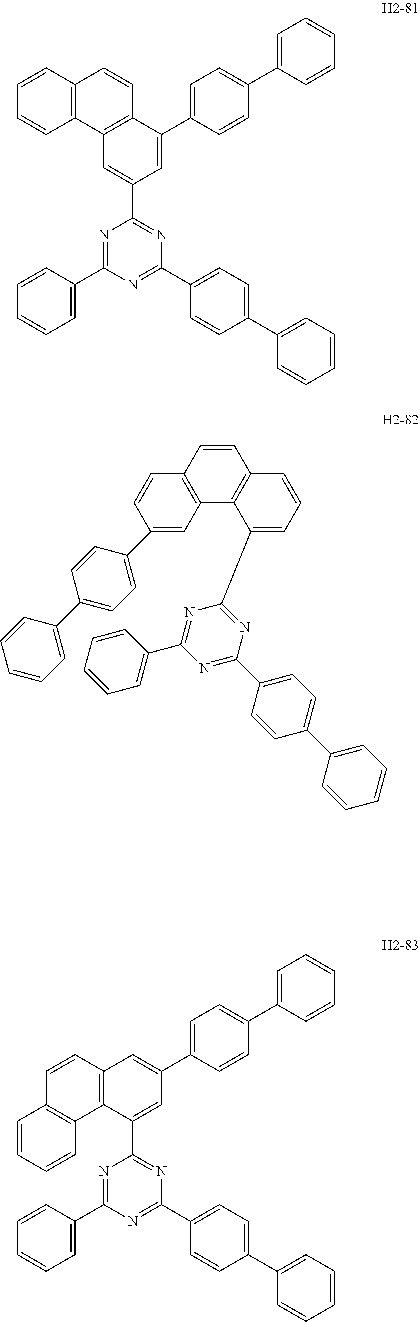 [SMILES: c1ccc(-c2ccc(-c3nc(-c4ccccc4)nc(-c4cc(-c5ccc(-c6ccccc6)cc5)c5ccc6ccccc6c5c4)n3)cc2)cc1 ; c1ccc(-c2ccc(-c3ccc4ccc5cccc(-c6nc(-c7ccccc7)nc(-c7ccc(-c8ccccc8)cc7)n6)c5c4c3)cc2)cc1 ; c1ccc(-c2ccc(-c3cc(-c4nc(-c5ccccc5)nc(-c5ccc(-c6ccccc6)cc5)n4)c4c(ccc5ccccc54)c3)cc2)cc1]

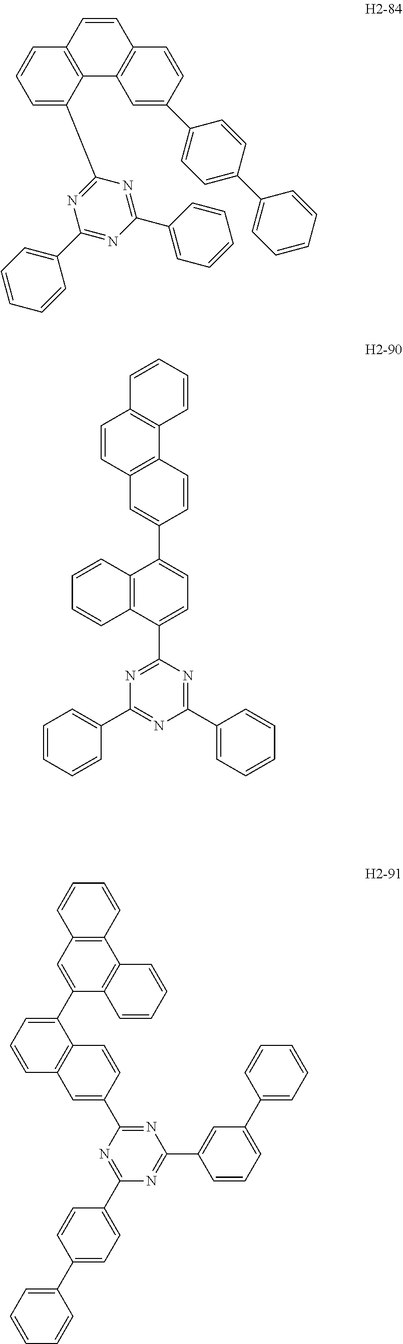 [SMILES: c1ccc(-c2ccc(-c3ccc4ccc5cccc(-c6nc(-c7ccccc7)nc(-c7ccccc7)n6)c5c4c3)cc2)cc1 ; c1ccc(-c2nc(-c3ccccc3)nc(-c3ccc(-c4ccc5c(ccc6ccccc65)c4)c4ccccc34)n2)cc1 ; c1ccc(-c2ccc(-c3nc(-c4cccc(-c5ccccc5)c4)nc(-c4ccc5c(-c6cc7ccccc7c7ccccc67)cccc5c4)n3)cc2)cc1]

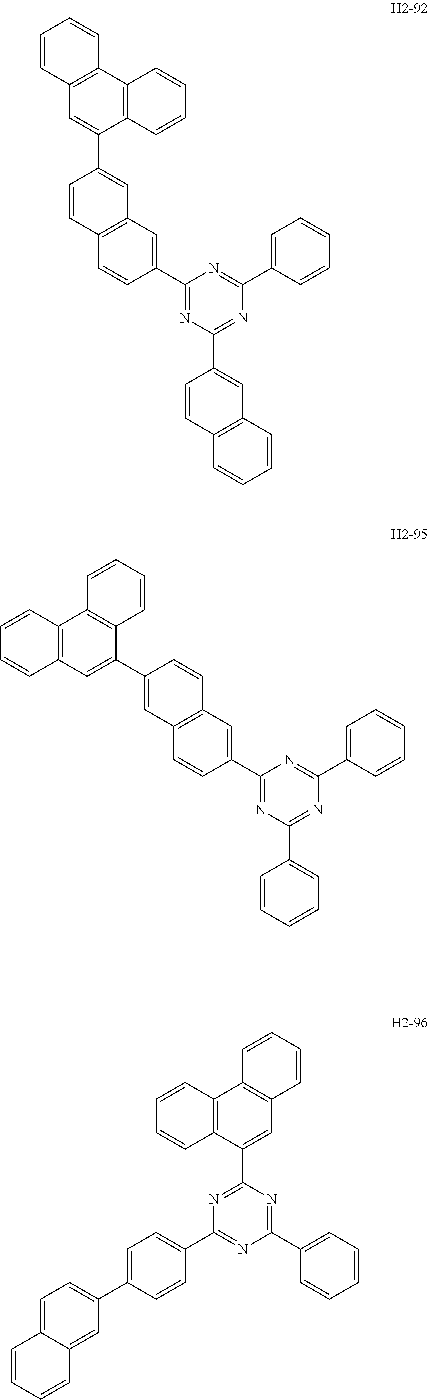 [SMILES: c1ccc(-c2nc(-c3ccc4ccccc4c3)nc(-c3ccc4ccc(-c5cc6ccccc6c6ccccc56)cc4c3)n2)cc1 ; c1ccc(-c2nc(-c3ccccc3)nc(-c3ccc4cc(-c5cc6ccccc6c6ccccc56)ccc4c3)n2)cc1 ; c1ccc(-c2nc(-c3ccc(-c4ccc5ccccc5c4)cc3)nc(-c3cc4ccccc4c4ccccc34)n2)cc1]

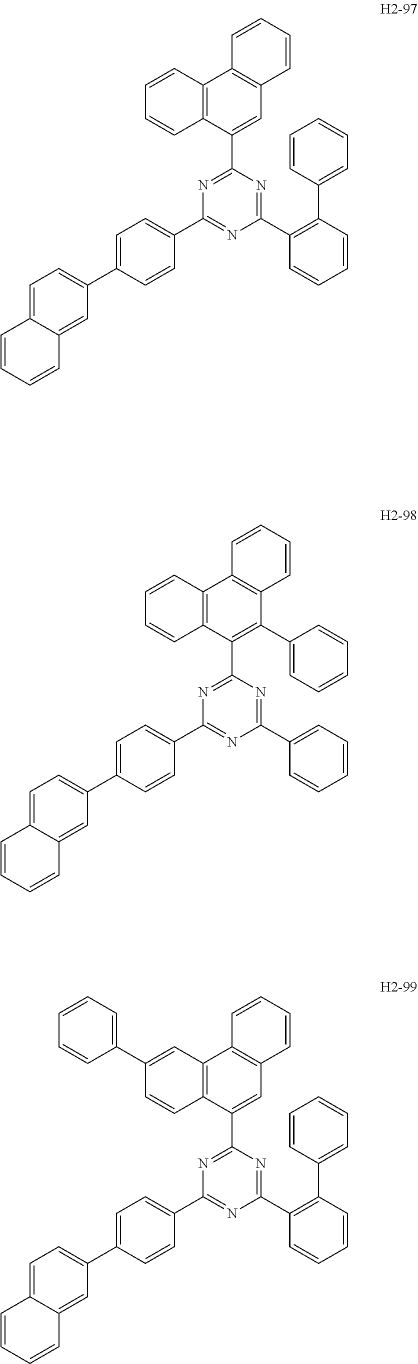 [SMILES: c1ccc(-c2ccccc2-c2nc(-c3ccc(-c4ccc5ccccc5c4)cc3)nc(-c3cc4ccccc4c4ccccc34)n2)cc1 ; c1ccc(-c2nc(-c3ccc(-c4ccc5ccccc5c4)cc3)nc(-c3c(-c4ccccc4)c4ccccc4c4ccccc34)n2)cc1 ; c1ccc(-c2ccc3c(-c4nc(-c5ccc(-c6ccc7ccccc7c6)cc5)nc(-c5ccccc5-c5ccccc5)n4)cc4ccccc4c3c2)cc1]

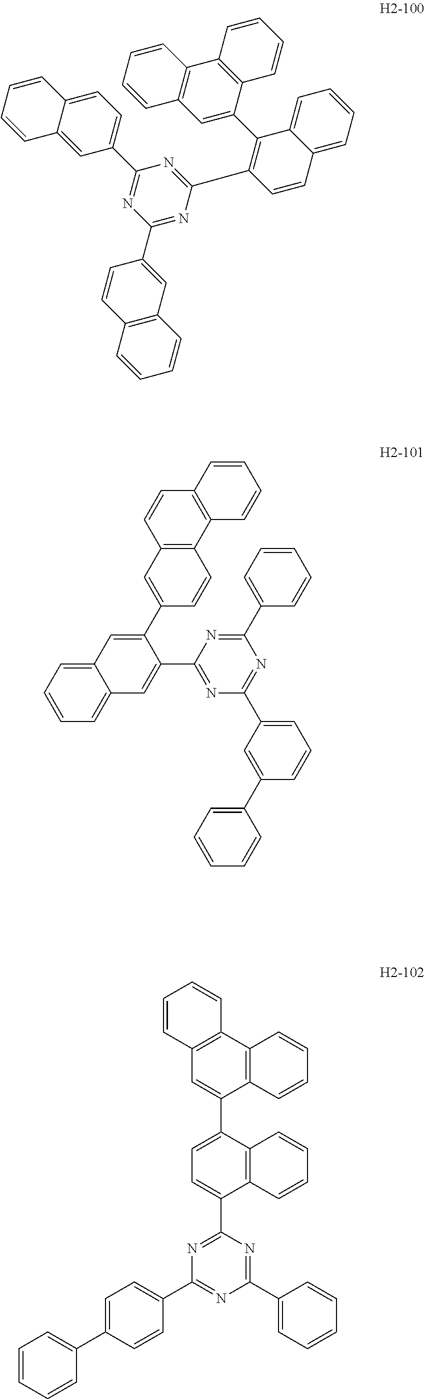 [SMILES: c1ccc2cc(-c3nc(-c4ccc5ccccc5c4)nc(-c4ccc5ccccc5c4-c4cc5ccccc5c5ccccc45)n3)ccc2c1 ; c1ccc(-c2cccc(-c3nc(-c4ccccc4)nc(-c4cc5ccccc5cc4-c4ccc5c(ccc6ccccc65)c4)n3)c2)cc1 ; c1ccc(-c2ccc(-c3nc(-c4ccccc4)nc(-c4ccc(-c5cc6ccccc6c6ccccc56)c5ccccc45)n3)cc2)cc1]

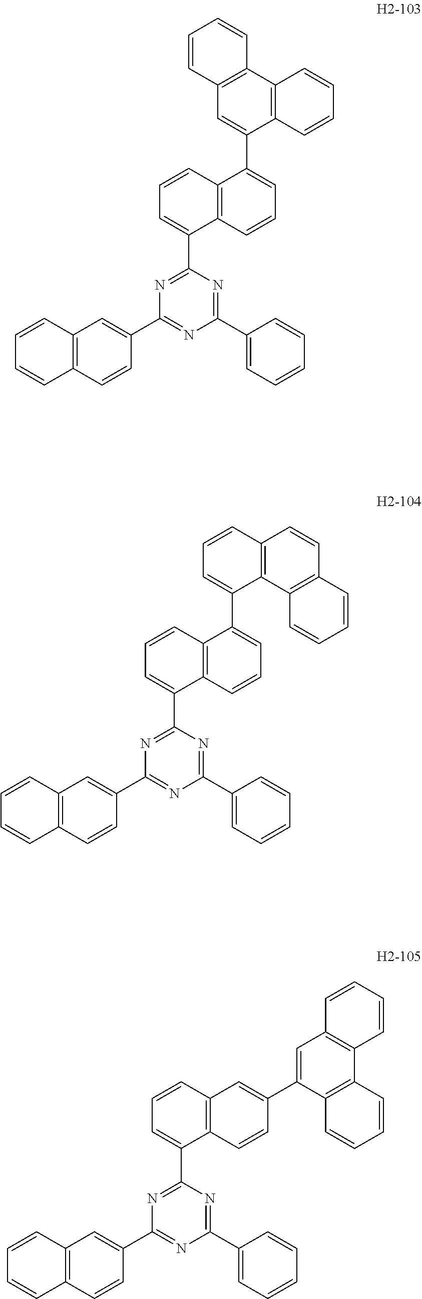 [SMILES: c1ccc(-c2nc(-c3ccc4ccccc4c3)nc(-c3cccc4c(-c5cc6ccccc6c6ccccc56)cccc34)n2)cc1 ; c1ccc(-c2nc(-c3ccc4ccccc4c3)nc(-c3cccc4c(-c5cccc6ccc7ccccc7c56)cccc34)n2)cc1 ; c1ccc(-c2nc(-c3ccc4ccccc4c3)nc(-c3cccc4cc(-c5cc6ccccc6c6ccccc56)ccc34)n2)cc1]

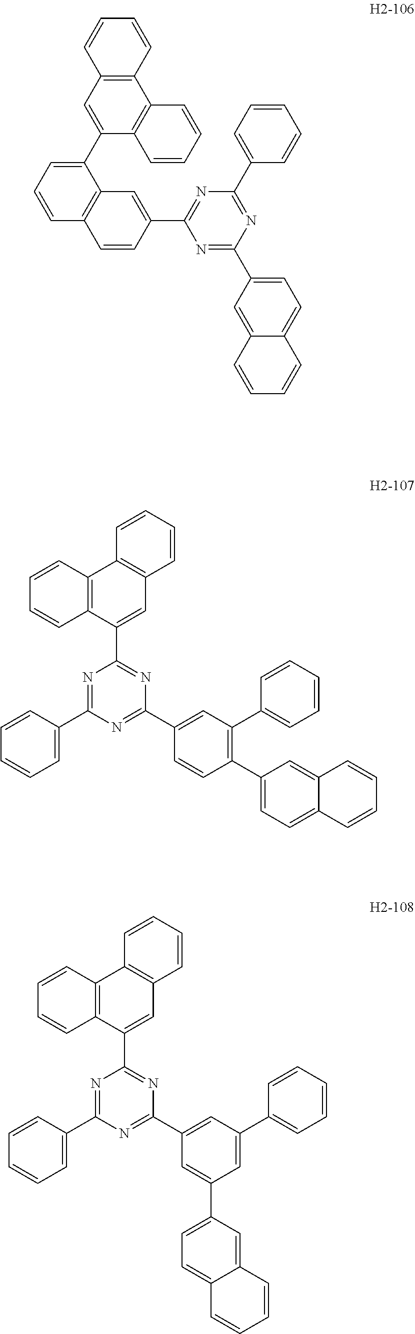 [SMILES: c1ccc(-c2nc(-c3ccc4ccccc4c3)nc(-c3ccc4cccc(-c5cc6ccccc6c6ccccc56)c4c3)n2)cc1 ; c1ccc(-c2nc(-c3ccc(-c4ccc5ccccc5c4)c(-c4ccccc4)c3)nc(-c3cc4ccccc4c4ccccc34)n2)cc1 ; c1ccc(-c2cc(-c3ccc4ccccc4c3)cc(-c3nc(-c4ccccc4)nc(-c4cc5ccccc5c5ccccc45)n3)c2)cc1]

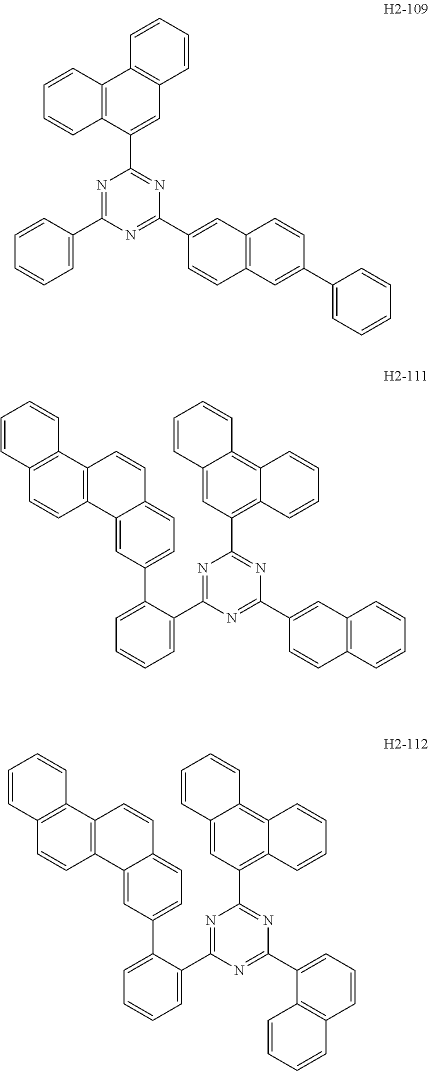 [SMILES: c1ccc(-c2ccc3cc(-c4nc(-c5ccccc5)nc(-c5cc6ccccc6c6ccccc56)n4)ccc3c2)cc1 ; c1ccc(-c2nc(-c3ccc4ccccc4c3)nc(-c3cc4ccccc4c4ccccc34)n2)c(-c2ccc3ccc4c5ccccc5ccc4c3c2)c1 ; c1ccc(-c2nc(-c3cccc4ccccc34)nc(-c3cc4ccccc4c4ccccc34)n2)c(-c2ccc3ccc4c5ccccc5ccc4c3c2)c1]

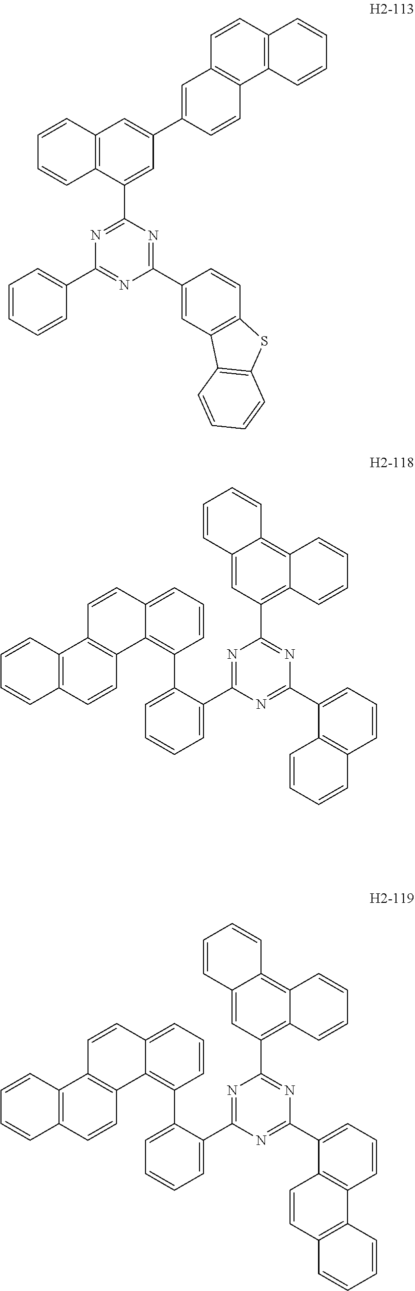 [SMILES: c1ccc(-c2nc(-c3ccc4sc5ccccc5c4c3)nc(-c3cc(-c4ccc5c(ccc6ccccc65)c4)cc4ccccc34)n2)cc1 ; c1ccc(-c2cccc3ccc4c5ccccc5ccc4c23)c(-c2nc(-c3cccc4ccccc34)nc(-c3cc4ccccc4c4ccccc34)n2)c1 ; c1ccc(-c2cccc3ccc4c5ccccc5ccc4c23)c(-c2nc(-c3cccc4c3ccc3ccccc34)nc(-c3cc4ccccc4c4ccccc34)n2)c1]

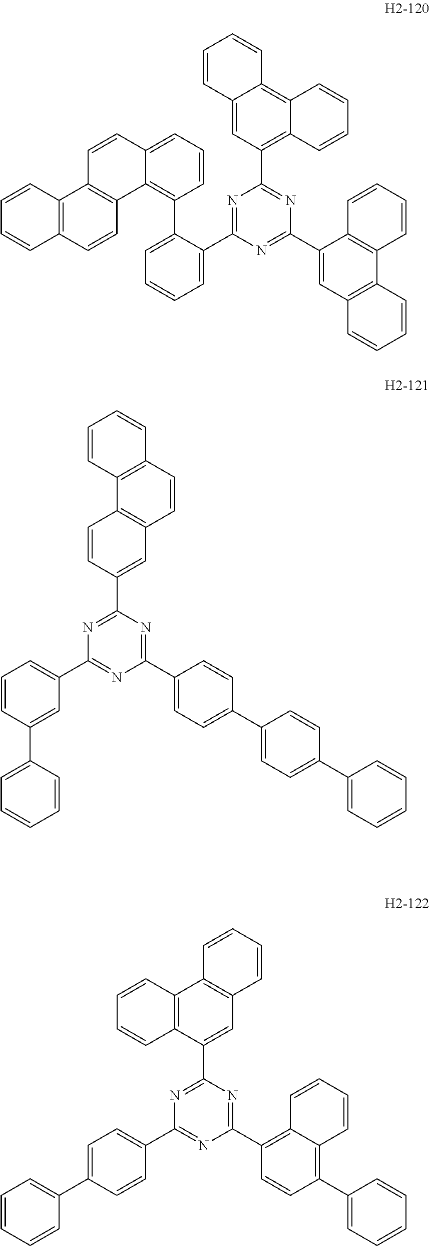 [SMILES: c1ccc(-c2cccc3ccc4c5ccccc5ccc4c23)c(-c2nc(-c3cc4ccccc4c4ccccc34)nc(-c3cc4ccccc4c4ccccc34)n2)c1 ; c1ccc(-c2ccc(-c3ccc(-c4nc(-c5cccc(-c6ccccc6)c5)nc(-c5ccc6c(ccc7ccccc76)c5)n4)cc3)cc2)cc1 ; c1ccc(-c2ccc(-c3nc(-c4ccc(-c5ccccc5)c5ccccc45)nc(-c4cc5ccccc5c5ccccc45)n3)cc2)cc1]

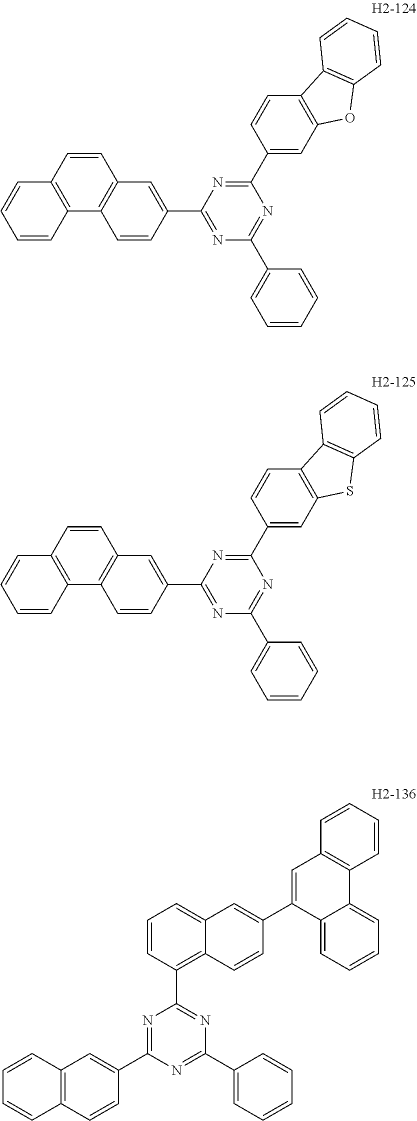 [SMILES: c1ccc(-c2nc(-c3ccc4c(ccc5ccccc54)c3)nc(-c3ccc4c(c3)oc3ccccc34)n2)cc1 ; c1ccc(-c2nc(-c3ccc4c(ccc5ccccc54)c3)nc(-c3ccc4c(c3)sc3ccccc34)n2)cc1 ; c1ccc(-c2nc(-c3ccc4ccccc4c3)nc(-c3cccc4cc(-c5cc6ccccc6c6ccccc56)ccc34)n2)cc1]

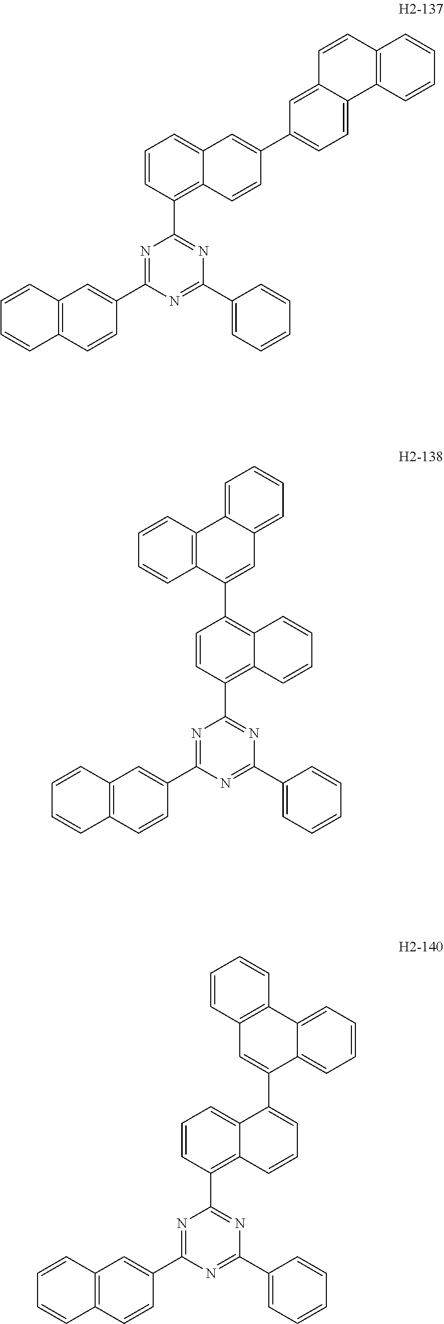 [SMILES: c1ccc(-c2nc(-c3ccc4ccccc4c3)nc(-c3cccc4cc(-c5ccc6c(ccc7ccccc76)c5)ccc34)n2)cc1 ; c1ccc(-c2nc(-c3ccc4ccccc4c3)nc(-c3ccc(-c4cc5ccccc5c5ccccc45)c4ccccc34)n2)cc1 ; c1ccc(-c2nc(-c3ccc4ccccc4c3)nc(-c3cccc4c(-c5cc6ccccc6c6ccccc56)cccc34)n2)cc1]

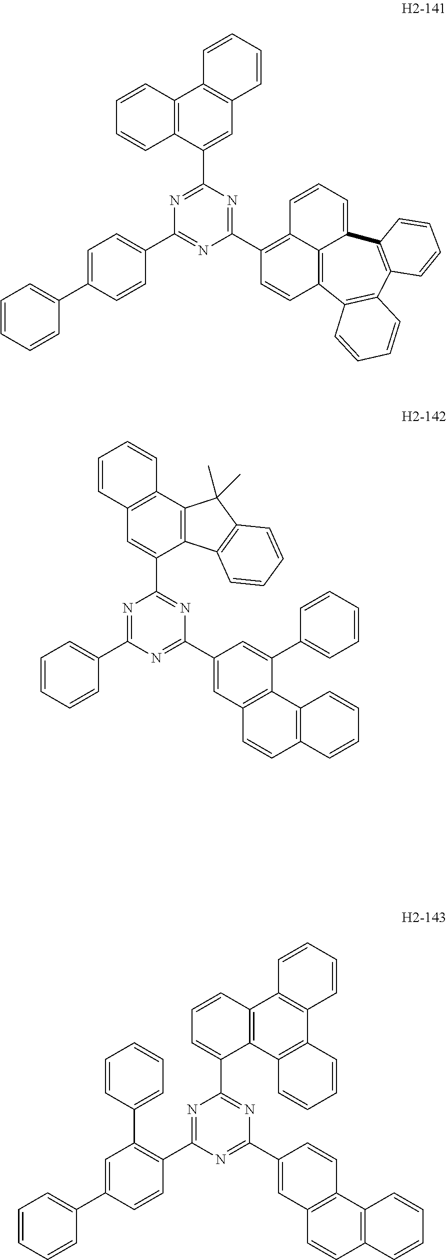

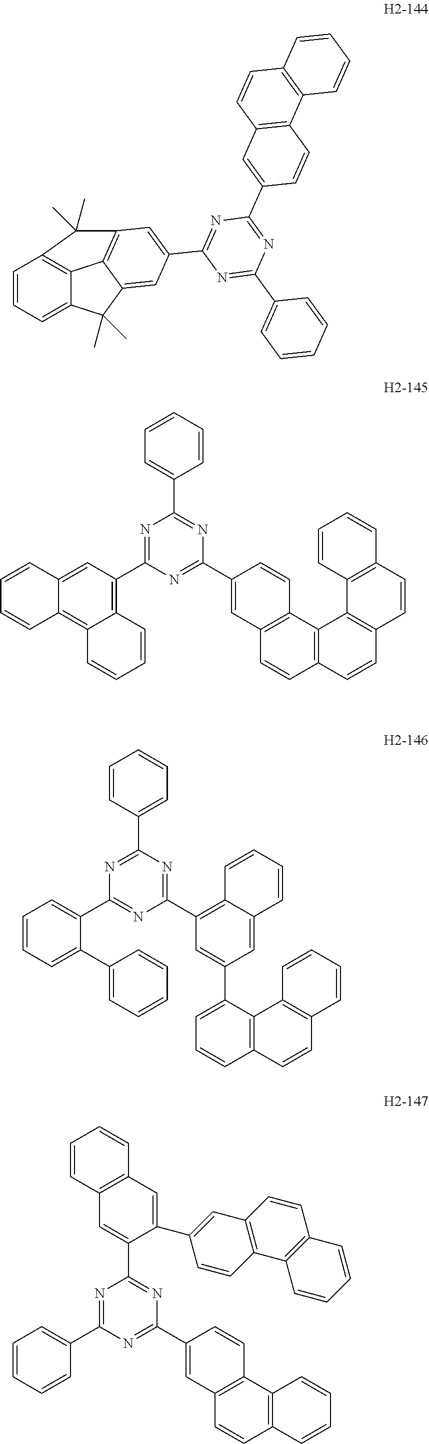 [SMILES: CC1(C)c2cccc3c2-c2c1cc(-c1nc(-c4ccccc4)nc(-c4ccc5c(ccc6ccccc65)c4)n1)cc2C3(C)C ; c1ccc(-c2nc(-c3ccc4c(ccc5ccc6ccc7ccccc7c6c54)c3)nc(-c3cc4ccccc4c4ccccc34)n2)cc1 ; c1ccc(-c2nc(-c3ccccc3-c3ccccc3)nc(-c3cc(-c4cccc5ccc6ccccc6c45)cc4ccccc34)n2)cc1 ; c1ccc(-c2nc(-c3ccc4c(ccc5ccccc54)c3)nc(-c3cc4ccccc4cc3-c3ccc4c(ccc5ccccc54)c3)n2)cc1]

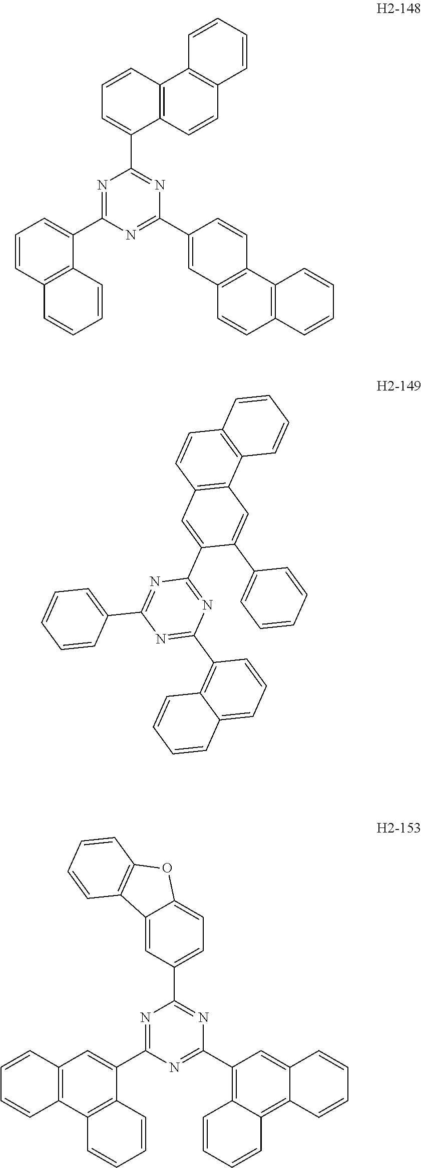 [SMILES: c1ccc2c(-c3nc(-c4ccc5c(ccc6ccccc65)c4)nc(-c4cccc5c4ccc4ccccc45)n3)cccc2c1 ; c1ccc(-c2nc(-c3cc4ccc5ccccc5c4cc3-c3ccccc3)nc(-c3cccc4ccccc34)n2)cc1 ; c1ccc2c(c1)cc(-c1nc(-c3ccc4oc5ccccc5c4c3)nc(-c3cc4ccccc4c4ccccc34)n1)c1ccccc12]

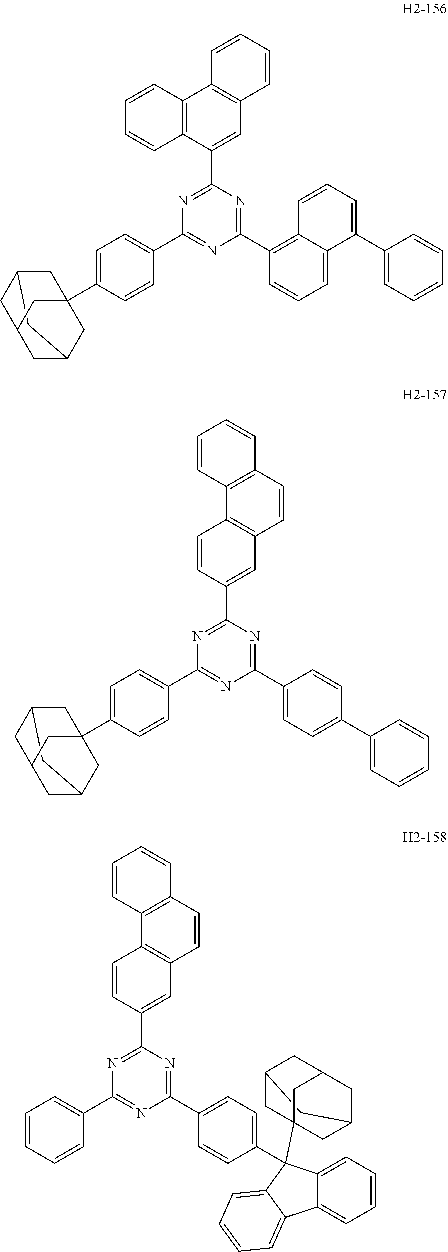 [SMILES: c1ccc(-c2cccc3c(-c4nc(-c5ccc(C67CC8CC(CC(C8)C6)C7)cc5)nc(-c5cc6ccccc6c6ccccc56)n4)cccc23)cc1 ; c1ccc(-c2ccc(-c3nc(-c4ccc(C56CC7CC(CC(C7)C5)C6)cc4)nc(-c4ccc5c(ccc6ccccc65)c4)n3)cc2)cc1 ; c1ccc(-c2nc(-c3ccc(C4(C56CC7CC(CC(C7)C5)C6)c5ccccc5-c5ccccc54)cc3)nc(-c3ccc4c(ccc5ccccc54)c3)n2)cc1]

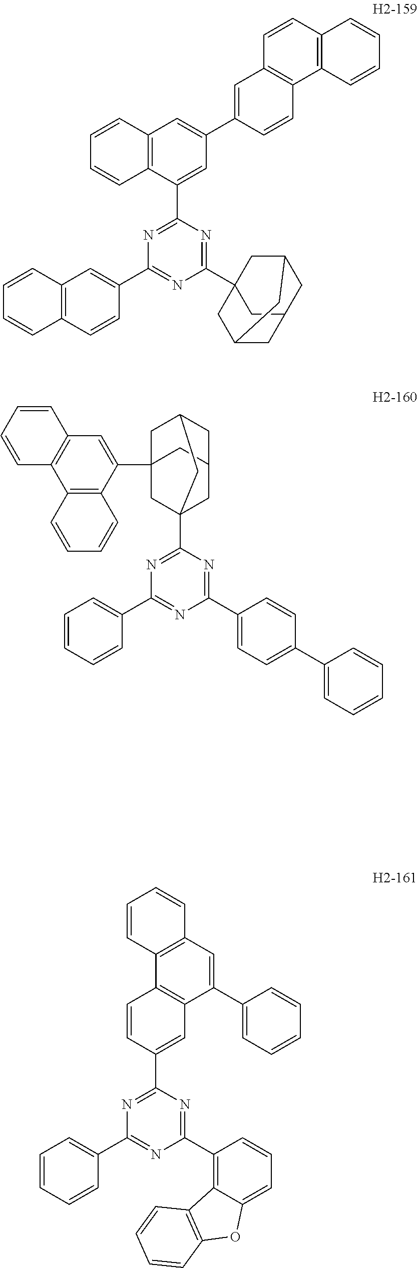 [SMILES: c1ccc2cc(-c3nc(-c4cc(-c5ccc6c(ccc7ccccc76)c5)cc5ccccc45)nc(C45CC6CC(CC(C6)C4)C5)n3)ccc2c1 ; c1ccc(-c2ccc(-c3nc(-c4ccccc4)nc(C45CC6CC(C4)CC(c4cc7ccccc7c7ccccc47)(C6)C5)n3)cc2)cc1 ; c1ccc(-c2nc(-c3ccc4c(c3)c(-c3ccccc3)cc3ccccc34)nc(-c3cccc4oc5ccccc5c34)n2)cc1]

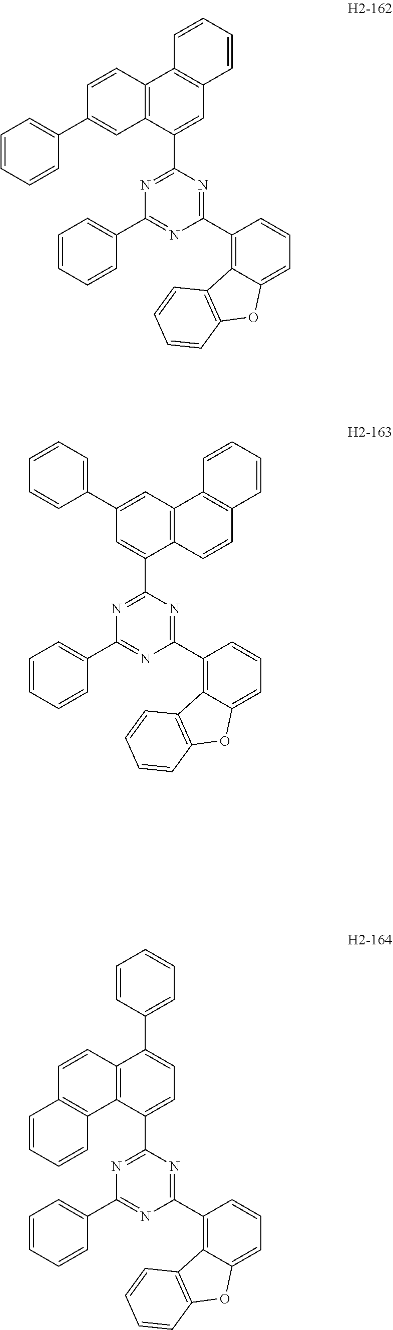 [SMILES: c1ccc(-c2ccc3c(c2)c(-c2nc(-c4ccccc4)nc(-c4cccc5oc6ccccc6c45)n2)cc2ccccc23)cc1 ; c1ccc(-c2cc(-c3nc(-c4ccccc4)nc(-c4cccc5oc6ccccc6c45)n3)c3ccc4ccccc4c3c2)cc1 ; c1ccc(-c2nc(-c3cccc4oc5ccccc5c34)nc(-c3ccc(-c4ccccc4)c4ccc5ccccc5c34)n2)cc1]

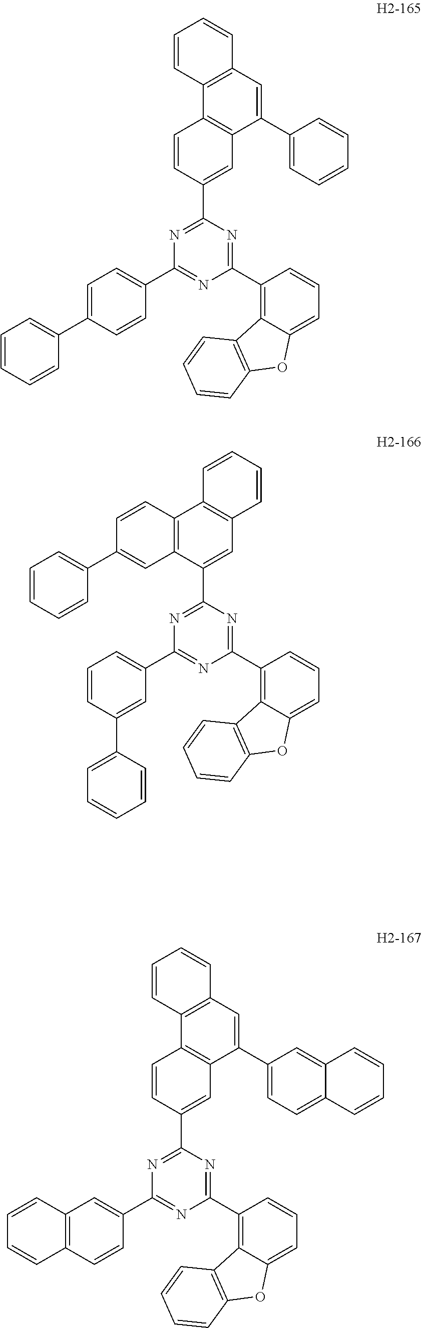 [SMILES: c1ccc(-c2ccc(-c3nc(-c4ccc5c(c4)c(-c4ccccc4)cc4ccccc45)nc(-c4cccc5oc6ccccc6c45)n3)cc2)cc1 ; c1ccc(-c2cccc(-c3nc(-c4cc5ccccc5c5ccc(-c6ccccc6)cc45)nc(-c4cccc5oc6ccccc6c45)n3)c2)cc1 ; c1ccc2cc(-c3nc(-c4ccc5c(c4)c(-c4ccc6ccccc6c4)cc4ccccc45)nc(-c4cccc5oc6ccccc6c45)n3)ccc2c1]

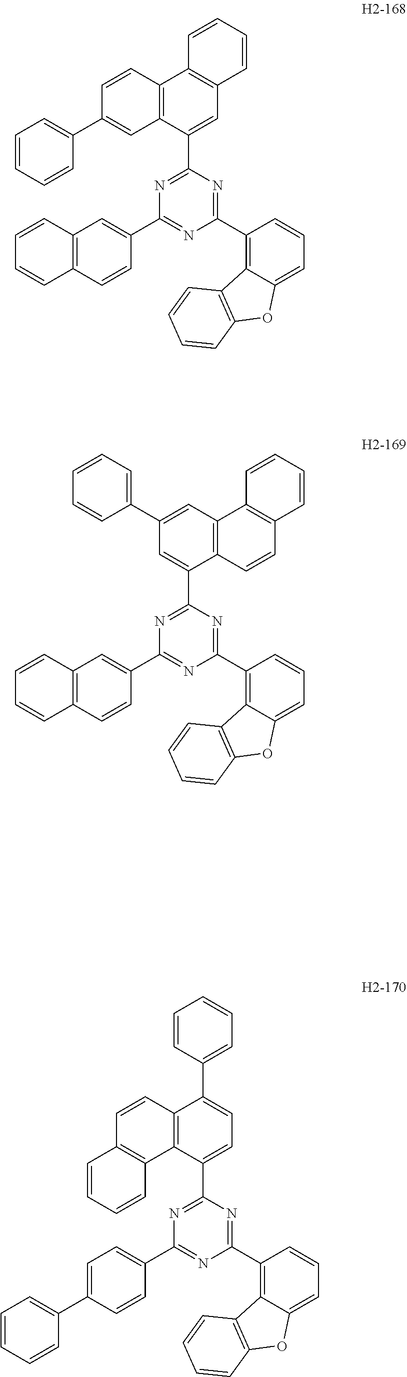 [SMILES: c1ccc(-c2ccc3c(c2)c(-c2nc(-c4ccc5ccccc5c4)nc(-c4cccc5oc6ccccc6c45)n2)cc2ccccc23)cc1 ; c1ccc(-c2cc(-c3nc(-c4ccc5ccccc5c4)nc(-c4cccc5oc6ccccc6c45)n3)c3ccc4ccccc4c3c2)cc1 ; c1ccc(-c2ccc(-c3nc(-c4cccc5oc6ccccc6c45)nc(-c4ccc(-c5ccccc5)c5ccc6ccccc6c45)n3)cc2)cc1]

In addition, the present disclosure provides an OLED comprising the organic electroluminescent compound represented by formula 2′. Herein, the organic electroluminescent compound represented by formula 2′ may be comprised in a light-emitting layer, an electron transport layer, or an electron buffer layer, but is not limited thereto.

The compound represented by formula 1 according to the present disclosure may be prepared by referring to a synthetic method known to one skilled in the art. For example, the compound represented by formula 1 can be prepared by referring to Korean Patent Application Laying-Open No. 10-2020-0007644 (published on Jan. 22, 2020), Korean Patent Application Laying-Open No. 10-2018-0099487 (published on Sep. 5, 2018), etc., but is not limited thereto.

The compound represented by formula 2 or 2′ according to the present disclosure may be prepared by referring to a synthetic method known to one skilled in the art. For example, the compound represented by formula 2 or 2′ according to the present disclosure may be prepared by referring to the following reaction scheme, but is not limited thereto.

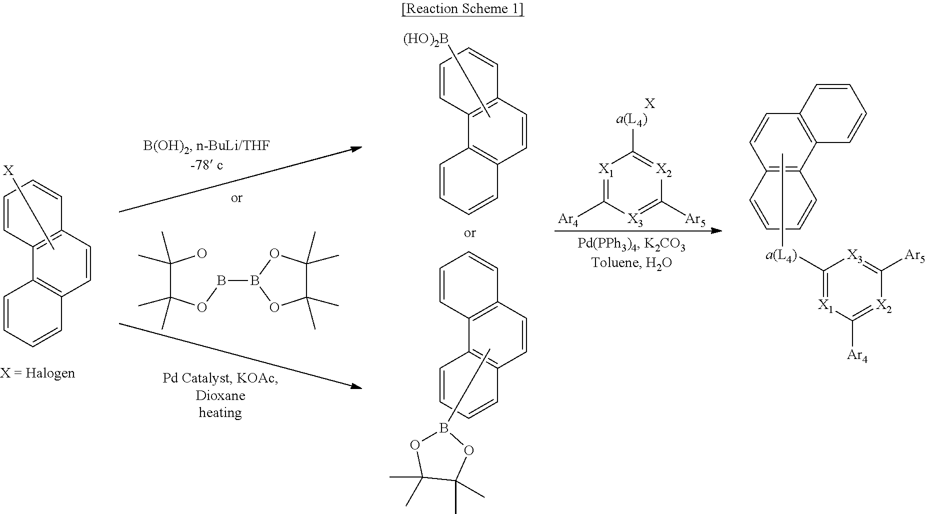

In reaction scheme 1, X₁ to X₃, Ar₄, Ar₅, L₄ and a are as defined in formula 2 or 2′.

Although illustrative synthesis examples of the compound represented by formula 2 or 2′ of the present disclosure are described above, one skilled in the art will be able to readily understand that all of them are based on a Buchwald-Hartwig cross-coupling reaction, an N-arylation reaction, H-mont-mediated etherification reaction, a Miyaura borylation reaction, a Suzuki cross-coupling reaction, an intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, a Phosphine-mediated reductive cyclization reaction, etc., and the reactions above proceed even when substituents, which are defined in formula 2 or 2′ but are not specified in the specific synthesis examples, are bonded.

The organic electroluminescent device according to the present disclosure may comprise an anode, a cathode, and at least one organic layer between the anode and cathode in which the organic layer may comprise a plurality of organic electroluminescent materials including the compound represented by formula 1 as the first organic electroluminescent material, and the compound represented by formula 2 as the second organic electroluminescent material. According to one embodiment of the present disclosure, the organic electroluminescent device according to the present disclosure may comprise an anode, a cathode, and at least one light-emitting layer between the anode and cathode in which the light-emitting layer may comprise the compound represented by formula 1 and the compound represented by formula 2.

The light-emitting layer includes a host and a dopant, in which the host includes a plurality of host materials, and the compound represented by formula 1 may be included as the first host compound of the plurality of host materials, and the compound represented by formula 2 may be included as the second host compound of the plurality of host materials. Herein, the weight ratio of the first host compound and the second host compound is about 1:99 to about 99:1, preferably about 10:90 to about 90:10, more preferably about 30:70 to about 70:30, even more preferably about 40:60 to about 60:40, and still more preferably about 50:50.

Herein, the light-emitting layer is a layer from which light is emitted, and may be a single layer or a multi-layer in which two or more layers are stacked. In the plurality of host materials of the present disclosure, all of the first host material and the second host material may be included in one layer, or the first host material and the second host material may be included in respective different light-emitting layers. According to one embodiment of the present disclosure, the doping concentration of the dopant compound with respect to the host compound in the light-emitting layer may be less than 20 wt %.

The organic electroluminescent device of the present disclosure may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer. According to one embodiment of the present disclosure, the organic electroluminescent device of the present disclosure may further comprise an amine-based compound besides the plurality of host materials of the present disclosure as at least one of a hole injection material, a hole transport material, a hole auxiliary material, a light-emitting material, a light-emitting auxiliary material, and an electron blocking material. In addition, according to one embodiment of the present disclosure, the organic electroluminescent device of the present disclosure may further comprise an azine-based compound besides the plurality of host materials of the present disclosure as at least one of an electron transport material, an electron injection material, an electron buffer material, and a hole blocking material.

The plurality of host materials according to the present disclosure may be used as a light-emitting material for a white organic light-emitting device. The white organic light-emitting device has been suggested to have various structures such as a side-by-side method, or a stacking method depending on the arrangement of R (Red), G (Green) or YG (yellowish green), and B (blue) light-emitting units, or CCM (color conversion material) method, etc. In addition, the plurality of host materials according to one embodiment of the present disclosure may also be applied to the organic electroluminescent device comprising a QD (quantum dot).

A hole injection layer, a hole transport layer, an electron blocking layer, or a combination thereof may be used between the anode and the light-emitting layer. The hole injection layer may be multilayers in order to lower the hole injection barrier (or hole injection voltage) from the anode to the hole transport layer or the electron blocking layer, wherein two compounds may be simultaneously used in each of the multilayers. In addition, the hole injection layer may be doped with p-dopant. The electron blocking layer may be placed between the hole transport layer (or hole injection layer) and the light-emitting layer, and may block the overflow of electrons from the light-emitting layer and confine the excitons in the light-emitting layer to prevent light leakage. The hole transport layer or the electron blocking layer may be multilayers, wherein a plurality of compounds may be used in each of the multilayers.

An electron buffer layer, a hole blocking layer, an electron transport layer, an electron injection layer, or a combination thereof may be used between the light-emitting layer and the cathode. The electron buffer layer may be multilayers for the purpose of controlling electron injection and improving interfacial properties between the light-emitting layer and the electron injection layer, wherein two compounds may be simultaneously used in each of the multilayers. The hole blocking layer or the electron transport layer may also be multilayers, wherein a plurality of compounds may be used in each of the multilayers. In addition, the electron injection layer may be doped with n-dopant.

Dopants comprised in the organic electroluminescent device of the present disclosure may be at least one phosphorescent or fluorescent dopant, preferably a phosphorescent dopant. The phosphorescent dopant materials applied to the organic electroluminescent device according to the present disclosure are not particularly limited, but may be a metallated complex compound(s) of a metal atom(s) selected from iridium (Ir), osmium (Os), copper (Cu), and platinum (Pt), and in some case, may be preferably an ortho-metallated complex compound(s) of a metal atom(s) selected from iridium (Ir), osmium (Os), copper (Cu), and platinum (Pt), and in some case, may be more preferably an ortho-metallated iridium complex compound(s).

The dopant comprised in the organic electroluminescent device of the present disclosure may be the compound represented by the following formula 101, but is not limited thereto.

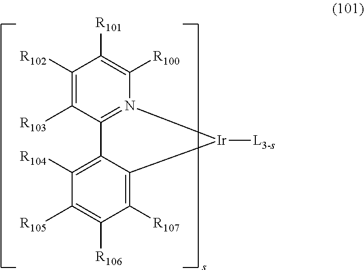

In formula 101,
L is selected from the following structures 1 to 3:

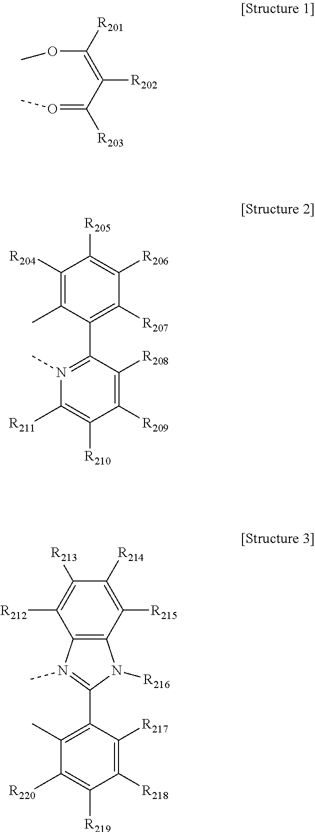

R₁₀₀ to R₁₀₃ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent(s) to form a ring(s), e.g., a substituted or unsubstituted quinoline, a substituted or unsubstituted isoquinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline, together with pyridine;
R₁₀₄ to R₁₀₇ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent(s) to form a ring(s), e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine, together with benzene;
R₂₀₁ to R₂₂₀ each independently represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent(s) to form a ring(s); and
s represents an integer of 1 to 3.

The specific examples of the dopant compound are as follows, but are not limited thereto.

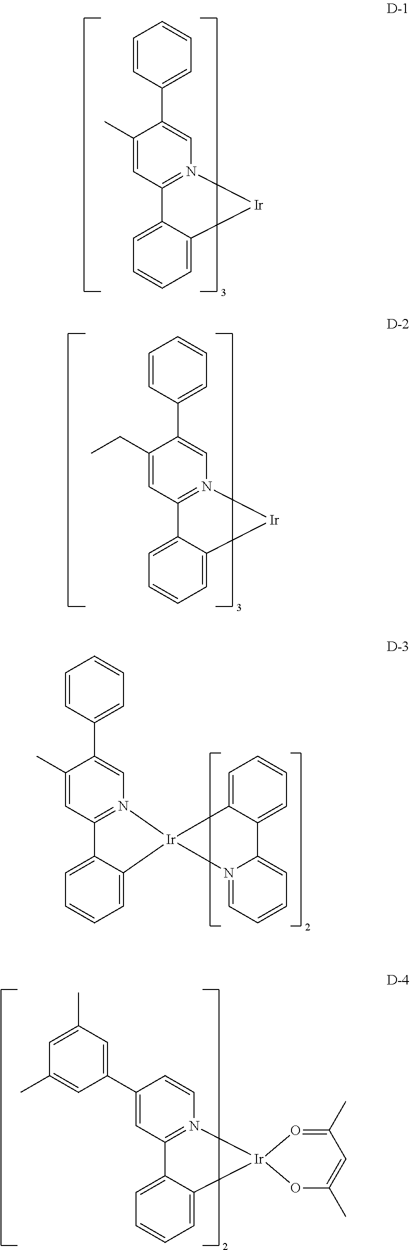

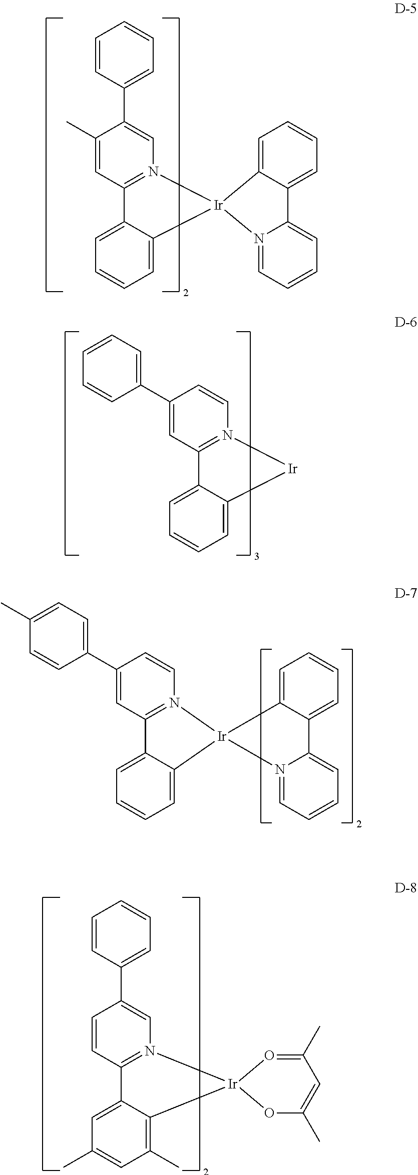

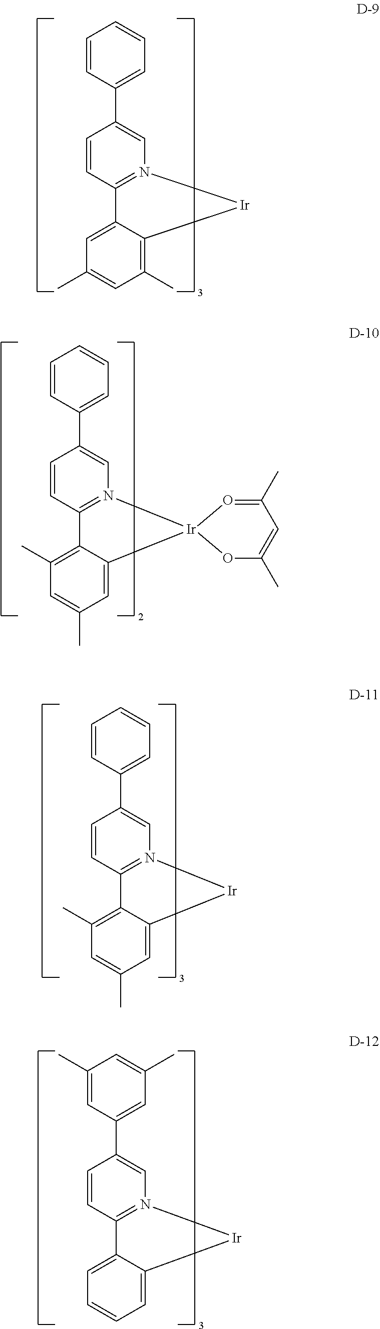

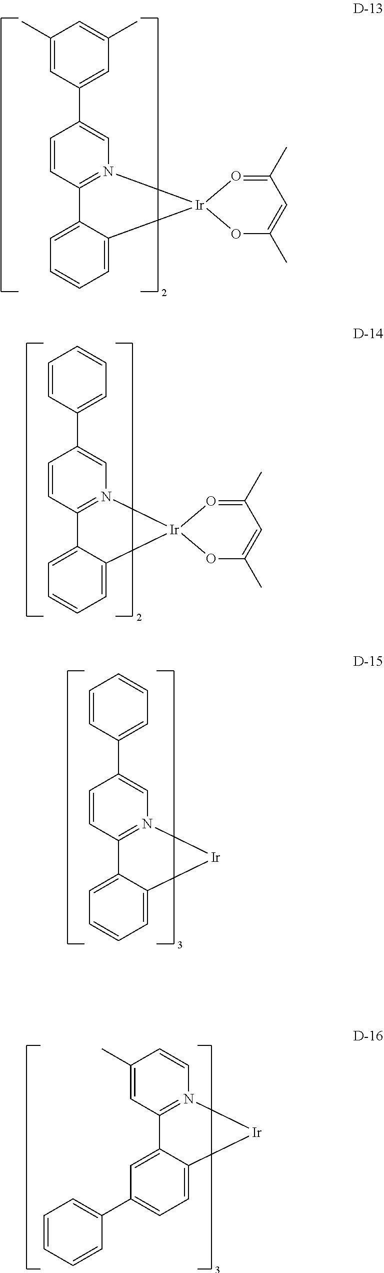

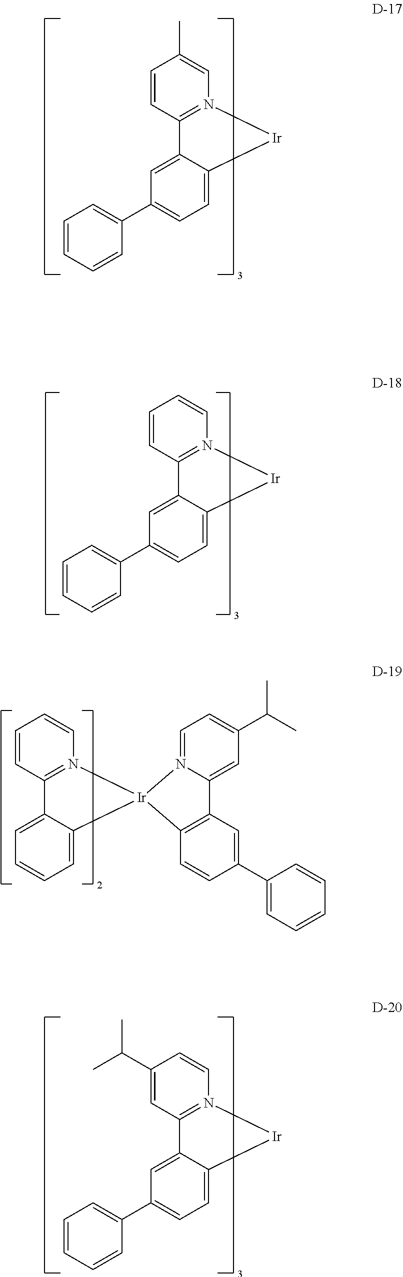

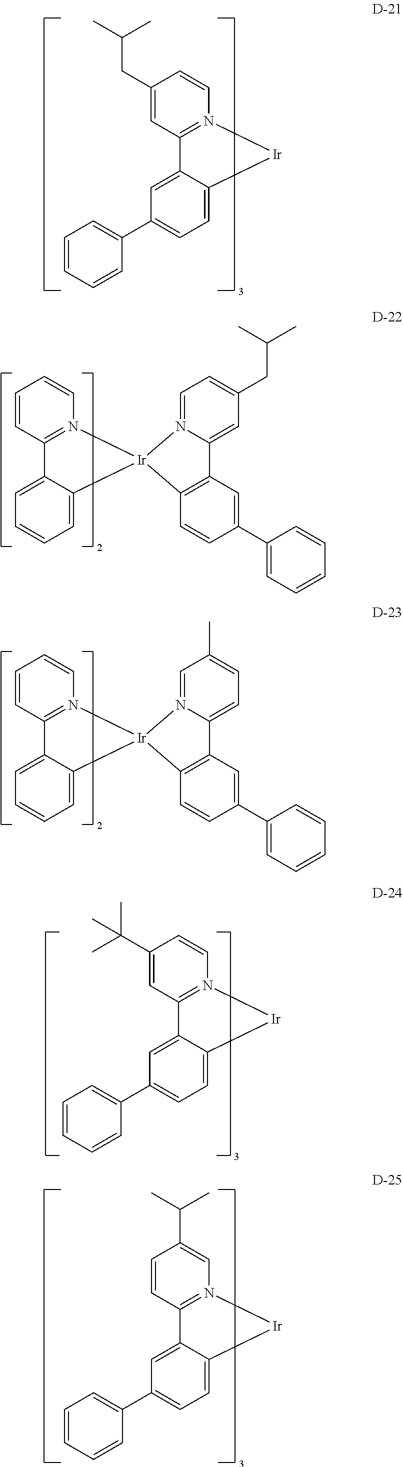

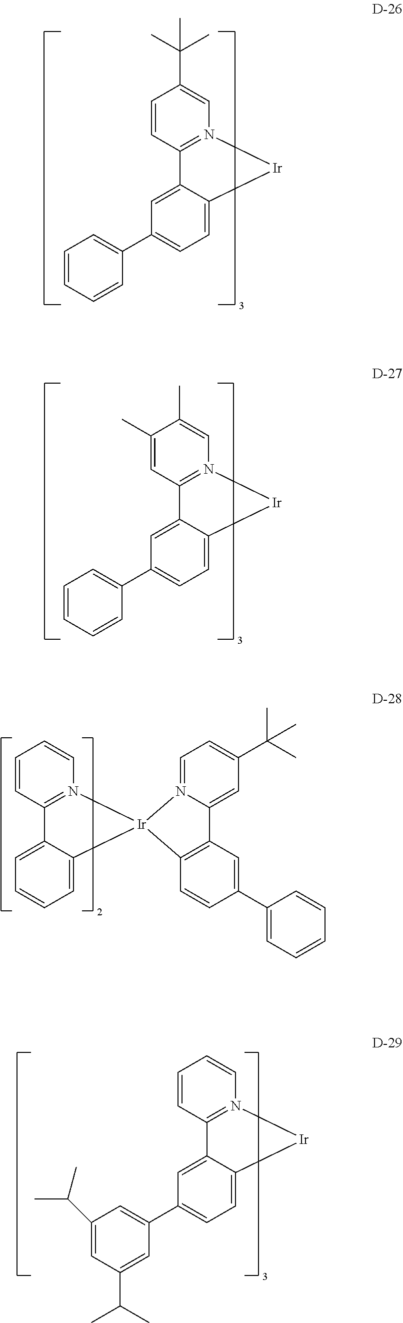

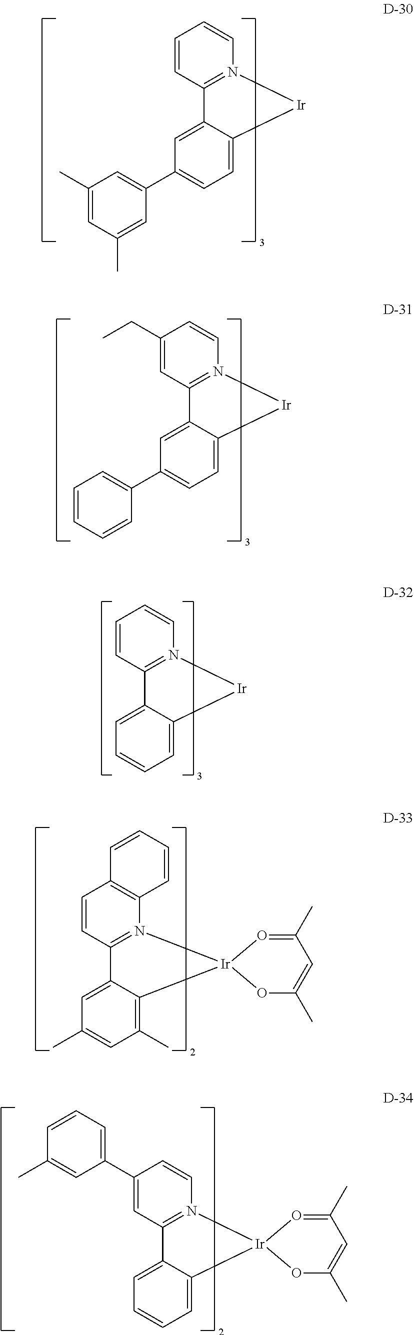

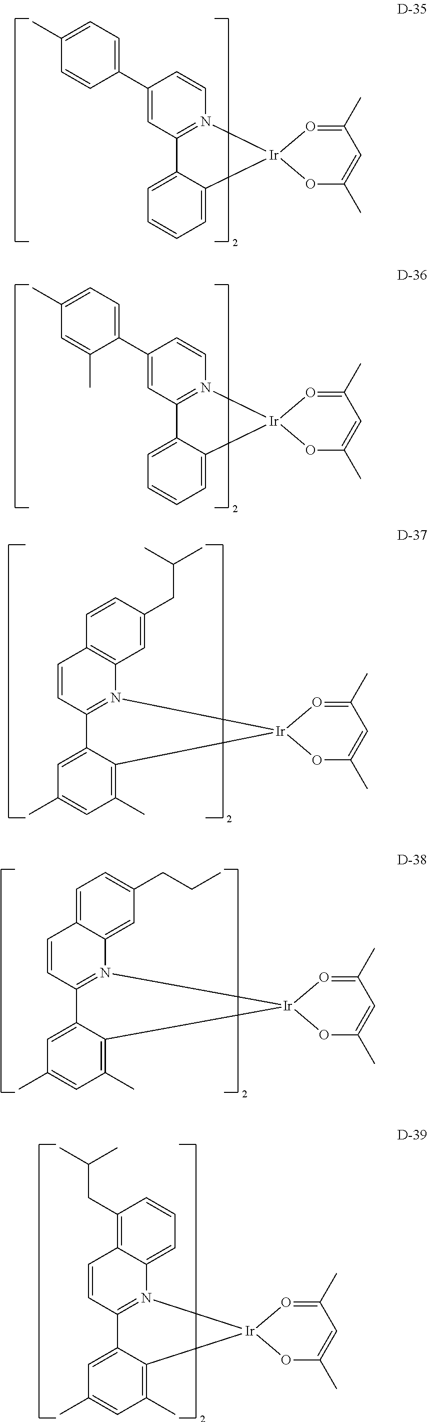

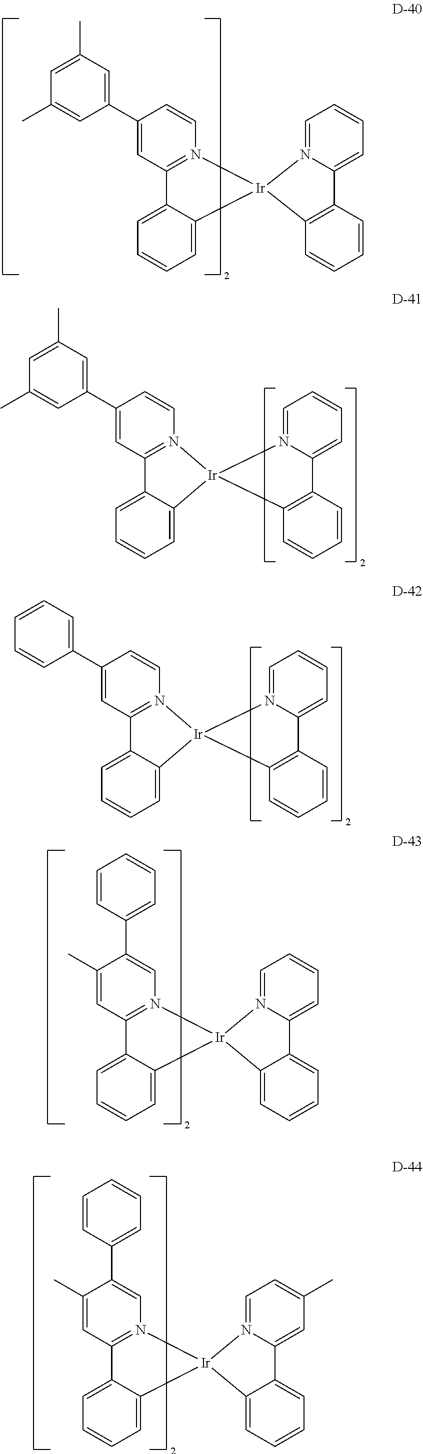

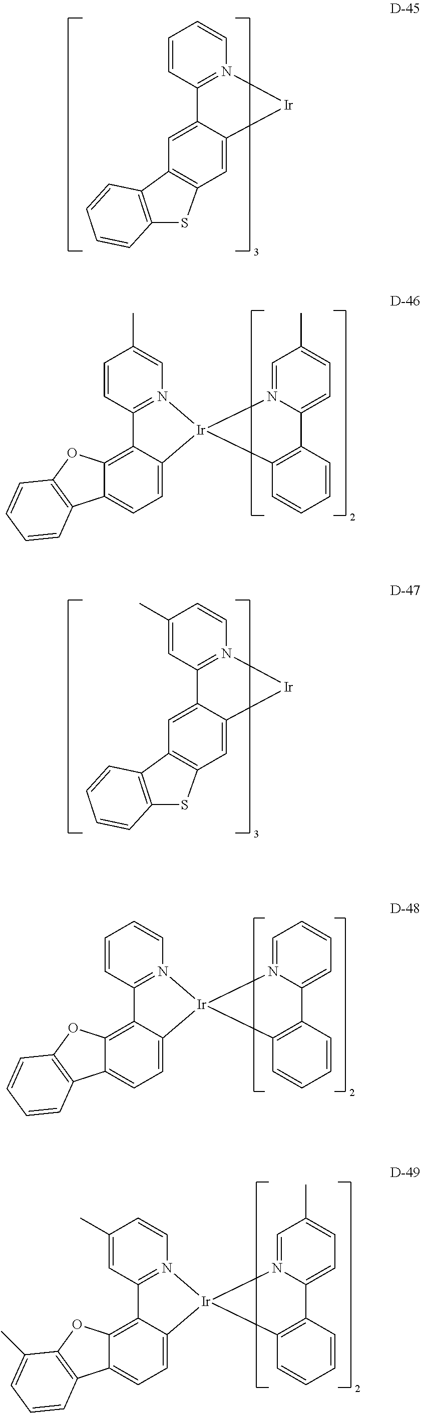

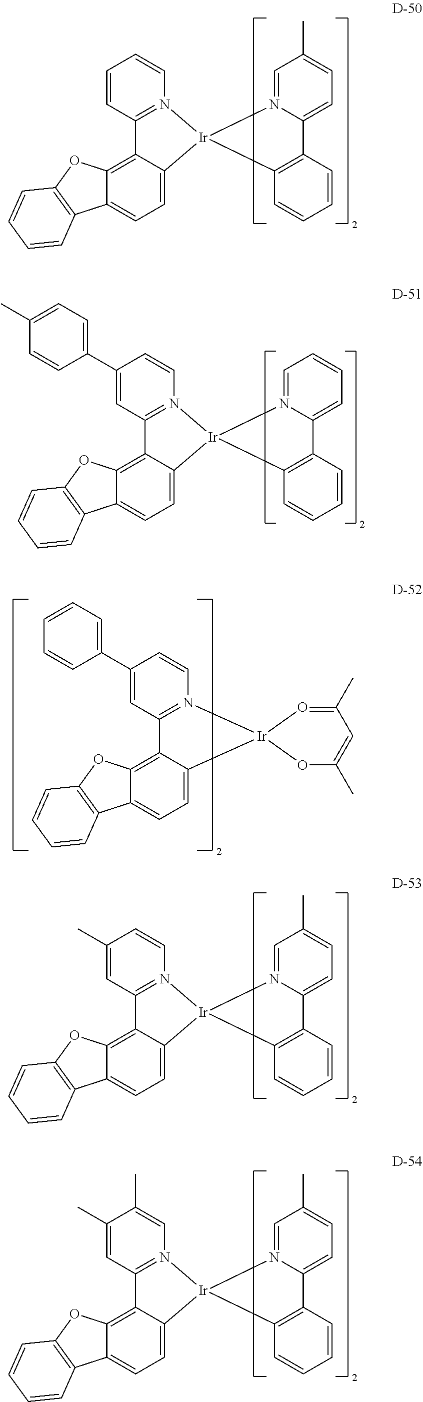

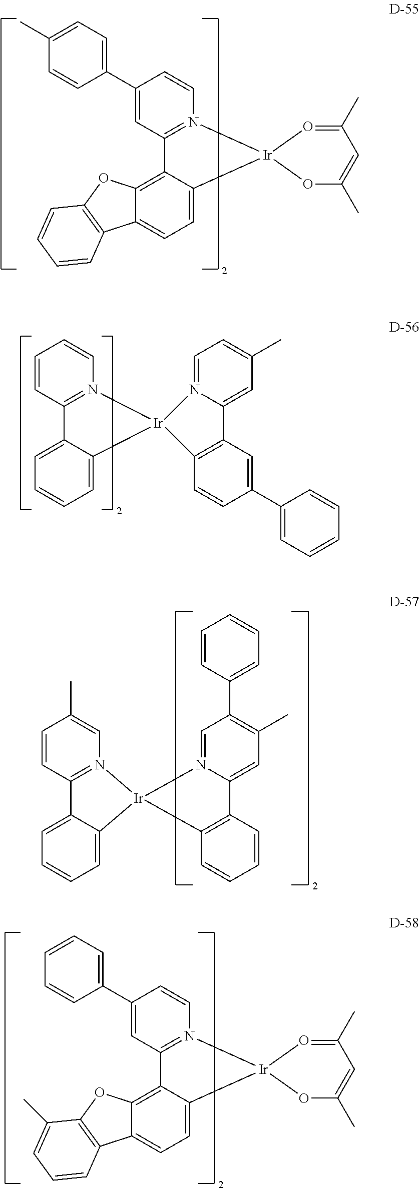

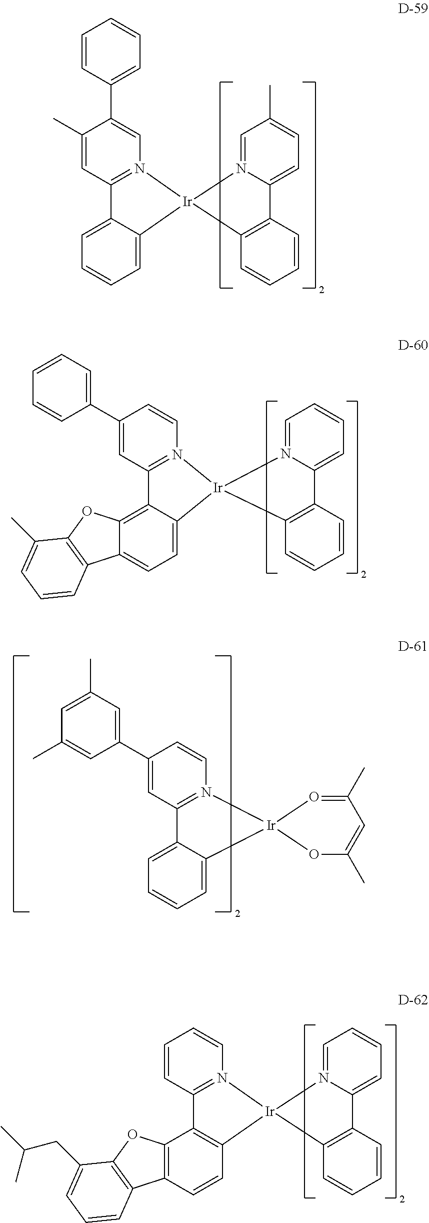

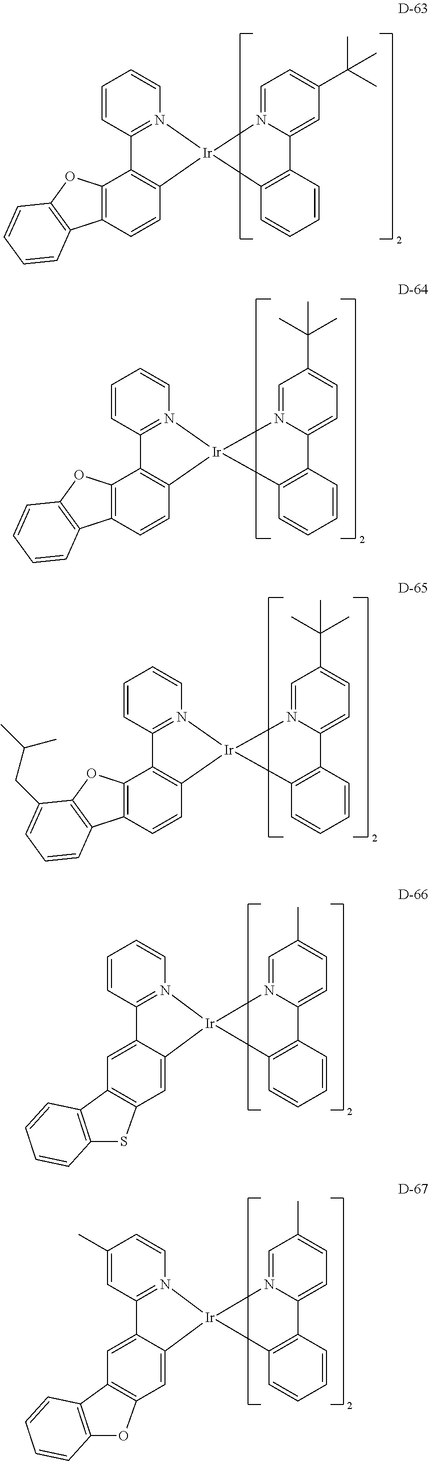

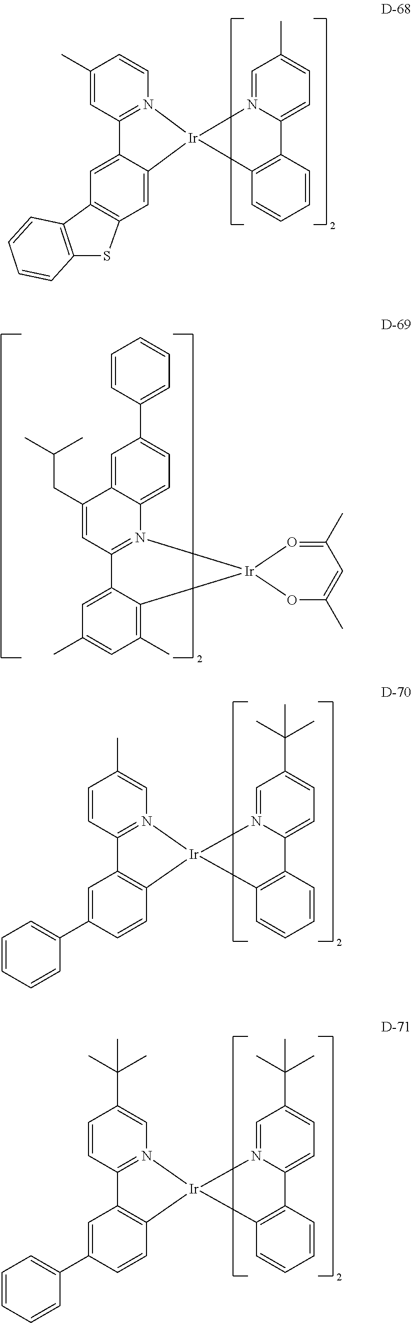

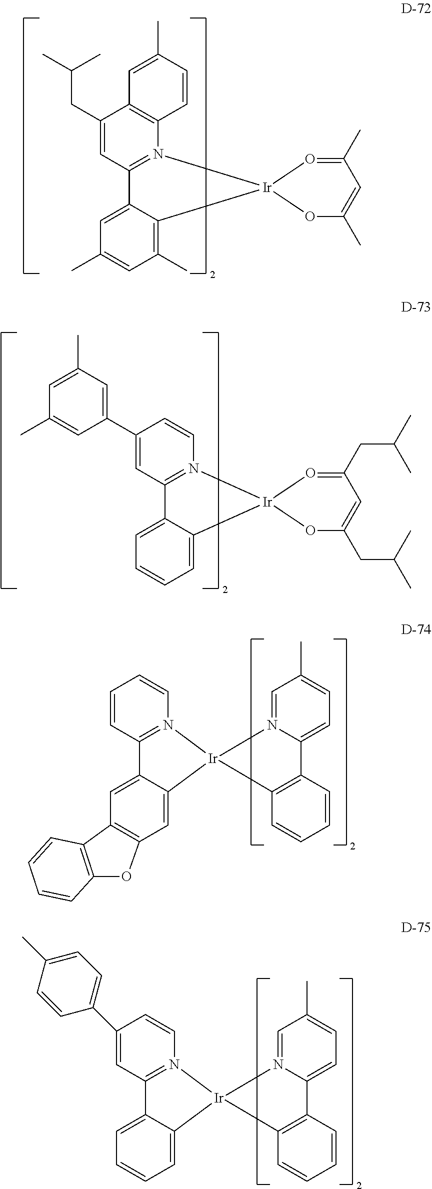

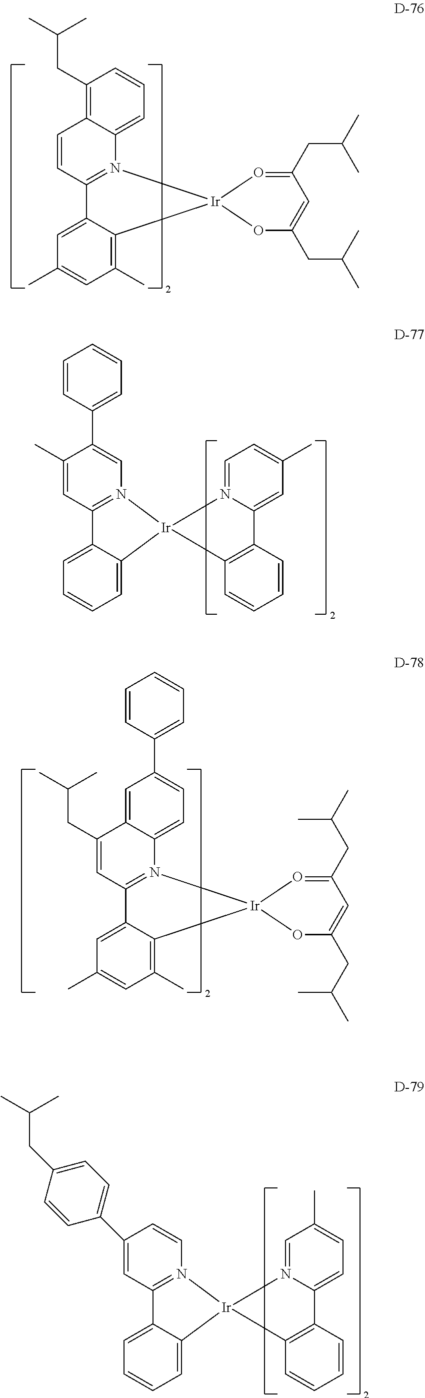

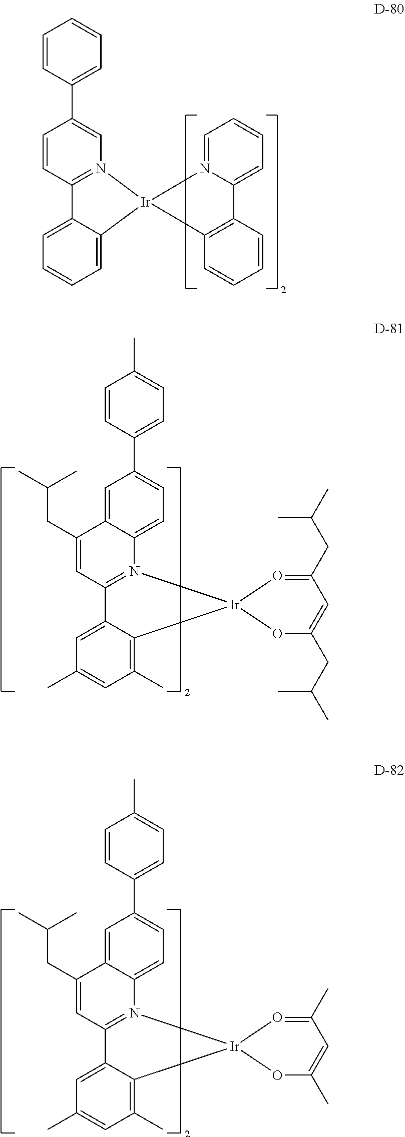

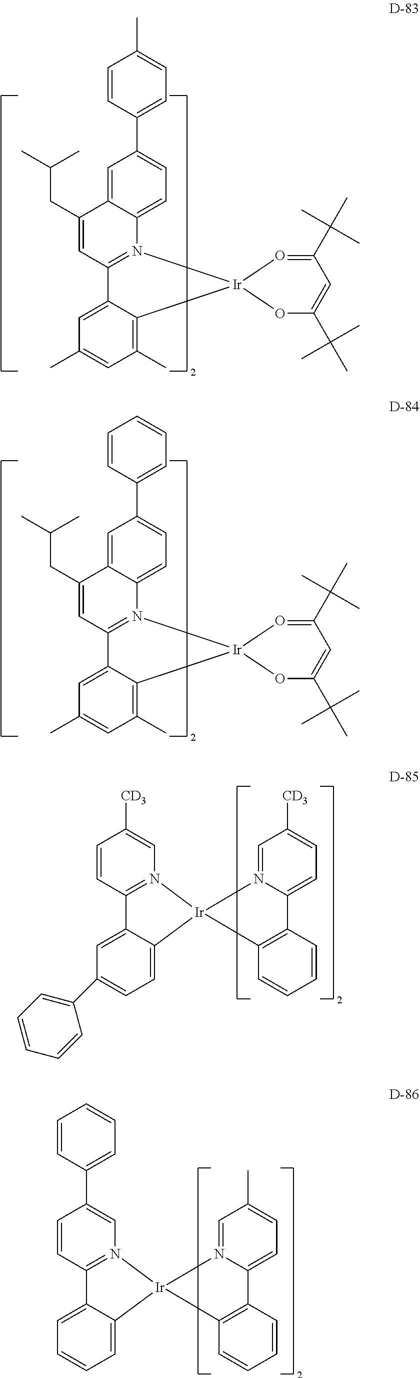

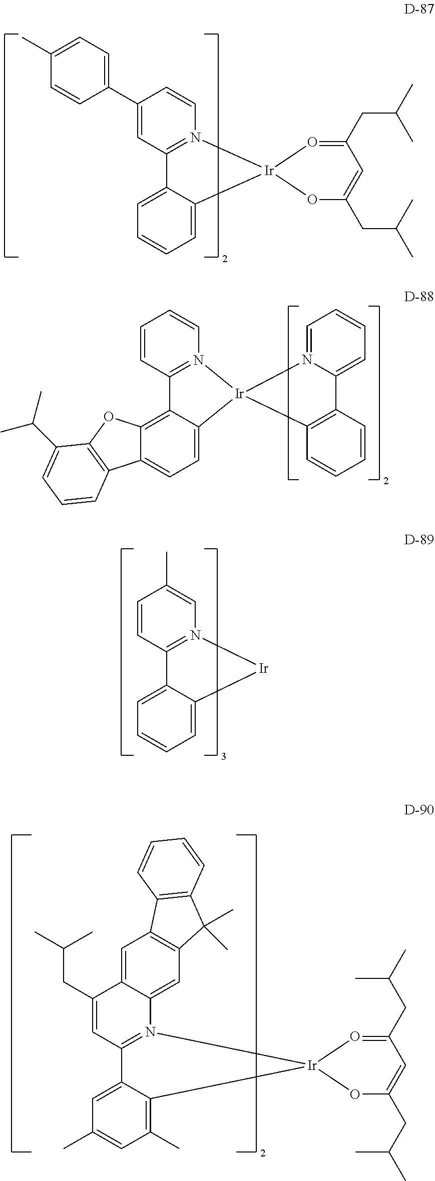

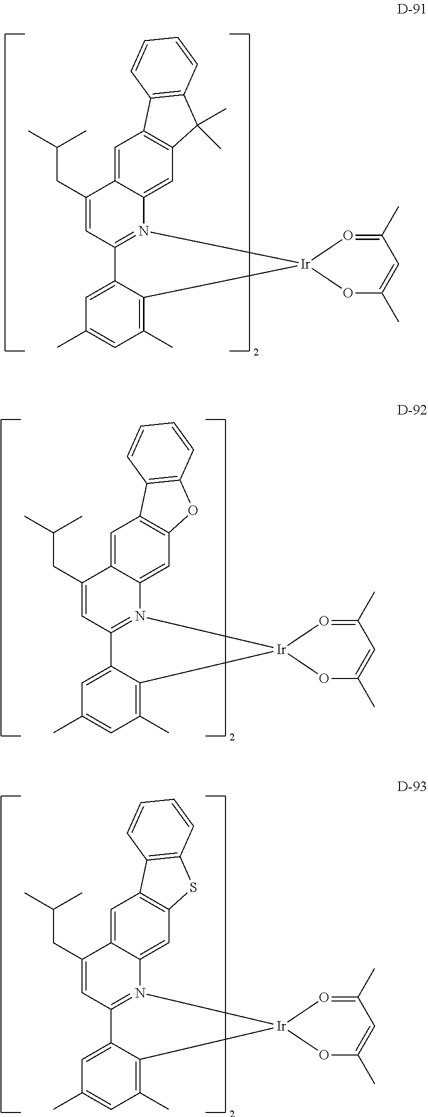

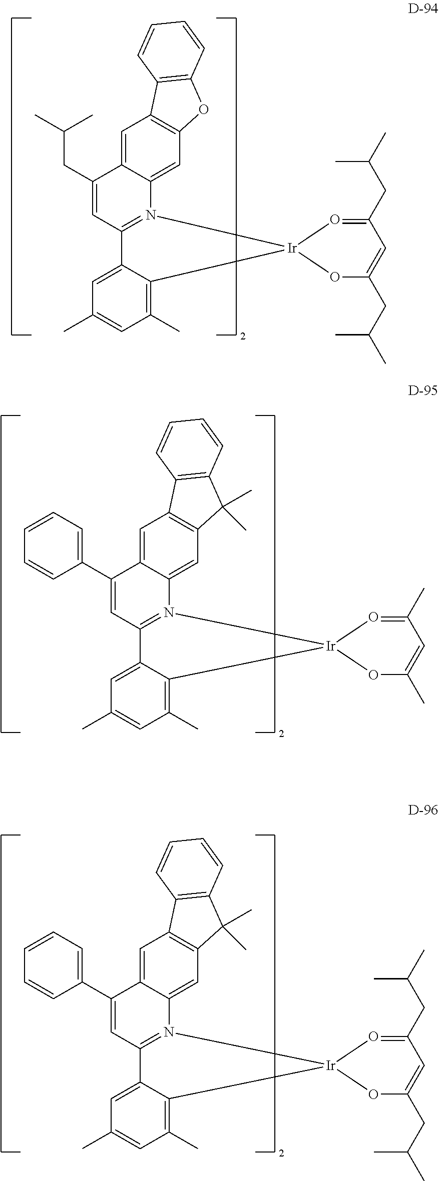

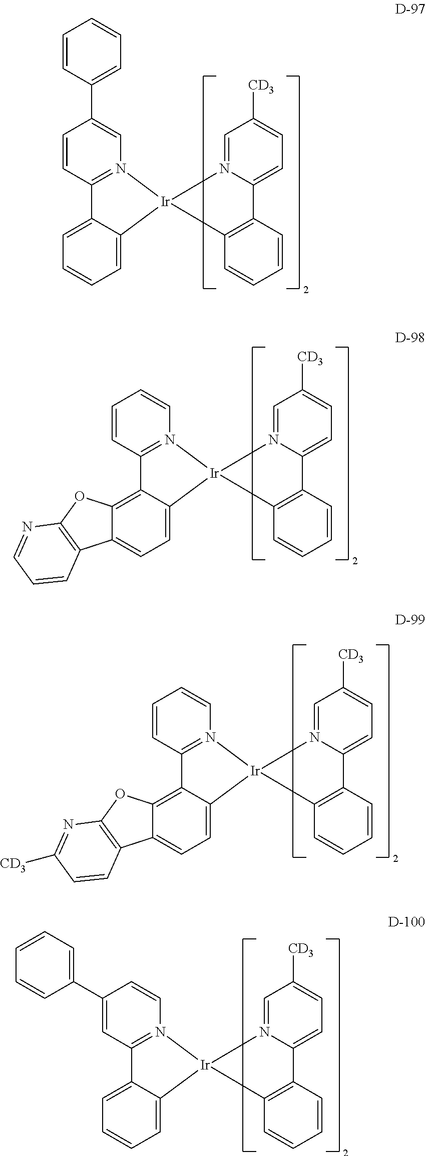

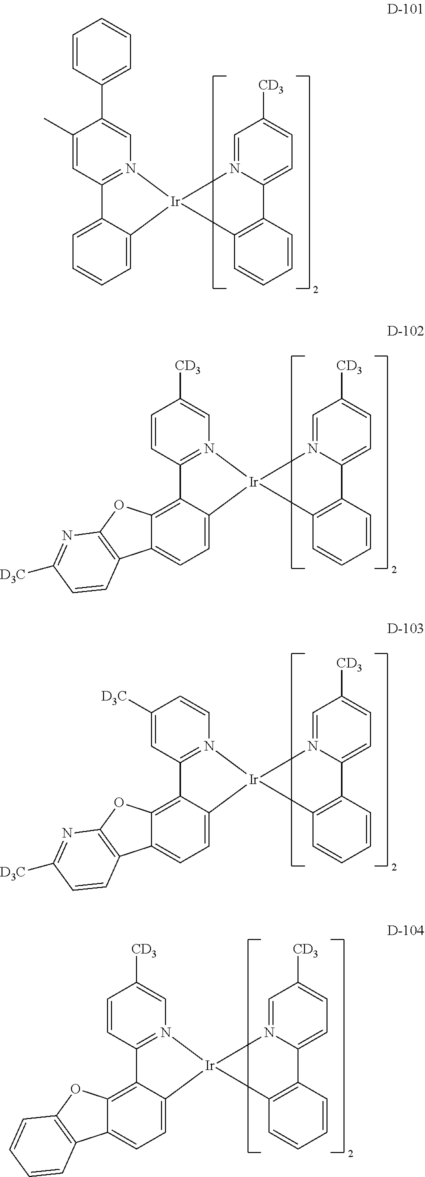

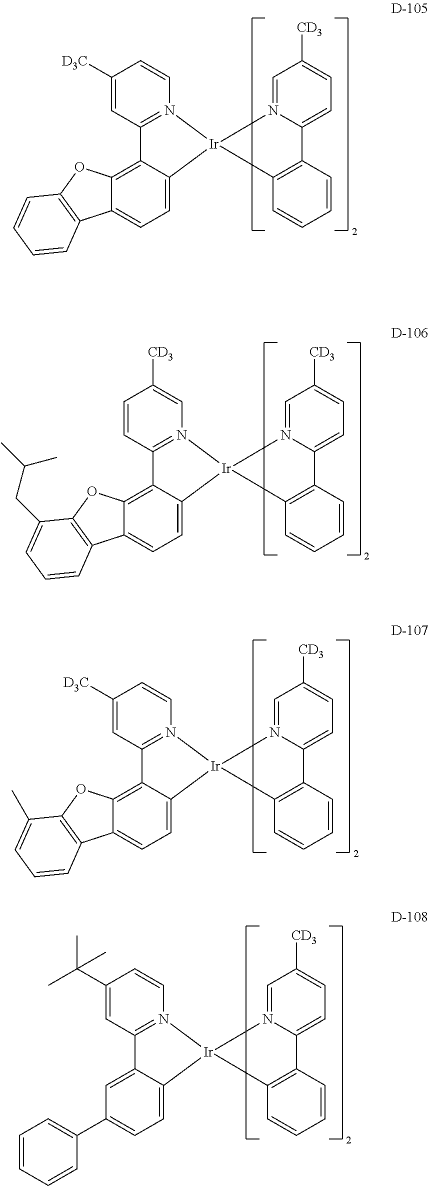

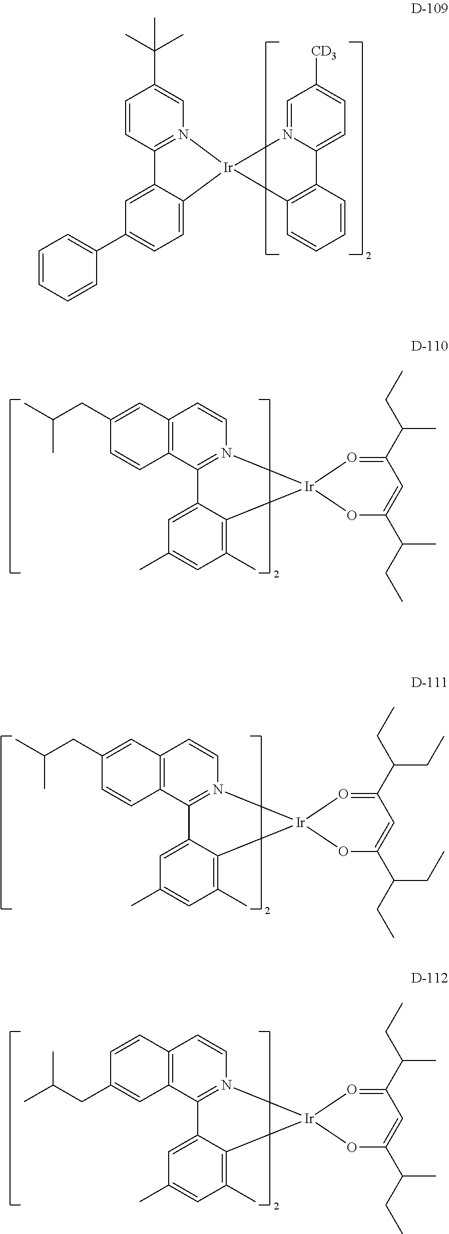

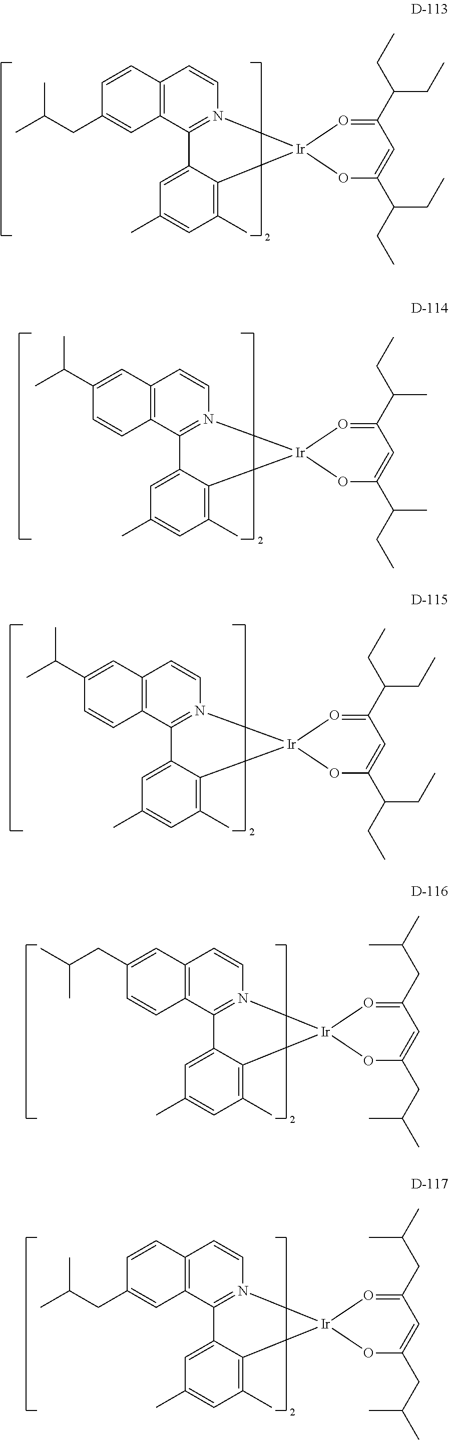

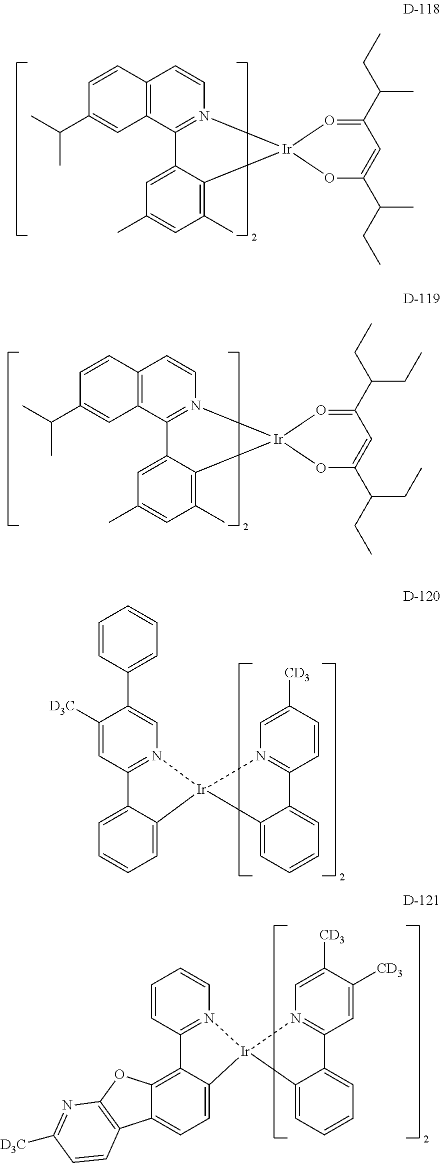

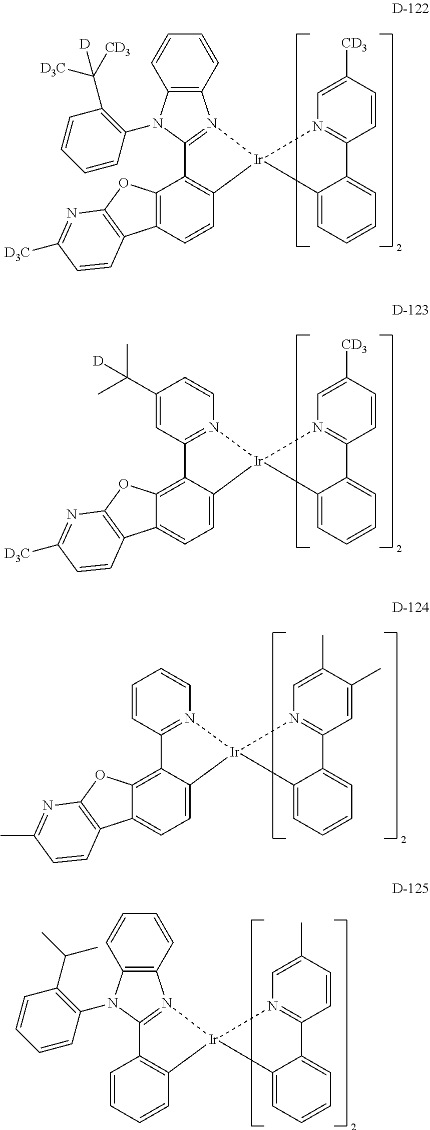

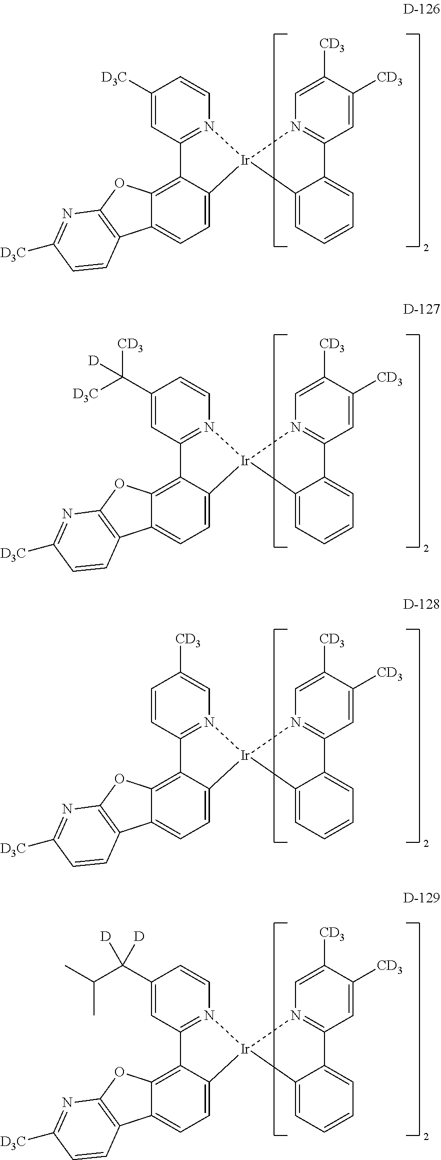

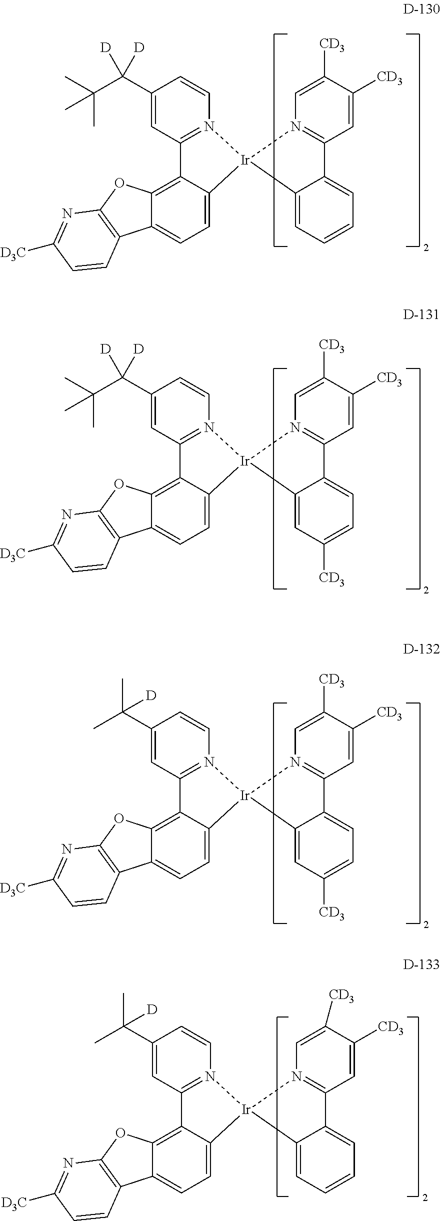

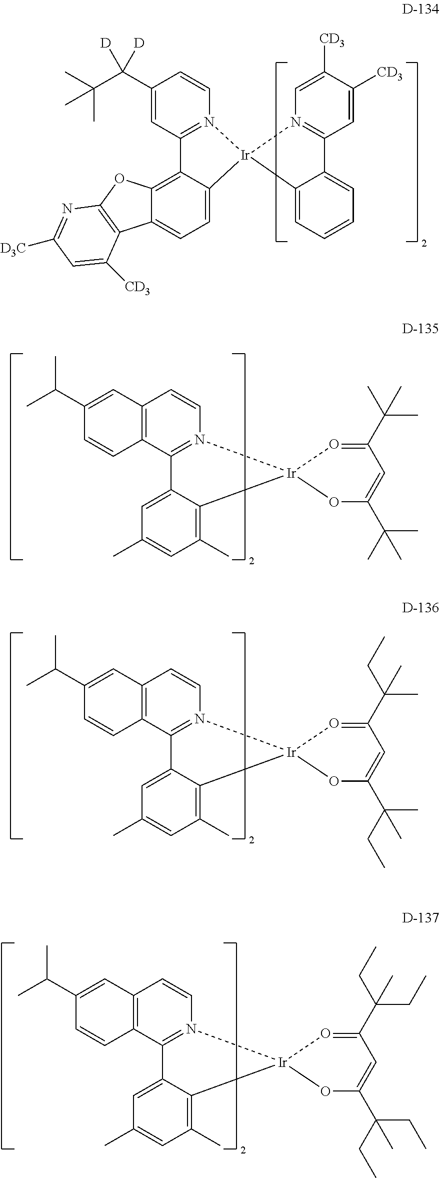

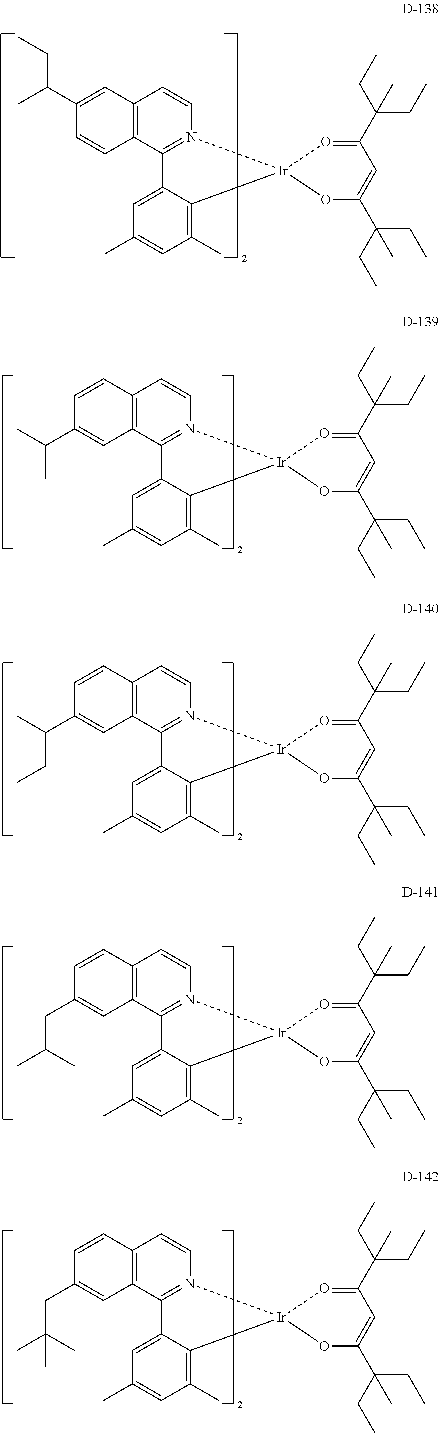

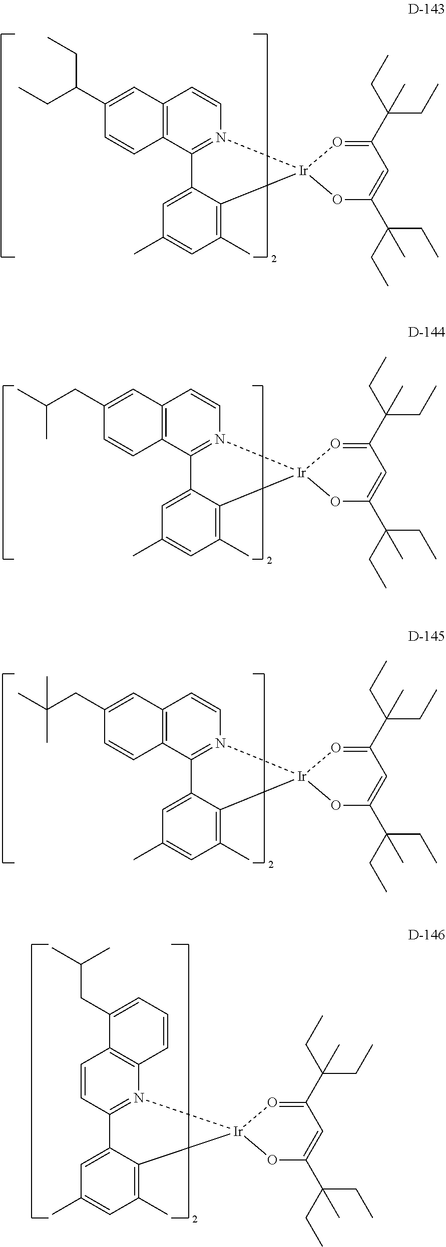

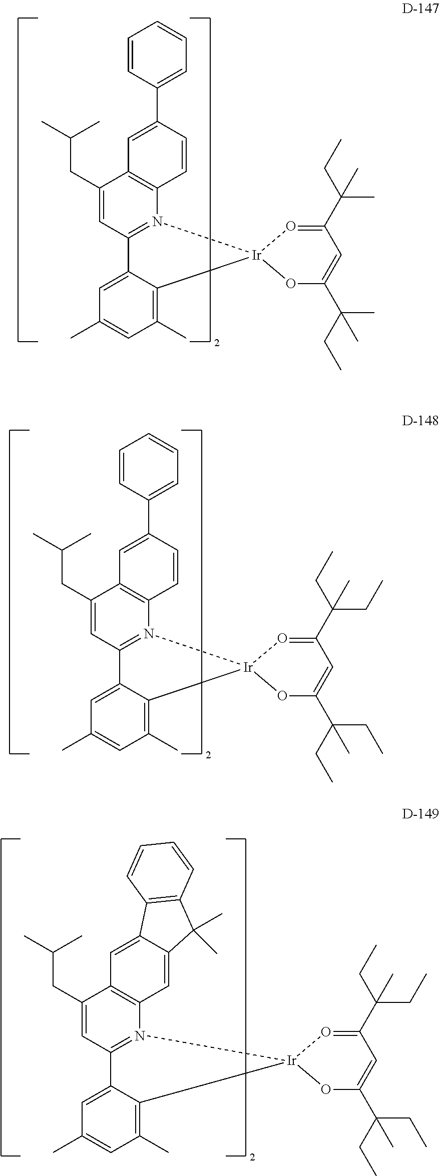

Each layer of the OLED of the present disclosure can be formed by either dry film-forming methods such as vacuum evaporation, sputtering, plasma, ion plating, etc., or wet film-forming methods such as ink jet printing, nozzle printing, slot coating, spin coating, dip coating, flow coating, etc.,

When using a wet film-forming method, a thin film can be formed by dissolving or diffusing the materials forming each layer into any suitable solvent such as ethanol, chloroform, tetrahydrofuran, dioxane, etc. The solvent is not particularly limited as long as the material forming each layer is soluble or dispersible in the solvents, which do not cause any problems in forming a film.

In addition, the first and the second host compounds of the present disclosure may be film-formed by the above-listed methods, commonly by a co-evaporation process or a mixture-evaporation process. The co-evaporation is a mixed deposition method in which two or more materials are placed in a respective individual crucible source and a current is applied to both cells at the same time to evaporate the materials. The mixture-evaporation is a mixed deposition method in which two or more materials are mixed in one crucible source before evaporating them, and a current is applied to one cell to evaporate the materials. In addition, if the first and the second host compounds are present in the same layer or different layers in an OLED, the two host compounds may individually form films. For example, the second host compound may be deposited after depositing the first host compound.

The present disclosure may provide a display device by using the plurality of host materials including the compound represented by formula 1 and the compound represented by formula 2. That is, it is possible to produce a display system and a lighting system by using the plurality of host materials of the present disclosure. Specifically, a display system, for example, a display system for white organic light emitting devices, smart phones, tablets, notebooks, PCs, TVs, or cars; or a lighting system, for example, an outdoor or indoor lighting system, can be produced by using the plurality of host materials of the present disclosure.

Hereinafter, the preparation method of the compound of the present disclosure, and the properties thereof, and the properties of the OLED comprising the plurality of host materials of the present disclosure will be explained in detail with reference to the representative compounds of the present disclosure. However, the following examples are only to describe the characteristics of the OLED device comprising the compound according to the present disclosure and the plurality of host materials according to the present disclosure for a detailed understanding of the present disclosure, but the present disclosure is not limited to the following examples.

### EXAMPLE 1

### Preparation of Compound H2-68

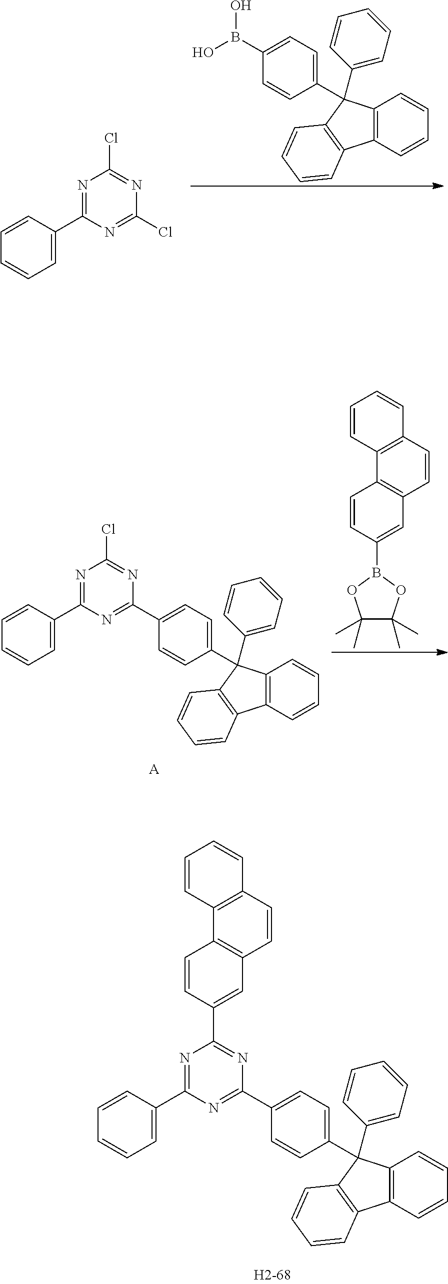

### Synthesis of Compound A

2,4-dichloro-6-phenyl-1,3,5-triazine (20 g, 55.2 mmol), (4-(9-phenyl-9H-fluorene-9-yl)phenyl)boronic acid (18.8 g, 83.1 mmol), Pd(PPh₃)₄ (4.8 g, 2.8 mmol), K₂CO₃ (23 g, 111 mmol), 140 ml of toluene, 40 mL of ethanol, and 40 ml of water were added to a flask, dissolved, and then refluxed at 120° C. for 2 hours. After completion of the reaction, the organic layer was extracted with ethyl acetate. After the residual moisture was removed with magnesium sulfate, the residue was dried, and separated by column chromatography to obtain 8 g of compound A (yield: 28%).

### Synthesis of Compound H2-68

Compound A (8 g, 15.7 mmol), 4,4,5,5-tetramethyl-2-(phenanthrene-2-yl)-1,3,2-dioxaborolane (4.1 g, 18.4 mmol), Pd (PPh₃)₄ (1 g, 0.8 mmol), K₂CO₃ (4.4 g, 114 mmol), 40 mL of toluene, 10 mL of ethanol, and 10 ml of water were added to a flask, dissolved, and then refluxed at 120° C. for 2 hours. After completion of the reaction, the organic layer was extracted with ethyl acetate. After the residual moisture was removed with magnesium sulfate, the residue was dried, and separated by column chromatography to obtain 1.5 g of compound H2-68 (yield: 15%).

| Compound | MW | M.P. |
|---|---|---|
| H2-68 | 649.80 | 234° C. |

### EXAMPLE 2

### Preparation of Compound H2-66

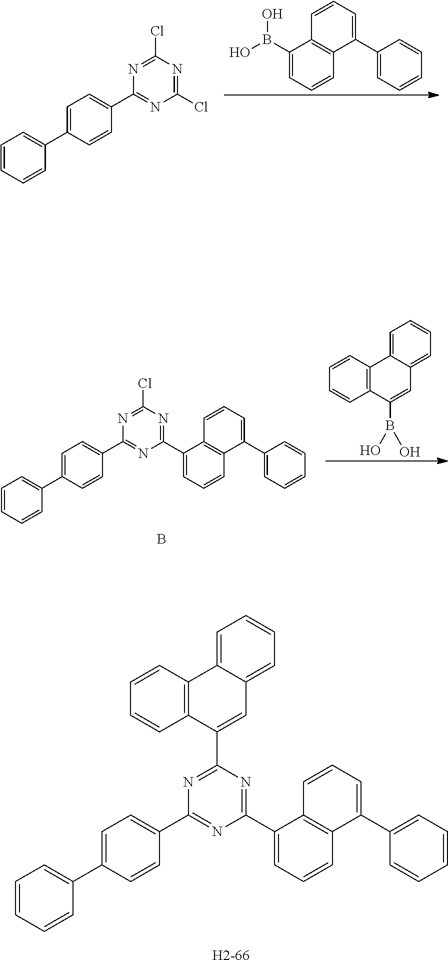

### Synthesis of Compound B

2-([1,1′-biphenyl]-4-yl)-4,6-dichloro-1,3,5-triazine (36.5 g, 120.7 mmol), (5-phenylnaphthalene-1-yl) boronic acid (15 g, 60.4 mmol), Pd(PPh₃)₄ (7 g, 3 mmol), K₂CO₃ (33 g, 120.8 mmol), 300 mL of toluene, 100 mL of ethanol, and 100 ml of water were added to a flask, dissolved, and then refluxed at 120° C. for 2 hours. After completion of the reaction, the organic layer was extracted with ethyl acetate. After the residual moisture was removed with magnesium sulfate, the residue was dried, and separated by column chromatography to obtain 22 g of compound B (yield: 77%).

### Synthesis of Compound H2-66

Compound B (22 g, 46.8 mmol), phenanthrene-9-yl boronic acid (15 g, 70.2 mmol), Pd(PPh₃)₄ (2.8 g, 2.5 mmol), K₂CO₃ (13 g, 94 mmol), 120 mL of toluene, 40 mL of ethanol, and 40 ml of water were added to a flask, dissolved, and then refluxed at 120° C. for 2 hours. After completion of the reaction, the organic layer was extracted with ethyl acetate. After the residual moisture was removed with magnesium sulfate, the residue was dried, and separated by column chromatography to obtain compound 5.2 g of H2-66 (yield: 18%).

| Compound | MW | M.P. |
|---|---|---|
| H2-66 | 611.75 | 222° C. |

### EXAMPLE 3

### Preparation of Compound H2-137

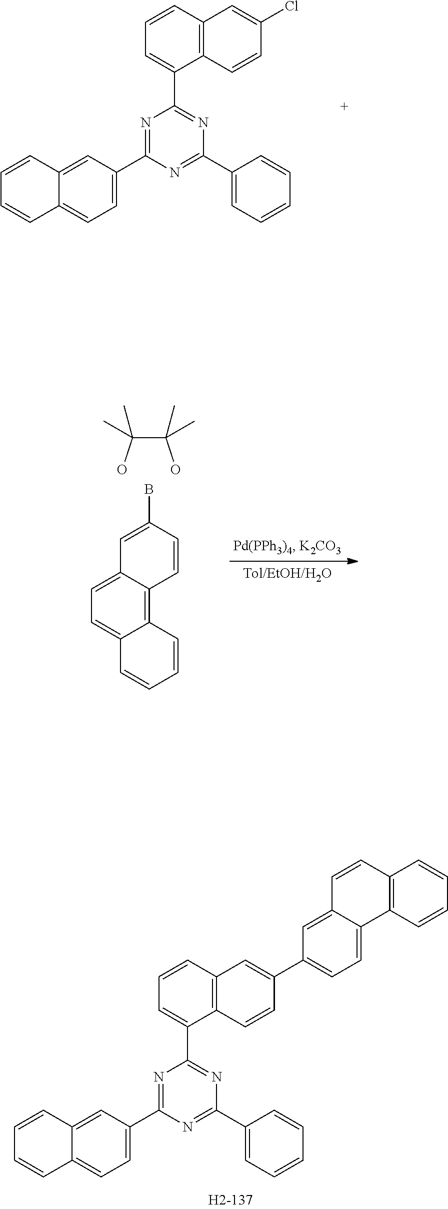

2-(6-chloronaphthalene-1-yl)-4-(naphthalene-2-yl)-6-phenyl-1,3,5-triazine (5 g, 11.28 mmol), 4,4,5,5-tetramethyl-2-(phenanthrene-2-yl)-1,3,2-dioxaborolane (4.1 g, 12.41 mmol), Pd(PPh₃)₄ (0.6 g, 0.56 mmol), K₂CO₃ (3.2 g, 23 mmol), 30 mL of toluene, 10 mL of ethanol, and 10 ml of water were added to a flask, dissolved, and then refluxed at 120° C. for 2 hours. After completion of the reaction, the mixture was cooled to room temperature, and the resulting solid was filtered under reduced pressure. The solid was dissolved in phenyl chloride, filtered through silica with methylene chloride (MC), and recrystallized with o-xylene to obtain compound 2.7 g of H2-137 (yield: 40%).

| Compound | MW | M.P. |
|---|---|---|
| H2-137 | 499.5 | 243.7° C. |

### EXAMPLE 4

### Preparation of Compound H2-3

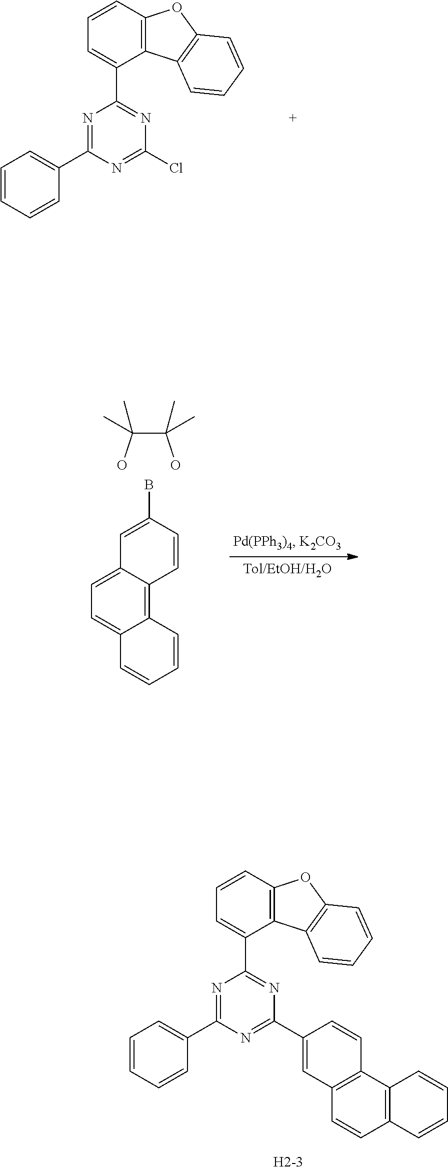

2-chloro-4-(dibenzo[b,d]furan-1-yl)-6-phenyl-1,3,5-triazine (8.7 g, 24.33 mmol), 4,4,5,5-tetramethyl-2-(phenanthrene-2-yl)-1,3,2-dioxaborolane (7.4 g, 24.33 mmol), Pd(PPh₃)₄ (1.4 g, 1.216 mmol), K₂CO₃ (10 g, 72.96 mmol), 148 mL of toluene, 37 mL of ethanol, and 37 mL of water were added to a flask, dissolved, and then refluxed at 140° C. for 2 hours. After completion of the reaction, the mixture was cooled to room temperature, and the resulting solid was filtered under reduced pressure. The solid was dissolved in phenyl chloride, filtered through silica with MC, and recrystallized with o-xylene to obtain 4.3 g of compound H2-3 (yield: 35%).

| Compound | MW | M.P. |
|---|---|---|
| H2-3 | 499.5 | 243.7° C. |

### EXAMPLE 5

### Preparation of Compound H2-2

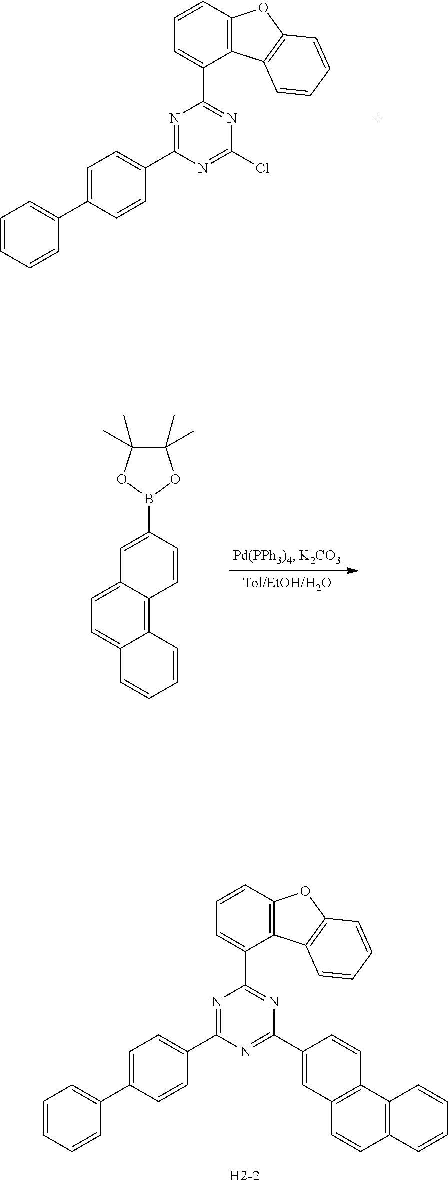 [SMILES: Clc1nc(-c2ccc(-c3ccccc3)cc2)nc(-c2cccc3oc4ccccc4c23)n1 ; CC1(C)OB(c2ccc3c(ccc4ccccc43)c2)OC1(C)C ; c1ccc(-c2ccc(-c3nc(-c4ccc5c(ccc6ccccc65)c4)nc(-c4cccc5oc6ccccc6c45)n3)cc2)cc1 ; "H2-2"]

2-([1,1′-biphenyl]-4-yl)-4-chloro-6-(dibenzo[b,d]furan-1-yl)-1,3,5-triazine (7.7 g, 17.75 mmol), 4,4,5,5-tetramethyl-2-(phenanthrene-2-yl)-1,3,2-dioxaborolane (5.4 g, 17.75 mmol), Pd(PPh₃)₄ (1 g, 0.887 mmol), K₂CO₃ (7.3 g, 53.27 mmol), 108 mL of toluene, 27 mL of ethanol, and 27 ml of water were added to a flask, dissolved, and then refluxed at 140° C. for 2 hours. After completion of the reaction, the mixture was cooled to room temperature, and the resulting solid was filtered under reduced pressure. The solid was dissolved in phenyl chloride, filtered through silica with MC, and recrystallized with o-xylene to obtain compound 5.2 g of H2-5 (yield: 50%).

| Compound | MW | M.P. |
|---|---|---|
| H2-2 | 575.6 | 295° C. |

### EXAMPLE 6

### Preparation of Compound H2-67

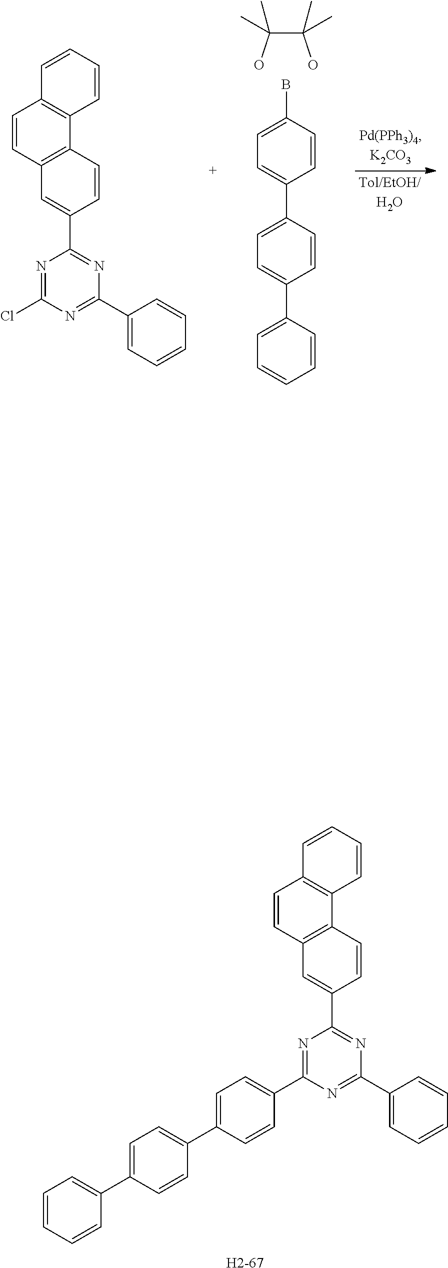

2-chloro-4-(phenanthrene-2-yl)-6-phenyl-1,3,5-triazine (4.5 g, 12.23 mmol), 2-([1,1′:4′,1″-terphenyl]-4-yl)-4,4,5,5-tetramethyl-1,3,2-dioxaborolane (4.3 g, 12.23 mmol), Pd(PPh₃)₄ (0.7 g, 0.611 mmol), K₂CO₃ (5 g, 36.70 mmol), 80 mL of toluene, 20 mL of ethanol, and 20 mL of water were added to a flask, dissolved, and then refluxed at 140° C. for 2 hours. After completion of the reaction, the mixture was cooled to room temperature, and the resulting solid was filtered under reduced pressure. The solid was dissolved in phenyl chloride, filtered through silica with MC, and recrystallized with o-xylene to obtain compound 3.3 g of H2-67 (yield: 48%).

| Compound | MW | M.P. |
|---|---|---|
| H2-67 | 561.6 | 325.6° C. |

### EXAMPLE 7

### Preparation of Compound H2-1

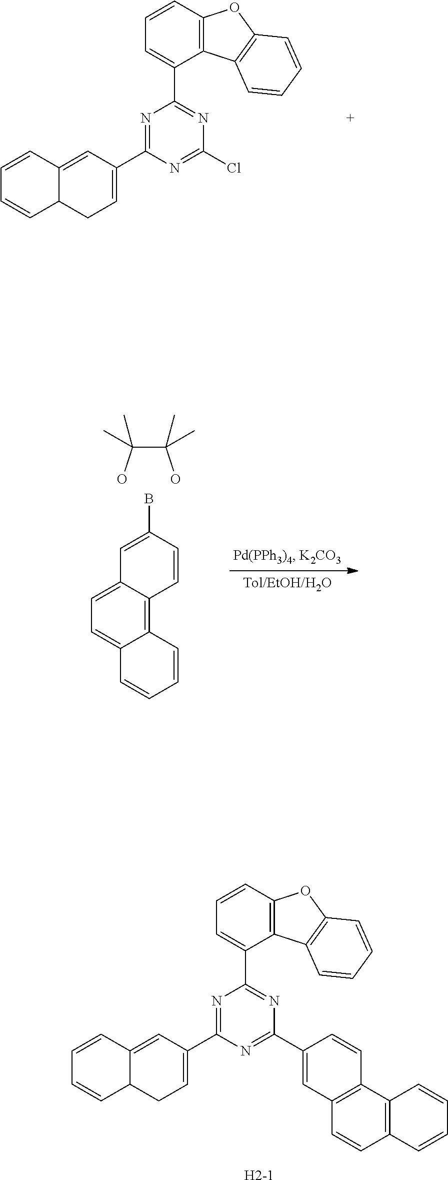

2-chloro-4-(dibenzo [b,d]furan-1-yl)-6-(4,4a-dihydronaphthalene-2-yl)-1,3,5-triazine (8.8 g, 21.61 mmol), 4,4,5,5-tetramethyl-2-(phenanthren-2-yl)1,3,2-dioxaborolane (7.2 g, 23.77 mmol), Pd(PPh₃)₄ (1.2 g, 1.080 mmol), K₂CO₃ (9 g, 64.85 mmol), 140 mL of toluene, 35 mL of ethanol, and 35 mL of water were added to a flask, dissolved, and then refluxed at 140° C. for 3 hours. After completion of the reaction, the mixture was cooled to room temperature, and the resulting solid was filtered under reduced pressure. The solid was dissolved in phenyl chloride, filtered through silica with MC, and recrystallized with o-xylene to obtain 6.8 g of compound H2-1 (yield: 57.6%).

| Compound | MW | M.P. |
|---|---|---|
| H2-1 | 549.6 | 271.5° C. |

### EXAMPLE 8

### Preparation of Compound H2-161

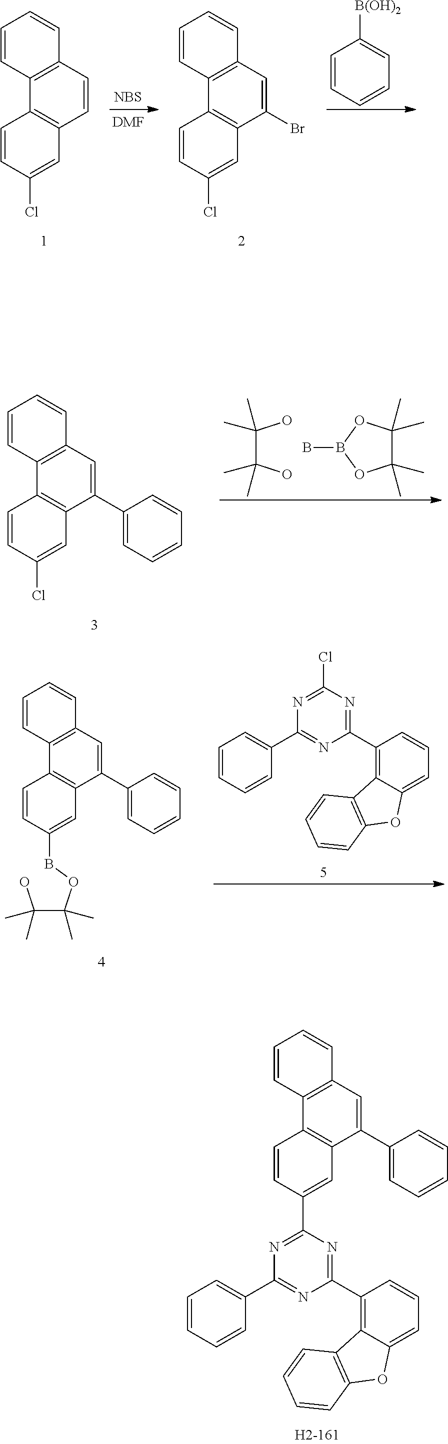

### Synthesis of Compound 2

Compound 1 (10 g, 47.1 mmol) and 300 mL of dimethylformamide were added to a flask, and dissolved, and then N-bromosuccinimide (67 g, 377 mmol) was slowly added, and refluxed at 150° C. for 1 hour. After completion of the reaction, the organic layer was extracted with ethyl acetate. After the residual moisture was removed with magnesium sulfate, the residue was dried, and separated by column chromatography to obtain 7 g of compound 2 (yield: 51%).

### Synthesis of Compound 3

compound 2 (9.2 g, 31.7 mmol), phenylboronic acid (4.3 g, 34.8 mmol), Pd(PPh₃)₄ (2 g, 1.6 mmol), K₂CO₃ (11 g, 80 mmol), 90 mL of toluene, 30 mL of ethanol, and 30 ml of water were added to a flask, dissolved, and then refluxed at 120° C. for 2 hours. After completion of the reaction, the organic layer was extracted with ethyl acetate. After the residual moisture was removed with magnesium sulfate, the residue was dried, and separated by column chromatography to obtain 7 g of compound 3 (yield: 76%).

### Synthesis of Compound 4

Compound 3 (9.5 g, 33 mmol), 4,4′,4′,5,5,5′,5′-octamethyl-2,2′-bi(1,3,2-dioxaborolane) (12.6 g, 49.5 mmol), potassium acetate (8.1 g, 82.5 mmol), 2-dicyclohexylphosphino-2′,6′-dimethoxybiphenyl (41 g, 99 mmol), Pd₂(dba)₃ (1.5 g, 1.65 mmol), and 500 ml of dioxane were added to a flask, dissolved, and then refluxed at 120° C. for 2 hours. After completion of the reaction, the organic layer was extracted with ethyl acetate. After the residual moisture was removed with magnesium sulfate, the residue was dried, and separated by column chromatography to obtain 10 g of compound 4 (yield: 79%).

### Synthesis of Compound H2-161

Compound 4 (15 g, 40 mmol), 2-chloro-4-(dibenzo[b,d]furan-1-yl)-6-phenyl-1,3,5-triazine (compound 5) (16 g, 44 mmol), Pd(PPh₃)₄ (2.4 g, 2 mmol), K₂CO₃ (11 g, 80 mmol), 100 mL of toluene, 20 mL of ethanol, and 20 ml of water were added to a flask, dissolved, and then refluxed at 120° C. for 2 hours. After completion of the reaction, the organic layer was extracted with ethyl acetate. After the residual moisture was removed with magnesium sulfate, the residue was dried, and separated by column chromatography to obtain compound 8 g of H2-161 (yield: 34%).

| Compound | MW | M.P. |
|---|---|---|
| H2-161 | 575.67 | 270° C. |

### DEVICE EXAMPLES 1 TO 16

### Producing a Red Light-Emitting OLED Deposited with a Plurality of Host Materials According to the Present Disclosure as a Host

OLEDs according to the present disclosure were produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 shown in Table 2 was introduced into a cell of the vacuum vapor deposition apparatus, and compound HT-1 shown in Table 2 was introduced into another cell. The two materials were evaporated at different rates to deposit a hole injection layer with a thickness of 10 nm by doping compound HI-1 in an amount of 3 wt % based on the total amount of compound HI-1 and compound HT-1. Subsequently, compound HT-1 was deposited on the hole injection layer to form a first hole transport layer having a thickness of 80 nm. Subsequently, compound HT-2 was introduced into another cell of the vacuum vapor deposition apparatus and was evaporated by applying an electric current to the cell, thereby depositing a second hole transport layer with a thickness of 60 nm on the first hole transport layer. After forming the hole injection layer and the hole transport layers, a light-emitting layer was deposited thereon as follows. The first host compounds and the second host compounds described in Table 1 were introduced into two cells of the vacuum vapor deposition apparatus as hosts, and compound D-39 was introduced into another cell as a dopant. The two host materials were evaporated at a rate of 1:1 and the dopant material was simultaneously evaporated at a different rate, and the dopant was deposited in a doping amount of 3 wt % based on the total amount of the hosts and the dopant to form a light-emitting layer having a thickness of 40 nm on the second hole transport layer. Subsequently, compound ETL-1 and compound EIL-1 were deposited at a weight ratio of 50:50 as electron transport materials to form an electron transport layer having a thickness of 35nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. All the materials used for producing the OLED were purified by vacuum sublimation at 10-6 torr.

### COMPARATIVE EXAMPLES 1 TO 4

### Producing a Red Light-Emitting OLED Deposited with a Plurality of Host Materials not in Accordance with the Present Disclosure as a Host

OLEDs were produced in the same manner as in Device Examples 1 to 16, except that the compounds described in Table 1 were used as a host of the light-emitting layer.

### COMPARATIVE EXAMPLES 5 AND 6

### Producing an OLED Comprising a Single Host

OLEDs were produced in the same manner as in Device Examples 1 to 16, except that the second host compound described in Table 1 was used alone as a host of the light-emitting layer.

The driving voltage, the luminous efficiency, and the light-emitting color at a luminance of 1,000 nit, and the time taken for luminance to decrease from 100% to 95% at a luminance of 5,000 nit (lifespan: T95) of the OLEDs produced in Device Examples 1 to 16, and Comparative Examples 1 to 6 are provided in Table 1 below:

TABLE 1

| | | | Driving | Luminous | Light- | |
|---|---|---|---|---|---|---|
| | First | Second | Voltage | Efficiency | Emitting | Lifespan |
| | Host | Host | (V) | (cd/A) | Color | (T95, hr) |
| Device | H1-46 | H2-1 | 3.1 | 34.7 | red | 879 |
| Example 1 | | | | | | |
| Device | H1-141 | H2-1 | 3.1 | 35.5 | red | 522 |
| Example 2 | | | | | | |
| Device | H1-28 | H2-1 | 3.0 | 35.2 | red | 629 |
| Example 3 | | | | | | |
| Device | H1-15 | H2-4 | 3.1 | 35.0 | red | 572 |
| Example 4 | | | | | | |
| Device | H1-12 | H2-4 | 2.9 | 34.7 | red | 509 |
| Example 5 | | | | | | |
| Device | H1-5 | H2-4 | 2.9 | 34.7 | red | 465 |
| Example 6 | | | | | | |
| Device | H1-28 | H2-4 | 3.0 | 35.1 | red | 444 |
| Example 7 | | | | | | |
| Device | H1-15 | H2-5 | 3.1 | 36.5 | red | 748 |
| Example 8 | | | | | | |
| Device | H1-142 | H2-5 | 2.9 | 36.6 | red | 527 |
| Example 9 | | | | | | |
| Device | H1-5 | H2-5 | 2.9 | 36.0 | red | 594 |
| Example 10 | | | | | | |
| Device | H1-143 | H2-5 | 3.0 | 36.4 | red | 405 |
| Example 11 | | | | | | |
| Device | H1-46 | H2-3 | 3.1 | 34.9 | red | 689 |
| Example 12 | | | | | | |
| Device | H1-46 | H2-2 | 3.1 | 35.3 | red | 584 |
| Example 13 | | | | | | |
| Device | H1-15 | H2-137 | 3.2 | 34.8 | red | 540 |
| Example 14 | | | | | | |
| Device | H1-15 | H2-151 | 3.3 | 35.7 | red | 518 |
| Example 15 | | | | | | |
| Device | H1-135 | H2-151 | 2.9 | 36.5 | red | 678 |
| Example 16 | | | | | | |
| Comparative | C-1 | H2-137 | 3.5 | 34.1 | red | 155 |
| Example 1 | | | | | | |
| Comparative | C-1 | H2-1 | 3.3 | 33.6 | red | 91 |
| Example 2 | | | | | | |
| Comparative | C-1 | H2-4 | 3.3 | 32.8 | red | 79 |
| Example 3 | | | | | | |
| Comparative | C-1 | H2-5 | 3.3 | 34.4 | red | 134 |
| Example 4 | | | | | | |
| Comparative | — | H2-4 | 3.5 | 28.9 | red | 14.0 |
| Example 5 | | | | | | |
| Comparative | — | H2-5 | 3.4 | 30.9 | red | 6.8 |
| Example 6 | | | | | | |

It can be seen that the OLEDs using the plurality of host materials according to the present disclosure exhibit excellent lifespan characteristics, while exhibiting a driving voltage and luminous efficiency equivalent or superior than the OLEDs including the conventional host or the conventional combination of host materials.

### DEVICE EXAMPLES 17 TO 19

### Producing an OLED Comprising an Organic Electroluminescent Compound According to the Present Disclosure as a Host

OLEDs were produced in the same manner as in Device Examples 1 to 16, except that the compounds described in Table 2 were used alone as a host of the light-emitting layer.

### COMPARATIVE EXAMPLES 7 AND 8

### Producing an OLED Comprising a Comparative Compound as a Host

OLEDs were produced in the same manner as in Device Examples 1 to 16, except that the compounds described in Table 2 were used alone as a host of the light-emitting layer.

The driving voltage, and the light-emitting color at a luminance of 1,000 nit, and the time taken for luminance to decrease from 100% to 95% at a luminance of 5,000 nit (lifespan: T95) of the OLEDs produced in Device Examples 17 to 19, and Comparative Examples 7 and 8 are provided in Table 2 below:

TABLE 2

| | | Driving | | |
|---|---|---|---|---|
| | | Voltage | Light-Emitting | Lifespan |
| | Host | (V) | Color | (T95, hr) |
| Device | H2-66 | 4.4 | red | 37 |
| Example 17 | | | | |
| Device | H2-137 | 3.8 | red | 25.2 |
| Example 18 | | | | |
| Device | H2-3 | 3.4 | red | 18.7 |
| Example 19 | | | | |
| Comparative | T-1 | 4.2 | red | 4.2 |
| Example 7 | | | | |
| Comparative | T-2 | 4.3 | red | 1.3 |
| Example 8 | | | | |

It can be seen that the OLEDs comprising the compounds according to the present disclosure exhibits excellent lifespan characteristics while exhibiting a driving voltage equivalent to the OLEDs including the conventional host compound.

The compounds used in the Device Examples and the Comparative Examples are shown in Table 3 below.

TABLE 3

| | |
|---|---|
| Hole Injection Layer/ Hole Transport Layer | 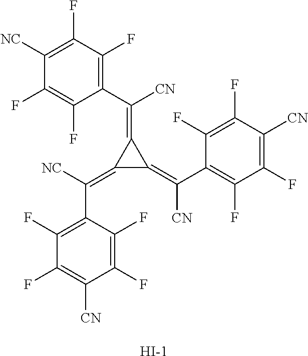 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 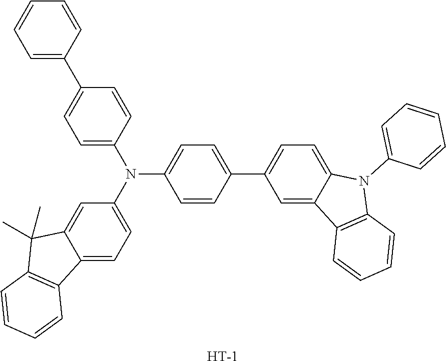 |
| | 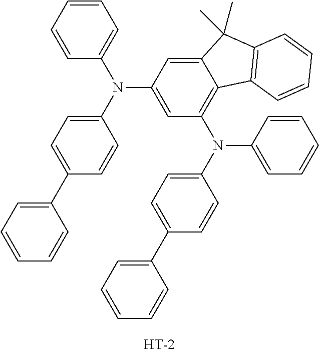 |
| Light- Emitting Layer | 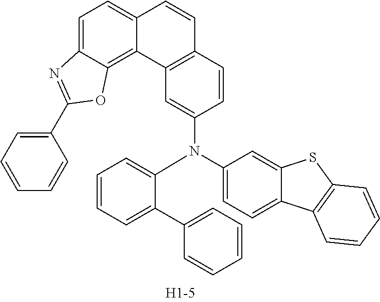 [SMILES: c1ccc(-c2nc3ccc4ccc5ccc(N(c6ccc7c(c6)sc6ccccc67)c6ccccc6-c6ccccc6)cc5c4c3o2)cc1] |
| | H1-12 |
| | 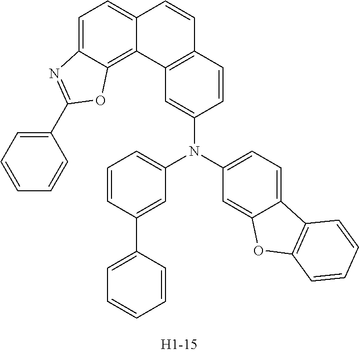 [SMILES: c1ccc(-c2cccc(N(c3ccc4c(c3)oc3ccccc34)c3ccc4ccc5ccc6nc(-c7ccccc7)oc6c5c4c3)c2)cc1] |
| | 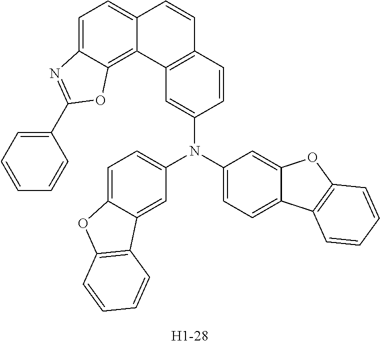 [SMILES: c1ccc(-c2nc3ccc4ccc5ccc(N(c6ccc7c(c6)oc6ccccc67)c6ccc7oc8ccccc8c7c6)cc5c4c3o2)cc1] |
| | 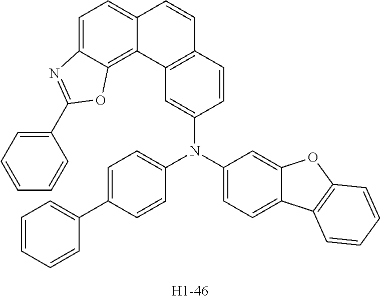 [SMILES: c1ccc(-c2ccc(N(c3ccc4c(c3)oc3ccccc34)c3ccc4ccc5ccc6nc(-c7ccccc7)oc6c5c4c3)cc2)cc1] |
| | 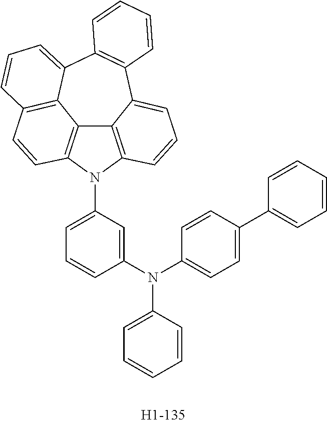 [SMILES: c1ccc(-c2ccc(N(c3ccccc3)c3cccc(-n4c5cccc6c5c5c7c(cccc7ccc54)-c4ccccc4-6)c3)cc2)cc1] |
| | 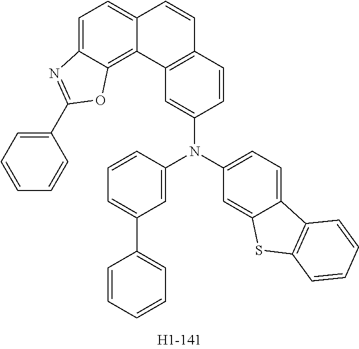 [SMILES: c1ccc(-c2cccc(N(c3ccc4c(c3)sc3ccccc34)c3ccc4ccc5ccc6nc(-c7ccccc7)oc6c5c4c3)c2)cc1] |
| | 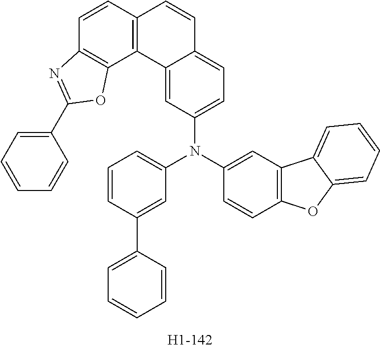 [SMILES: c1ccc(-c2cccc(N(c3ccc4oc5ccccc5c4c3)c3ccc4ccc5ccc6nc(-c7ccccc7)oc6c5c4c3)c2)cc1] |
| | 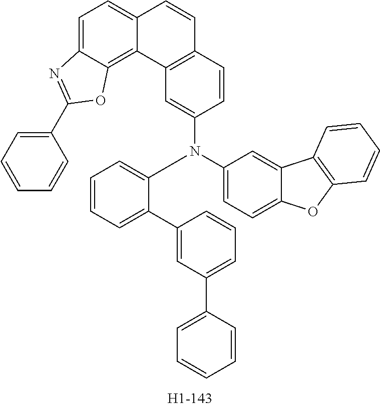 [SMILES: c1ccc(-c2cccc(-c3ccccc3N(c3ccc4oc5ccccc5c4c3)c3ccc4ccc5ccc6nc(-c7ccccc7)oc6c5c4c3)c2)cc1] |
| | 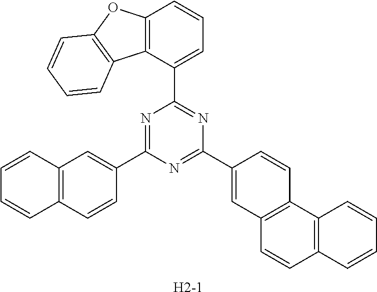 [SMILES: c1ccc2cc(-c3nc(-c4ccc5c(ccc6ccccc65)c4)nc(-c4cccc5oc6ccccc6c45)n3)ccc2c1 ; "H2-1"] |
| | 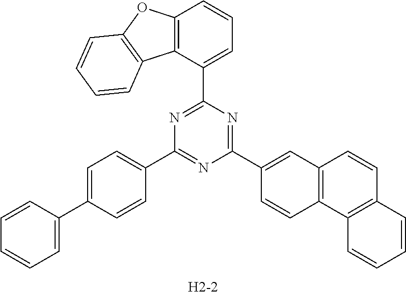 [SMILES: c1ccc(-c2ccc(-c3nc(-c4ccc5c(ccc6ccccc65)c4)nc(-c4cccc5oc6ccccc6c45)n3)cc2)cc1 ; "H2-2"] |
| | 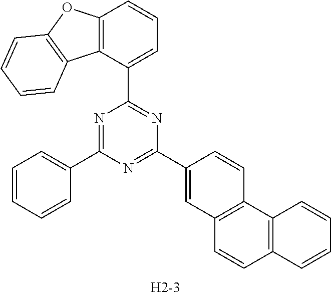 [SMILES: c1ccc(-c2nc(-c3ccc4c(ccc5ccccc54)c3)nc(-c3cccc4oc5ccccc5c34)n2)cc1] |
| | 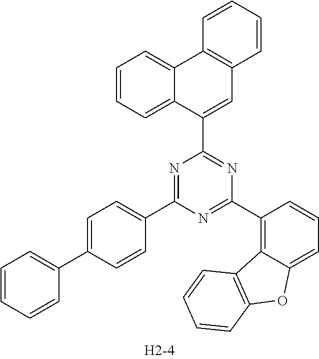 [SMILES: c1ccc(-c2ccc(-c3nc(-c4cc5ccccc5c5ccccc45)nc(-c4cccc5oc6ccccc6c45)n3)cc2)cc1] |
| | 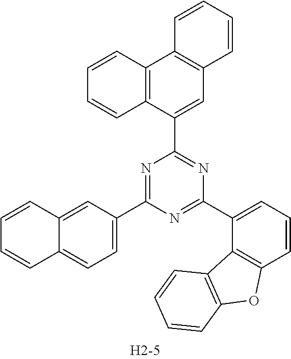 [SMILES: c1ccc2cc(-c3nc(-c4cc5ccccc5c5ccccc45)nc(-c4cccc5oc6ccccc6c45)n3)ccc2c1] |
| | 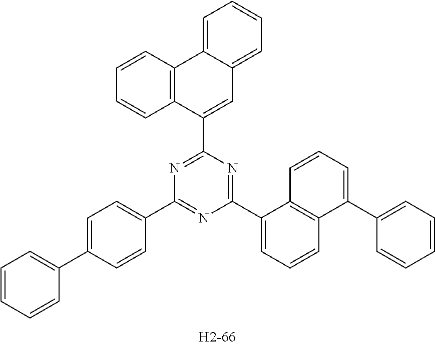 [SMILES: c1ccc(-c2ccc(-c3nc(-c4cccc5c(-c6ccccc6)cccc45)nc(-c4cc5ccccc5c5ccccc45)n3)cc2)cc1] |
| | 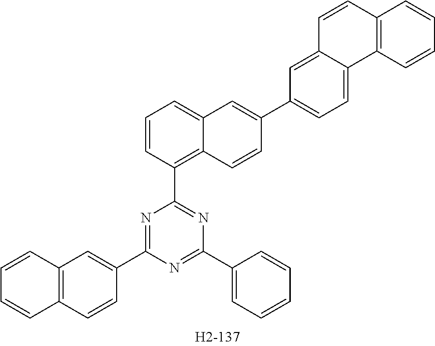 [SMILES: c1ccc(-c2nc(-c3ccc4ccccc4c3)nc(-c3cccc4cc(-c5ccc6c(ccc7ccccc76)c5)ccc34)n2)cc1] |
| | 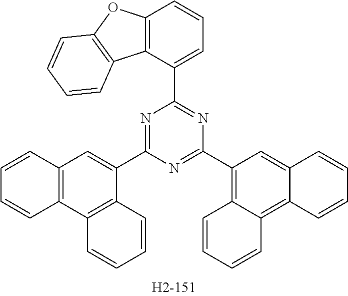 [SMILES: c1ccc2c(c1)cc(-c1nc(-c3cc4ccccc4c4ccccc34)nc(-c3cccc4oc5ccccc5c34)n1)c1ccccc12] |
| | 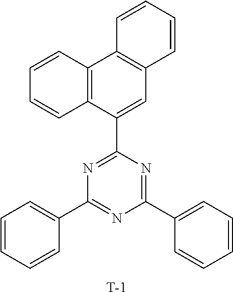 |
| | 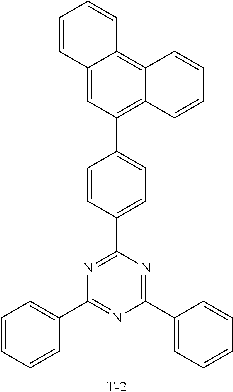 |
| | 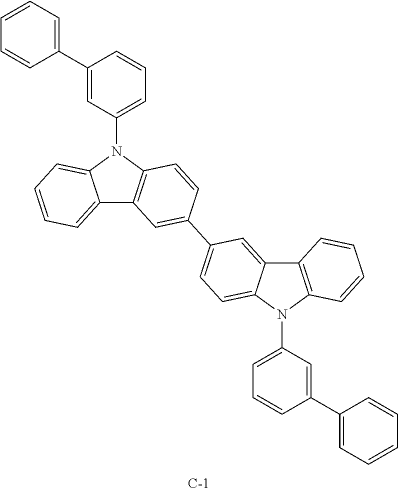 |
| | 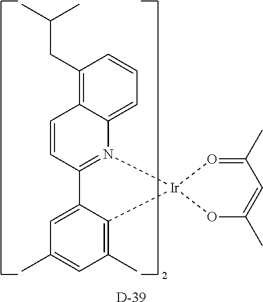 |
| Electron Transport Layer/ Electron Injection Layer | 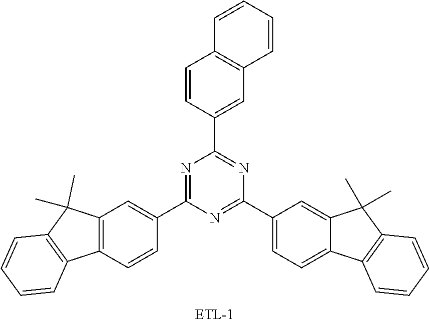 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 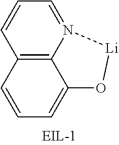 |

1. An organic electroluminescent compound represented by the following formula 2′: 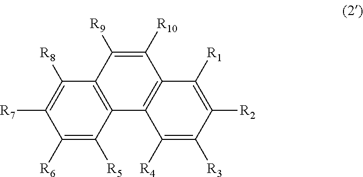
in formula 2′,
R₁ to R₁₀ are each independently represented by 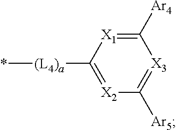
or represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s), with a proviso that at least one of R₁ to R₁₀ is represented by 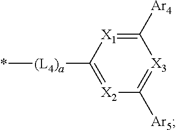
L₄ represents a single bond, a substituted or unsubstituted (C6-C30)cycloalkylene, or a substituted or unsubstituted (C10)arylene;
all of X₁ to X₃ represent N;
Ar₄ and Ar₅ each independently represent a substituted or unsubstituted (C6-C30)cycloalkyl, a substituted or unsubstituted (C6-C30)aryl, a substituted or unsubstituted (3- to 30-membered)heteroaryl, or a substituted or unsubstituted fused ring group of a (C3-C30) aliphatic ring(s) and a (C6-C30) aromatic ring(s), and Ar₄ and Ar₅ are the same as or different from each other;
with a proviso that when each of Ar₄ and Ar₅ represents an unsubstituted phenyl, an unsubstituted naphthyl, an unsubstituted biphenyl, an unsubstituted terphenyl, an unsubstituted phenanthrenyl, an unsubstituted triphenylenyl, an unsubstituted 1-dibenzofuranyl, an unsubstituted 1-dibenzothiophenyl, a phenyl substituted with a 1-dibenzofuranyl(s), a phenyl substituted with a 1-dibenzothiophenyl(s), a phenyl substituted with a 9-carbazolyl(s), an unsubstituted carbazolyl, a substituted or unsubstituted dibenzodioxynyl, a dibenzothiophenyl fused to benzofuran, or a dibenzothiophenyl fused to benzothiophene, and L₄ represents a single bond, L₄ is not linked at the position of R₉ or R₁₀;
with a proviso that when L₄ represents a single bond and is linked at the position of R₂ or R₇, Ar₄ and Ar₅ each independently represent a phenyl unsubstituted or substituted with a (C6-C30)cycloalkyl(s); a naphthyl unsubstituted or substituted with a dibenzofuranyl(s), a dibenzothiophenyl(s) or a phenanthrenyl(s); an unsubstituted biphenyl; an unsubstituted terphenyl (except for the structure 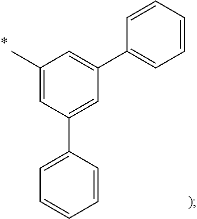 [SMILES: Cc1cc(-c2ccccc2)cc(-c2ccccc2)c1]
an unsubstituted phenanthrenyl; an unsubstituted triphenylenyl; an unsubstituted chrysenyl; an unsubstituted (C22)aryl; an unsubstituted 9,9-diphenylfluorenyl; an unsubstituted 7,7-dimethylbenzofluorenyl; an unsubstituted 9,9-diphenylsilafluorenyl; an fluorenyl substituted with a phenyl(s) and a (C6-C30)cycloalkyl(s); a fused ring group of a (C3-C10) aliphatic ring(s) and a (C6-C12) aromatic ring(s) substituted with a methyl(s); an unsubstituted phenylnaphthyl; an unsubstituted naphthylphenyl; an unsubstituted dibenzofuranyl; or an unsubstituted dibenzothiophenyl; and
a represents an integer of 1 to 3, where if a is an integer of 2 or more, each of L₄ may be the same or different.

2. The organic electroluminescent compound according to claim 1, wherein the compound represented by formula 2′ is selected from the following compounds:

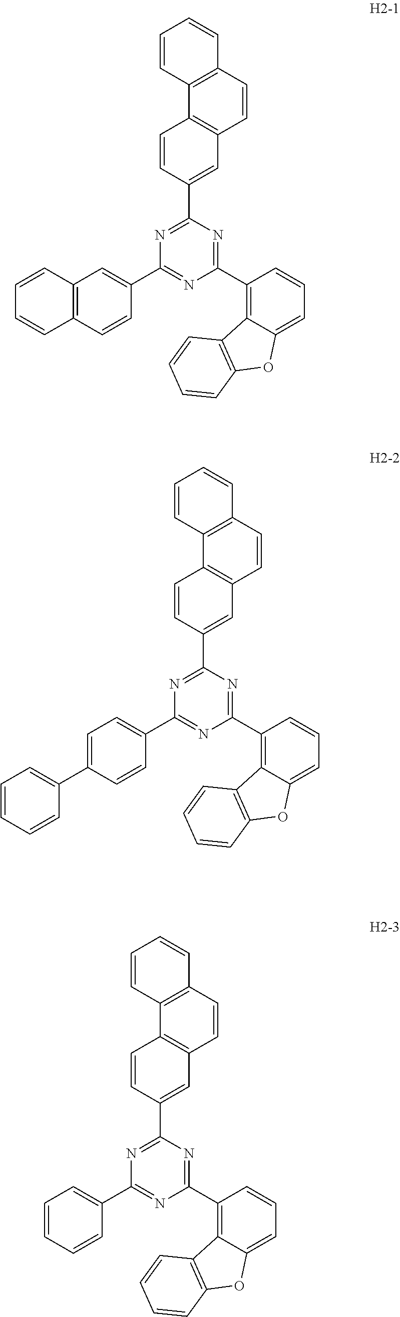 [SMILES: c1ccc2cc(-c3nc(-c4ccc5c(ccc6ccccc65)c4)nc(-c4cccc5oc6ccccc6c45)n3)ccc2c1 ; c1ccc(-c2ccc(-c3nc(-c4ccc5c(ccc6ccccc65)c4)nc(-c4cccc5oc6ccccc6c45)n3)cc2)cc1 ; c1ccc(-c2nc(-c3ccc4c(ccc5ccccc54)c3)nc(-c3cccc4oc5ccccc5c34)n2)cc1]

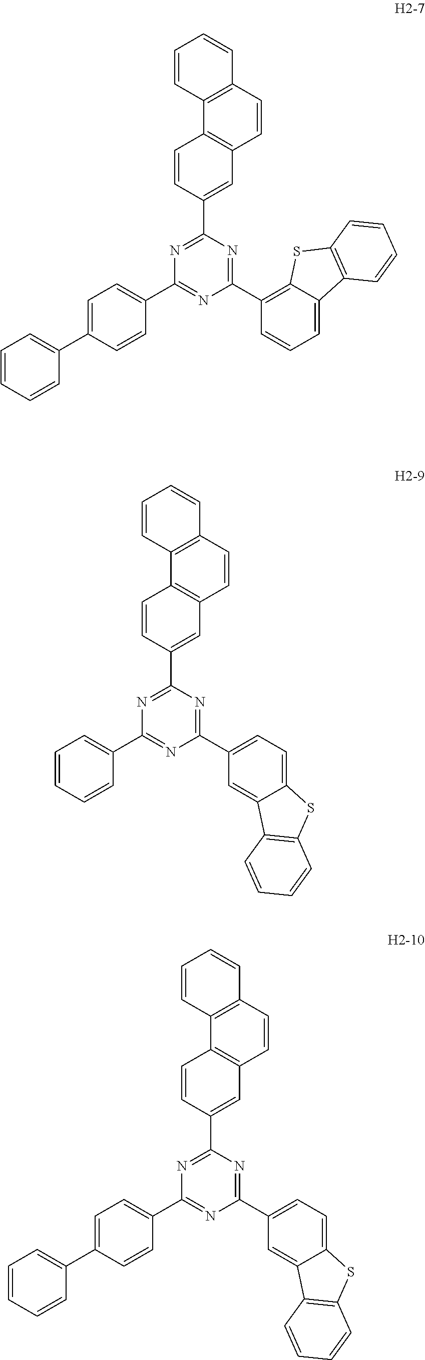 [SMILES: c1ccc(-c2ccc(-c3nc(-c4ccc5c(ccc6ccccc65)c4)nc(-c4cccc5c4sc4ccccc45)n3)cc2)cc1 ; c1ccc(-c2nc(-c3ccc4c(ccc5ccccc54)c3)nc(-c3ccc4sc5ccccc5c4c3)n2)cc1 ; c1ccc(-c2ccc(-c3nc(-c4ccc5c(ccc6ccccc65)c4)nc(-c4ccc5sc6ccccc6c5c4)n3)cc2)cc1]

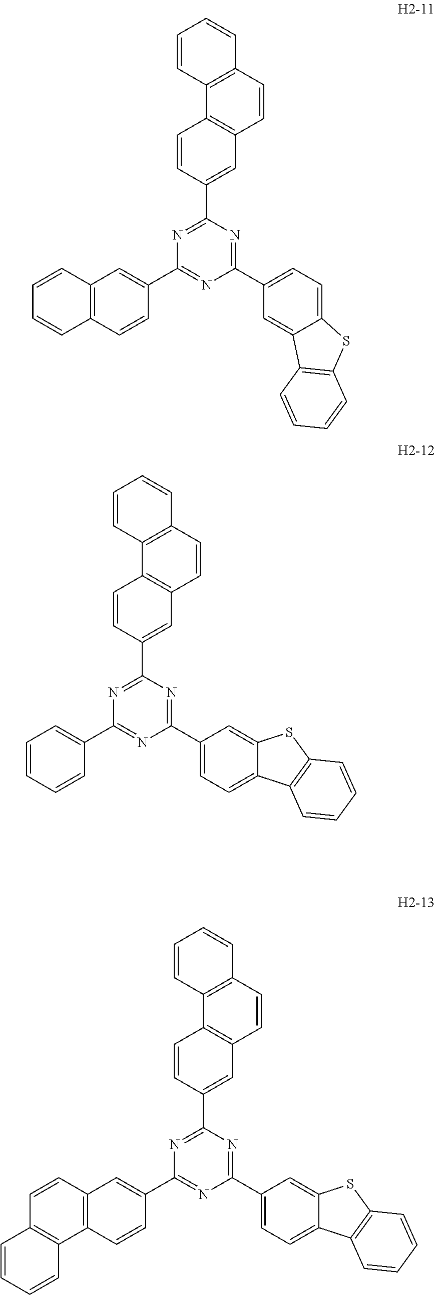 [SMILES: c1ccc2cc(-c3nc(-c4ccc5c(ccc6ccccc65)c4)nc(-c4ccc5sc6ccccc6c5c4)n3)ccc2c1 ; c1ccc(-c2nc(-c3ccc4c(ccc5ccccc54)c3)nc(-c3ccc4c(c3)sc3ccccc34)n2)cc1 ; c1ccc2c(c1)ccc1cc(-c3nc(-c4ccc5c(ccc6ccccc65)c4)nc(-c4ccc5c(c4)sc4ccccc45)n3)ccc12]

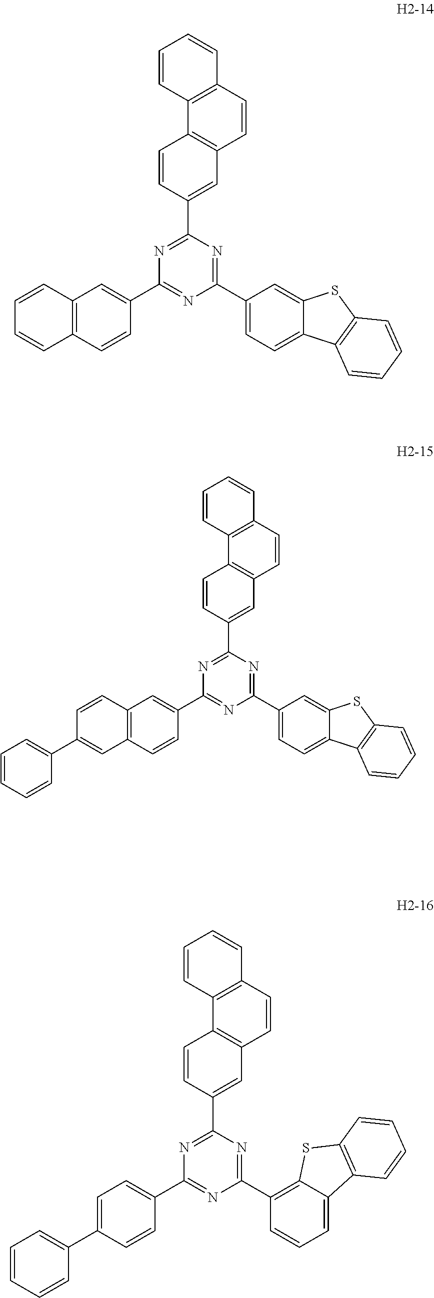 [SMILES: c1ccc2cc(-c3nc(-c4ccc5c(ccc6ccccc65)c4)nc(-c4ccc5c(c4)sc4ccccc45)n3)ccc2c1 ; c1ccc(-c2ccc3cc(-c4nc(-c5ccc6c(ccc7ccccc76)c5)nc(-c5ccc6c(c5)sc5ccccc56)n4)ccc3c2)cc1 ; c1ccc(-c2ccc(-c3nc(-c4ccc5c(ccc6ccccc65)c4)nc(-c4cccc5c4sc4ccccc45)n3)cc2)cc1]

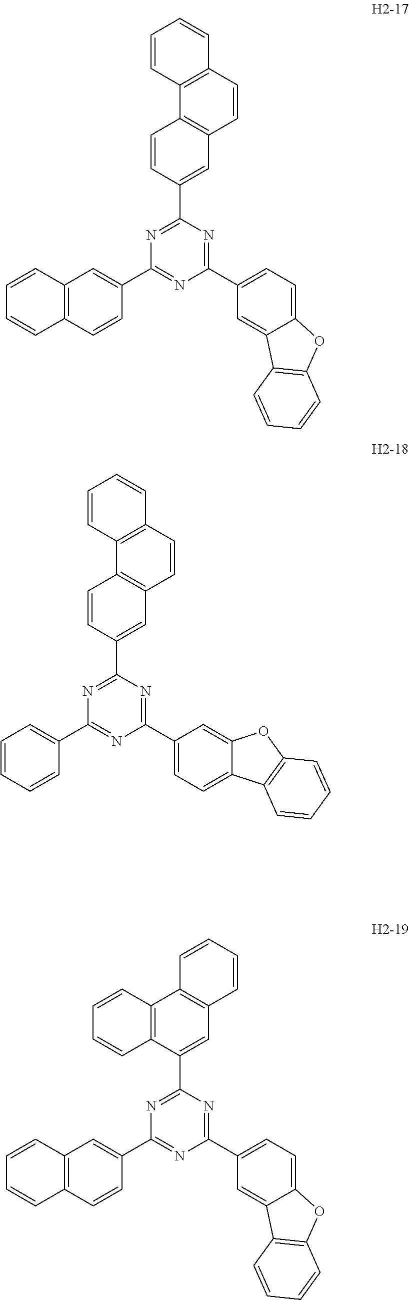 [SMILES: c1ccc2cc(-c3nc(-c4ccc5c(ccc6ccccc65)c4)nc(-c4ccc5oc6ccccc6c5c4)n3)ccc2c1 ; c1ccc(-c2nc(-c3ccc4c(ccc5ccccc54)c3)nc(-c3ccc4c(c3)oc3ccccc34)n2)cc1 ; c1ccc2cc(-c3nc(-c4ccc5oc6ccccc6c5c4)nc(-c4cc5ccccc5c5ccccc45)n3)ccc2c1]

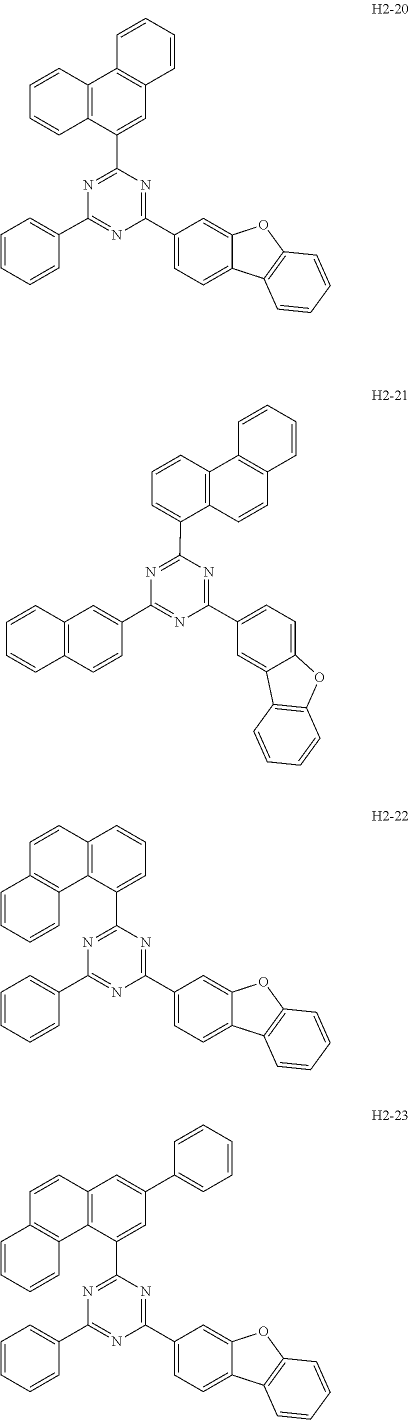 [SMILES: c1ccc(-c2nc(-c3ccc4c(c3)oc3ccccc34)nc(-c3cc4ccccc4c4ccccc34)n2)cc1 ; c1ccc2cc(-c3nc(-c4ccc5oc6ccccc6c5c4)nc(-c4cccc5c4ccc4ccccc45)n3)ccc2c1 ; c1ccc(-c2nc(-c3ccc4c(c3)oc3ccccc34)nc(-c3cccc4ccc5ccccc5c34)n2)cc1 ; c1ccc(-c2cc(-c3nc(-c4ccccc4)nc(-c4ccc5c(c4)oc4ccccc45)n3)c3c(ccc4ccccc43)c2)cc1]

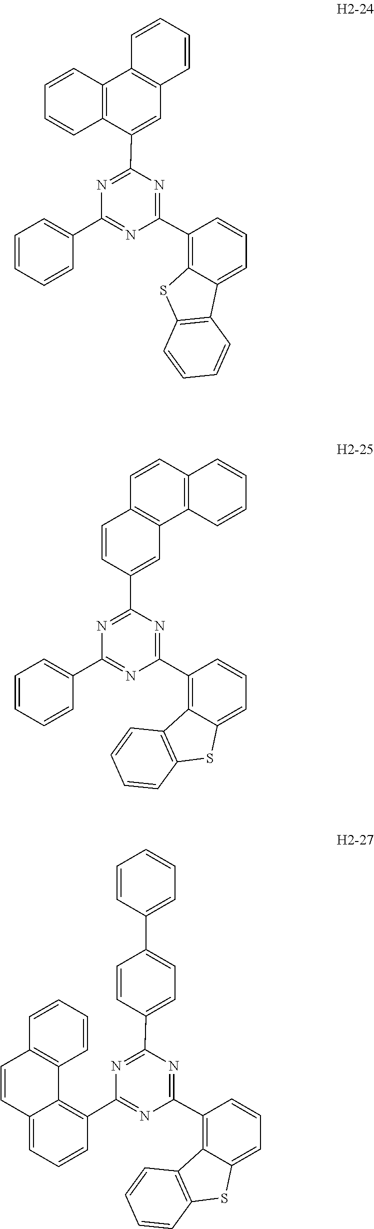 [SMILES: c1ccc(-c2nc(-c3cc4ccccc4c4ccccc34)nc(-c3cccc4c3sc3ccccc34)n2)cc1 ; c1ccc(-c2nc(-c3ccc4ccc5ccccc5c4c3)nc(-c3cccc4sc5ccccc5c34)n2)cc1 ; c1ccc(-c2ccc(-c3nc(-c4cccc5ccc6ccccc6c45)nc(-c4cccc5sc6ccccc6c45)n3)cc2)cc1]

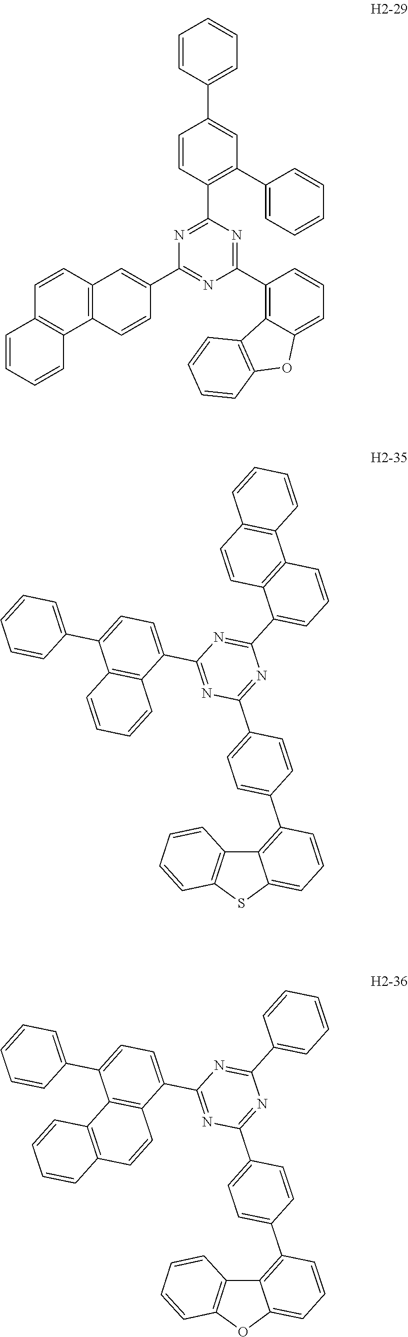 [SMILES: c1ccc(-c2ccc(-c3nc(-c4ccc5c(ccc6ccccc65)c4)nc(-c4cccc5oc6ccccc6c45)n3)c(-c3ccccc3)c2)cc1 ; c1ccc(-c2ccc(-c3nc(-c4ccc(-c5cccc6sc7ccccc7c56)cc4)nc(-c4cccc5c4ccc4ccccc45)n3)c3ccccc23)cc1 ; c1ccc(-c2nc(-c3ccc(-c4cccc5oc6ccccc6c45)cc3)nc(-c3ccc(-c4ccccc4)c4c3ccc3ccccc34)n2)cc1]

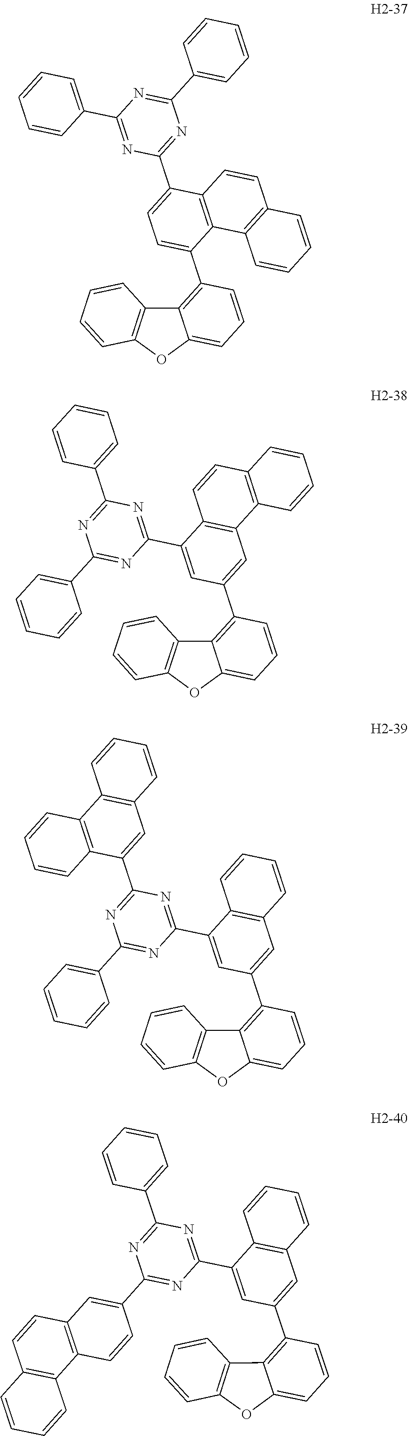 [SMILES: c1ccc(-c2nc(-c3ccccc3)nc(-c3ccc(-c4cccc5oc6ccccc6c45)c4c3ccc3ccccc34)n2)cc1 ; c1ccc(-c2nc(-c3ccccc3)nc(-c3cc(-c4cccc5oc6ccccc6c45)cc4c3ccc3ccccc34)n2)cc1 ; c1ccc(-c2nc(-c3cc(-c4cccc5oc6ccccc6c45)cc4ccccc34)nc(-c3cc4ccccc4c4ccccc34)n2)cc1 ; c1ccc(-c2nc(-c3ccc4c(ccc5ccccc54)c3)nc(-c3cc(-c4cccc5oc6ccccc6c45)cc4ccccc34)n2)cc1]

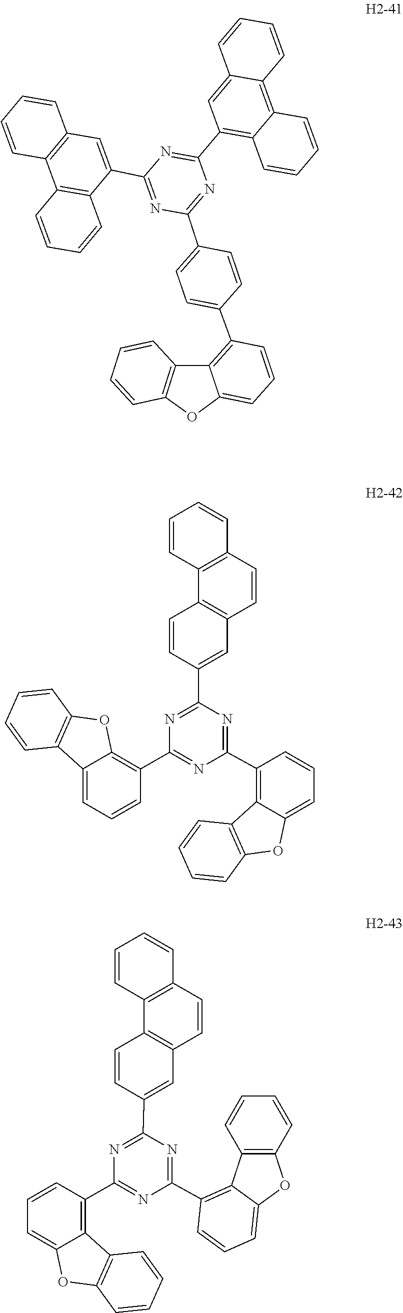 [SMILES: c1ccc2c(c1)cc(-c1nc(-c3ccc(-c4cccc5oc6ccccc6c45)cc3)nc(-c3cc4ccccc4c4ccccc34)n1)c1ccccc12 ; c1ccc2c(c1)ccc1cc(-c3nc(-c4cccc5c4oc4ccccc45)nc(-c4cccc5oc6ccccc6c45)n3)ccc12 ; c1ccc2c(c1)ccc1cc(-c3nc(-c4cccc5oc6ccccc6c45)nc(-c4cccc5oc6ccccc6c45)n3)ccc12]

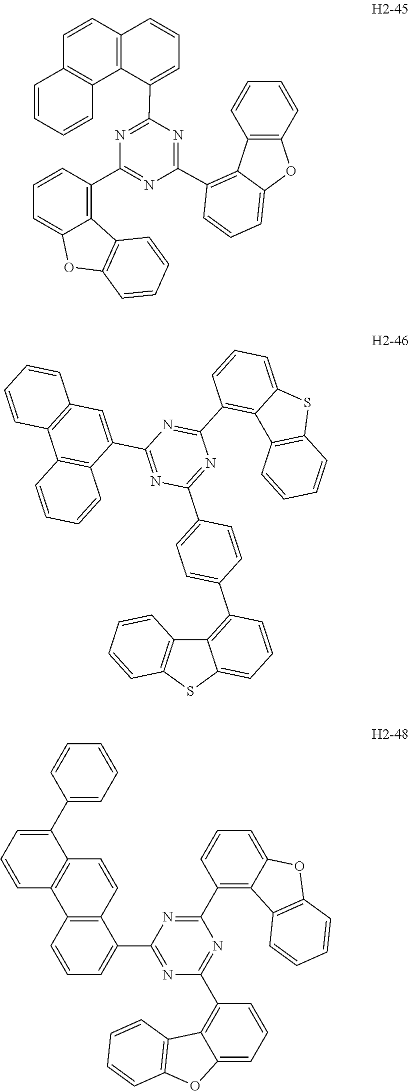 [SMILES: c1ccc2c(c1)ccc1cccc(-c3nc(-c4cccc5oc6ccccc6c45)nc(-c4cccc5oc6ccccc6c45)n3)c12 ; c1ccc2c(c1)cc(-c1nc(-c3ccc(-c4cccc5sc6ccccc6c45)cc3)nc(-c3cccc4sc5ccccc5c34)n1)c1ccccc12 ; c1ccc(-c2cccc3c2ccc2c(-c4nc(-c5cccc6oc7ccccc7c56)nc(-c5cccc6oc7ccccc7c56)n4)cccc23)cc1]

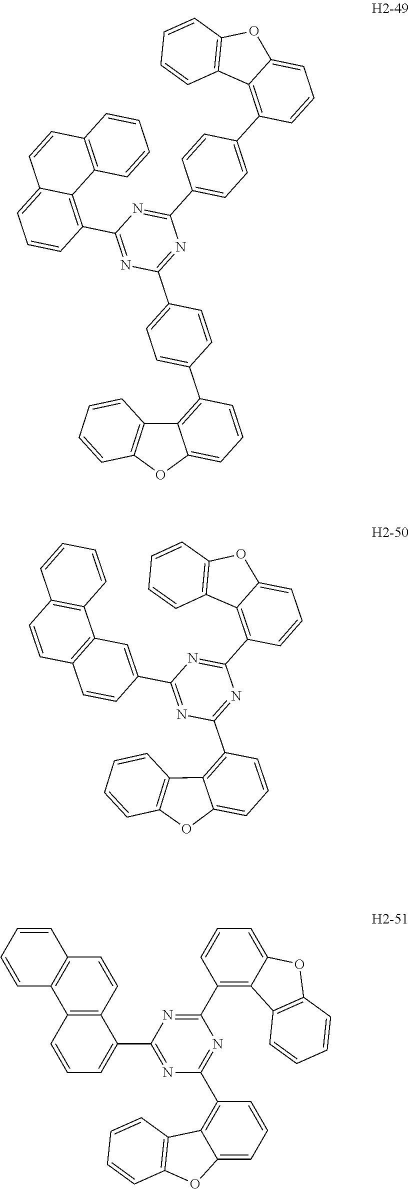 [SMILES: c1ccc2c(c1)ccc1cccc(-c3nc(-c4ccc(-c5cccc6oc7ccccc7c56)cc4)nc(-c4ccc(-c5cccc6oc7ccccc7c56)cc4)n3)c12 ; c1ccc2c(c1)ccc1ccc(-c3nc(-c4cccc5oc6ccccc6c45)nc(-c4cccc5oc6ccccc6c45)n3)cc12 ; c1ccc2c(c1)ccc1c(-c3nc(-c4cccc5oc6ccccc6c45)nc(-c4cccc5oc6ccccc6c45)n3)cccc12]

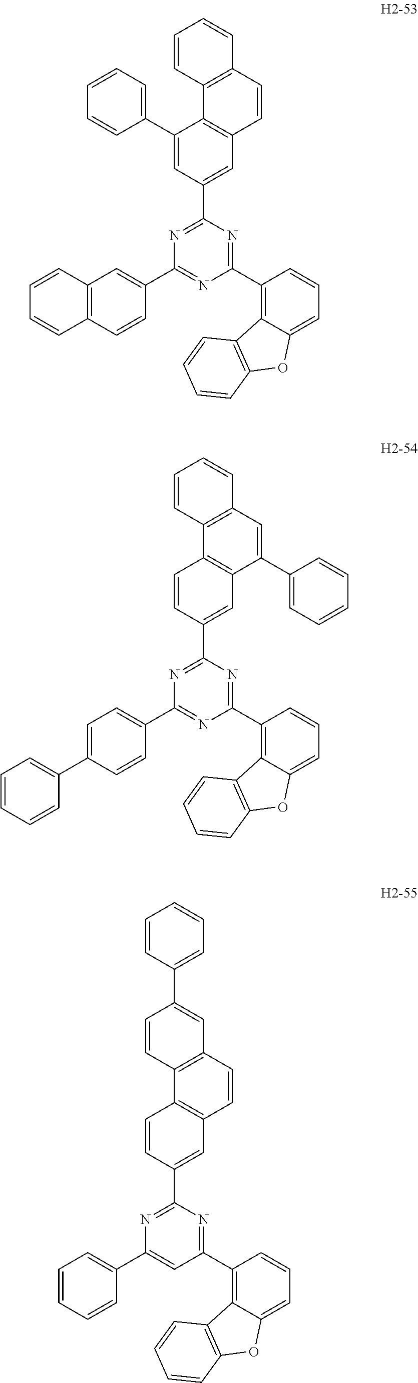 [SMILES: c1ccc(-c2cc(-c3nc(-c4ccc5ccccc5c4)nc(-c4cccc5oc6ccccc6c45)n3)cc3ccc4ccccc4c23)cc1 ; c1ccc(-c2ccc(-c3nc(-c4ccc5c(c4)c(-c4ccccc4)cc4ccccc45)nc(-c4cccc5oc6ccccc6c45)n3)cc2)cc1 ; c1ccc(-c2ccc3c(ccc4cc(-c5nc(-c6ccccc6)cc(-c6cccc7oc8ccccc8c67)n5)ccc43)c2)cc1]

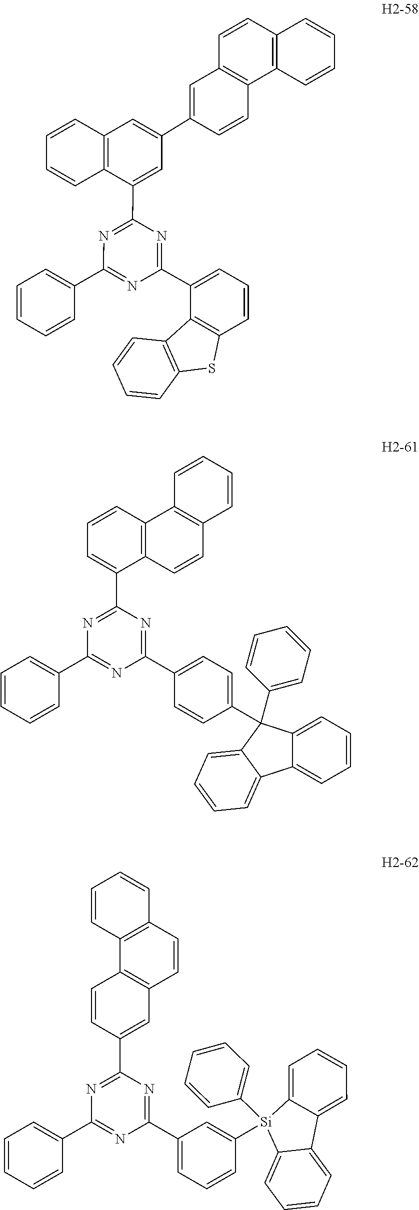 [SMILES: c1ccc(-c2nc(-c3cc(-c4ccc5c(ccc6ccccc65)c4)cc4ccccc34)nc(-c3cccc4sc5ccccc5c34)n2)cc1 ; c1ccc(-c2nc(-c3ccc(C4(c5ccccc5)c5ccccc5-c5ccccc54)cc3)nc(-c3cccc4c3ccc3ccccc34)n2)cc1 ; c1ccc(-c2nc(-c3cccc([Si]4(c5ccccc5)c5ccccc5-c5ccccc54)c3)nc(-c3ccc4c(ccc5ccccc54)c3)n2)cc1]

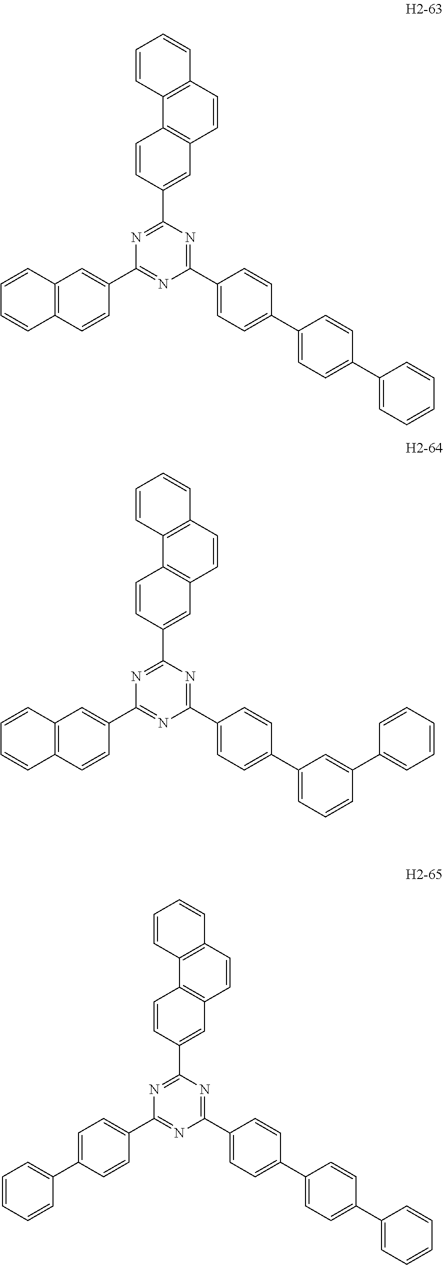 [SMILES: c1ccc(-c2ccc(-c3ccc(-c4nc(-c5ccc6ccccc6c5)nc(-c5ccc6c(ccc7ccccc76)c5)n4)cc3)cc2)cc1 ; c1ccc(-c2cccc(-c3ccc(-c4nc(-c5ccc6ccccc6c5)nc(-c5ccc6c(ccc7ccccc76)c5)n4)cc3)c2)cc1 ; c1ccc(-c2ccc(-c3ccc(-c4nc(-c5ccc(-c6ccccc6)cc5)nc(-c5ccc6c(ccc7ccccc76)c5)n4)cc3)cc2)cc1]

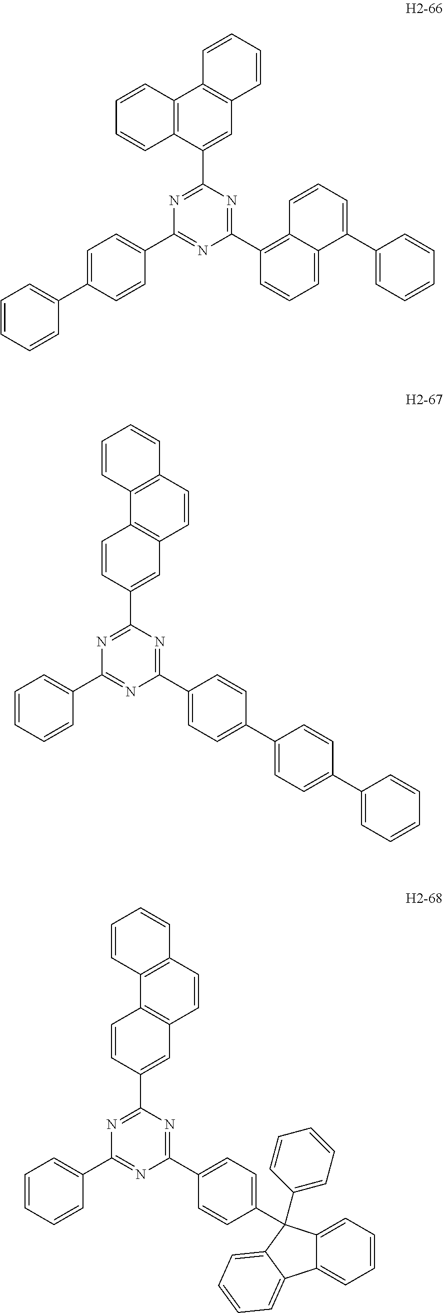 [SMILES: c1ccc(-c2ccc(-c3nc(-c4cccc5c(-c6ccccc6)cccc45)nc(-c4cc5ccccc5c5ccccc45)n3)cc2)cc1 ; c1ccc(-c2ccc(-c3ccc(-c4nc(-c5ccccc5)nc(-c5ccc6c(ccc7ccccc76)c5)n4)cc3)cc2)cc1 ; c1ccc(-c2nc(-c3ccc(C4(c5ccccc5)c5ccccc5-c5ccccc54)cc3)nc(-c3ccc4c(ccc5ccccc54)c3)n2)cc1]

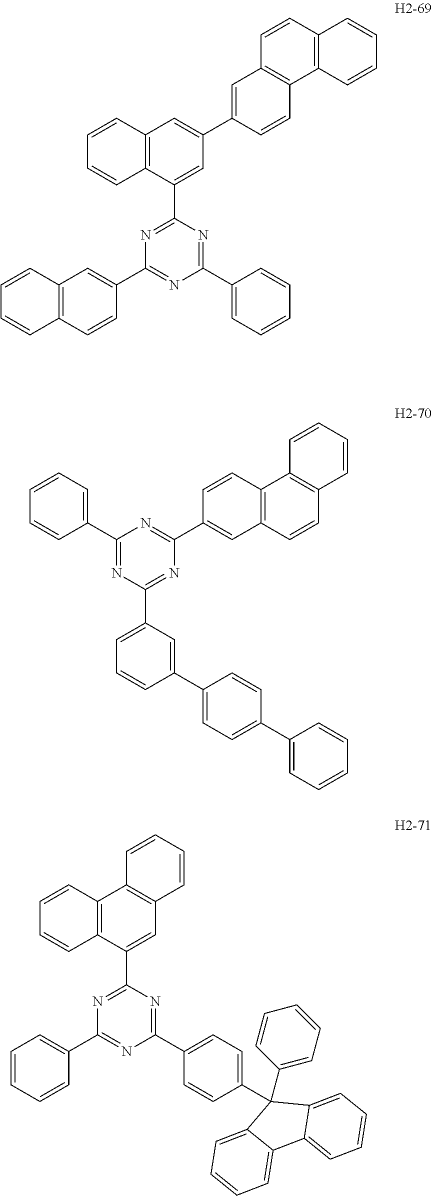 [SMILES: c1ccc(-c2nc(-c3ccc4ccccc4c3)nc(-c3cc(-c4ccc5c(ccc6ccccc65)c4)cc4ccccc34)n2)cc1 ; c1ccc(-c2ccc(-c3cccc(-c4nc(-c5ccccc5)nc(-c5ccc6c(ccc7ccccc76)c5)n4)c3)cc2)cc1 ; c1ccc(-c2nc(-c3ccc(C4(c5ccccc5)c5ccccc5-c5ccccc54)cc3)nc(-c3cc4ccccc4c4ccccc34)n2)cc1]

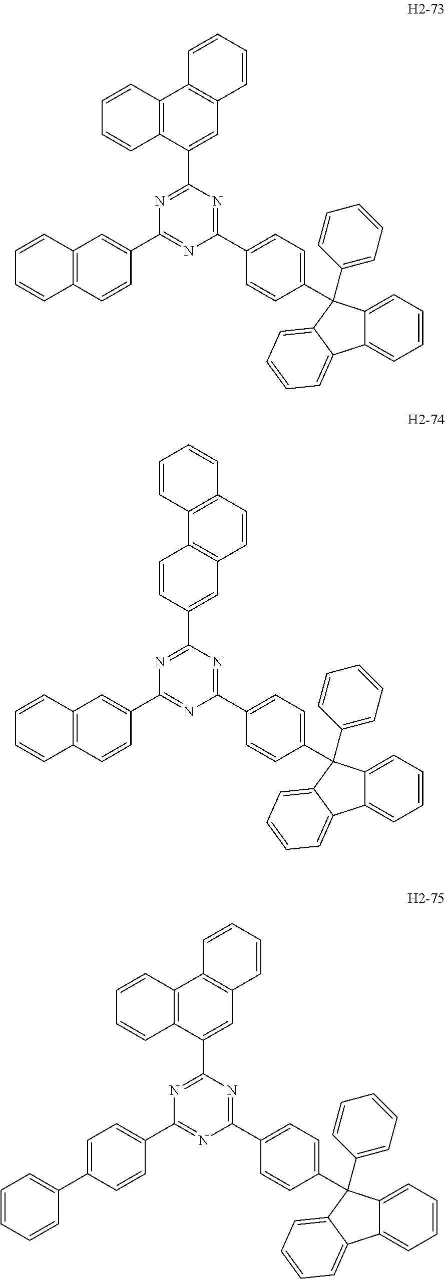 [SMILES: c1ccc(C2(c3ccc(-c4nc(-c5ccc6ccccc6c5)nc(-c5cc6ccccc6c6ccccc56)n4)cc3)c3ccccc3-c3ccccc32)cc1 ; c1ccc(C2(c3ccc(-c4nc(-c5ccc6ccccc6c5)nc(-c5ccc6c(ccc7ccccc76)c5)n4)cc3)c3ccccc3-c3ccccc32)cc1 ; c1ccc(-c2ccc(-c3nc(-c4ccc(C5(c6ccccc6)c6ccccc6-c6ccccc65)cc4)nc(-c4cc5ccccc5c5ccccc45)n3)cc2)cc1]

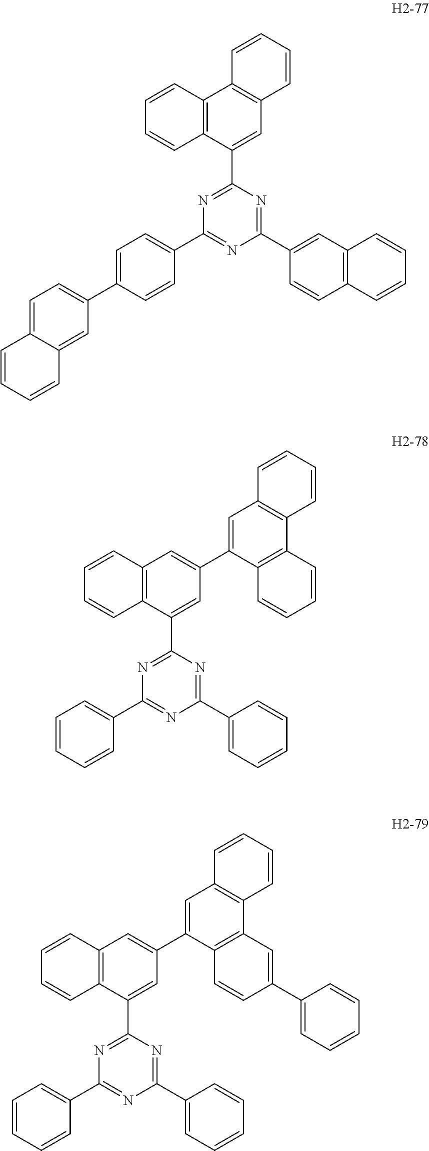 [SMILES: c1ccc2cc(-c3ccc(-c4nc(-c5ccc6ccccc6c5)nc(-c5cc6ccccc6c6ccccc56)n4)cc3)ccc2c1 ; c1ccc(-c2nc(-c3ccccc3)nc(-c3cc(-c4cc5ccccc5c5ccccc45)cc4ccccc34)n2)cc1 ; c1ccc(-c2ccc3c(-c4cc(-c5nc(-c6ccccc6)nc(-c6ccccc6)n5)c5ccccc5c4)cc4ccccc4c3c2)cc1]

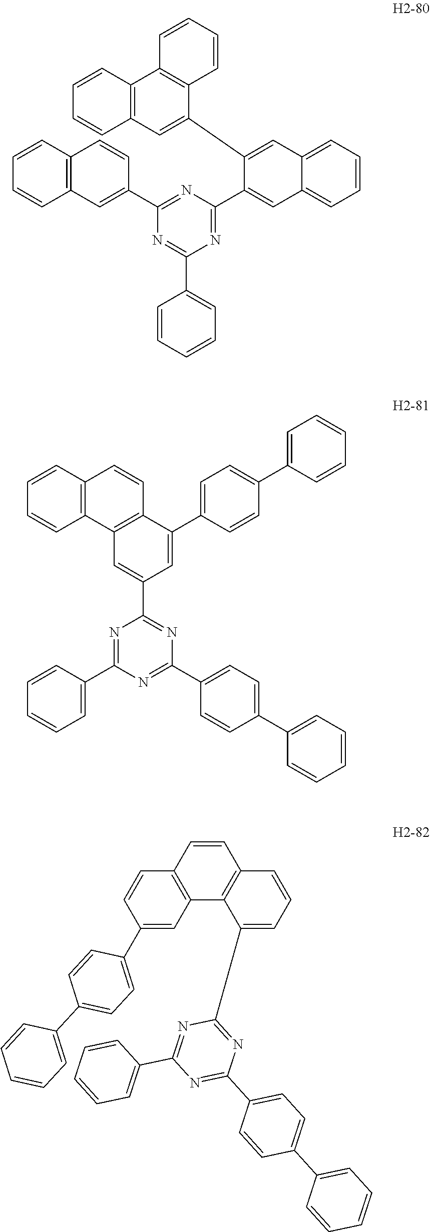 [SMILES: c1ccc(-c2nc(-c3ccc4ccccc4c3)nc(-c3cc4ccccc4cc3-c3cc4ccccc4c4ccccc34)n2)cc1 ; c1ccc(-c2ccc(-c3nc(-c4ccccc4)nc(-c4cc(-c5ccc(-c6ccccc6)cc5)c5ccc6ccccc6c5c4)n3)cc2)cc1 ; c1ccc(-c2ccc(-c3ccc4ccc5cccc(-c6nc(-c7ccccc7)nc(-c7ccc(-c8ccccc8)cc7)n6)c5c4c3)cc2)cc1]

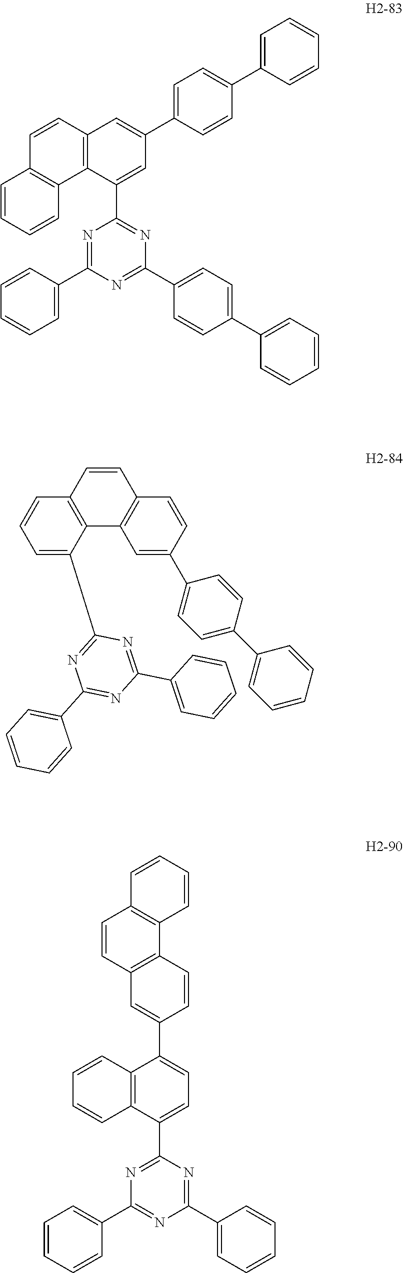 [SMILES: c1ccc(-c2ccc(-c3cc(-c4nc(-c5ccccc5)nc(-c5ccc(-c6ccccc6)cc5)n4)c4c(ccc5ccccc54)c3)cc2)cc1 ; c1ccc(-c2ccc(-c3ccc4ccc5cccc(-c6nc(-c7ccccc7)nc(-c7ccccc7)n6)c5c4c3)cc2)cc1 ; c1ccc(-c2nc(-c3ccccc3)nc(-c3ccc(-c4ccc5c(ccc6ccccc65)c4)c4ccccc34)n2)cc1]

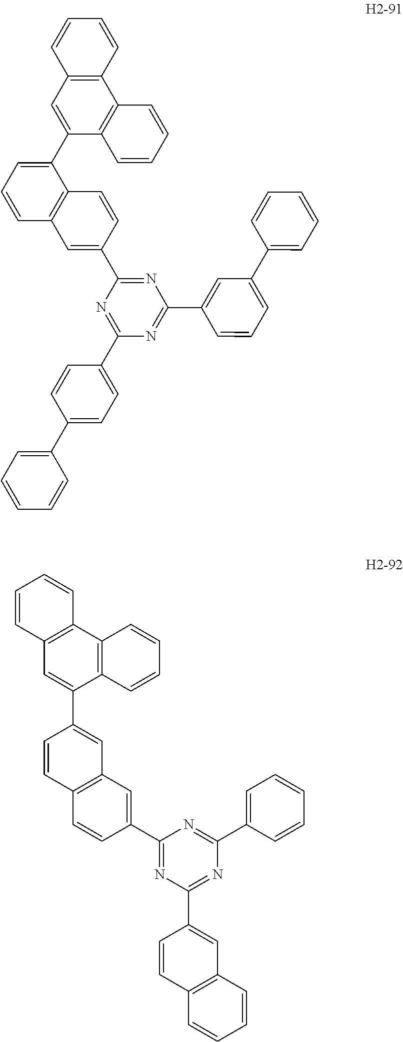 [SMILES: c1ccc(-c2ccc(-c3nc(-c4cccc(-c5ccccc5)c4)nc(-c4ccc5c(-c6cc7ccccc7c7ccccc67)cccc5c4)n3)cc2)cc1 ; c1ccc(-c2nc(-c3ccc4ccccc4c3)nc(-c3ccc4ccc(-c5cc6ccccc6c6ccccc56)cc4c3)n2)cc1]

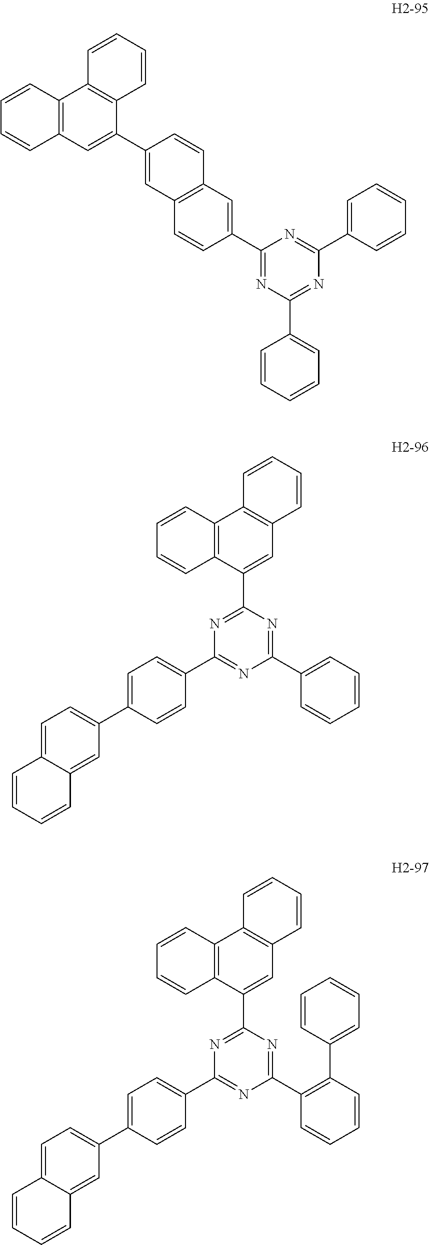 [SMILES: c1ccc(-c2nc(-c3ccccc3)nc(-c3ccc4cc(-c5cc6ccccc6c6ccccc56)ccc4c3)n2)cc1 ; c1ccc(-c2nc(-c3ccc(-c4ccc5ccccc5c4)cc3)nc(-c3cc4ccccc4c4ccccc34)n2)cc1 ; c1ccc(-c2ccccc2-c2nc(-c3ccc(-c4ccc5ccccc5c4)cc3)nc(-c3cc4ccccc4c4ccccc34)n2)cc1]

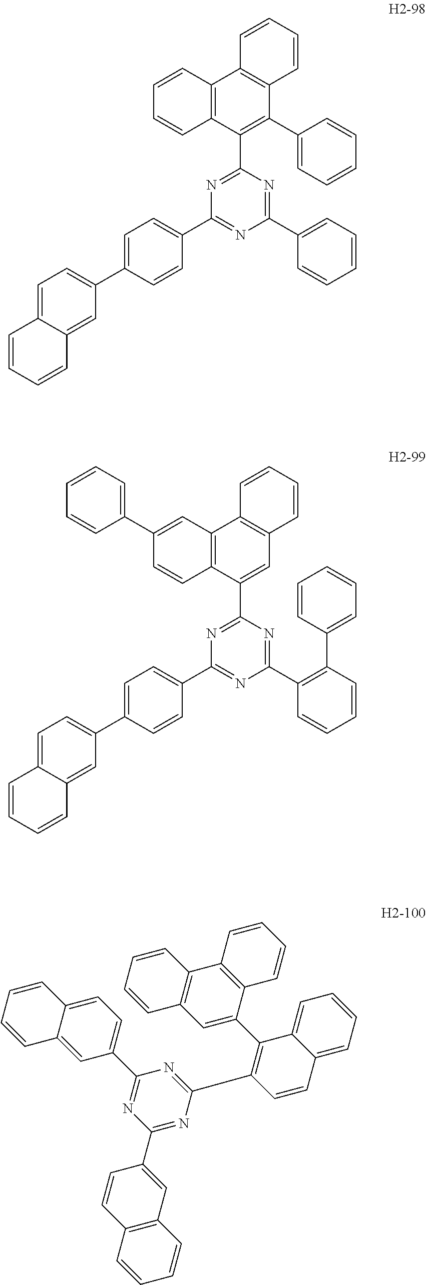 [SMILES: c1ccc(-c2nc(-c3ccc(-c4ccc5ccccc5c4)cc3)nc(-c3c(-c4ccccc4)c4ccccc4c4ccccc34)n2)cc1 ; c1ccc(-c2ccc3c(-c4nc(-c5ccc(-c6ccc7ccccc7c6)cc5)nc(-c5ccccc5-c5ccccc5)n4)cc4ccccc4c3c2)cc1 ; c1ccc2cc(-c3nc(-c4ccc5ccccc5c4)nc(-c4ccc5ccccc5c4-c4cc5ccccc5c5ccccc45)n3)ccc2c1]

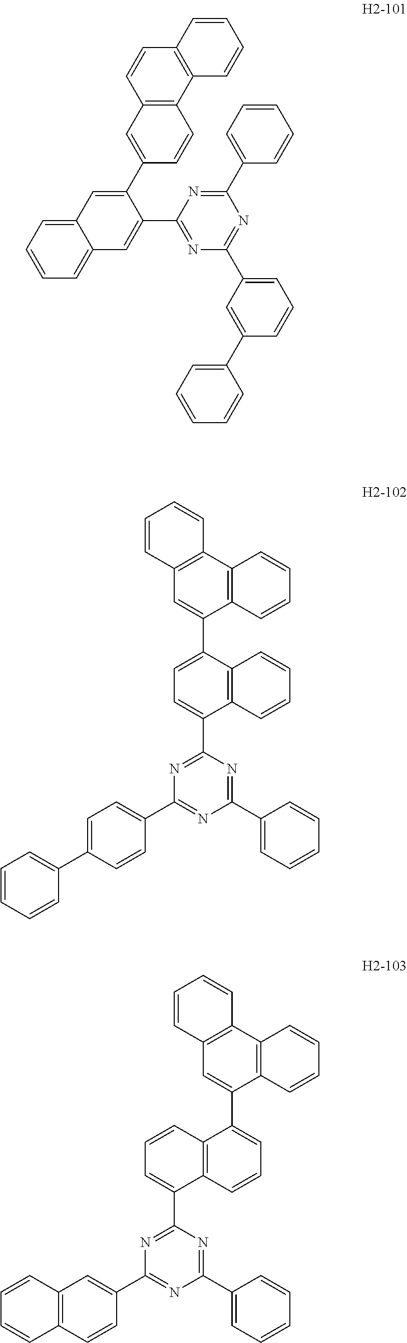 [SMILES: c1ccc(-c2cccc(-c3nc(-c4ccccc4)nc(-c4cc5ccccc5cc4-c4ccc5c(ccc6ccccc65)c4)n3)c2)cc1 ; c1ccc(-c2ccc(-c3nc(-c4ccccc4)nc(-c4ccc(-c5cc6ccccc6c6ccccc56)c5ccccc45)n3)cc2)cc1 ; c1ccc(-c2nc(-c3ccc4ccccc4c3)nc(-c3cccc4c(-c5cc6ccccc6c6ccccc56)cccc34)n2)cc1]

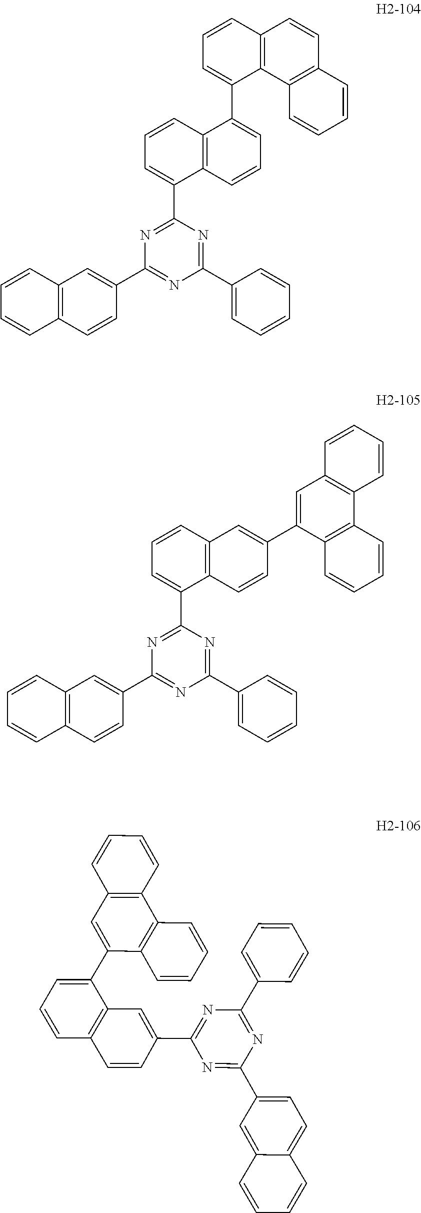 [SMILES: c1ccc(-c2nc(-c3ccc4ccccc4c3)nc(-c3cccc4c(-c5cccc6ccc7ccccc7c56)cccc34)n2)cc1 ; c1ccc(-c2nc(-c3ccc4ccccc4c3)nc(-c3cccc4cc(-c5cc6ccccc6c6ccccc56)ccc34)n2)cc1 ; c1ccc(-c2nc(-c3ccc4ccccc4c3)nc(-c3ccc4cccc(-c5cc6ccccc6c6ccccc56)c4c3)n2)cc1]

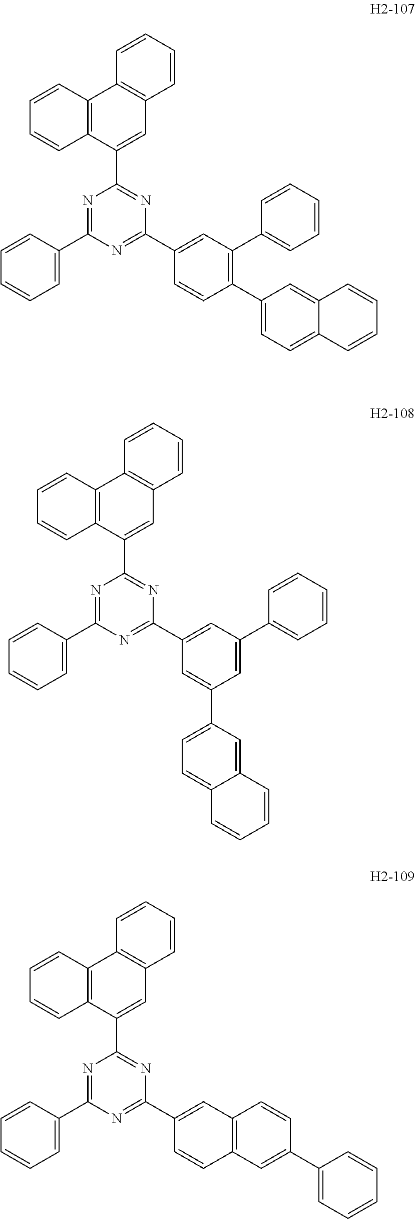 [SMILES: c1ccc(-c2nc(-c3ccc(-c4ccc5ccccc5c4)c(-c4ccccc4)c3)nc(-c3cc4ccccc4c4ccccc34)n2)cc1 ; c1ccc(-c2cc(-c3ccc4ccccc4c3)cc(-c3nc(-c4ccccc4)nc(-c4cc5ccccc5c5ccccc45)n3)c2)cc1 ; c1ccc(-c2ccc3cc(-c4nc(-c5ccccc5)nc(-c5cc6ccccc6c6ccccc56)n4)ccc3c2)cc1]

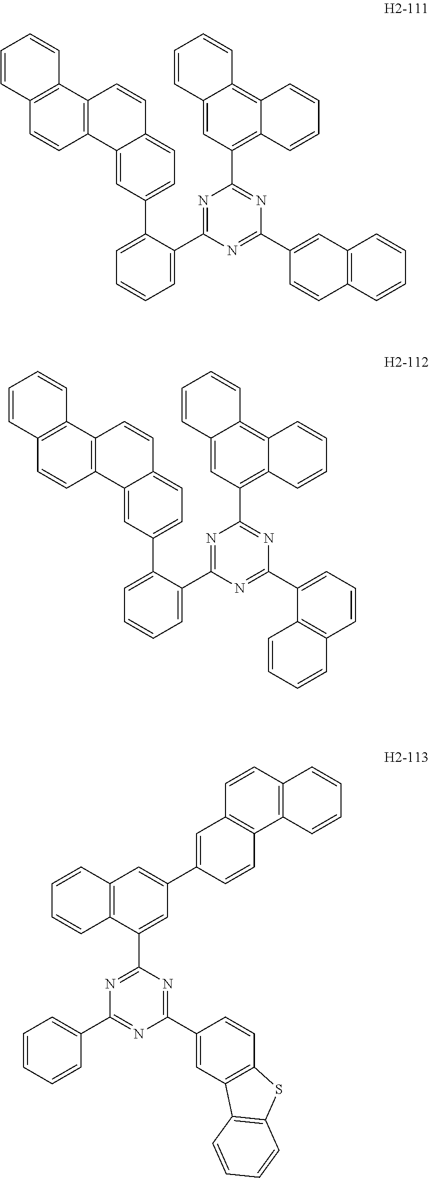 [SMILES: c1ccc(-c2nc(-c3ccc4ccccc4c3)nc(-c3cc4ccccc4c4ccccc34)n2)c(-c2ccc3ccc4c5ccccc5ccc4c3c2)c1 ; c1ccc(-c2nc(-c3cccc4ccccc34)nc(-c3cc4ccccc4c4ccccc34)n2)c(-c2ccc3ccc4c5ccccc5ccc4c3c2)c1 ; c1ccc(-c2nc(-c3ccc4sc5ccccc5c4c3)nc(-c3cc(-c4ccc5c(ccc6ccccc65)c4)cc4ccccc34)n2)cc1]

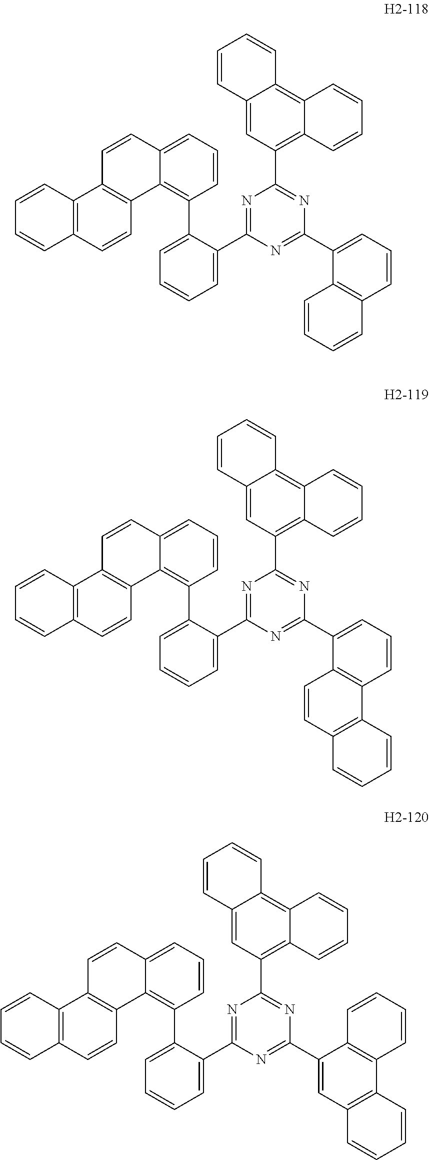 [SMILES: c1ccc(-c2cccc3ccc4c5ccccc5ccc4c23)c(-c2nc(-c3cccc4ccccc34)nc(-c3cc4ccccc4c4ccccc34)n2)c1 ; c1ccc(-c2cccc3ccc4c5ccccc5ccc4c23)c(-c2nc(-c3cccc4c3ccc3ccccc34)nc(-c3cc4ccccc4c4ccccc34)n2)c1 ; c1ccc(-c2cccc3ccc4c5ccccc5ccc4c23)c(-c2nc(-c3cc4ccccc4c4ccccc34)nc(-c3cc4ccccc4c4ccccc34)n2)c1]

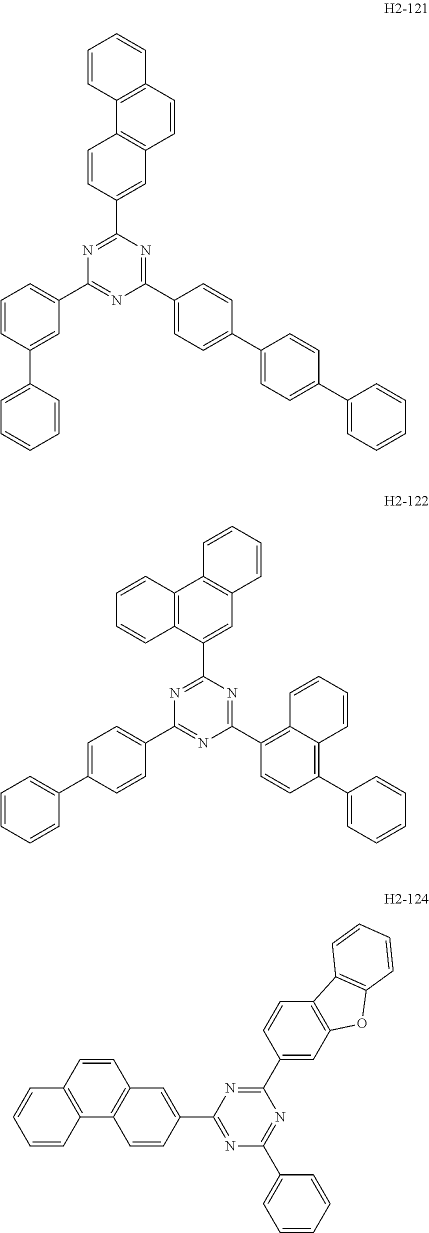 [SMILES: c1ccc(-c2ccc(-c3ccc(-c4nc(-c5cccc(-c6ccccc6)c5)nc(-c5ccc6c(ccc7ccccc76)c5)n4)cc3)cc2)cc1 ; c1ccc(-c2ccc(-c3nc(-c4ccc(-c5ccccc5)c5ccccc45)nc(-c4cc5ccccc5c5ccccc45)n3)cc2)cc1 ; c1ccc(-c2nc(-c3ccc4c(ccc5ccccc54)c3)nc(-c3ccc4c(c3)oc3ccccc34)n2)cc1]

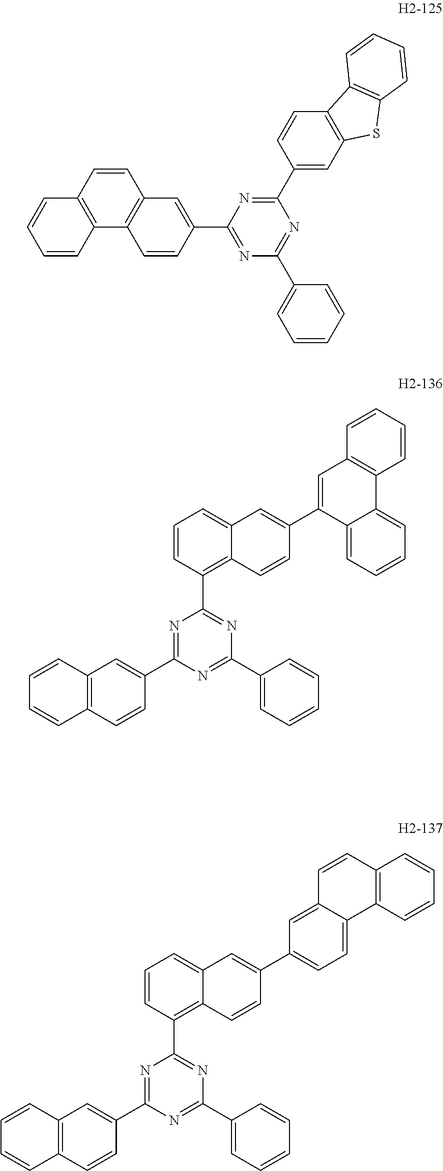 [SMILES: c1ccc(-c2nc(-c3ccc4c(ccc5ccccc54)c3)nc(-c3ccc4c(c3)sc3ccccc34)n2)cc1 ; c1ccc(-c2nc(-c3ccc4ccccc4c3)nc(-c3cccc4cc(-c5cc6ccccc6c6ccccc56)ccc34)n2)cc1 ; c1ccc(-c2nc(-c3ccc4ccccc4c3)nc(-c3cccc4cc(-c5ccc6c(ccc7ccccc76)c5)ccc34)n2)cc1]

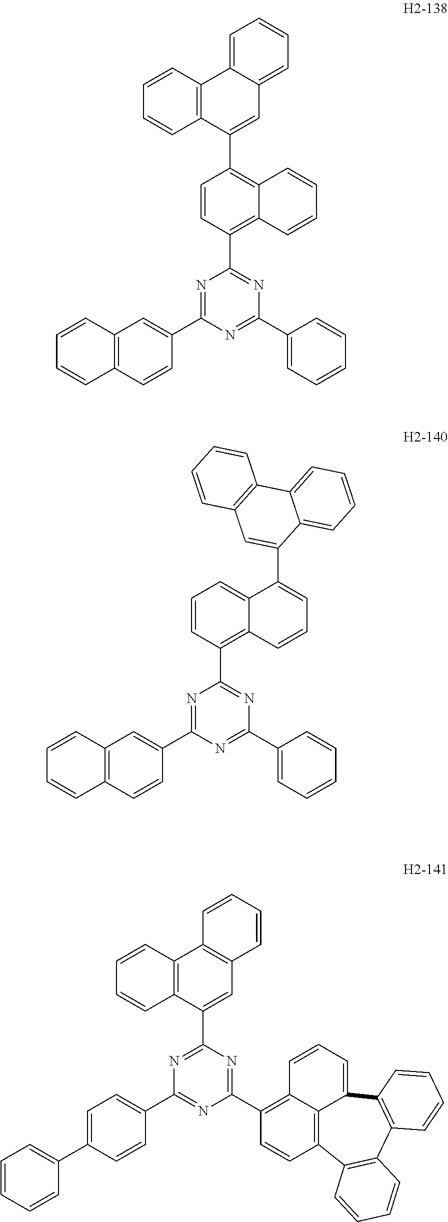

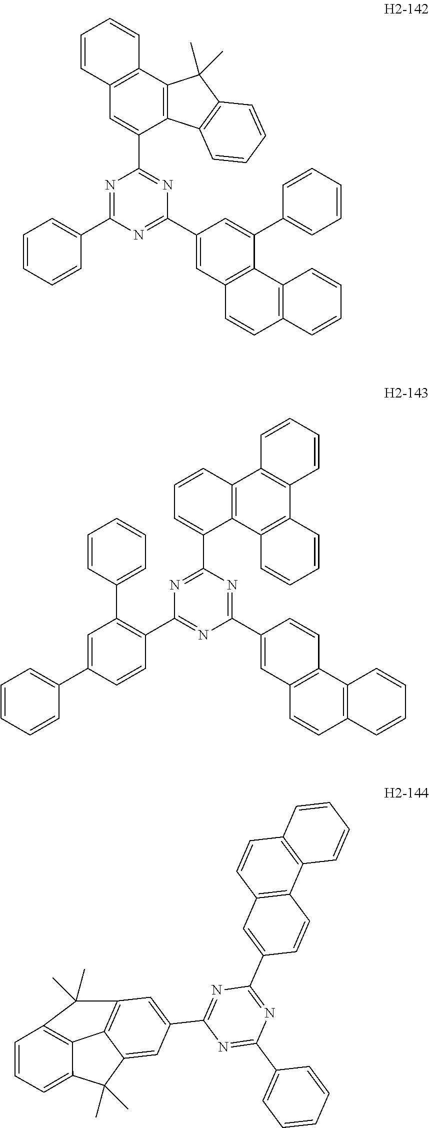 [SMILES: CC1(C)c2ccccc2-c2c(-c3nc(-c4ccccc4)nc(-c4cc(-c5ccccc5)c5c(ccc6ccccc65)c4)n3)cc3ccccc3c21 ; c1ccc(-c2ccc(-c3nc(-c4ccc5c(ccc6ccccc65)c4)nc(-c4cccc5c6ccccc6c6ccccc6c45)n3)c(-c3ccccc3)c2)cc1 ; CC1(C)c2cccc3c2-c2c1cc(-c1nc(-c4ccccc4)nc(-c4ccc5c(ccc6ccccc65)c4)n1)cc2C3(C)C]

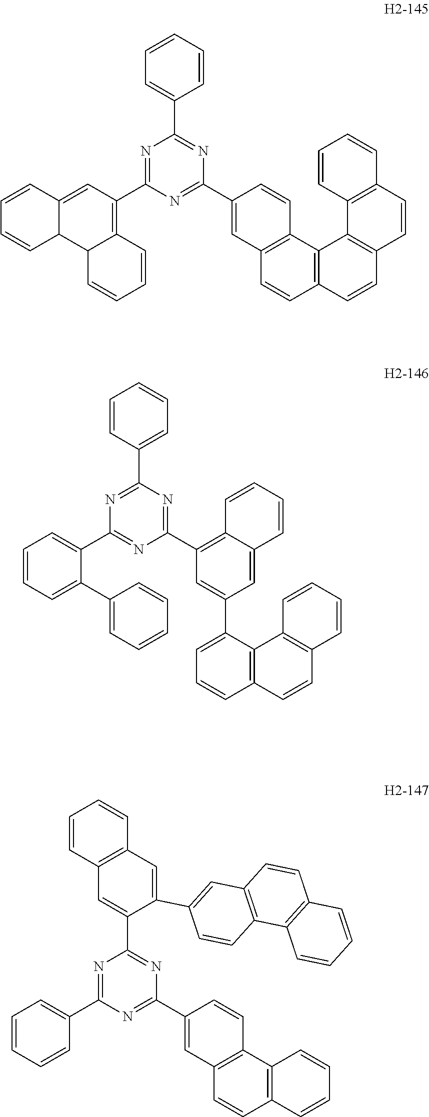 [SMILES: C1=CC2=CC(c3nc(-c4ccccc4)nc(-c4ccc5c(ccc6ccc7ccc8ccccc8c7c65)c4)n3)=C3C=CC=CC3C2C=C1 ; c1ccc(-c2nc(-c3ccccc3-c3ccccc3)nc(-c3cc(-c4cccc5ccc6ccccc6c45)cc4ccccc34)n2)cc1 ; c1ccc(-c2nc(-c3ccc4c(ccc5ccccc54)c3)nc(-c3cc4ccccc4cc3-c3ccc4c(ccc5ccccc54)c3)n2)cc1]

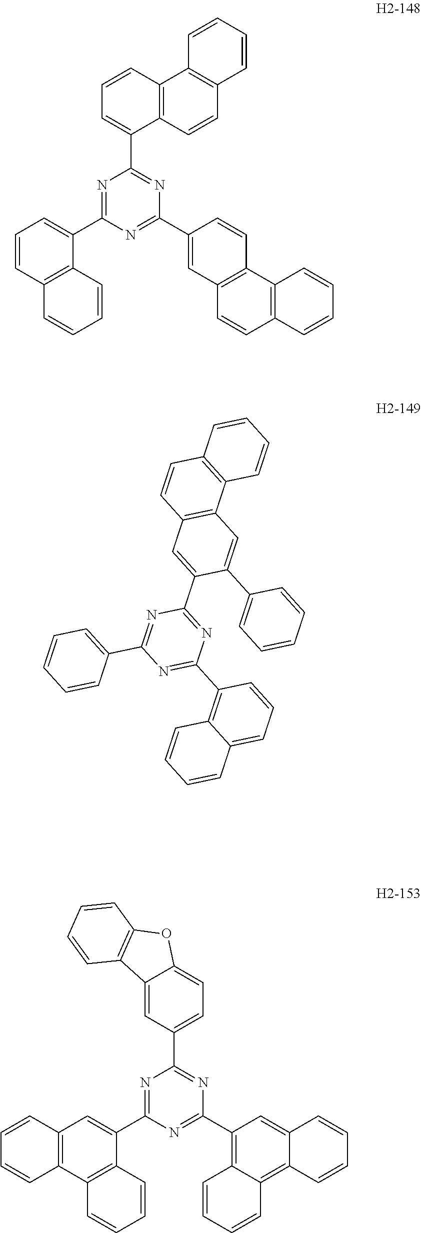 [SMILES: c1ccc2c(-c3nc(-c4ccc5c(ccc6ccccc65)c4)nc(-c4cccc5c4ccc4ccccc45)n3)cccc2c1 ; c1ccc(-c2nc(-c3cc4ccc5ccccc5c4cc3-c3ccccc3)nc(-c3cccc4ccccc34)n2)cc1 ; c1ccc2c(c1)cc(-c1nc(-c3ccc4oc5ccccc5c4c3)nc(-c3cc4ccccc4c4ccccc34)n1)c1ccccc12]

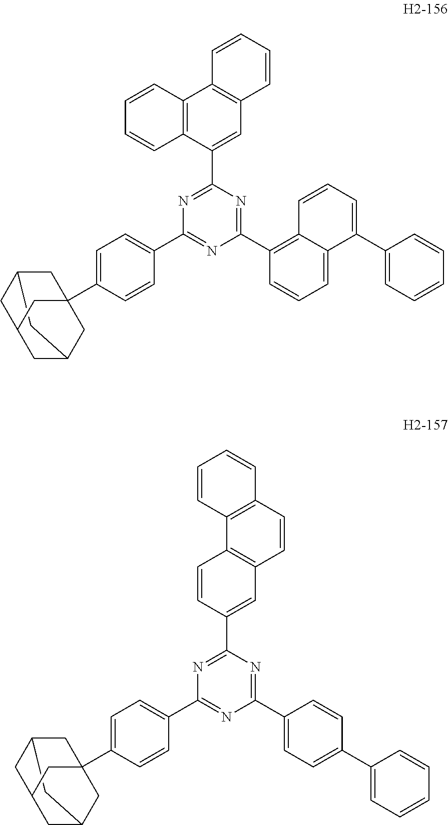 [SMILES: c1ccc(-c2cccc3c(-c4nc(-c5ccc(C67CC8CC(CC(C8)C6)C7)cc5)nc(-c5cc6ccccc6c6ccccc56)n4)cccc23)cc1 ; c1ccc(-c2ccc(-c3nc(-c4ccc(C56CC7CC(CC(C7)C5)C6)cc4)nc(-c4ccc5c(ccc6ccccc65)c4)n3)cc2)cc1]

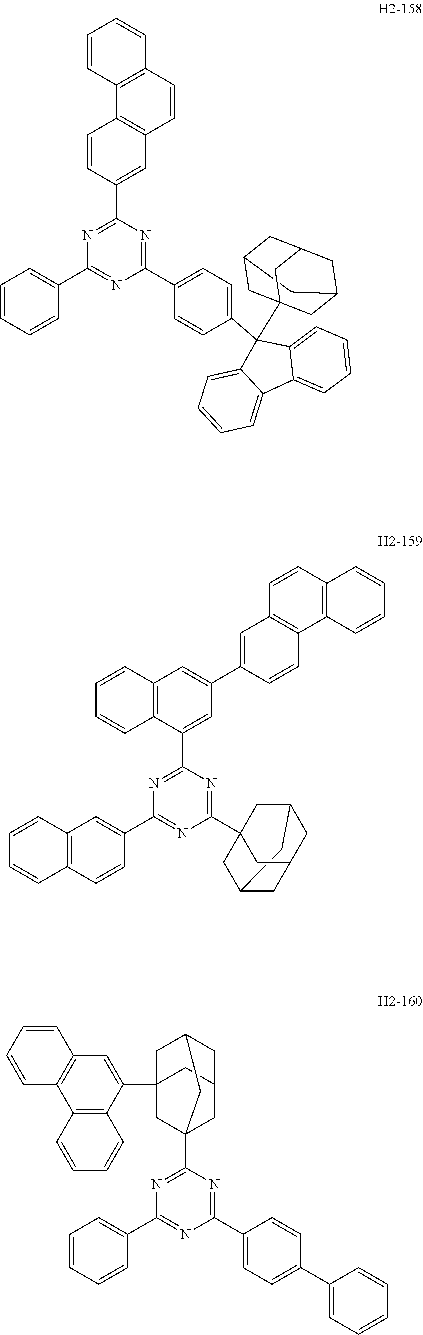 [SMILES: c1ccc(-c2nc(-c3ccc(C4(C56CC7CC(CC(C7)C5)C6)c5ccccc5-c5ccccc54)cc3)nc(-c3ccc4c(ccc5ccccc54)c3)n2)cc1 ; c1ccc2cc(-c3nc(-c4cc(-c5ccc6c(ccc7ccccc76)c5)cc5ccccc45)nc(C45CC6CC(CC(C6)C4)C5)n3)ccc2c1 ; c1ccc(-c2ccc(-c3nc(-c4ccccc4)nc(C45CC6CC(C4)CC(c4cc7ccccc7c7ccccc47)(C6)C5)n3)cc2)cc1]

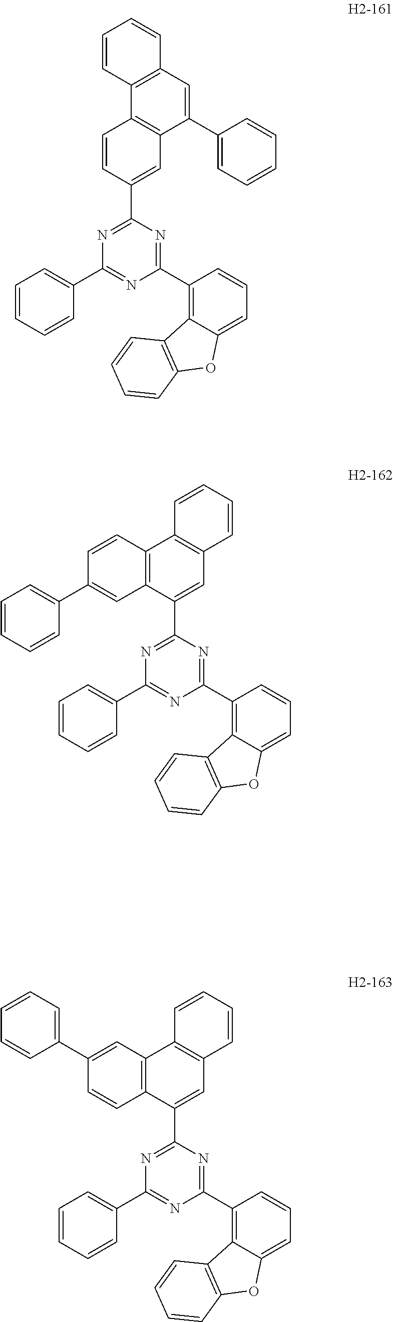 [SMILES: c1ccc(-c2nc(-c3ccc4c(c3)c(-c3ccccc3)cc3ccccc34)nc(-c3cccc4oc5ccccc5c34)n2)cc1 ; c1ccc(-c2ccc3c(c2)c(-c2nc(-c4ccccc4)nc(-c4cccc5oc6ccccc6c45)n2)cc2ccccc23)cc1 ; c1ccc(-c2ccc3c(-c4nc(-c5ccccc5)nc(-c5cccc6oc7ccccc7c56)n4)cc4ccccc4c3c2)cc1]

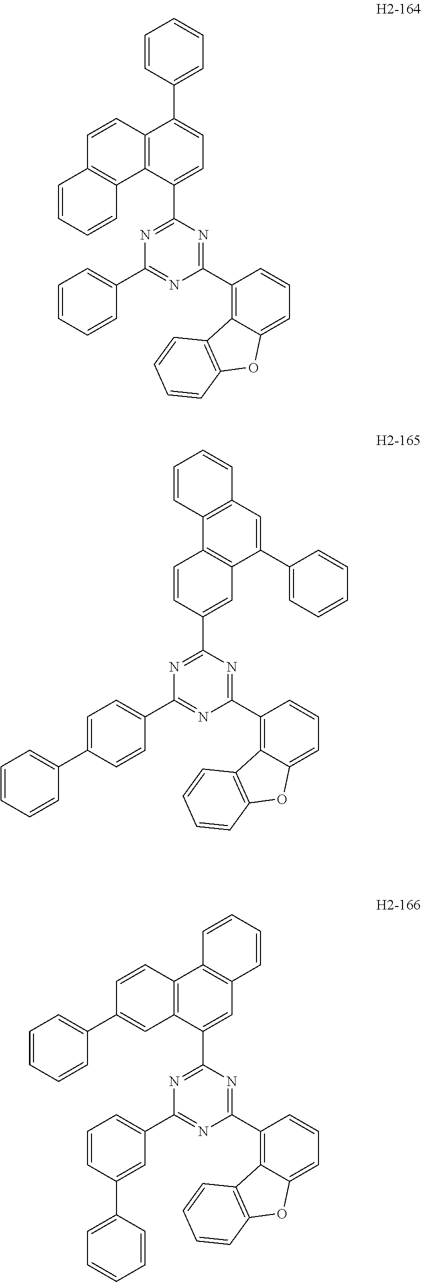 [SMILES: c1ccc(-c2nc(-c3cccc4oc5ccccc5c34)nc(-c3ccc(-c4ccccc4)c4ccc5ccccc5c34)n2)cc1 ; c1ccc(-c2ccc(-c3nc(-c4ccc5c(c4)c(-c4ccccc4)cc4ccccc45)nc(-c4cccc5oc6ccccc6c45)n3)cc2)cc1 ; c1ccc(-c2cccc(-c3nc(-c4cc5ccccc5c5ccc(-c6ccccc6)cc45)nc(-c4cccc5oc6ccccc6c45)n3)c2)cc1]

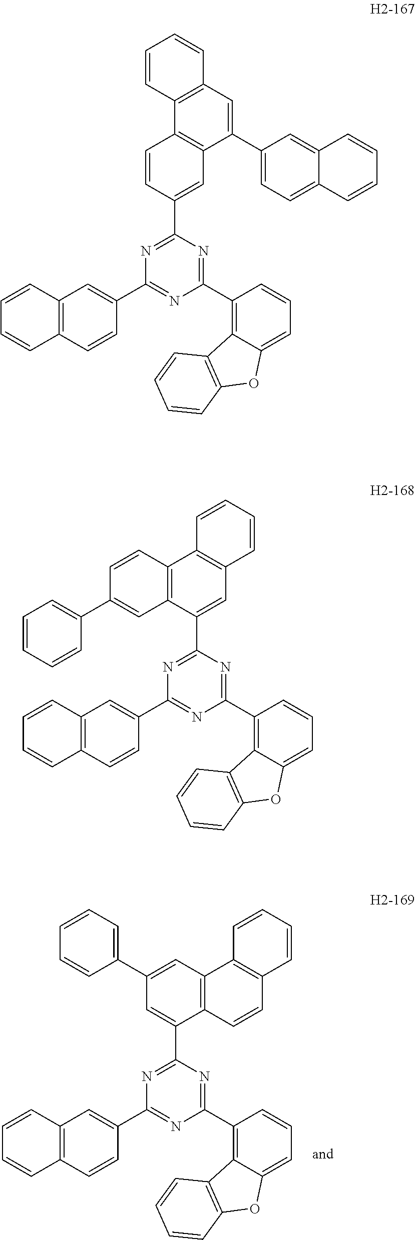 [SMILES: c1ccc2cc(-c3nc(-c4ccc5c(c4)c(-c4ccc6ccccc6c4)cc4ccccc45)nc(-c4cccc5oc6ccccc6c45)n3)ccc2c1 ; c1ccc(-c2ccc3c(c2)c(-c2nc(-c4ccc5ccccc5c4)nc(-c4cccc5oc6ccccc6c45)n2)cc2ccccc23)cc1 ; c1ccc(-c2cc(-c3nc(-c4ccc5ccccc5c4)nc(-c4cccc5oc6ccccc6c45)n3)c3ccc4ccccc4c3c2)cc1]

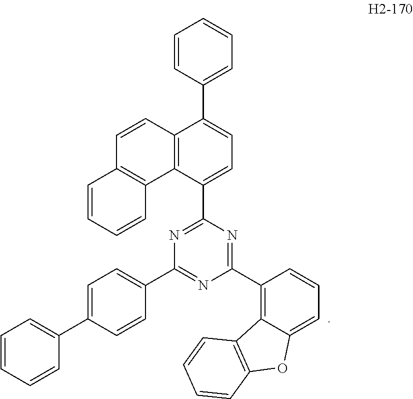 [SMILES: c1ccc(-c2ccc(-c3nc(-c4cccc5oc6ccccc6c45)nc(-c4ccc(-c5ccccc5)c5ccc6ccccc6c45)n3)cc2)cc1]

3. An organic electroluminescent device comprising the organic electroluminescent compound according to claim 1.
